(19) United States
(12) Patent Application Publication
(10) Pub. No.: US 2023/0014151 A1
(43) Pub. Date: Jan. 19, 2023

(54) COMBINATION ANTIVIRAL THERAPY FOR MEASLES

(71) Applicants: The Trustees of Columbia University in the City of New York, New York, NY (US); Cornell University, Ithaca, NY (US)

(72) Inventors: Matteo POROTTO, New York, NY (US); Anne MOSCONA, New York, NY (US); Christopher ALABI, Ithaca, NY (US)

(21) Appl. No.: 17/640,327

(22) PCT Filed: Sep. 4, 2020
(86) PCT No.: PCT/US2020/049473

§ 371 Date: Mar. 3, 2022

Related U.S. Application Data
(60) Provisional application No. 63/009,883, filed on Apr. 14, 2020.
(60) Provisional application No. 62/988,286, filed on Mar. 11, 2020.
(60) Provisional application No. 62/895,752, filed on Sep. 4, 2019.

(51) Int. Cl. A61K 39/21; A61K 39/165; A61P 31/14; A61P 31/18

(52) U.S. Cl. CPC A61K 39/21; A61K 39/165; A61P 31/14; A61P 31/18; A61K 2039/6075

(57) ABSTRACT

Described herein are peptides, compositions, and method of treating measles or HIV infection with antiviral peptide conjugates comprising a fusion inhibitory peptide (FIP) conjugated to a C-terminal heptad repeat (HRC) peptide. Also described herein are soluble stabilized measles F proteins, compositions, and method of preventing measles infection with the stabilized F protein, which can be administered alone, or in combination with the antiviral peptide conjugates described herein.

This application claims the benefit of and priority to U.S. Ser. No. 62/895,752 filed Sep. 4, 2019; U.S. Ser. No. 62/988,286 filed Mar. 11, 2020; and U.S. Ser. No. 63/009,883 filed Apr. 14, 2020, the contents of each of which are hereby incorporated by reference in their entireties.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT SUPPORT

This invention was made with government support under grants NS091263, NS105699, AI121349, and AI119762 awarded by the National Institutes of Health. The government has certain rights in the invention.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 49,187-byte ASCII (text) file named “0019240_01172WO1_SL” created on Jul. 11, 2022.

### BACKGROUND OF THE INVENTION

Measles remains a challenge for global health. While the measles vaccine was introduced in 1963, there are no FDA approved antiviral treatments for already infected individuals. Current approaches are limited in their ability to prevent the measles virus (“MV” or “MeV”) from fusing to and entering host cells. Current recommendations advise administering the vaccine or immunoglobulin (IG) within 72 hours of exposure. Antiviral compounds can target different components of viral activity, for example preventing viral replication or stopping the virus from entering host cells.

### SUMMARY OF THE INVENTION

In certain aspects, the invention described herein is directed to antiviral peptides comprising a combination of a C terminal heptad repeat (HRC) peptide and a fusion inhibitor peptide (FIP). Unexpectedly, the combination of HRC and FIP demonstrated synergism, by being more effective than either approach alone. Together, this combination prevents MeV from activating and also blocks re-folding and fusion for already activated viruses.

In one aspect, the invention provides for an antiviral peptide conjugate comprising a fusion inhibitory peptide (FIP) and a C-terminal heptad repeat (HRC) peptide (FIP-HRC). In some embodiments, the antiviral peptide conjugate further comprises a membrane localizing moiety region. In some embodiments, the membrane localizing moiety region comprises a membrane localizing moiety selected from the group consisting of cholesterol, tocopherol, and palmityl. In some embodiments, the membrane localizing moiety region is conjugated to the C-terminus of the HRC peptide. In some embodiments, the antiviral peptide conjugate further comprises a linker region. In some embodiments, the linker region comprises polyethylene glycol (PEG). In some embodiments, the PEG is 4 ethylene glycol units in length (PEG4). In some embodiments, the PEG is 11 ethylene glycol units in length (PEG11). In some embodiments, the linker region is conjugated to the C-terminus of the HRC peptide. In some embodiments, the antiviral peptide conjugate further comprises a membrane localizing moiety region and a linker region. In some embodiments, the linker region is conjugated to the C-terminus of the HRC peptide and the membrane localizing moiety region is conjugated to the linker region. In some embodiments, the linker region comprises polyethylene glycol (PEG). In some embodiments, the PEG is 4 ethylene glycol units in length (PEG4). In some embodiments, the PEG is 11 ethylene glycol units in length (PEG11). In some embodiments, the PEG is 12 ethylene glycol units in length (PEG12). In some embodiments, the PEG is 14 ethylene glycol units in length (PEG14). In some embodiments, the antiviral peptide comprises a dimer of the FIP region and the HRC peptide region. In some embodiments, the antiviral peptide comprises a first FIP-HRC peptide conjugated to the linker region and a second FIP-HRC peptide conjugated to the linker region. In some embodiments, the peptide further comprises a serine-glycine linker. In some embodiments, the serine-glycine linker is located between the FIP and HRC peptide. In some embodiments, the serine-glycine linker is located at the C-terminus of the HRC peptide. In some embodiments, the serine-glycine linker is located between the FIP region and HRC peptide region and further comprises a second serine-glycine linker located at the C-terminus of the HRC peptide region. In some embodiments, the serine-glycine linker comprises of the amino acid sequence GSGSG. In some embodiments, a first phenylalanine residue of the FIP of the antiviral peptide conjugate is a D-amino acid. In some embodiments, the N-terminus of the antiviral peptide conjugate further comprises a benzyloxycarbonyl group. In some embodiments, the FIP peptide comprises the amino acid sequence FFG. In some embodiments, the HRC peptide comprises the amino acid sequence PPISLERLDVGTNLGNAIAKLEDAKELLESSDQILR (SEQ ID NO. 8). In some embodiments, the HRC peptide conjugate comprises the amino acid sequence PPISLERLDVGTN (SEQ ID NO. 9). In some embodiments, the antiviral peptide comprises the amino acid sequence FFGPPISLERLDVGTNLGNAIAKLEDAKELLESSDQILR (SEQ ID NO. 10). In some embodiments, the antiviral peptide comprises the amino acid sequence FFGPPISLERLDVGTN (SEQ ID NO. 11).

In another aspect, the invention provides for a nanoparticle comprising any of the antiviral peptide conjugates described herein. In some embodiments, the nanoparticle has a diameter of between about 50 nm and about 150 nm.

In another aspect, the invention provides for a composition comprising any of the antiviral peptide conjugates described herein.

In another aspect, the invention provides for a prophylactic composition comprising an antiviral peptide conjugate comprising a fusion inhibitory peptide (FIP) and a C-terminal heptad repeat (HRC) peptide (FIP-HRC), wherein said measles antiviral peptide conjugate prevents membrane fusion of measles virus.

In another aspect, the invention provides for a nanoparticle comprising a fusion inhibitory (FIP) peptide and a C-terminal heptad repeat (HRC) peptide (FIP-HRC).

In another aspect, the invention provides for a method of post-infection measles prophylaxis comprising administering to a subject in need thereof an antiviral peptide conjugate comprising a fusion inhibitory peptide (FIP) and a C-terminal heptad (HRC) peptide (FIP-HRC). In some embodiments, the antiviral peptide conjugate further comprises a linker region. In some embodiments, the antiviral peptide conjugate further comprises a membrane-localizing moiety region. In some embodiments, the antiviral peptide conjugate further comprises a linker region and a membrane-localizing moiety region. In some embodiments, the administration comprises intranasal inhalation or oral inhalation. In some embodiments, the antiviral peptide conjugate is administered via a device selected from the group consisting of a nebulizer, an aerosolizer, and an inhaler. In some embodiments, the administration comprises subcutaneous administration. In some embodiments, the subject has been exposed to a measles virus comprising a wild type fusion glycoprotein. In some embodiments, the subject has been exposed to a measles virus comprising one or more mutations of a fusion glycoprotein selected from the group consisting of N462K, L454W, T461I, E455G, E170G, G506E, M337L, D538G, G168R, S262G, A440P, R520C, and L550P.

In another aspect, the invention provides for a recombinant protein comprising a soluble stabilized measles F protein comprising an E445G mutation.

In another aspect, the invention provides for a recombinant protein comprising a soluble stabilized measles F protein comprising a E170G and a E455G double mutation.

In another aspect, the invention provides for a recombinant protein comprising the amino acid sequence of SEQ ID NO: 3 or 4.

In another aspect, the invention provides for a recombinant protein comprising the amino acid sequence of SEQ ID NO: 5 or 6.

In another aspect, the invention provides for an immunogenic composition comprising any one of the recombinant proteins described herein. In some embodiments, the invention provides for a method of preventing a measles infection prior to measles exposure by administering to a subject the immunogenic composition. In some embodiments, the invention provides for a method of inducing an immune response to a measles virus by administering to a subject the immunogenic composition.

In another aspect, the invention provides for an immunogenic composition comprising the amino acid sequence of SEQ ID NO: 1 or 2, and further comprising any of the antiviral peptide conjugates described herein.

In another aspect, the invention provides for a method of inducing an immune response to a measles virus by administering to a subject any of the immunogenic compositions described herein and further administering any of the antiviral peptide conjugates described herein.

In another aspect, the invention provides for a method of producing a recombinant protein comprising the amino acid sequence of SEQ ID NO: 3 or 4.

In another aspect, the invention provides for a method of producing a recombinant protein comprising the amino acid sequence of SEQ ID NO: 5 or 6.

In another aspect, the invention provides for a cell line expressing a recombinant protein comprising the amino acid sequence of SEQ ID NO: 3 or 4.

In another aspect, the invention provides for a cell line expressing a recombinant protein comprising the amino acid sequence of SEQ ID NO: 5 or 6.

In another aspect, the invention provides for an antiviral peptide conjugate comprising a fusion inhibitory peptide (FIP) and a C-terminal heptad repeat (HRC) peptide (FIP-HRC), wherein the HRC peptide is derived from HIV-GP41 (C34). In some embodiments, the HRC peptide comprises of the amino acid sequence WMEWDREINNYTSLIHSLIEESQNQQEKNEQELL (SEQ ID NO. 12).

In another aspect, the invention provides for a method of post-infection HIV prophylaxis comprising administering to a subject in need thereof an antiviral peptide conjugate comprising a fusion inhibitory peptide (FIP) and a C-terminal heptad repeat (HRC) peptide (FIP-HRC). In some embodiments, the HRC peptide is derived from HIV-GP41 (C34).

### BRIEF DESCRIPTION OF THE FIGURES

The patent or application file contains at least one drawing in color. To conform to the requirements for PCT patent applications, many of the figures presented herein are black and white representations of images originally created in color.

FIG. 1 shows that MV infection starts in the respiratory tract.

FIG. 2 shows viremia and egress of MV pathogenesis.

FIG. 3 shows that measles can also cause severe complications.

FIG. 4 shows data for HIV-infected patients with MV encephalitis.

FIG. 5 shows a schematic representation of the measles virus.

FIG. 6 shows that the MV F gene in two patients contained the same nucleotide mutation.

FIG. 7 shows that viral entry for wild-type measles into the CNS is tightly regulated.

FIG. 8 shows methodology of fusion complex analysis.

FIGS. 9A-9C show levels of fusion. FIGS. 9A-B show that in the presence of a known receptor, all F proteins demonstrate similar levels of fusion. FIG. 9C shows that F L454W induces fusion in the absence of a known receptor.

FIG. 10 shows two thermal states of the F protein.

FIG. 11 shows cell-to-cell fusion induced by recombinant viruses.

FIG. 12 shows 90-day old brain organoids infected with measles wild-type F virus and L454W F bearing virus.

FIG. 13 shows ex vivo tissue from mice (no receptor).

FIG. 14 shows MV data in cotton rats.

FIGS. 15A-15B show survival rate data in mice.

FIG. 16 shows viral entry as a therapeutic target.

FIG. 17 shows F glycoprotein derived peptides.

FIG. 18 shows that F glycoprotein derived peptides inhibit viral entry.

FIG. 19 shows targeting of HRC peptides toward lipid membranes.

FIG. 20 shows improving HRC peptides' avidity towards F protein.

FIG. 21 shows another embodiment of targeting HRC peptides towards lipid membranes.

FIGS. 22A-22B show that conjugated peptides are potent in vivo. The model used is cotton rats in FIG. 22A and SLAM:IFNARKO mice in FIG. 22B.

FIGS. 23A-23C show that MV HRC4 peptide blocks viral spread ex vivo.

FIG. 24 shows that intranasal administration of MV HRC4 protects suckling mice from lethal infection with virus bearing L454W F.

FIG. 25 shows that peptide particle size is in the nanomolar range.

FIG. 26 shows that amphipathic structure drives self-assembly and nanoparticle formation.

FIG. 27 shows that nanoparticles (dis)assemble at lipid membrane interfaces with peptide retention.

FIG. 28 shows that nanoparticles are able to cross the HAE barrier and are bioavailable in vivo. By reaching relevant sites of infection, these prevent measles virus multiplication.

FIG. 29 shows that conjugated peptides have improved biodistribution in the cotton rat model with intranasal administration route at 6 mg/kg.

FIG. 30 shows combinatorial strategy where fusion inhibitory peptide (FIP) binds to the fusion protein and stabilizes the pre-fusion state of the measles F protein.

FIG. 31 shows that the isobologram curve shows synergism between FIP and HRC peptides.

FIG. 32 shows that FIP added to the HRC region enhances the antiviral activity.

FIG. 33 shows that when FIP and HRC are in the same structure, the potency transcends the synergism of the two inhibitors added together.

FIG. 34 shows additional data that when FIP and HRC are in the same structure, the potency transcends the synergism of the two inhibitors added together.

FIG. 35 shows that FIP added to the HRC region enhances the antiviral activity.

FIG. 36 shows additional data that FIP added to the HRC region enhances the antiviral activity.

FIG. 37 shows survival proportions.

FIG. 38 shows various MVs.

FIG. 39 shows that MV HRC4 peptide and RNA polymerase inhibitor block MV wild-type infection in human motor neurons.

FIG. 40 shows HRC4 peptide and RNA polymerase inhibitor: wild-type and CNS-adapted viruses.

FIG. 41 shows MV HRC4 peptide and RNA polymerase inhibitor vs. CNS-adapted MV in human motor neurons.

FIGS. 42A-42B show steps in entry.

FIGS. 43A-43C also show steps in entry.

FIGS. 44A-44C show that H-F interaction is altered in L454W F.

FIG. 45 shows that intranasal administration of MV HRC4 protects IFNAR KO mice from lethal MV encephalitis.

FIG. 46 shows a schematic of the various organoids that can be grown from pluripotent stem cells and the developmental signals that are employed.

FIGS. 47A-47C shows a “mini-brain” generated from pluripotent stem cells. FIG. 47A shows that a complex morphology with heterogeneous regions containing neural progenitors (SOX2, red) and neurons (TUJ1, green) is apparent. FIG. 47B shows an immunofluorescent image of an entire kidney organoid grown from pluripotent stem cells with patterned nephrons. Podocytes of the forming glomeruli (NPHS1, yellow), early proximal tubules (lotus tetragonolobus lectin, pink), and distal tubules/collecting ducts (E-Cadherin, green). FIG. 47C shows 3D reconstruction of the midsection of a human aSC-derived lung organoid stained for intermediate filaments of basal cells (green), the actin cytoskeleton (red), and nuclei (blue) and imaged by confocal microscopy.

FIG. 48 shows reported cases of measles over time.

FIG. 49 shows a schematic representation of the measles virus.

FIG. 50 shows measles virus entry into a cell.

FIG. 51 shows steps in measles virus entry into a cell.

FIG. 52 shows measles virus entry as a potential therapeutic target.

FIG. 53 shows the structure of the measles fusion inhibitory peptide (FIP).

FIG. 54 shows a combination strategy for preventing the measles virus from entering cells.

FIG. 55 shows the design of lipid-peptide conjugates.

FIG. 56 shows the advantages of lipid-peptide conjugates.

FIGS. 57-58 show examples of lipid-peptide structures.

FIGS. 59-60 show reaction schemes for monomer lipid-peptide conjugate synthesis.

FIG. 61 shows a list of lipid-peptide conjugates.

FIG. 62 shows purification and characterization data of a lipid-peptide conjugate.

FIG. 63 shows the process of beta-galactosidase complementation-based fusion assay.

FIG. 64 shows data for inhibitory activity of measles lipid-peptide conjugates in fusion assay.

FIG. 65 shows the best lipid-peptide conjugate candidate based on inhibitory activity from fusion assay data.

FIG. 66 shows the process of MTT cytotoxicity assay.

FIG. 67 shows MTT assay data.

FIG. 68 shows the process of thermostability studies of measles fusion protein.

FIG. 69 shows data from thermostability studies of measles fusion protein.

FIG. 70 shows F stabilization properties of measles lipid-peptide conjugates.

FIG. 71 shows the best lipid-peptide conjugate candidate based on F stabilization properties of measles lipid-peptide conjugates.

FIG. 72 shows quantitating each stage of fusion activation.

FIGS. 73-75 shows measles virus binding activity to red blood cells (RBCs) in the presence of various concentrations of different lipid-peptide conjugates (FIG. 73: HRC; FIG. 74: FIP-HRC; FIG. 75: FIP).

FIG. 76 shows a schematic of in vivo efficacy of measles lipid-peptide conjugates.

FIG. 77 data for in vivo efficacy of measles lipid-peptide conjugates.

FIG. 78 shows particle size measurement data of measles lipid-peptide conjugates.

FIG. 79 shows a schematic of interactions between lipid-peptide conjugates.

FIG. 80 shows data from an isobologram analysis.

FIG. 81 shows quantitation of isobologram analysis.

FIG. 82 shows a schematic of a mechanism of action of a measles fusion inhibitory conjugate.

FIG. 83 shows a schematic of various designs for different lipid-peptide conjugates.

FIG. 84 shows in vivo efficacy data.

FIGS. 85A-85C show FIP-HRC targets MV F expressing cells.

FIG. 86 shows that FIP-HRC stabilizes the measles F in its pre-fusion state.

FIG. 87 shows F-stabilization properties of the MeV peptides.

FIGS. 88-90 show stabilization properties of the MeV peptides on soluble F.

FIG. 91 shows that FIP-HRC prevents F activation (totally different mechanism from HRC that prevents F refolding).

FIG. 92 shows that FIP-HRC targets MeV F expressing cells.

FIG. 93 shows data from three different experiments demonstrating that FIP-HRC targets MeV F expressing cells.

FIG. 94 shows stabilization properties of the MeV peptides.

FIG. 95 shows cytotoxicity of the MeV peptides.

FIG. 96 shows synergy data from isobologram analysis for HRC+FIP conjugate.

FIG. 97 shows potency of a FIP-HRC with 12 amino acids derived from the measles HRC.

FIG. 98 shows inhibition data for BG505 (human immunodeficiency virus type 1 (HIV-1) strain) using various lipid-peptide conjugates and positive and negative controls.

FIG. 99 shows inhibition data for B41 (HIV-1 strain) using various lipid-peptide conjugates and positive and negative controls.

FIG. 100 shows inhibition data for 16055 (HIV-1 strain) using various lipid-peptide conjugates and positive and negative controls.

FIG. 101 shows inhibition data for MN (HIV-1 strain) using various lipid-peptide conjugates and positive and negative controls.

FIG. 102 shows inhibition data for vesicular stomatitis virus (VSV) using various lipid-peptide conjugates and positive and negative controls.

FIG. 103 shows inhibition data for murine leukemia viruses (MLV) using various lipid-peptide conjugates.

FIGS. 104A-104B show location of substitutions within the F protein from CNS-adapted virus.

FIGS. 105A-105I show ex vivo infection with wild type (wt) vs. virus bearing the L454W F: the CNS-adapted virus outcompetes the wt virus in organotypic brain cultures (OBC).

FIGS. 106A-D show CNS adapted MeV variants spread efficiently in human pluripotent stem cell (hiPSC) derived brain organoids.

FIGS. 107A-107H show fusion activity and thermal stability of MeV fusion (F) proteins bearing the indicated mutations that the additional mutations in the L454W F background stabilize the pre-fusion state of the F protein.

FIG. 108 shows inhibition of spread of the L454W F bearing virus in highly susceptible CD150xIFNARKO CNS.

FIGS. 109A-109B show induction of interferon stimulated genes by MeV F L454W in mouse brain slice cultures compared to wild type MeV.

FIGS. 110A-110B show infection of human brain organoids in the presence of fusion inhibitors.

FIGS. 111A-111B show induction of host antiviral genes in brain organoids infected with fusion protein mutant MeVs.

FIG. 112 shows a correlation heatmap of gene-level RPKM values between brain organoids and the BrainSpan atlas.

FIG. 113 shows KEGG pathway analysis of genes differentially expressed upon L454W infection.

FIG. 114 shows a Longitudinal Analysis of Viral Alleles (LAVA) plot for L454W and L454W/G506E Fusion protein mutant viral populations.

FIG. 115 shows a LAVA plot for L454W and L454W/E455G Fusion protein mutant viral populations.

FIG. 116 shows a LAVA plot for wild type Fusion protein viral population.

FIGS. 117A-117E show (A) Soluble MeV-FE170G-E455G has been incubated at 4° C. or 55° C. for the indicated time with or without FIP-HRC 1 mM (dimer without cholesterol). Following the incubation the F has been immunoprecipitated using a mouse pre-fusion specific antibody. The immunoprecipitated protein was run on a SDS-PAGE reducing gel, transferred to a PVDF membrane and incubated with α-MV-F-HRC biotin (1:1000). Streptavidin alkaline phosphatase conjugate has been used as secondary antibody (1:1000 in pbs). (B) Quantification of WB bands of the experiment described in (A) has been performed using ImageJ software. Results represent the average of three independent experiments ±SEM. (C) Soluble MeV-FE170G-E455G has been incubated at 4° C., 25° C. or 37° C. for 1 week. The immunoprecipitation has been performed as described in (A). (D) Soluble MeV-FWT or MeV-FE170G-E455G have been incubated at 4° C. or 55° C. for 60 or 120 minutes with or without FIP-HRC, FIP commercial, FIP dimer or 3 g (1 mM). (E) Quantification of WB bands of the experiment described in (D) has been performed using ImageJ software. Results represent the average of three independent experiments ±SEM.

### DETAILED DESCRIPTION OF THE INVENTION

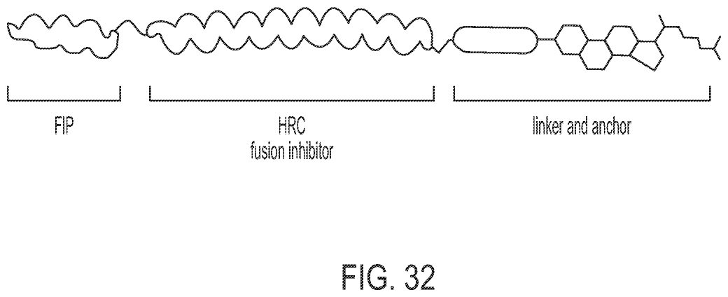

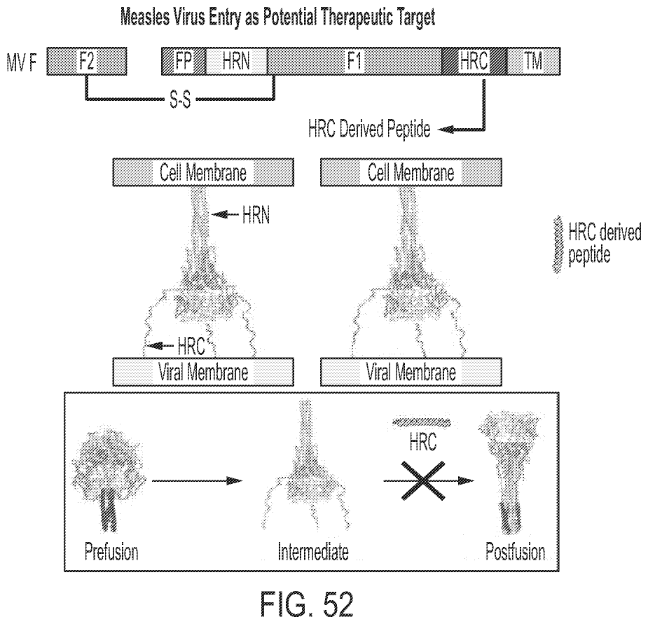

Despite the introduction of a vaccine over 50 years ago, measles remains a challenge for global health. There are no FDA approved antiviral treatments for already infected individuals, and current approaches are limited in their ability to prevent measles virus (MeV) from fusing to and entering host cells. The subject matter disclosed herein, relates in one embodiment to the combination of two existing MeV antiviral approaches that target different modes of action. The first approach includes targeting the terminal heptad repeat (HRC) regions of the MeV fusion protein (F) using an HRC-derived peptide, which interferes with the structural rearrangements required for viral fusion (e.g., prevents refolding) during infection. The second approach includes targeting the heptad repeat B (HRB) region of MeV F using a fusion inhibitor peptide (FIP), which stabilizes the MeV F in a prefusion state in which it cannot fuse (e.g., see FIGS. 52-32). This combination approach is demonstrated to be more effective than either approach alone in several in vitro, ex vivo, and in vivo models. Together, this combination prevents MeV from activating and also blocks re-folding and fusion for already activated viruses.

Furthermore, the subject matter disclosed herein relates in one embodiment to the combination of two antiviral methods to prevent measles virus fusion with a cell membrane and entry into a cell by targeting C-terminal heptad repeat (HRC) regions of MeV F, using an HRC-derived peptide, which interferes with the structural rearrangements required for viral fusion during infection and by targeting the heptad repeat B (HRB) region of the MeV F, using a fusion inhibitor peptide (FIP), which stabilizes the MeV F in a pre-activated state. This technology demonstrates in vitro, ex vivo, and in vivo that the combination of both approaches is more effective than either approach alone.

The invention relates to a measles antiviral peptide for administration either by the respiratory or subcutaneous route. The invention also relates to the combination of two distinct peptide domains that act by different mechanisms to prevent entry: (1) A peptide domain (“HRC”) that is complementary to a heptad repeat on the measles F protein that is critical for the F-refolding step of viral entry that occurs once the F protein has been activated and inserted into the target cell membrane. This refolding is what leads to virus-cell membrane fusion and entry, and therefore blocking it by binding of the HRC peptide to F protein prevents viral entry and infection; (2) A peptide domain (“FIP”) that binds to a different region of the F protein (e.g., HRB region) and stabilizes the F protein in the pre-activated conformation, preventing F's activation to the fusion-competent state. If the F protein is not activated to the fusion competent state, none of the subsequent steps in entry can occur. The combination of (1) and (2) results in a peptide that first stabilizes F protein in its pre-fusion conformation so that it is not fusion competent—and then, for F protein that has been activated to fuse, the peptide prevents re-folding and fusion. The efficacy of this combination has been studied in vitro and in vivo and is markedly enhanced over either approach alone.

### REFERENCES

1. Plemper R K, Snyder J P. Measles control—can measles virus inhibitors make a difference? Curr Opin Investig Drugs. 2009 August; 10(8): pp. 811-20.
2. Woo T M. Postexposure management of vaccine-preventable diseases. J Pediatr Health Care. 2016 March; 30(2): pp. 173-82.
3. Welsch J C, Talekar A, Mathieu C, Pessi A, Moscona A, Horvat B, Porotto M. Fatal measles virus infection prevented by brain-penetrant fusion inhibitors. J Virol. 2013 December; 87(24): pp. 13785-94.
4. Hashiguchi T, Fukuda Y, Matsuoka R, Kuroda D, Kubota M, Shirogane Y, Watanabe S, Tsumoto K, Kohda D, Plemper R K, Yanagi Y. Structures of the prefusion form of measles virus fusion protein in complex with inhibitors. Proc Natl Acad Sci USA. 2018 March; 115(10): pp. 2496-501.

### Antiviral Peptide Conjugates

In certain aspects the invention provides an antiviral peptide conjugate comprising a fusion inhibitory peptide (FIP) and a C-terminal heptad repeat (HRC) peptide.

Measles virus is a paramyxovirus belonging to the genus Morbillivirus. It is a pleomorphic virus ranging in diameter from 100 to 300 nm. The measles genome consists of six genes, each encoding a single structural protein referred to as N (Nucleocapsid), P (Phosphoprotein), M (Matrix Protein), F (Fusion Protein), H (Hemagglutinin), and L (Large Protein). One of these genes, the phosphoprotein (P) gene, also encodes two non-structural proteins (V and C). There are distinct genetic lineages of wild-type measles viruses based on the nucleotide sequence of the nucleoprotein.

### Fusion Inhibitory Peptides (FIP)

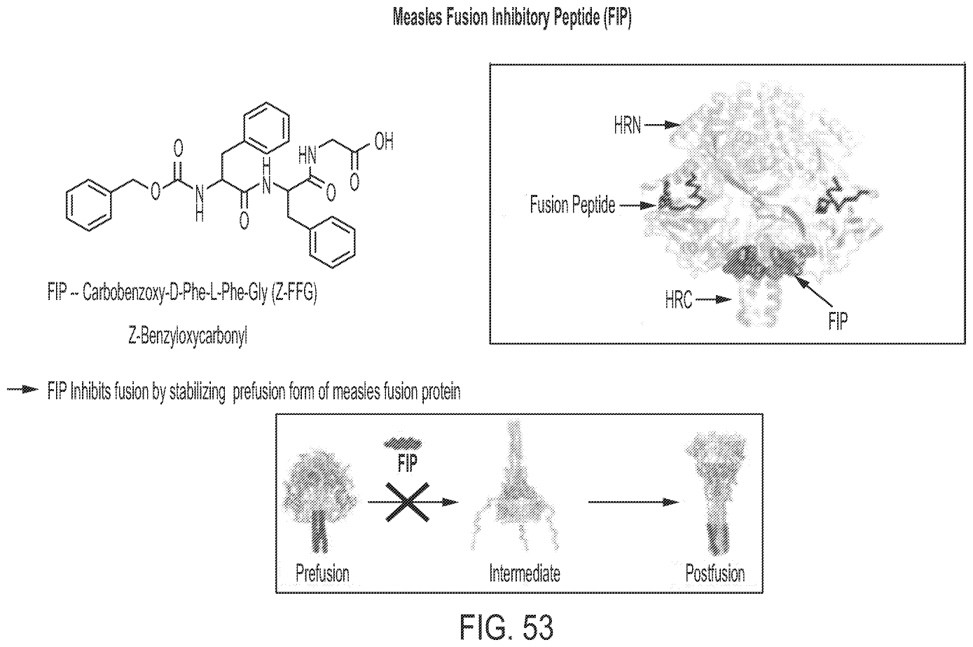

In some embodiments, the FIP is a fusion inhibitory peptide comprising the amino acid sequence phenylalanine-phenylalanine-glycine (FFG). In some embodiments, the FIP is a fusion inhibitory peptide consisting of the amino acid sequence phenylalanine-phenylalanine-glycine (FFG). In some embodiments, the first phenylalanine residue of the measles FIP is a D-amino acid and the second phenylalanine residue and the glycine residue are L-amino acids (i.e., D-FFG). D-Amino acids are amino acids where the stereogenic carbon alpha to the amino group has the D-configuration. In some embodiments, the N-terminus of the FIP further comprises a benzyloxycarbonyl group as shown in FIG. 53 (i.e., Z-D-FFG). In some embodiments, the FIP further comprises one or more serine-glycine linkers. In some embodiments, the serine-glycine linker comprises the amino acid sequence GSGSG (SEQ ID NO. 7). In some embodiments, the serine-glycine linker consists of the amino acid sequence GSGSG (SEQ ID NO. 7). In some embodiments, the FIP comprises a serine-glycine linker at the C-terminus of the FIP (e.g., FFG-GSGSG (SEQ ID NO. 13), D-FFG-GSGSG (SEQ ID NO. 14), Z-D-GSGSG (SEQ ID NO. 15)). In some embodiments, the FIP binds to the HRB regions of the MeV F to prevent viral entry into host cells.

### C-Terminal Heptad Repeat (HRC) Peptides

In some embodiments, the C-terminal heptad repeat (HRC) peptide is derived from a measles virus F protein. The HRC peptide is conserved between measles strains and can be derived from any measles strain F protein. In some embodiments, measles virus F protein derived C-terminal heptad repeat peptide is derived from measles virus strain B3. In some embodiments, the measles virus derived C-terminal heptad repeat peptide is derived from measles virus strain G954. Nucleotide and amino acid sequences of measles virus genome and proteins encoded therein are publicly available in databases known to a person of skill in the art, for example, but not limited to GenBank and ViPR (www.viprbrc.org).

In some embodiments, the HRC is a measles virus F protein derived C-terminal heptad repeat peptide. In some embodiments, the HRC comprises residues 450 to 485 of a measles virus derived F protein C-terminal heptad repeat peptide. In some embodiments, the HRC comprises the amino acid sequence PPISLERLDVGTNLGNAIAKLEDAKELLESSDQILR (SEQ ID NO. 8). In some embodiments the HRC consists of the amino acid sequence PPISLERLDVGTNLGNAIAKLEDAKELLESSDQILR (SEQ ID NO. 8) In some embodiments, the HRC comprises an amino acid sequence comprising about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% sequence identity to PPISLERLDVGTNLGNAIAKLEDAKELLESSDQILR (SEQ ID NO. 8). In some embodiments, the HRC comprises residues 450 to 462 of a measles virus derived F protein C-terminal heptad repeat peptide. In some embodiments, the HRC comprises the amino acid sequence PPISLERLDVGTN (SEQ ID NO. 9). In some embodiments the HRC consists of the amino acid sequence PPISLERLDVGTN (SEQ ID NO. 9). In some embodiments, the HRC comprises an amino acid sequence comprising about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% sequence identity to PPISLERLDVGTN (SEQ ID NO. 9). In some embodiments, the HRC comprises residues 450 to 463, 464, 465, 466, 467, 468, 469, 470, 471, 472, 473, 474, 475, 476, 477, 478, 479, 480, 481, 482, 483, or 484 of a measles virus derived C-terminal heptad repeat peptide. In some embodiments, the HRC comprises any amino acid sequence between PPISLERLDVGTNLGNAIAKLEDAKELLESSDQILR (SEQ ID NO. 8) and PPISLERLDVGTN (SEQ ID NO. 9) in length. In some embodiments, the HRC peptide further comprises one or more serine-glycine linkers. In some embodiments, the serine-glycine linker comprises the amino acid sequence GSGSG (SEQ ID NO.7). In some embodiments, the serine-glycine linker consists of the amino acid sequence GSGSG (SEQ ID NO. 7). In some embodiments, the HRC peptide comprises a serine-glycine linker at the C-terminus of the HRC peptide (e.g., PPISLERLDVGTNLGNAIAKLEDAKELLESSDQILR-GSGSG (SEQ ID NO. 16), PPISLERLDVGTN-GSGSG (SEQ ID NO. 17)).

Human immunodeficiency virus (HIV) is a lentivirus within the family of retroviridae, subfamily orthoretrovirinae. On the basis of genetic characteristics and differences in the viral antigens, HIV is classified into the types 1 and 2 (HIV-1, HIV-2). The HIV genome includes the gag gene which encodes the proteins of the outer core membrane (MA, p17), the capsid protein (CA, p24), the nucleocapsid (NC, p′7) and a smaller, nucleic acid-stabilizing protein. The gag reading frame is followed by the pol reading frame coding for the enzymes protease (PR, p12), reverse transcriptase (RT, p51) and RNase H (p15) or RT plus RNase H (together p66) and integrase (IN, p32). Adjacent to the pol gene, the env reading frame follows from which the two envelope glycoproteins gp120 (surface protein, SU) and gp41 (transmembrane protein, TM) are derived. In addition to the structural proteins, the HIV genome codes for several regulatory proteins: Tat (transactivator protein) and Rev (RNA splicing-regulator) are necessary for the initiation of HIV replication, while the other regulatory proteins Nef (negative regulating factor), Vif (viral infectivity factor), Vpr (virus protein r) and Vpu (virus protein unique) have an impact on viral replication, virus budding and pathogenesis. In some embodiments, the C-terminal heptad repeat (HRC) peptide is derived from a HIV-1 virus gp41 protein. The gp41 HRC peptide is conserved between HIV strains and can be derived from any HIV-1 strain gp41. In some embodiments, the gp41 HRC peptide is the “C34” peptide as described in Pessi et al., A General Strategy to Endow Natural Fusion-protein-Derived Peptides with Potent Antiviral Activity, PLoS One, 2012, 7(5): e36833, the content of which is hereby incorporated by reference in its entirety. Nucleotide and amino acid sequences of HIV-1 virus genome and proteins encoded therein are publicly available in databases known to a person of skill in the art, for example, but not limited to GenBank and HIV Sequence Database at Los Alamos National Laboratory (www.hiv.lanl.gov).

In some embodiments, the HRC peptide is derived from HIV-gp41 (also known as “C34” peptide). In some embodiments, the HRC comprises residues 117 to 150 of HIV-gp41. In some embodiments, the HRC comprises the amino acid sequence WMEWDREINNYTSLIHSLIEESQNQQEKNEQELL (SEQ ID NO. 12). In some embodiments, the HRC consists of the amino acid sequence WMEWDREINNYTSLIHSLIEESQNQQEKNEQELL (SEQ ID NO. 12). In some embodiments, the HRC comprises an amino acid sequence comprising about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% sequence identity to WMEWDREINNYTSLIHSLIEESQNQQEKNEQELL (SEQ ID NO. 12). In some embodiments, the HRC peptide further comprises one or more serine-glycine linkers. In some embodiments, the serine-glycine linker comprises the amino acid sequence GSGSG. In some embodiments, the serine-glycine linker consists of the amino acid sequence GSGSG. In some embodiments, the HRC peptide comprises a serine-glycine linker at the C-terminus of the HRC peptide (e.g. WMEWDREINNYTSLIHSLIEESQNQQEKNEQELL-GSGSG (SEQ ID NO. 18)).

In some embodiments, the antiviral peptide conjugate comprises a fusion inhibitory peptide (FIP) and a C-terminal heptad repeat (HRC) peptide comprising the amino acid sequence FFG-PPISLERLDVGTNLGNAIAKLEDAKELLESSDQILR (SEQ ID NO. 10). In some embodiments, the antiviral peptide conjugate comprises a fusion inhibitory peptide (FIP) and a C-terminal heptad repeat (HRC) peptide consisting of the amino acid sequence FFG-PPISLERLDVGTNLGNAIAKLEDAKELLESSDQILR (SEQ ID NO. 10). In some embodiments the antiviral peptide conjugate further comprises one or more serine-glycine linkers. In some embodiments, the serine-glycine linker comprises the amino acid sequence GSGSG (SEQ ID NO. 7). In some embodiments, the serine-glycine linker consists of the amino acid sequence GSGSG (SEQ ID NO. 7). In some embodiments, the antiviral peptide conjugate comprises a serine-glycine linker between the FIP and HRC peptides (e.g., FFG-GSGSG-PPISLERLDVGTNLGNAIAKLEDAKELLESSDQILR (SEQ ID NO. 19)). In some embodiments, the antiviral peptide conjugate comprises a serine-glycine linker at the C-terminus of the HRC peptide (e.g., FFG-PPISLERLDVGTNLGNAIAKLEDAKELLESSDQILR-GSGSG (SEQ ID NO. 20)). In some embodiments, the antiviral peptide conjugate comprises a serine-glycine linker between the FIP and HRC peptides and at the C-terminus of the HRC peptide (e.g., FFG-GSGSG-PPISLERLDVGTNLGNAIAKLEDAKELLESSDQILR-GSGSG (SEQ ID NO. 21)). In some embodiments, antiviral peptide conjugate comprises an amino acid sequence comprising about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% sequence identity to FFG-PPISLERLDVGTNLGNAIAKLEDAKELLESSDQILR (SEQ ID NO. 10), FFG-GSGSG-PPISLERLDVGTNLGNAIAKLEDAKELLESSDQIL (SEQ ID NO. 22), FFG-PPISLERLDVGTNLGNAIAKLEDAKELLESSDQILR-GSGSG (SEQ ID NO. 20)_or FFG-GSGSG-PPISLERLDVGTNLGNAIAKLEDAKELLESSDQILR-GSGSG (SEQ ID NO. 21). In some embodiments, the first phenylalanine residue of the FIP is a D-amino acid (i.e., D-FFG- . . . ) In some embodiments, the N-terminus of the antiviral peptide conjugate further comprises a benzyloxycarbonyl group (i.e., Z-D-FFG- . . . ) In some embodiments, the antiviral peptide conjugate comprises a C-terminal cysteine residue for use to conjugate the FIP-HRC peptide to linkers and membrane localizing moieties described herein (e.g., FFG-PPISLERLDVGTNLGNAIAKLEDAKELLESSDQILR-C(SEQ ID NO. 23), FFG-GSGSG-PPISLERLDVGTNLGNAIAKLEDAKELLESSDQIL-C(SEQ ID NO. 24), FFG-PPISLERLDVGTNLGNAIAKLEDAKELLESSDQILR-GSGSG-C(SEQ ID NO. 25) or FFG-GSGSG-PPISLERLDVGTNLGNAIAKLEDAKELLESSDQILR-GSGSG-C(SEQ ID NO. 26)).

In some embodiments, the antiviral peptide conjugate comprises a fusion inhibitory peptide (FIP) and a C-terminal heptad repeat (HRC) peptide comprising the amino acid sequence FFG-PPISLERDVGTN (SEQ ID NO. 11). In some embodiments, the antiviral peptide conjugate comprises a fusion inhibitory peptide (FIP) and a C-terminal heptad repeat (HRC) peptide consisting of the amino acid sequence FFG-PPISLERLDVGTN (SEQ ID NO. 11). In some embodiments the antiviral peptide conjugate further comprises one or more serine-glycine linkers. In some embodiments, the serine-glycine linker comprises the amino acid sequence GSGSG (SEQ ID NO. 7). In some embodiments, the serine-glycine linker consists of the amino acid sequence GSGSG (SEQ ID NO. 7). In some embodiments, the antiviral peptide conjugate comprises a serine-glycine linker between the FIP and HRC peptides (e.g., FFG-GSGSG-PPISLERLDVGTN (SEQ ID NO. 28)). In some embodiments, the antiviral peptide conjugate comprises a serine-glycine linker at the C-terminus of the HRC peptide (e.g., FFG-PPISLERLDVGTN-GSGSG (SEQ ID NO. 29)). In some embodiments, the antiviral peptide conjugate comprises a serine-glycine linker between the FIP and HRC peptides and at the C-terminus of the HRC peptide (e.g., FFG-GSGSG-PPISLERLDVGTN-GSGSG (SEQ ID NO. 30)). In some embodiments, antiviral peptide conjugate comprises an amino acid sequence comprising about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% sequence identity to FFG-PPISLERLDVGTN (SEQ ID NO. 11), FFG-GSGSG-PPISLERLDVGTN (SEQ ID NO. 28), FFG-PPISLERLDVGTN-GSGSG (SEQ ID NO. 29) or FFG-GSGSG-PPISLERLDVGTN-GSGSG (SEQ ID NO. 30). In some embodiments, the first phenylalanine residue of the FIP is a D-amino acid (i.e., D-FFG- . . . ) In some embodiments, the N-terminus of the antiviral peptide conjugate further comprises a benzyloxycarbonyl group (i.e., Z-D-FFG- . . . ) In some embodiments, the antiviral peptide conjugate comprises a C-terminal cysteine residue for use to conjugate the FIP-HRC peptide to linkers and membrane localizing moieties described herein (e.g., FFG-PPISLERLDVGTN-C(SEQ ID NO. 33), FFG-GSGSG-PPISLERLDVGTN-C(SEQ ID NO. 34), FFG-PPISLERLDVGTN-GSGSG-C (SEQ ID NO. 35) or FFG-GSGSG-PPISLERLDVGTN-GSGSG-C(SEQ ID NO. 36)).

In some embodiments, the antiviral peptide conjugate comprises a fusion inhibitory peptide (FIP) and a C-terminal heptad repeat (HRC) peptide comprising the amino acid sequence FFG-WMEWDREINNYTSLIHSLIEESQNQQEKNEQELL (SEQ ID NO. 37). In some embodiments, the antiviral peptide conjugate comprises a fusion inhibitory peptide (FIP) and a C-terminal heptad repeat (HRC) peptide consisting of the amino acid sequence FFG-WMEWDREINNYTSLIHSLIEESQNQQEKNEQELL (SEQ ID NO. 37). In some embodiments the antiviral peptide conjugate further comprises one or more serine-glycine linkers. In some embodiments, the serine-glycine linker comprises the amino acid sequence GSGSG (SEQ ID NO. 7). In some embodiments, the serine-glycine linker consists of the amino acid sequence GSGSG (SEQ ID NO. 7). In some embodiments, the antiviral peptide conjugate comprises a serine-glycine linker between the FIP and HRC peptides (e.g., FFG-GSGSG-WMEWDREINNYTSLIHSLIEESQNQQEKNEQELL (SEQ ID NO. 38)). In some embodiments, the antiviral peptide conjugate comprises a serine-glycine linker at the C-terminus of the HRC peptide (e.g., FFG-WMEWDREINNYTSLIHSLIEESQNQQEKNEQELL-GSGSG (SEQ ID NO. 39)). In some embodiments, the antiviral peptide conjugate comprises a serine-glycine linker between the FIP and HRC peptides and at the C-terminus of the HRC peptide (e.g., FFG-GSGSG-WMEWDREINNYTSLIHSLIEESQNQQEKNEQELL-GSGSG (SEQ ID NO. 40)). In some embodiments, antiviral peptide conjugate comprises an amino acid sequence comprising about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% sequence identity to FFG-WMEWDREINNYTSLIHSLIEESQNQQEKNEQELL (SEQ ID NO. 37), FFG-GSGSG-WMEWDREINNYTSLIHSLIEESQNQQEKNEQELL (SEQ ID NO. 38), FFG-WMEWDREINNYTSLIHSLIEESQNQQEKNEQELL-GSGSG (SEQ ID NO. 39)_or FFG-GSGSG-WMEWDREINNYTSLIHSLIEESQNQQEKNEQELL-GSGSG (SEQ ID NO. 40). In some embodiments, the first phenylalanine residue of the FIP is a D-amino acid (i.e., D-FFG- . . . ) In some embodiments, the N-terminus of the antiviral peptide conjugate further comprises a benzyloxycarbonyl group (i.e., Z-D-FFG- . . . ) In some embodiments, the antiviral peptide conjugate comprises a C-terminal cysteine residue for use to conjugate the FIP-HRC peptide to linkers and membrane localizing moieties described herein (e.g., FFG-WMEWDREINNYTSLIHSLIEESQNQQEKNEQELL-C(SEQ ID NO. 41), FFG-GSGSG-WMEWDREINNYTSLIHSLIEESQNQQEKNEQELL-C(SEQ ID NO. 42), FFG-WMEWDREINNYTSLIHSLIEESQNQQEKNEQELL-GSGSG-C(SEQ ID NO. 43) or FFG-GSGSG-WMEWDREINNYTSLIHSLIEESQNQQEKNEQELL-GSGSG-C(SEQ ID NO. 44)).

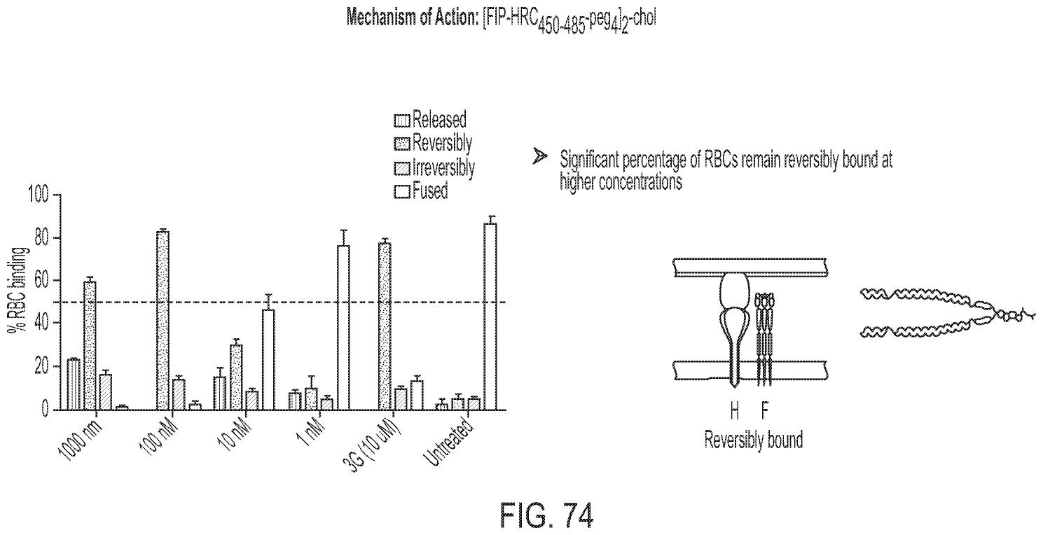

Without intending to be bound by any theory, the ability of a viral fusion protein to refold and reach a post-fusion state (i.e., to enter cells) relies on the interaction between two complementary heptad repeat (HR) regions localized at the N and C-termini of the protein (HRN and HRC, respectively). In some embodiments, the antiviral peptide conjugates comprising FIP and HRC regions stabilize the pre-fusion state of the fusion protein (e.g., reversibly bound, indicating that the fusion protein is still in its pre-fusion sate, see FIG. 74). In some embodiments, the antiviral peptide conjugates comprising FIP and HRC regions stabilize the measles fusion protein in a pre-fusion state. In some embodiments, the antiviral peptide conjugates comprising FIP and HRC regions stabilize the HIV envelope protein in a pre-fusion state.

As described herein, the glycine serine linker comprises the amino acid sequence GSGSG. However, shorter or longer glycine serine linkers can be used. In one embodiment, the glycine serine linker has the formula (GS)ₙ, or G(SG)ₙ, or S(GS)ₙ where n is 1, 2, 3, 4, 5, 6, 7, 8, 9 and 10.

### Linker

In some embodiments, the antiviral peptide conjugate further comprises a linker. In some embodiments, the linker is a component is based on its documented properties of biocompatibility, solubility, and low immunogenicity or antigenicity. In some embodiments, the linker is a polyethylene glycol (PEG) linker. PEG refers to a chemical compound composed of repeating ethylene glycol units. Covalent conjugation of peptides to PEG, known as PEGylation, is well known in the art. See e.g. Turecek et al., PEGylation of Biopharmaceuticals: A Review of Chemistry and Nonclinical Safety Information of Approved Drugs, Journal of Pharmaceutical Sciences 105 (2016, 460-475, the contents of which are hereby incorporated by reference in its entirety). PEG linker compounds can be attached to peptides via functional group linkages attached to the PEG moiety and/or the peptide. Covalent attachment of PEG, including linear and branched PEG polymers, to biologically active molecules can be achieved using amino groups of biologically active molecules as sites of attachment. Covalent attachment of PEG, including linear and branched PEG polymers, to biologically active molecules can be achieved using thiol groups of biologically active molecules as sites of attachment. Alternatively, the biologically active molecule can itself be modified to comprise functional groups (e.g., amines, thiols) to provide a site of attachment to linear and branched PEG polymers.

In some embodiments, the PEG linker is bifunctional. Bifunctional PEG linker compounds have two functional groups and thus can be attached to two biologically active molecules, such as the antiviral peptides described herein, via each functional group to generate a conjugate comprising two antiviral peptides. In some embodiments, the two antiviral peptides of the antiviral peptide conjugate are the same (i.e., a homobivalent conjugate). In some embodiments, the two antiviral peptides of the antiviral peptide conjugate are different (i.e., a heterobivalent conjugate). In some embodiments, the PEG linker can be branched and/or have multiple functional groups, for example, but not limited to three, four, five, or more functional groups. Such multivalent PEG linkers can be attached to multiple biologically active molecules, such as the antiviral peptides described herein, via each functional group to generate a conjugate comprising multiple antiviral peptides, for example, but not limited to three, four, five, or more antiviral peptides. In some embodiments, the antiviral peptides of the multivalent antiviral peptide conjugate are the same (i.e., a homomultivalent conjugate). In some embodiments, the antiviral peptides of the antiviral peptide conjugate are different (i.e., a heteromultivalent conjugate). The multivalent antiviral peptide conjugates of the invention can comprise various combinations of FIP and HRC peptides (for example, but not limited to FIG. 83). For example, in one embodiment the multivalent antiviral peptide conjugate comprises FIP-HRC conjugates, further conjugated to a branched PEG linker, optionally further comprising a membrane localizing moiety as described here. The FIP-HRC conjugates can be the same or different. In another embodiment, the multivalent antiviral peptide conjugate comprises multiple FIPs conjugated to a branched PEG linker, further conjugated to a HRC peptide, optionally further comprising a membrane localizing moiety. In some embodiments, the multivalent antiviral peptide conjugate comprises multiple FIPs conjugated to a branched PEG linker, optionally further comprising a membrane localizing moiety.

In some embodiments, the PEG linker has between 0 and 50 glycol units. In some embodiments, the PEG linker has 1 glycol units (i.e., the linker is PEG₁). In some embodiments, the PEG linker has 2 glycol units (i.e., the linker is PEG₂). In some embodiments, the PEG linker has 3 glycol units (i.e., the linker is PEG₃). In some embodiments, the PEG linker has 4 glycol units (i.e., the linker is PEG₄). In some embodiments, the PEG linker has 5 glycol units (i.e., the linker is PEG₅). In some embodiments, the PEG linker has 6 glycol units (i.e., the linker is PEG₆). In some embodiments, the PEG linker has 7 glycol units (i.e., the linker is PEG-7). In some embodiments, the PEG linker has 8 glycol units (i.e., the linker is PEG₈). In some embodiments, the PEG linker has 9 glycol units (i.e., the linker is PEG₉). In some embodiments, the PEG linker has 10 glycol units (i.e., the linker is PEG₁₀). In some embodiments, the PEG linker has 11 glycol units (i.e., the linker is PEG₁₁). In some embodiments, the PEG linker has 12 glycol units (i.e., the linker is PEG₁₂). In some embodiments, the PEG linker has 13 glycol units (i.e., the linker is PEG₁₃). In some embodiments, the PEG linker has 14 glycol units (i.e., the linker is PEG₁₄). In some embodiments, the PEG linker has 15 glycol units (i.e., the linker is PEG₁₅). In some embodiments, the PEG linker has 16 glycol units (i.e., the linker is PEG-16). In some embodiments, the PEG linker has 17 glycol units (i.e., the linker is PEG₁₇). In some embodiments, the PEG linker has 18 glycol units (i.e., the linker is PEG₁₈). In some embodiments, the PEG linker has 19 glycol units (i.e., the linker is PEG₁₉). In some embodiments, the PEG linker has 20 glycol units (i.e., the linker is PEG₂₀). In some embodiments, the PEG linker has 21 glycol units (i.e., the linker is PEG₂₁). In some embodiments, the PEG linker has 22 glycol units (i.e., the linker is PEG₂₂). In some embodiments, the PEG linker has 23 glycol units (i.e., the linker is PEG₂₃). In some embodiments, the PEG linker has 24 glycol units (i.e., the linker is PEG₂₄). In some embodiments, the PEG linker has 25 glycol units (i.e., the linker is PEG₂₅). In some embodiments, the PEG linker has 26 glycol units (i.e., the linker is PEG₂₆). In some embodiments, the PEG linker has 27 glycol units (i.e., the linker is PEG₂₇). In some embodiments, the PEG linker has 28 glycol units (i.e., the linker is PEG₂₈). In some embodiments, the PEG linker has 29 glycol units (i.e., the linker is PEG₂₉). In some embodiments, the PEG linker has 30 glycol units (i.e., the linker is PEG₃₀). In some embodiments, the PEG linker has 31 glycol units (i.e., the linker is PEG₃₁). In some embodiments, the PEG linker has 32 glycol units (i.e., the linker is PEG₃₂). In some embodiments, the PEG linker has 33 glycol units (i.e., the linker is PEG₃₃). In some embodiments, the PEG linker has 34 glycol units (i.e., the linker is PEG₃₄). In some embodiments, the PEG linker has 35 glycol units (i.e., the linker is PEG₃₅). In some embodiments, the PEG linker has 36 glycol units (i.e., the linker is PEG₃₆). In some embodiments, the PEG linker has 50 glycol units (i.e., the linker is PEG₅₀). In some embodiments, the PEG linker has between 4 and 12 glycol units. In some embodiments, the linker has between 4 and 24 glycol units. In some embodiments, the PEG linker is PEG 5000, which is a polyethylene glycol polymer with an average molecular weight of about 5000 Da. PEG 5000 comprises about 114 glycol units, thus, in some embodiments, the PEG linker has about 114 glycol units (i.e., the linker is PEG₁₁₄). In some embodiments, the PEG linker is PEG 40,000, which is a polyethylene glycol polymer with an average molecular weight of about 40,000 Da. PEG 40,000 comprises about 910 glycol units, thus, in some embodiments, the PEG linker has about 910 glycol units (i.e., the linker is PEG₉₁₀).

In some embodiments, the antiviral peptide conjugate comprises two FIP-HRC peptides optionally conjugated to a PEG linker, the conjugate having the formula:

[FIP]-Gₓ-[HRC]Gx-L-Pn-L-Gx-[HRC]-Gₓ-[FIP]

wherein [FIP] comprises a fusion inhibitory peptide as described herein;
G comprises a GSGSG (SEQ ID NO. 7) linker as described herein, wherein x for any one of the G groups=0 or 1;
[HRC] comprises a C-terminal heptad repeat peptide as described herein;
L is one or more a functional group linkages; and
P is a PEG moiety, wherein n is the number of glycol units and n=0-50. In some embodiments, n is 4. In some embodiments, n is 11. In some embodiments, n is 12. In some embodiments, n is 24. In some embodiments, shorter or longer glycine serine linkers can be used. In some embodiments, the glycine serine linker has the formula (GS)ₙ, or G(SG)ₙ, or S(GS)ₙ where n is 1, 2, 3, 4, 5, 6, 7, 8, 9 and 10.

In some embodiments, L comprises one or more sulfide moieties. In some embodiments, the one or more sulfide moieties are derived from one or more thiol moieties. In some embodiments, the one or more sulfide moieties are derived from cysteine. In some embodiments, the one or more sulfide moieties are conjugated to the C-terminus of the HRC peptide. In some embodiments, the one or more thiol moieties are conjugated to the C-terminus of the HRC peptide. In some embodiments, the one or more cysteine moieties comprise the C-terminus of the HRC peptide. In some embodiments, L comprises one or more pyrrolidinedione moieties. In some embodiments, the one or more pyrrolidinedione moieties are derived from one or more maleimide moieties. In some embodiments, the one or more pyrrolidinedione moieties are conjugated to the PEG. In some embodiments, the one or more maleimide moieties are conjugated to the PEG. In some embodiments, L is formed by coupling the one or more thiol moieties to the one or more maleimide moieties. In some embodiments, L is formed by coupling the one or more thiol moieties conjugated to the C-terminus of the HRC peptide to the one or more maleimide moieties conjugated to the PEG. In some embodiments, L is formed by coupling the one or more cysteine moieties at the C-terminus of the HRC peptide to the one or more maleimide moieties conjugated to the PEG.

In some embodiments, the C-terminal cysteine residue of the unconjugated FIP-HRC peptide terminates in a thiol (general structure “R—SH”, wherein R is the FIP-HRC peptide). A cross-coupling reaction between the thiol and a maleimide links the two components to form a sulfide (general structure “R1—S—R2”, wherein R1 is the FIP-HRC peptide and R2 is the PEG linker).

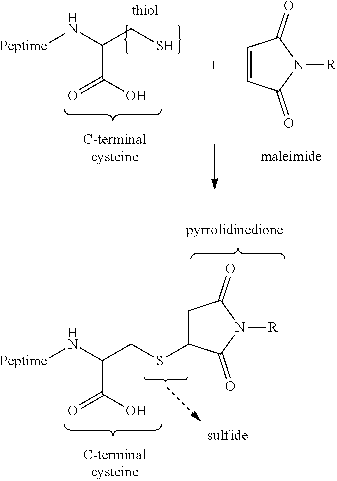

In some embodiments, L comprises

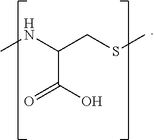

In some embodiments, L comprises

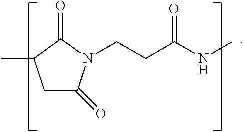

In some embodiments, the antiviral peptide conjugate comprises two FIP-HRC peptides optionally conjugated to a PEG linker, the conjugate having the formula:

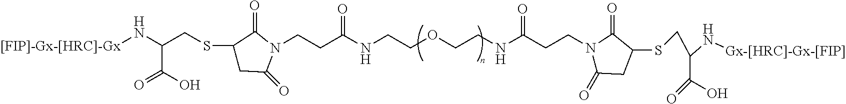

wherein [FIP] comprises a fusion inhibitory peptide as described herein;
G comprises a GSGSG (SEQ ID NO. 7) linker as described herein, wherein x for any one of the G groups=0 or 1;
[HRC] comprises a C-terminal heptad repeat peptide as described herein; and
wherein n=0-50. In some embodiments, n is 4. In some embodiments, n is 11. In some embodiments, n is 12. In some embodiments, n is 24. In some embodiments, shorter or longer glycine serine linkers can be used. In some embodiments, the glycine serine linker has the formula (GS)ₙ, or G(SG)ₙ, or S(GS)ₙ where n is 1, 2, 3, 4, 5, 6, 7, 8, 9 and 10.

Membrane Localizing Moieties/Anchors

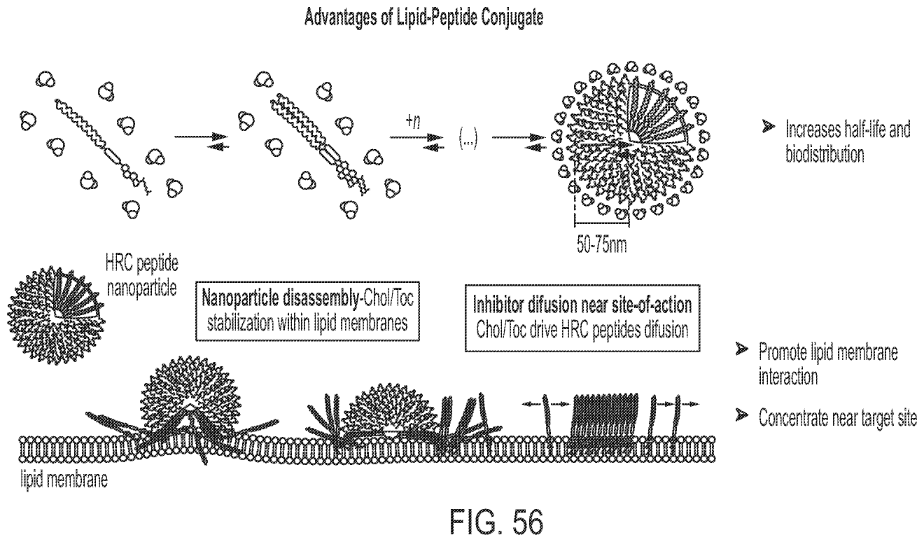

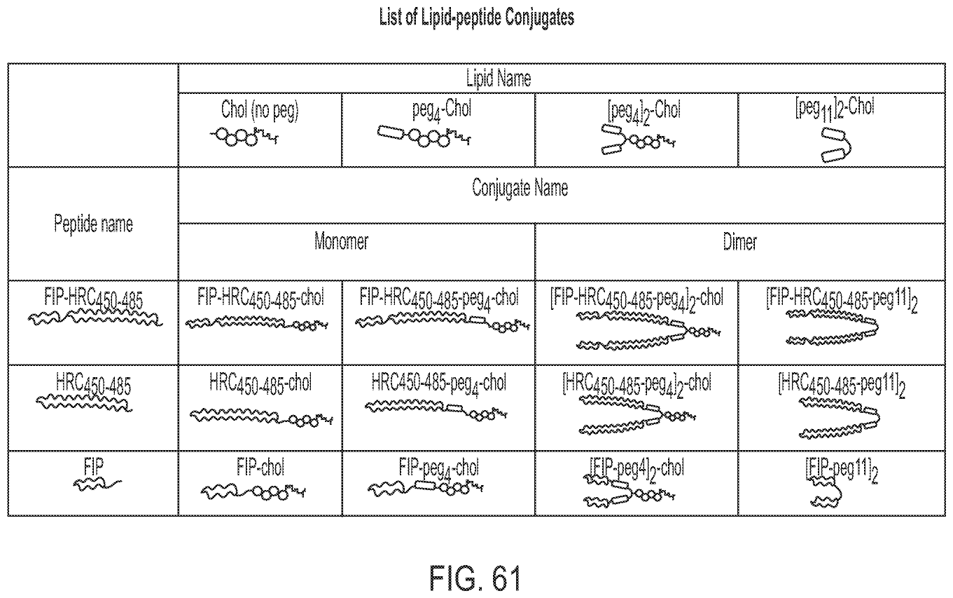

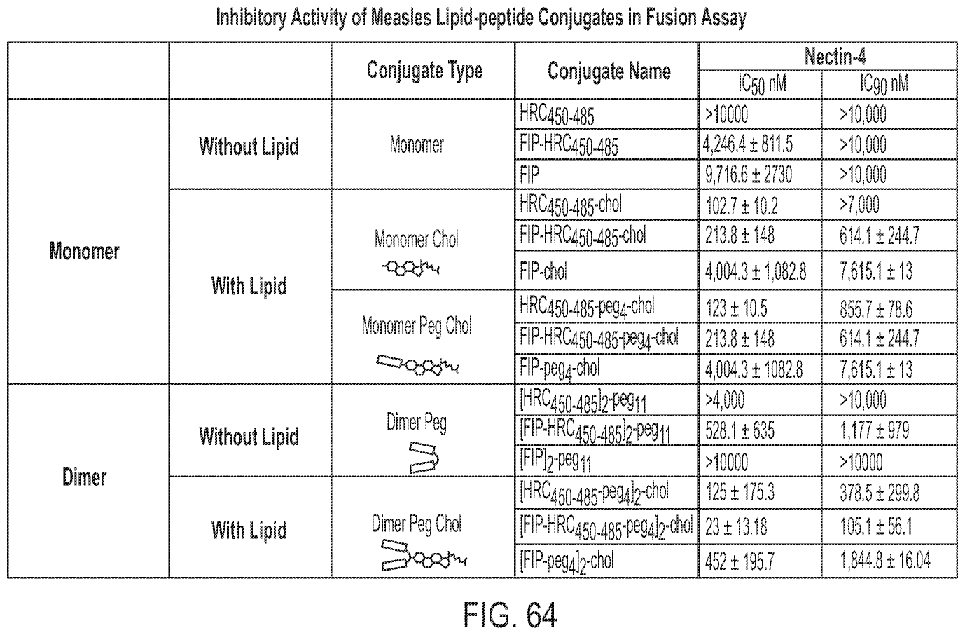

In some embodiments, the antiviral peptide conjugate comprises a fusion inhibitory peptide (FIP) and a C-terminal heptad repeat (HRC) peptide as described herein and further comprises a membrane localizing moiety (also referred to herein as an anchor). A membrane localizing moiety is any component which increases the peptide's ability to localize at the target therapeutic location. In some embodiments, the membrane localizing moiety is hydrophobic (or otherwise increases the hydrophobicity of the antiviral peptide conjugate) which increases the peptide's tendency to localize at, and/or insert into, the lipid membrane (e.g., see FIG. 56). In some embodiments, the membrane localizing moiety is a lipid. In some embodiments, the membrane localizing moiety is cholesterol, tocopherol, or palmityl. In some embodiments, the membrane localizing moiety is conjugated to the C-terminus of the antiviral peptide (see FIGS. 61 and 64). In some embodiments, the membrane localizing moiety is conjugated to a linker (e.g., PEG₄ or PEG₁₁) and the linker is conjugated to the C-terminus of the peptide (see FIGS. 61 and 64). The membrane localizing moieties of the invention can be attached to peptides via functional group linkages attached to the membrane localizing moiety and/or the peptide. Covalent attachment of membrane localizing moieties to biologically active molecules can be achieved using amino groups of biologically active molecules as sites of attachment. Alternatively, the biologically active molecule can itself be modified to comprise functional groups to provide a site of attachment to the membrane localizing moiety.

In some embodiments, the antiviral peptide conjugate comprises a FIP-HRC peptide optionally conjugated to a PEG linker and conjugated to a membrane localizing moiety, the conjugate having the formula:

[FIP]-Gₓ-[HRC]-Gₓ-L-Pₙ-L-MLM

wherein [FIP] comprises a fusion inhibitory peptide as described herein;
G comprises a GSGSG (SEQ ID NO. 7) linker as described herein, wherein x for any one of the G groups=0 or 1;
[HRC] comprises a C-terminal heptad repeat peptide as described herein;
L is one or more functional group linkages;
P is a PEG moiety, wherein n is the number of glycol units and n=0-50; and MLM is a membrane localizing moiety. In some embodiments, n is 4. In some embodiments, n is 11. In some embodiments, n is 12. In some embodiments, n is 24. In some embodiments MLM is cholesterol. In some embodiments MLM is tocopherol. In some embodiments MLM is palmityl. In some embodiments, shorter or longer glycine serine linkers can be used. In some embodiments, the glycine serine linker has the formula (GS)ₙ, or G(SG)ₙ, or S(GS)ₙ where n is 1, 2, 3, 4, 5, 6, 7, 8, 9 and 10.

In some embodiments, L comprises

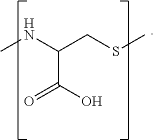

In some embodiments, L comprises

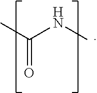

In some embodiments, L comprises

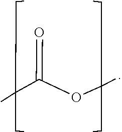

In some embodiments, the MLM comprises

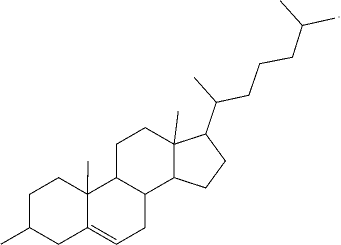 [SMILES: CC(C)CCCC(C)C1CCC2C3CC=C4CC(C)CCC4(C)C3CCC12C]

In some embodiments, the MLM comprises

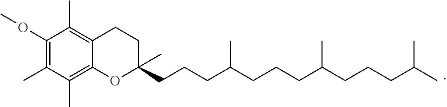 [SMILES: COc1c(C)c(C)c2c(c1C)CC[C@@](C)(CCCC(C)CCCC(C)CCCC(C)C)O2]

In some embodiments, the MLM comprises

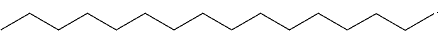 [SMILES: CCCCCCCCCCCCCCCC]

In some embodiments, the antiviral peptide conjugate comprises a FIP-HRC peptide conjugated to a membrane localizing moiety linker, the conjugate having the formula:

[FIP]-Gx-[HRC]Gx-L-MLM

wherein [FIP] comprises a fusion inhibitory peptide as described herein;
G comprises a GSGSG (SEQ ID NO. 7) linker as described herein, wherein x for any one of the G groups=0 or 1;
[HRC] comprises a C-terminal heptad repeat peptide as described herein;
L is one or more functional group linkage; and
MLM is a membrane localizing moiety. In some embodiments MLM is cholesterol. In some embodiments MLM is tocopherol. In some embodiments MLM is palmityl. In some embodiments, shorter or longer glycine serine linkers can be used. In some embodiments, the glycine serine linker has the formula (GS)ₙ, or G(SG)ₙ, or S(GS)ₙ where n is 1, 2, 3, 4, 5, 6, 7, 8, 9 and 10.

In some embodiments, L comprises

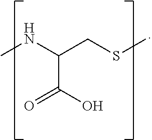

In some embodiments, L comprises

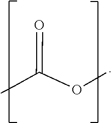

In some embodiments, L comprises

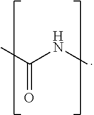

In some embodiments, the MLM comprises

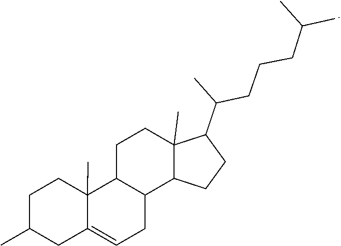 [SMILES: CC(C)CCCC(C)C1CCC2C3CC=C4CC(C)CCC4(C)C3CCC12C]

In some embodiments, the MLM comprises

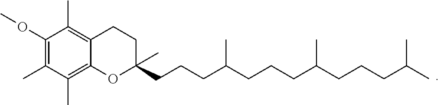 [SMILES: COc1c(C)c(C)c2c(c1C)CC[C@@](C)(CCCC(C)CCCC(C)CCCC(C)C)O2]

In some embodiments, the MLM comprises

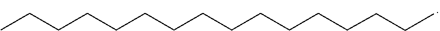 [SMILES: CCCCCCCCCCCCCCCC]

In some embodiments, L comprises one or more sulfide moieties. In some embodiments, the one or more sulfide moieties are derived from one or more thiol moieties. In some embodiments, the one or more sulfide moieties are derived from cysteine. In some embodiments, the one or more sulfide moieties are conjugated to the C-terminus of the HRC peptide. In some embodiments, the one or more thiol moieties are conjugated to the C-terminus of the HRC peptide. In some embodiments, the one or more cysteine moieties comprise the C-terminus of the HRC peptide. In some embodiments, L comprises one or more amide moieties. In some embodiments, the one or more amide moieties are derived from one or more bromoamides. In some embodiments, L comprises one or more ester moieties. In some embodiments, the one or more ester moieties are derived from one or more bromoesters. In some embodiments, the one or more amide, bromoamide, ester, or bromoester moieties are conjugated to the MLM. In some embodiments, the MLM is conjugated to a PEG linker, which in turn comprises the one or more amide, bromoamide, ester, or bromoester moieties. In some embodiments, L is formed by coupling the one or more thiol moieties to the one or more bromoamide or bromoester moieties. In some embodiments, L is formed by coupling the one or more thiol moieties conjugated to the C-terminus of the HRC peptide to the one or more bromoamide or bromoester moieties conjugated to the MLM or PEG linker. In some embodiments, L is formed by coupling the one or more cysteine moieties at the C-terminus of the HRC peptide to the one or more bromoamide or bromoester moieties conjugated to the MLM or PEG linker.

In some embodiments, the antiviral peptide conjugate comprises a FIP-HRC peptide optionally conjugated to a PEG linker and conjugated to a membrane localizing moiety, the conjugate having the formula:

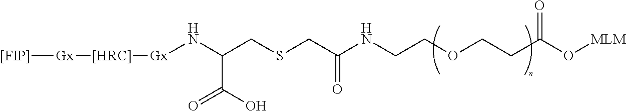

wherein [FIP] comprises a fusion inhibitory peptide as described herein;
G comprises a GSGSG (SEQ ID NO. 7) linker as described herein, wherein x for any one of the G groups=0 or 1;
[HRC] comprises a C-terminal heptad repeat peptide as described herein;
wherein n=0-50; and MLM is a membrane localizing moiety. In some embodiments, n is 4. In some embodiments, n is 11. In some embodiments, n is 12. In some embodiments, n is 24. In some embodiments MLM is cholesterol. In some embodiments MLM is tocopherol. In some embodiments MLM is palmityl. In some embodiments, shorter or longer glycine serine linkers can be used. In some embodiments, the glycine serine linker has the formula (GS)ₙ, or G(SG)ₙ, or S(GS)ₙ where n is 1, 2, 3, 4, 5, 6, 7, 8, 9 and 10.

In some embodiments, the MLM comprises

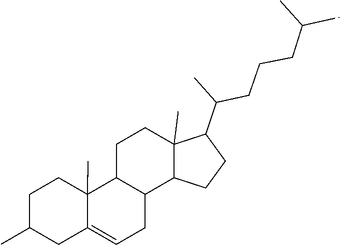 [SMILES: CC(C)CCCC(C)C1CCC2C3CC=C4CC(C)CCC4(C)C3CCC12C]

In some embodiments, the MLM comprises

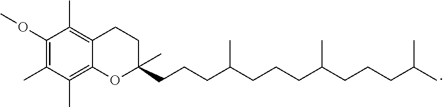 [SMILES: COc1c(C)c(C)c2c(c1C)CC[C@@](C)(CCCC(C)CCCC(C)CCCC(C)C)O2]

In some embodiments, the MLM comprises

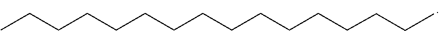 [SMILES: CCCCCCCCCCCCCCCC]

In some embodiments, the antiviral peptide conjugate comprises a FIP-HRC peptide conjugated to a membrane localizing moiety linker, the conjugate having the formula:

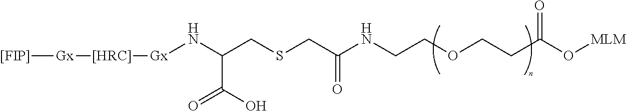

wherein [FIP] comprises a fusion inhibitory peptide as described herein;
G comprises a GSGSG (SEQ ID NO. 7) linker as described herein, wherein x for any one of the G groups=0 or 1;
[HRC] comprises a C-terminal heptad repeat peptide as described herein; and
MLM is a membrane localizing moiety. In some embodiments MLM is cholesterol. In some embodiments MLM is tocopherol. In some embodiments MLM is palmityl. In some embodiments, shorter or longer glycine serine linkers can be used. In some embodiments, the glycine serine linker has the formula (GS)ₙ, or G(SG)ₙ, or S(GS)ₙ where n is 1, 2, 3, 4, 5, 6, 7, 8, 9 and 10.

In some embodiments, the MLM comprises

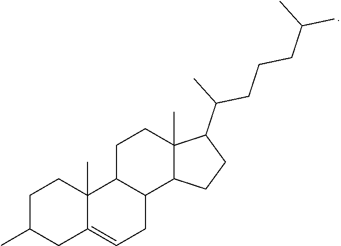 [SMILES: CC(C)CCCC(C)C1CCC2C3CC=C4CC(C)CCC4(C)C3CCC12C]

In some embodiments, the MLM comprises

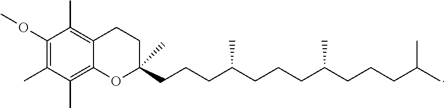 [SMILES: COc1c(C)c(C)c2c(c1C)CC[C@@](C)(CCC[C@H](C)CCC[C@H](C)CCCC(C)C)O2]

In some embodiments, the MLM comprises

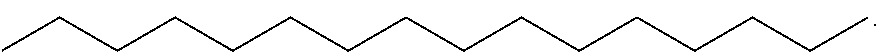 [SMILES: CCCCCCCCCCCCCCCC]

In some embodiments, the antiviral peptide conjugate comprises two FIP-HRC peptides each optionally conjugated to a PEG linker and conjugated to a membrane localizing moiety, the conjugate having the formula:

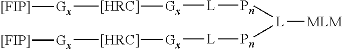

wherein [FIP] comprises a fusion inhibitory peptide as described herein;
G comprises a GSGSG (SEQ ID NO. 7) linker as described herein, wherein x for any one of the G groups=0 or 1;
[HRC] comprises a C-terminal heptad repeat peptide as described herein;
L is one or more functional group linkage groups;
P is a PEG moiety, wherein n is the number of glycol units and n=0-50; and
MLM is a membrane localizing moiety. In some embodiments MLM is cholesterol. In some embodiments MLM is tocopherol. In some embodiments MLM is palmityl. In some embodiments, n is 4. In some embodiments, n is 11. In some embodiments, n is 12. In some embodiments, n is 24. In some embodiments, shorter or longer glycine serine linkers can be used. In some embodiments, the glycine serine linker has the formula (GS)ₙ, or G(SG)ₙ, or S(GS)ₙ where n is 1, 2, 3, 4, 5, 6, 7, 8, 9 and 10.

In some embodiments, the function group linkage comprises

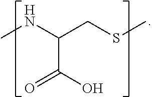

In some embodiments, the function group linkage comprises

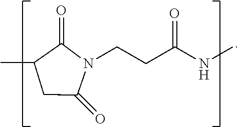

In some embodiments, the function group linkage comprises

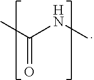

In some embodiments, the function group linkage comprises

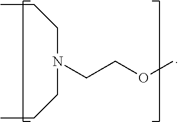

In some embodiments, the MLM comprises

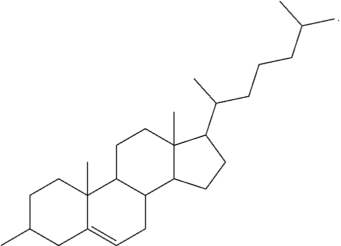 [SMILES: CC(C)CCCC(C)C1CCC2C3CC=C4CC(C)CCC4(C)C3CCC12C]

In some embodiments, the MLM comprises

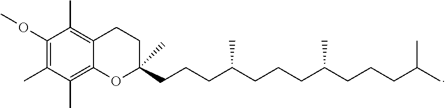 [SMILES: COc1c(C)c(C)c2c(c1C)CC[C@@](C)(CCC[C@H](C)CCC[C@H](C)CCCC(C)C)O2]

In some embodiments, the MLM comprises

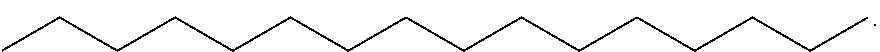 [SMILES: CCCCCCCCCCCCCCCC]

In some embodiments, L comprises one or more sulfide moieties. In some embodiments, the one or more sulfide moieties are derived from one or more thiol moieties. In some embodiments, the one or more sulfide moieties are derived from cysteine. In some embodiments, the one or more sulfide moieties are conjugated to the C-terminus of the HRC peptide. In some embodiments, the one or more thiol moieties are conjugated to the C-terminus of the HRC peptide. In some embodiments, the one or more cysteine moieties comprise the C-terminus of the HRC peptide. In some embodiments, L comprises one or more pyrrolidinedione moieties. In some embodiments, the one or more pyrrolidinedione moieties are derived from one or more maleimide moieties. In some embodiments, the one or more pyrrolidinedione moieties are conjugated to one or more PEGs, which in turn may each be conjugated to a branched linker via, for example, ester or amide bonds. In some embodiments, the PEG-branched linker moiety is conjugated to the MLM via, for example, an ether bond. In some embodiments, the one or more maleimide moieties are conjugated to the one or more PEGs or PEG-branched linker moiety. In some embodiments, L is formed by coupling the one or more thiol moieties to the one or more maleimide moieties. In some embodiments, L is formed by coupling the one or more thiol moieties conjugated to the C-terminus of the HRC peptide to the one or more maleimide moieties conjugated to the one or more PEGs or PEG-branched linker moiety. In some embodiments, L is formed by coupling the one or more cysteine moieties at the C-terminus of the HRC peptide to the one or more maleimide moieties conjugated to the one or more PEGs or PEG-branched linker moiety.

In some embodiments, the antiviral peptide conjugate comprises two FIP-HRC peptides each optionally conjugated to a PEG linker and conjugated to a membrane localizing moiety, the conjugate having the formula:

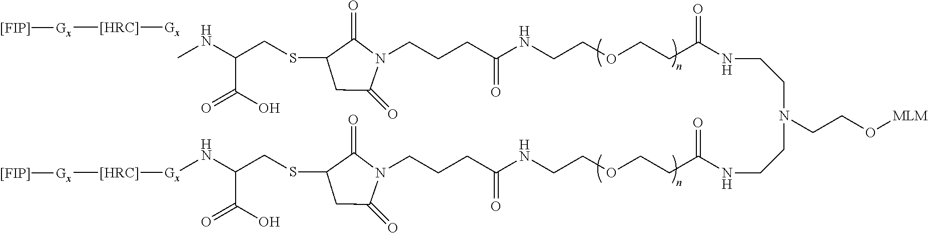

wherein [FIP] comprises a fusion inhibitory peptide as described herein;
G comprises a GSGSG (SEQ ID NO. 7) linker as described herein, wherein x for any one of the G groups=0 or 1;
[HRC] comprises a C-terminal heptad repeat peptide as described herein;
wherein n=0-50; and
MLM is a membrane localizing moiety. In some embodiments MLM is cholesterol. In some embodiments MLM is tocopherol. In some embodiments MLM is palmityl. In some embodiments, n is 4. In some embodiments, n is 11. In some embodiments, n is 12. In some embodiments, n is 24. In some embodiments, shorter or longer glycine serine linkers can be used. In some embodiments, the glycine serine linker has the formula (GS)ₙ, or G(SG)ₙ, or S(GS)ₙ where n is 1, 2, 3, 4, 5, 6, 7, 8, 9 and 10.

In some embodiments, the MLM comprises

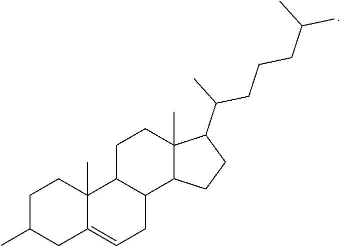 [SMILES: CC(C)CCCC(C)C1CCC2C3CC=C4CC(C)CCC4(C)C3CCC12C]

In some embodiments, the MLM comprises

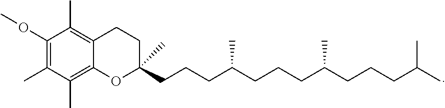 [SMILES: COc1c(C)c(C)c2c(c1C)CC[C@@](C)(CCC[C@H](C)CCC[C@H](C)CCCC(C)C)O2]

In some embodiments, the MLM comprises

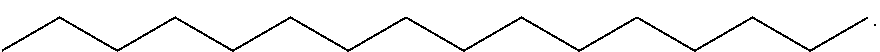 [SMILES: CCCCCCCCCCCCCCCC]

In some embodiments, the antiviral peptide conjugate comprises a fusion inhibitory peptide (FIP) and a C-terminal heptad repeat (HRC) peptide as described herein, in monomer form (“FIP-HRC₄₅₀₋₄₈₅,” see FIG. 64).

In some embodiments, the antiviral peptide conjugate comprises a fusion inhibitory peptide (FIP) and a C-terminal heptad repeat (HRC) peptide as described herein, in monomer form and further comprises a lipid (e.g., cholesterol) conjugated to the C-terminus of the peptide (“FIP-HRC₄₅₀₋₄₈₅-chol,” see FIG. 64).

In some embodiments, the antiviral peptide conjugate comprises a fusion inhibitory peptide (FIP) and a C-terminal heptad repeat (HRC) peptide as described herein, in monomer form and further comprises a linker (e.g., PEG₄) and a lipid (e.g., cholesterol) conjugated to the C-terminus of the peptide (“FIP-HRC₄₅₀₋₄₈₅-peg₄-chol,” see FIG. 64).

In some embodiments, the antiviral peptide conjugate comprises a fusion inhibitory peptide (FIP) and a C-terminal heptad repeat (HRC) peptide as described herein, in dimer form and further comprises a linker (e.g., PEG₄) and a lipid (e.g., cholesterol) conjugated to the C-terminus of the peptide (“[FIP-HRC₄₅₀₋₄₈₅-peg₄]₂-chol,” see FIG. 64).

In some embodiments, the antiviral peptide conjugate comprises a fusion inhibitory peptide (FIP) and a C-terminal heptad repeat (HRC) peptide as described herein, in dimer form and further comprises a linker (e.g., PEG₁₁) conjugated to the C-terminus of the peptide (“[FIP-HRC₄₅₀₋₄₈₅-peg₁₁]₂,” see FIG. 64).

### Antiviral Peptide Conjugate Nanoparticles

In certain aspects the invention provides a nanoparticle comprising the antiviral peptide conjugates described herein. In some embodiments, the nanoparticles of the invention have a diameter of between about 50 nm and about 150 nm. In some embodiments, the invention provides a composition comprising a plurality of nanoparticles comprising a plurality of the any of the antiviral peptide conjugates described herein. In some embodiments, the nanoparticle is composed of the antiviral peptide conjugates and further comprises other fusogenic or natural lipids (e.g. 1-palmitoyl-2-oleoyl-glycero-3-phosphocholine (POPC) and phosphatidylglycerol (POPG)). In some embodiments, the nanoparticle size is suitable for a delivery in a pharmaceutical composition. In some embodiments, the nanoparticle is encapsulated in a hydrogel that is used for controlled localized and slow delivery. In some embodiments, the nanoparticle diameter is suitable for inhalation, intranasal administration, or direct instillation into the lungs (e.g., using delivery via an inhaler, aerosolizer, or nebulizer).

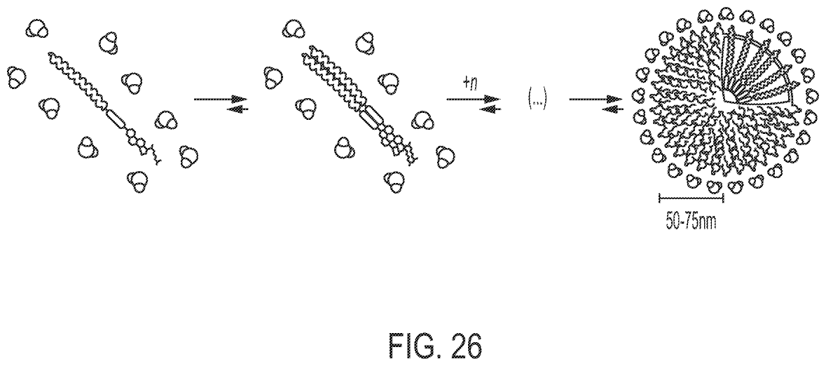

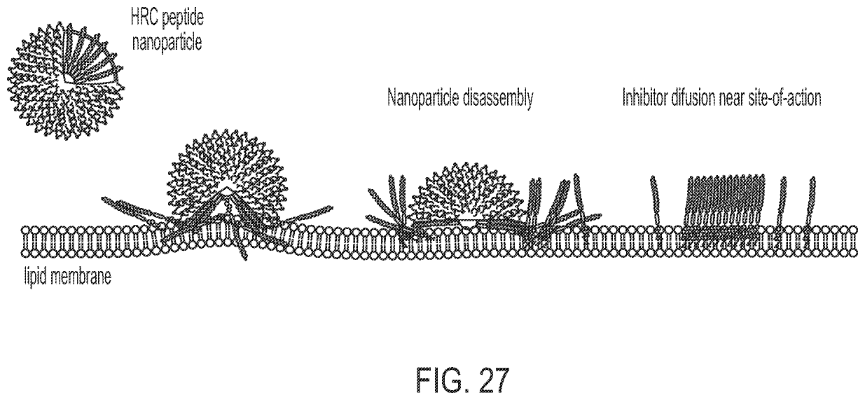

In some embodiments, when the antiviral peptide conjugates are placed in an aqueous solution, they self-assemble into nanoparticles such that the hydrophobic regions of the peptides (e.g. membrane localizing moiety) associate to form a hydrophobic core, while the hydrophilic regions of the peptides (e.g., FIP-HRC peptide) extend outwards (see e.g., FIG. 26). In some embodiments, when the nanoparticles come into proximity of a lipid bilayer, such as the host cell membrane, they disassemble as the hydrophobic regions (e.g., membrane localizing moiety) will interact with the lipid membrane while the hydrophilic regions (e.g., FIP-HRC peptide) will face toward the aqueous solution (see e.g. FIG. 27).

### Soluble Stabilized Measles F Protein

Previously, soluble F always flipped into its post-fusion state unless engineered with disulfide bonds to maintain the pre-fusion state, as for the respiratory syncytial virus F vaccine candidate. Described herein, is a mutation in measles F that stabilizes the soluble F protein in its pre-fusion state. Both human parainfluenza virus type 3 wild type (wt) F protein and measles wt F protein when produced in a soluble form, with the ablation of the intracytoplasmic and transmembrane domains, are folded in the post-fusion state, even if a foldomer is added at the C-terminal to maintain trimeric oligomerization. If the wt measles F protein is expressed with media supplemented with the antiviral peptide conjugates described herein, such as, but not limited to FIP-HRC-PEG₁₁-dimer, wt F protein remains in its pre-fusion state. In some embodiments, soluble F protein comprises SEQ ID NO:1 or SEQ ID NO: 2. In some embodiments, soluble F protein consists of SEQ ID NO:1 or SEQ ID NO:2. In some embodiments, soluble F protein comprises an amino acid sequence comprising about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% sequence identity to SEQ ID NO:1 or SEQ ID NO:2. MGLKVNVSAIFMAVLLTLQTPTGQIHWGNLSKIGVVGIGSASYKVMTRSSHQSLVIKLM PNITLLNNCTRVEIAEYRRLLRTVLEPIRDALNAMTQNIRPVQSVASSRRHKRFAGVVLA GAALGVATAAQITAGIALHQSMLNSQAIDNLRASLETTNQAIEAIRQAGQEMILAVQGV QDYINNELIPSMNQLSCDLIGQKLGLKLLRYYTEILSLFGPSLRDPISAEISIQALSYALGG DINKVLEKLGYSGGDLLGILESRGIKARITHVDTESYFIVLSIAYPTLSEIKGVIVHRLEGV SYNIGSQEWYTTVPKYVATQGYLISNFDESSCTFMPEGTVCSQNALYPMSPLLQECLRG STKSCARTLVSGSFGNRFILSQGNLIANCASILCKCYTTGTIINQDPDKILTYIAADHCPVV EVNGVTIQVGSRRYPDAVYLHRIDLGPPISLERLDVGTNLGNAIAKLEDAKELLESSDQIL RSMKGLSSTS (SEQ ID NO:1).

In some embodiments, the soluble F protein optionally comprises a Tobacco Etch Virus protease site (shown in italics), a linker (shown in bold), a foldomer domain (shown as double underlined), and a 6xHis tag (shown in bolded italics).

(SEQ ID NO: 2)
MGLKVNVSAIFMAVLLTLQTPTGQIHWGNLSKIGVVGIGSASYKVMTRS
SHQSLVIKLMPNITLLNNCTRVEIAEYRRLLRTVLEPIRDALNAMTQNI
RPVQSVASSRRHKRFAGVVLAGAALGVATAAQITAGIALHQSMLNSQAI
DNLRASLETTNQAIEAIRQAGQEMILAVQGVQDYINNELIPSMNQLSCD
LIGQKLGLKLLRYYTEILSLFGPSLRDPISAEISIQALSYALGGDINKV
LEKLGYSGGDLLGILESRGIKARITHVDTESYFIVLSIAYPTLSEIKGV
IVHRLEGVSYNIGSQEWYTTVPKYVATQGYLISNFDESSCTFMPEGTVC
SQNALYPMSPLLQECLRGSTKSCARTLVSGSFGNRFILSQGNLIANCAS
ILCKCYTTGTIINQDPDKILTYIAADHCPVVEVNGVTIQVGSRRYPDAV
YLHRIDLGPPISLERLDVGTNLGNAIAKLEDAKELLESSDQILRSMKGL
SSTSGRENLYFQGGGGGSGYIPEAPRDOAYVRKDGEWVLLSTFLGGTEG
R

[symbol]

[symbol]

In certain aspects, the invention provides a recombinant polypeptide comprising soluble measles F protein comprising one or more mutations that result in a stabilized protein without the need for engineered disulfide bonds. In some embodiments, the mutation is E455G. In some embodiments, one or more mutations is E170G and E455G. In certain aspects, the technology provides nucleic acids encoding these recombinant polypeptides.

In some embodiments, the stabilized soluble F protein comprising mutation E455G comprises SEQ ID NO:3 or SEQ ID NO:4. In some embodiments, the stabilized soluble F protein comprising mutation E455G consists of SEQ ID NO:3 or SEQ ID NO:4. In some embodiments, stabilized soluble F protein comprises an amino acid sequence comprising about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% sequence identity to SEQ ID NO:3 or SEQ ID NO:4, wherein the amino acid at position 455 is G.

(SEQ ID NO: 3)
MGLKVNVSAIFMAVLLTLQTPTGQIHWGNLSKIGVVGIGSASYKVMTRSSH
QSLVIKLMPNITLLNNCTRVEIAEYRRLLRTVLEPIRDALNAMTQNIRPVQ
SVASSRRHKRFAGVVLAGAALGVATAAQITAGIALHQSMLNSQAIDNLRAS
LETTNQAIEAIRQAGQEMILAVQGVQDYINNELIPSMNQLSCDLIGQKLGL
KLLRYYTEILSLFGPSLRDPISAEISIQALSYALGGDINKVLEKLGYSGGD
LLGILESRGIKARITHVDTESYFIVLSIAYPTLSEIKGVIVHRLEGVSYNI
GSQEWYTTVPKYVATQGYLISNFDESSCTFMPEGTVCSQNALYPMSPLLQE
CLRGSTKSCARTLVSGSFGNRFILSQGNLIANCASILCKCYTTGTIINQDP

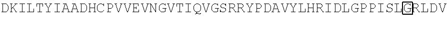

GTNLGNAIAKLEDAKELLESSDQILRSMKGLSSTS.

In some embodiments, the stabilized soluble F protein comprising mutation E455G optionally comprises a Tobacco Etch Virus protease site (shown in italics), a linker (shown in bold), a foldomer domain (shown as double underlined), and a 6xHis tag (shown in bolded italics).

(SEQ ID NO: 4)
MGLKVNVSAIFMAVLLTLQTPTGQIHWGNLSKIGVVGIGSASYKVMTRSSH
QSLVIKLMPNITLLNNCTRVEIAEYRRLLRTVLEPIRDALNAMTQNIRPVQ
SVASSRRHKRFAGVVLAGAALGVATAAQITAGIALHQSMLNSQAIDNLRAS
LETTNQAIEAIRQAGQEMILAVQGVQDYINNELIPSMNQLSCDLIGQKLGL
KLLRYYTEILSLFGPSLRDPISAEISIQALSYALGGDINKVLEKLGYSGGD
LLGILESRGIKARITHVDTESYFIVLSIAYPTLSEIKGVIVHRLEGVSYNI
GSQEWYTTVPKYVATQGYLISNFDESSCTFMPEGTVCSQNALYPMSPLLQE
CLRGSTKSCARTLVSGSFGNRFILSQGNLIANCASILCKCYTTGTIINQDP

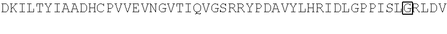

GTNLGNAIAKLEDAKELLESSDQILRSMKGLSSTSGRENLYFQGGGGGSGY
IPEAPRDQAYVRKDGEWVLLSTFLGGTEGR [symbol] [symbol] .

In some embodiments, the stabilized soluble F protein comprising double mutant E170G and E455G comprises SEQ ID NO:5 or SEQ ID NO:6. In some embodiments, the stabilized soluble F protein comprising double mutant E170G and E455G consists of SEQ ID NO:5 or SEQ ID NO:6. In some embodiments, stabilized soluble F protein comprising double mutation E170G and E455G comprises an amino acid sequence comprising about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% sequence identity to SEQ ID NO:5 or SEQ ID NO:6, wherein the amino acid at position 170 is G and the amino acid at position 455 is G.

(SEQ ID NO: 5)
MGLKVNVSAIFMAVLLTLQTPTGQIHWGNLSKIGVVGIGSASYKVMTRSSH
QSLVIKLMPNITLLNNCTRVEIAEYRRLLRTVLEPIRDALNAMTQNIRPVQ
SVASSRRHKRFAGVVLAGAALGVATAAQITAGIALHQSMLNSQAIDNLRAS

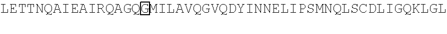

KLLRYYTEILSLFGPSLRDPISAEISIQALSYALGGDINKVLEKLGYSGGD
LLGILESRGIKARITHVDTESYFIVLSIAYPTLSEIKGVIVHRLEGVSYNI
GSQEWYTTVPKYVATQGYLISNFDESSCTFMPEGTVCSQNALYPMSPLLQE
CLRGSTKSCARTLVSGSFGNRFILSQGNLIANCASILCKCYTTGTIINQDP

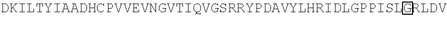

GTNLGNAIAKLEDAKELLESSDQILRSMKGLSSTS.

In some embodiments, the stabilized soluble F protein comprising mutation double mutation E170G and E455G optionally comprises a Tobacco Etch Virus protease site (shown in italics), a linker (shown in bold), a foldomer domain (shown as double underlined), and a 6xHis tag (shown in bolded italics).

(SEQ ID NO: 6)
MGLKVNVSAIFMAVLLTLQTPTGQIHWGNLSKIGVVGIGSASYKVMTRSSH
QSLVIKLMPNITLLNNCTRVEIAEYRRLLRTVLEPIRDALNAMTQNIRPVQ
SVASSRRHKRFAGVVLAGAALGVATAAQITAGIALHQSMLNSQAIDNLRAS

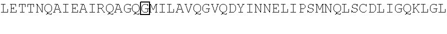

KLLRYYTEILSLFGPSLRDPISAEISIQALSYALGGDINKVLEKLGYSGGD
LLGILESRGIKARITHVDTESYFIVLSIAYPTLSEIKGVIVHRLEGVSYNI
GSQEWYTTVPKYVATQGYLISNFDESSCTFMPEGTVCSQNALYPMSPLLQE
CLRGSTKSCARTLVSGSFGNRFILSQGNLIANCASILCKCYTTGTIINQDP

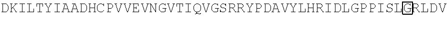

GTNLGNAIAKLEDAKELLESSDQILRSMKGLSSTSGRENLYFQGGGGGSGY
IPEAPRDQAYVRKDGEWVLLSTFLGGTEGR [symbol] [symbol] .

In certain aspects, the invention provides a cell comprising a nucleic acid encoding any one of the soluble F proteins of the invention (e.g. SEQ ID NOs: 1, 2, 3, 4, 5, or 6) suitable for recombinant expression. In certain aspects, the invention provides a clonally derived population of cells encoding any one of the soluble F proteins of the invention (e.g. SEQ ID NOs: 1, 2, 3, 4, 5, or 6) suitable for recombinant expression. In certain aspects, the invention provides a stable pool of cells encoding any one of the soluble F proteins of the invention (e.g. SEQ ID NOs: 1, 2, 3, 4, 5, or 6) suitable for recombinant expression. In some embodiments, nucleic acid sequences are codon optimized for optimal expression in a host cell, for example a mammalian cell, or any other suitable expression system.

A person of skill in the art understands that during recombinant expression of the soluble F proteins of the invention (e.g. SEQ ID NOs: 1, 2, 3, 4, 5, or 6), the signal peptide (sometimes referred to as signal sequence, targeting signal, localization signal, localization sequence, transit peptide, leader sequence or leader peptide) present at the N-terminus of the majority of newly synthesized proteins that are destined toward the secretory pathway can be cleaved from the final recombinant protein. In some embodiments, the recombinant soluble F protein therefore comprises amino acids starting at or about amino acid 25 onwards (i.e. signal peptide MGLKVNVSAIFMAVLLTLQTPTGQ (SEQ ID NO. 48) is cleaved from the recombinant protein).

In certain aspects, the invention provides a method for producing measles F protein in a pre-fusion state. In certain aspects, the invention provides culturing a cell line that expresses any one of the soluble F proteins of the invention (e.g. SEQ ID NOs: 1, 2, 3, 4, 5, or 6) in a culture medium comprising any of the antiviral peptide conjugates described herein, thereby producing a stabilized, soluble F protein in a pre-fusion state.

Various expression systems for producing recombinant proteins are known in the art, and include, prokaryotic (e.g., bacteria), plant, insect, yeast, and mammalian expression systems. Suitable cell lines, can be transformed, transduced, or transfected with nucleic acids containing coding sequences for the soluble F protein of the invention in order to produce the molecule of interest. Expression vectors containing such a nucleic acid sequence, which can be linked to at least one regulatory sequence in a manner that allows expression of the nucleotide sequence in a host cell, can be introduced via methods known in the art. Practitioners in the art understand that designing an expression vector can depend on factors, such as the choice of host cell to be transfected and/or the type and/or amount of desired protein to be expressed. Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication. For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest is stably integrated into the genome of eukaryotic cells (for example mammalian cells), resulting in the stable expression of transfected genes. A gene that encodes a selectable marker (for example, resistance to antibiotics or drugs) can be introduced into host cells along with the gene of interest in order to identify and select clones that stably express a gene encoding a protein of interest. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired protein molecule.

A host cell strain, which modulates the expression of the inserted sequences, or modifies and processes the nucleic acid in a specific fashion desired also may be chosen. Such modifications (for example, glycosylation and other post-translational modifications) and processing (for example, cleavage) of protein products may be important for the function of the protein. Different host cell strains have characteristic and specific mechanisms for the post-translational processing and modification of proteins and gene products. As such, appropriate host systems or cell lines can be chosen to ensure the correct modification and processing of the foreign protein expressed. Thus, eukaryotic host cells possessing the cellular machinery for proper processing of the primary transcript, glycosylation, and phosphorylation of the gene product may be used.

Various culturing parameters can be used with respect to the host cell being cultured. Appropriate culture conditions for mammalian cells are well known in the art (Cleveland W L, et al., J Immunol Methods, 1983, 56(2): 221-234) or can be determined by the skilled artisan (see, for example, Animal Cell Culture: A Practical Approach 2nd Ed., Rickwood, D. and Hames, B. D., eds. (Oxford University Press: New York, 1992)). Cell culturing conditions can vary according to the type of host cell selected. Commercially available medium can be utilized.

Soluble F proteins of the invention (e.g. SEQ ID NOs: 1, 2, 3, 4, 5, or 6) can be purified from any human or non-human cell which expresses the polypeptide, including those which have been transfected with expression constructs that express soluble F proteins of the invention. For protein recovery, isolation and/or purification, the cell culture medium or cell lysate is centrifuged to remove particulate cells and cell debris. The desired polypeptide molecule is isolated or purified away from contaminating soluble proteins and polypeptides by suitable purification techniques. Non-limiting purification methods for proteins include: size exclusion chromatography; affinity chromatography; ion exchange chromatography; ethanol precipitation; reverse phase HPLC; chromatography on a resin, such as silica, or cation exchange resin, e.g., DEAE; chromatofocusing; SDS-PAGE; ammonium sulfate precipitation; gel filtration using, e.g., Sephadex G-75, Sepharose; protein A sepharose chromatography for removal of immunoglobulin contaminants; and the like. Other additives, such as protease inhibitors (e.g., PMSF or proteinase K) can be used to inhibit proteolytic degradation during purification. Purification procedures that can select for carbohydrates can also be used, e.g., ion-exchange soft gel chromatography, or HPLC using cation- or anion-exchange resins, in which the more acidic fraction(s) is/are collected.

### Compositions

In some embodiments, the antiviral peptide conjugates are administered in a pharmaceutical composition comprising the antiviral peptide conjugates and a pharmaceutically acceptable carrier. In some embodiments, the stabilized F protein is administered in a pharmaceutical composition comprising the antiviral peptide conjugates and a pharmaceutically acceptable carrier. In some embodiments, the pharmaceutical composition is in the form of a spray, aerosol, gel, solution, emulsion, or suspension.

The composition is preferably administered to the mammal with a pharmaceutically acceptable carrier. Typically, in some embodiments, an appropriate amount of a pharmaceutically acceptable salt is used in the formulation, which in some embodiments can render the formulation isotonic.

In certain embodiments, the antiviral peptide conjugate is provided as an immunogenic composition comprising any one of the antiviral peptide conjugates described herein and a pharmaceutically acceptable carrier. In certain embodiments, the stabilized F protein is provided as an immunogenic composition comprising any one of the stabilized F proteins described herein and a pharmaceutically acceptable carrier. In certain embodiments, the immunogenic composition further comprises an adjuvant.

In some embodiments, the pharmaceutically acceptable carrier is selected from the group consisting of saline, Ringer's solution, dextrose solution, and a combination thereof. Other suitable pharmaceutically acceptable carriers known in the art are contemplated. Suitable carriers and their formulations are described in Remington's Pharmaceutical Sciences, 2005, Mack Publishing Co. The pH of the solution is preferably from about 5 to about 8, and more preferably from about 7 to about 7.5. The formulation may also comprise a lyophilized powder. Further carriers include sustained release preparations such as semipermeable matrices of solid hydrophobic polymers, which matrices are in the form of shaped articles, e.g., films, liposomes or microparticles. It will be apparent to those persons skilled in the art that certain carriers may be more preferable depending upon, for instance, the route of administration and concentration of antiviral peptide conjugates being administered.

The phrase pharmaceutically acceptable carrier as used herein means a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier is acceptable in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term carrier denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency. The composition may also include additional agents such as an isotonicity agent, a preservative, a surfactant, and, a divalent cation, preferably, zinc.

The composition can also include an excipient, or an agent for stabilization of an antiviral peptide conjugate composition, such as a buffer, a reducing agent, a bulk protein, amino acids (such as e.g., glycine or praline) or a carbohydrate. Bulk proteins useful in formulating antiviral peptide conjugate compositions include albumin. Typical carbohydrates useful in formulating antiviral peptide conjugates include but are not limited to sucrose, mannitol, lactose, trehalose, or glucose.

Surfactants may also be used to prevent soluble and insoluble aggregation and/or precipitation of peptides or proteins included in the composition. Suitable surfactants include but are not limited to sorbitan trioleate, soya lecithin, and oleic acid. In certain cases, solution aerosols are preferred using solvents such as ethanol. Thus, formulations including antiviral peptide conjugates or stabilized F protein can also include a surfactant that can reduce or prevent surface-induced aggregation of antiviral peptide conjugates or stabilized F protein caused by atomization of the solution in forming an aerosol. Various conventional surfactants can be employed, such as polyoxyethylene fatty acid esters and alcohols, and polyoxyethylene sorbitol fatty acid esters. Amounts will generally range between 0.001% and 4% by weight of the formulation. Especially preferred surfactants for purposes of this invention are polyoxyethylene sorbitan mono-oleate, polysorbate 80, polysorbate 20. Additional agents known in the art can also be included in the composition.

In some embodiments, the pharmaceutical compositions and dosage forms further comprise one or more compounds that reduce the rate by which an active ingredient will decay, or the composition will change in character. So called stabilizers or preservatives may include, but are not limited to, amino acids, antioxidants, pH buffers, or salt buffers. Nonlimiting examples of antioxidants include butylated hydroxy anisole (BHA), ascorbic acid and derivatives thereof, tocopherol and derivatives thereof, butylated hydroxy anisole and cysteine. Nonlimiting examples of preservatives include parabens, such as methyl or propyl p-hydroxybenzoate and benzalkonium chloride. Additional nonlimiting examples of amino acids include glycine or proline.

The present invention also teaches the stabilization (preventing or minimizing thermally or mechanically induced soluble or insoluble aggregation and/or precipitation of an inhibitor protein) of liquid solutions containing antiviral peptide conjugates at neutral pH or less than neutral pH by the use of amino acids including proline or glycine, with or without divalent cations resulting in clear or nearly clear solutions that are stable at room temperature or preferred for pharmaceutical administration.

In one embodiment, the composition is a pharmaceutical composition of single unit or multiple unit dosage forms. Pharmaceutical compositions of single unit or multiple unit dosage forms of the invention comprise a prophylactically or therapeutically effective amount of one or more compositions (e.g., a compound of the invention, or other prophylactic or therapeutic agent), typically, one or more vehicles, carriers, or excipients, stabilizing agents, and/or preservatives. Preferably, the vehicles, carriers, excipients, stabilizing agents and preservatives are pharmaceutically acceptable.

In some embodiments, the pharmaceutical compositions and dosage forms comprise anhydrous pharmaceutical compositions and dosage forms. Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprise a primary or secondary amine are preferably anhydrous if substantial contact with moisture and/or humidity during manufacturing, packaging, and/or storage is expected. An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Suitable vehicles are well known to those skilled in the art of pharmacy, and non-limiting examples of suitable vehicles include glucose, sucrose, starch, lactose, gelatin, rice, silica gel, glycerol, talc, sodium chloride, dried skim milk, propylene glycol, water, sodium stearate, ethanol, and similar substances well known in the art. Saline solutions and aqueous dextrose and glycerol solutions can also be employed as liquid vehicles. Whether a particular vehicle is suitable for incorporation into a pharmaceutical composition or dosage form depends on a variety of factors well known in the art including, but not limited to, the way in which the dosage form will be administered to a patient and the specific active ingredients in the dosage form. Pharmaceutical vehicles can be sterile liquids, such as water and oils, including those of petroleum, animal, vegetable or synthetic origin, such as peanut oil, soybean oil, mineral oil, sesame oil and the like.

A pharmaceutical composition of the invention is formulated to be compatible with its intended route of administration. Examples of routes of administration within the lower airways include, but are not limited to, oral or nasal inhalation (e.g., inhalation of sufficiently small particles to be deposited expressly within the lower airways). In various embodiments, the pharmaceutical compositions or single unit dosage forms are sterile and in suitable form for administration to a subject, preferably an animal subject, more preferably a mammalian subject, and most preferably a human subject.

The composition, shape, and type of dosage forms of the invention will typically vary depending on their use. Non limiting examples of dosage forms include powders; solutions; aerosols (e.g., sprays, metered or nonmetered dose atomizers, oral or nasal inhalers including metered dose inhalers (MDI)); liquid dosage forms suitable for mucosal administration to a patient, including suspensions (e.g., aqueous or non-aqueous liquid suspensions, oil-in-water emulsions, or a water-in-oil liquid emulsions), solutions, and sterile solids (e.g., crystalline or amorphous solids) that can also be reconstituted to provide liquid dosage forms suitable for lower airways administration. Formulations in the form of powders or granulates may be prepared using the ingredients mentioned above in a conventional manner using, e.g., a mixer, a fluid bed apparatus or a spray drying equipment.

The invention also provides that a pharmaceutical composition can be packaged in a hermetically sealed container such as an ampoule or sachette indicating the quantity. In one embodiment, the pharmaceutical composition can be supplied as a dry sterilized lyophilized powder in a delivery device suitable for administration to the lower airways of a patient. The pharmaceutical compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for administration may be in the form of powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a compound of the present invention (e.g., antiviral peptide conjugates) as an active ingredient.

A liquid composition herein can be used as such with a delivery device, or they can be used for the preparation of pharmaceutically acceptable formulations comprising antiviral peptide conjugates that are prepared for example by the method of spray drying. The methods of spray freeze-drying proteins for pharmaceutical administration disclosed in Maa et al., Curr. Pharm. Biotechnol., 2001, 1, 283-302, are incorporated herein. In another embodiment, the liquid solutions herein are freeze spray dried and the spray-dried product is collected as a dispersible antiviral peptide conjugate-containing powder that is therapeutically effective when administered into the lower airways of an individual.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another antiviral agent).

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The current invention provides for dosage forms comprising antiviral peptide conjugates (e.g., FIP-HRC peptides) suitable for treating measles or HIV infection. The dosage forms can be formulated, e.g., as sprays, aerosols, nanoparticles, liposomes, or other forms known to one of skill in the art. See, e.g., Remington's Pharmaceutical Sciences; Remington: The Science and Practice of Pharmacy supra; Pharmaceutical Dosage Forms and Drug Delivery Systems by Howard C., Ansel et al., Lippincott Williams & Wilkins; 7th edition (Oct. 1, 1999).

The current invention also provides for dosage forms comprising stabilized F protein suitable for treating or preventing measles infection. The dosage forms can be formulated, e.g., as sprays, aerosols, nanoparticles, liposomes, or other forms known to one of skill in the art. See, e.g., Remington's Pharmaceutical Sciences; Remington: The Science and Practice of Pharmacy supra; Pharmaceutical Dosage Forms and Drug Delivery Systems by Howard C., Ansel et al., Lippincott Williams & Wilkins; 7th edition (Oct. 1, 1999).

Generally, a dosage form used in the acute treatment of an infection/disorder may contain larger amounts of one or more of the active ingredients it comprises than a dosage form used in the chronic treatment of the same disease. In addition, the prophylactically and therapeutically effective dosage form may vary among different conditions. For example, a therapeutically effective dosage form may contain antiviral peptide conjugates that has an appropriate antiviral action when intending to treat an existing measles or HIV infection. On the other hand, a different effective dosage may contain antiviral peptide conjugates that has an appropriate immunogenic action when intending to use the peptides of the invention as a prophylactic (e.g., vaccine) against measles or HIV infection. A therapeutically effective dosage form may contain stabilized F protein that has an appropriate antiviral action when intending to treat an existing measles infection. On the other hand, a different effective dosage may contain stabilized F protein that has an appropriate immunogenic action when intending to use stabilized F protein of the invention as a prophylactic (e.g., vaccine) against measles infection. These and other ways in which specific dosage forms encompassed by this invention will vary from one another and will be readily apparent to those skilled in the art. See, e.g., Remington's Pharmaceutical Sciences, 2005, Mack Publishing Co.; Remington: The Science and Practice of Pharmacy by Gennaro, Lippincott Williams & Wilkins; 20th edition (2003); Pharmaceutical Dosage Forms and Drug Delivery Systems by Howard C. Ansel et al., Lippincott Williams & Wilkins; 7th edition (Oct. 1, 1999); and Encyclopedia of Pharmaceutical Technology, edited by Swarbrick, J. & J. C. Boylan, Marcel Dekker, Inc., New York, 1988, which are incorporated herein by reference in their entirety.

The pH of a pharmaceutical composition or dosage form may also be adjusted to improve delivery and/or stability of one or more active ingredients. Similarly, the polarity of a solvent carrier, its ionic strength, or tonicity can be adjusted to improve delivery. Compounds such as stearates can also be added to pharmaceutical compositions or dosage forms to alter advantageously the hydrophilicity or lipophilicity of one or more active ingredients to improve delivery. In this regard, stearates can also serve as a lipid vehicle for the formulation, as an emulsifying agent or surfactant, and as a delivery enhancing or penetration-enhancing agent. Different salts, hydrates, or solvates of the active ingredients can be used to adjust further the properties of the resulting composition.

Compositions can be formulated with appropriate carriers and adjuvants using techniques to yield compositions suitable for immunization. The compositions can include an adjuvant, such as, for example but not limited to, alum, poly IC, MF-59, squalene-based adjuvants, or liposomal based adjuvants suitable for immunization.

In some embodiments, the compositions and methods comprise any suitable agent or immune modulation which could modulate mechanisms of host immune tolerance and release of the induced antibodies. In certain embodiments, an immunomodulatory agent is administered in at time and in an amount sufficient for transient modulation of the subject's immune response so as to induce an immune response which comprises antibodies against measles F protein or HIV-1 envelope.

### Methods of Treatment

In one embodiment, the subject matter disclosed herein relates to a preventive medical treatment started after exposure to MV in order to prevent the infection from occurring or worsening. In one embodiment, the subject matter disclosed herein relates to prophylaxis of subjects who have come into contact with MV or are suspected to have come into contact with MV. In one embodiment, said subjects can be administered post-exposure prophylaxis comprising the antiviral peptide conjugates described herein or pharmaceutical compositions thereof. In some embodiments, the antiviral peptide conjugate comprises an FIP conjugated to an HRC peptide derived from measles virus as a preventative for measles infection. The inventions contemplates using any of the antiviral peptide conjugates described herein. In some embodiments, the FIP conjugated HRC peptide is in monomer form (e.g., “FIP-HRC₄₅₀₋₄₈₅,” see FIG. 64). In some embodiments, the FIP conjugated HRC peptide is in monomer form and further comprises a lipid (e.g., cholesterol) conjugate to the C-terminus of the peptide (e.g. “FIP-HRC₄₅₀₋₄₈₅-chol,” see FIG. 64). In some embodiments, the FIP conjugated HRC peptide is in monomer form and further comprises a linker (e.g., PEG₄) and a lipid (e.g., cholesterol) conjugate to the C-terminus of the peptide (e.g., “FIP-HRC₄₅₀₋₄₈₅-peg₄-chol,” see FIG. 64). In some embodiments, the FIP conjugated HRC peptide is in dimer form and further comprises a linker (e.g., PEG₄) and a lipid (e.g., cholesterol) conjugate to the C-terminus of the peptide (e.g., “[FIP-HRC₄₅₀₋₄₈₅-peg_{4]2}-chol,” see FIG. 64). In some embodiments, the FIP conjugated HRC peptide is in dimer form and further comprises a linker (e.g., PEG₄) conjugate to the C-terminus of the peptide (e.g., “[FIP-HRC₄₅₀₋₄₈₅-peg₁₁]₂,” see FIG. 64). In one embodiment, the antiviral peptide conjugates described herein can be administered in the form of a nanoparticle. In one embodiment, the antiviral peptide conjugates described herein can be administered intranasally via an intranasal spray or any other suitable method know in the art. In one embodiment, the antiviral peptide conjugates described herein can be administered subcutaneously via syringe or any other suitable method know in the art. In one embodiment, the subject matter disclosed herein can be adapted and applied to post-exposure prophylaxis for paramyxoviruses other than MV, such as mumps. In one embodiment, the subject matter disclosed herein relates to a post-exposure prophylaxis approach of any virus by inhibiting viral fusion.

In one embodiment, the subject matter disclosed herein relates to a preventive medical treatment started after exposure to HIV in order to prevent the infection from occurring or worsening. In one embodiment, the subject matter disclosed herein relates to prophylaxis of subjects who have come into contact with HIV or are suspected to have come into contact with HIV. In one embodiment, said subjects can be administered post-exposure prophylaxis comprising the antiviral peptide conjugates described herein or pharmaceutical compositions thereof. In some embodiments, the antiviral peptide conjugate comprises an FIP conjugated to an HRC peptide derived from HIV-gp41 (“C34”) as a preventative for HIV infection. In some embodiments, the antiviral peptide conjugate comprises an FIP conjugated to an HRC peptide derived from HIV-gp41 (“C34”) can also be used as a preventative for MV infection. In some embodiments, the FIP conjugated HRC peptide is in monomer form and further comprises a lipid (e.g., cholesterol) conjugate to the C-terminus of the peptide. In some embodiments, the FIP conjugated HRC peptide is in monomer form and further comprises a linker (e.g., PEG₄) and a lipid (e.g., cholesterol) conjugate to the C-terminus of the peptide. In some embodiments, the FIP conjugated HRC peptide is in dimer form and further comprises a linker (e.g., PEG₄) and a lipid (e.g., cholesterol) conjugate to the C-terminus of the peptide. In some embodiments, the FIP conjugated HRC peptide is in dimer form and further comprises a linker (e.g., PEG₄) conjugate to the C-terminus of the peptide. In one embodiment, the antiviral peptide conjugates described herein can be administered in the form of a nanoparticle. In one embodiment, the antiviral peptide conjugates described can be administered intranasally via an internasal spray or any other suitable method know in the art. In one embodiment, the antiviral peptide conjugates described can be administered subcutaneously via syringe or any other suitable method know in the art. In one embodiment, the subject matter disclosed herein relates to a post-exposure prophylaxis approach of any virus by inhibiting viral fusion.

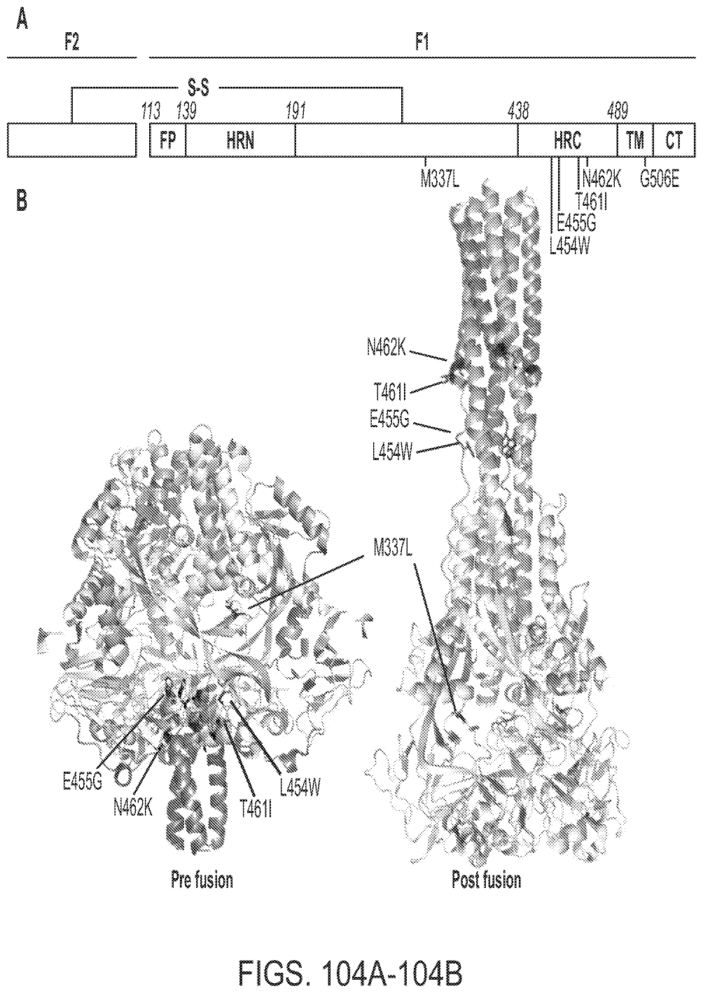

In some embodiments, the subject has been exposed to a measles virus comprising a wild type fusion glycoprotein. In some embodiments, the subject has been exposed to a measles virus comprising one or more mutations of a fusion glycoprotein selected from the group consisting of N462K, L454W, T461I, E455G, E170G, G506E, M337L, D538G, G168R, S262G, A440P, R520C, and L550P (e.g., see FIG. 104).

The compound(s) or combination of compounds disclosed herein, or pharmaceutical compositions may be administered to a cell, mammal, or human by any suitable means. Non-limiting examples of methods of administration include, among others, (a) administration though oral pathways, which includes administration in capsule, tablet, granule, spray, syrup, or other such forms; (b) administration through non-oral pathways such as intraocular, intranasal, intraauricular, rectal, vaginal, intraurethral, transmucosal, buccal, or transdermal, which includes administration as an aqueous suspension, an oily preparation or the like or as a drip, spray, suppository, salve, ointment or the like; (c) administration via injection, including subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation; (e) administration topically; as deemed appropriate by those of skill in the art for bringing the compound or combination of compounds disclosed herein into contact with living tissue; (f) administration via inhalation, including through aerosolized, nebulized, and powdered formulations; and (g) administration through implantation.

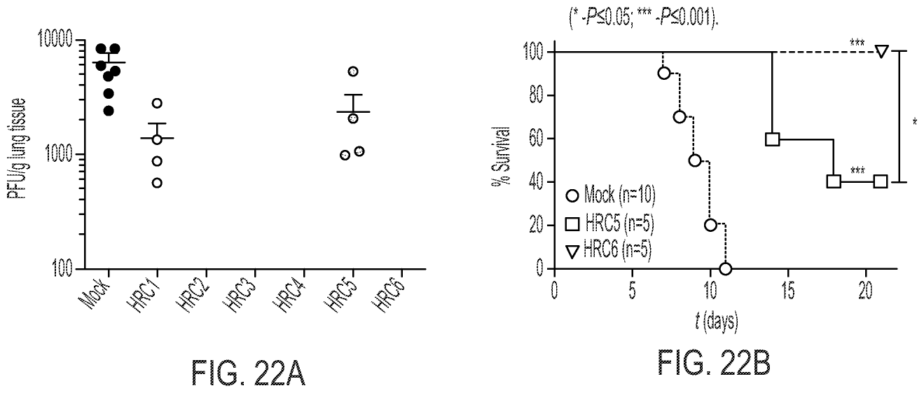

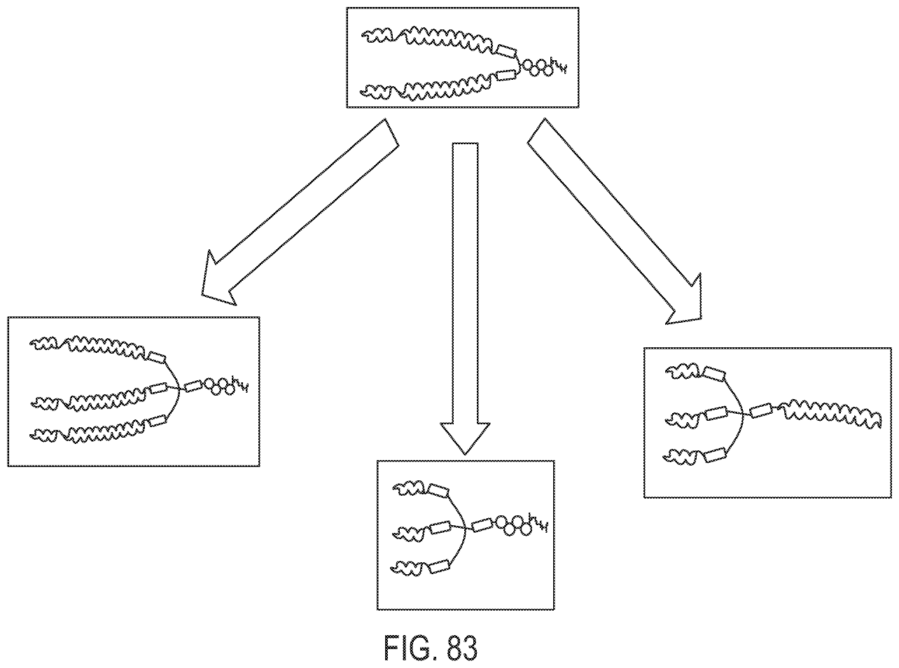

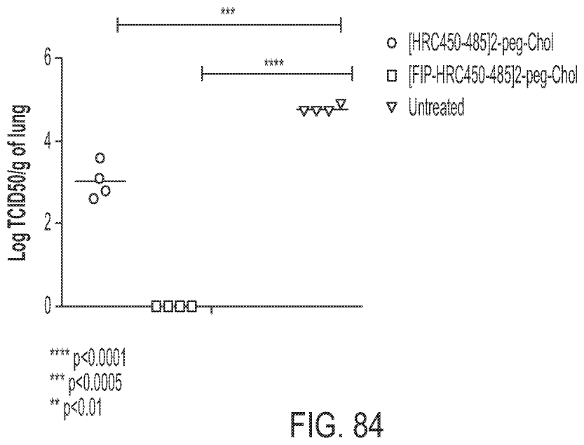

As will be readily apparent to one skilled in the art, the effective in vivo dose to be administered and the particular mode of administration will vary depending upon the age, weight and species treated, and the specific use for which the compound or combination of compounds disclosed herein are employed. The determination of effective dose levels, that is the dose levels necessary to achieve the desired result, can be accomplished by one skilled in the art using routine pharmacological methods. Typically, human clinical applications of products are commenced at lower dose levels, with dose level being increased until the desired effect is achieved. Alternatively, acceptable in vitro studies can be used to establish useful doses and routes of administration of the compositions identified by the present methods using established pharmacological methods. Effective animal doses from in vivo studies (e.g., 6 mg/kg in cotton rats; see FIGS. 22 and 84) can be converted to appropriate human doses using conversion methods known in the art (e.g., see Nair A B, Jacob S. A simple practice guide for dose conversion between animals and human. Journal of basic and clinical pharmacy. 2016 March; 7(2):27.)

### Methods of Prevention

Without being bound by theory, the antiviral peptide conjugates described herein stabilize soluble measles F protein in a pre-fusion state. Therefore, in some embodiments the antiviral peptide conjugates of the invention can be used with soluble measles F protein (e.g. SEQ ID NOs: 1, 2, 3, 4, 5, or 6) as a vaccine to promote an immune response against measles pre-fusion F protein. In some embodiments the antiviral peptide conjugates of the invention can be administered with soluble measles F protein (e.g. SEQ ID NOs: 1, 2, 3, 4, 5, or 6) to elicit a protective immune response against measles. In some embodiments, the invention provides a method of inducing an immune response in a subject comprising administering an immunogenic composition comprising any one of the antiviral peptide conjugates of the invention and any one of the soluble measles F protein (e.g. SEQ ID NOs: 1, 2, 3, 4, 5, or 6). In some embodiments, the antiviral peptide conjugates of the invention can also be used with the soluble measles F protein (e.g. SEQ ID NOs: 1, 2, 3, 4, 5, or 6) as a prophylactic treatment of a subject infected with measles virus.

In some embodiments, the stabilized soluble F protein (e.g. SEQ ID NOs: 3, 4, 5, or 6) can be used as a vaccine to promote an immune response against measles pre-fusion F protein (i.e., without the addition of the antiviral peptide conjugates described herein). In some embodiments, the stabilized soluble F protein (e.g. SEQ ID NOs: 3, 4, 5, or 6) can be administered to elicit a protective immune response against measles. In some embodiments, the invention provides a method of inducing an immune response in a subject comprising administering an immunogenic composition comprising any one of the stabilized soluble F proteins described herein (e.g. SEQ ID NOs: 3, 4, 5, or 6).

In some embodiments, stabilized F protein is administered alone or in combination with any of the antiviral peptide conjugates described herein. In some embodiments, the antiviral peptide conjugates of the invention can be used with the soluble measles F protein (e.g. SEQ ID NOs: 1, 2, 3, 4, 5, or 6) as a prophylactic treatment of a subject infected with measles virus.

In some embodiments, the invention provides compositions and methods for induction of immune response, for example induction of antibodies to measles virus or to HIV. In some embodiments, the antibodies are broadly neutralizing antibodies. In some embodiments, the method induces antibodies to measles F protein or HIV-1 envelope. In some embodiments, the methods use compositions comprising stabilized F protein and/or any of the antiviral peptide conjugates described herein. In some embodiments, the methods further comprise administering an adjuvant. In some embodiments, the invention provides compositions and methods for induction of immune response to a measles virus comprising a wild type fusion glycoprotein. In some embodiments, the invention provides compositions and methods for induction of immune response to a measles virus comprising one or more mutations of a fusion glycoprotein selected from the group consisting of N462K, L454W, T461I, E455G, E170G, G506E, M337L, D538G, G168R, S262G, A440P, R520C, and L550P (e.g., see FIG. 104).

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

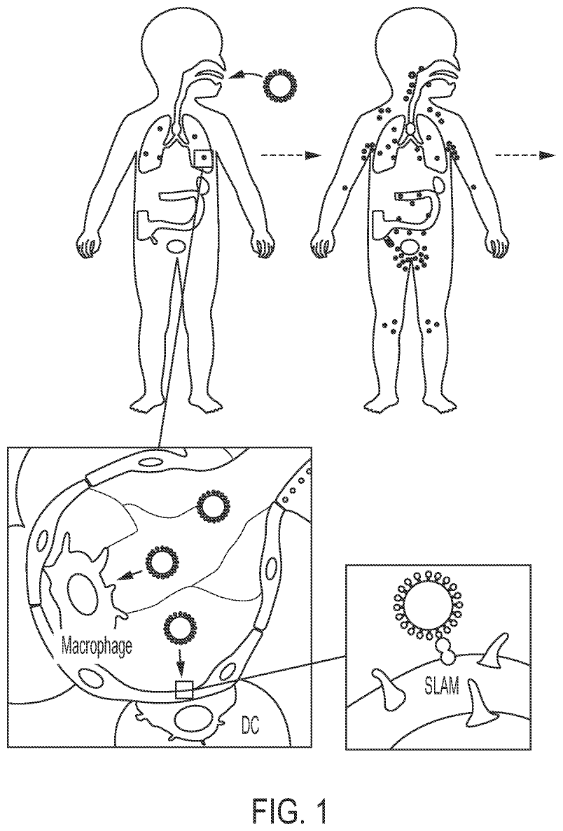

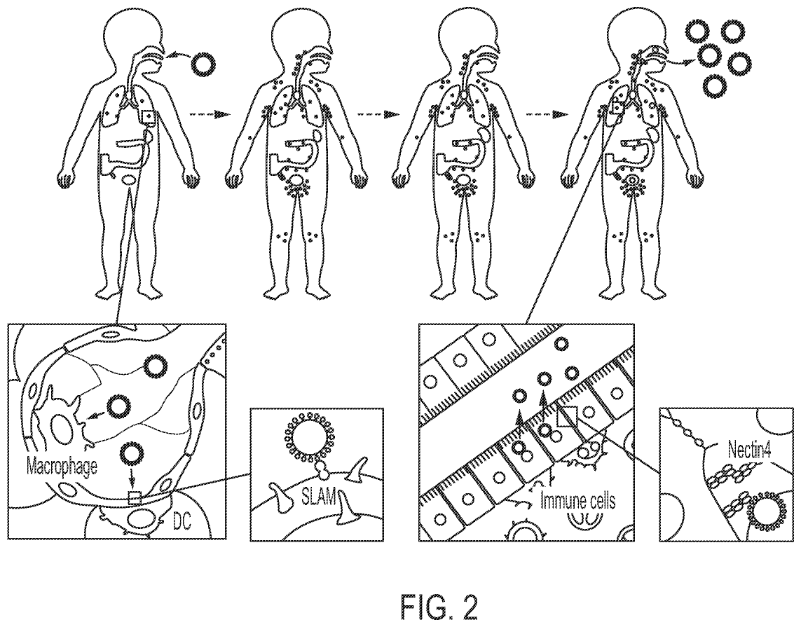

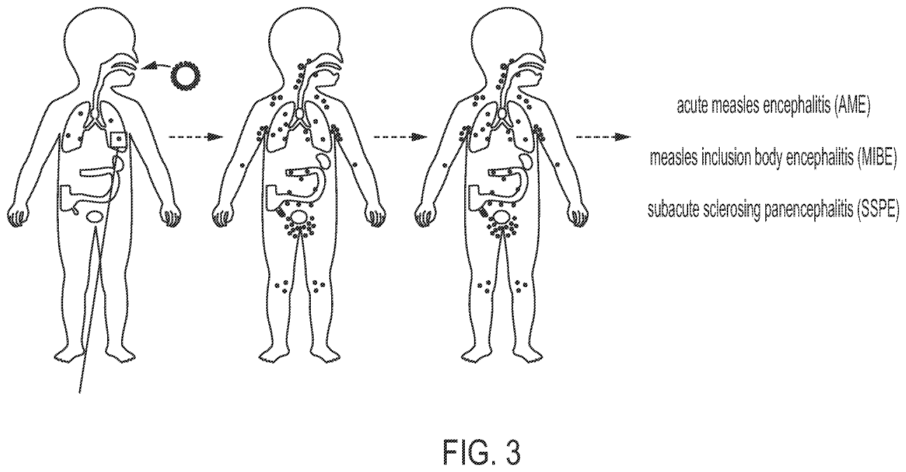

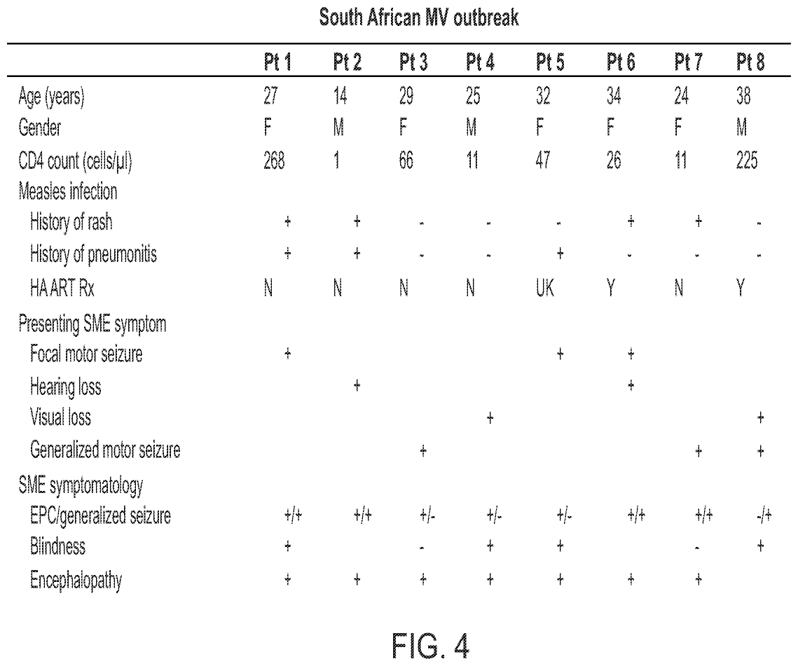

An MeV infection can start in the respiratory tract as shown in FIG. 1. The alveolar macrophages and dendritic cells (DC) are the primary targets that express the MeV receptor signaling lymphocyte activation molecule (SLAM, also called CD150). Attachment of the MeV receptor binding protein hemagglutinin (H) to CD150 leads to infection of these cells, which then transmit the virus to bronchus-associated lymphoid tissues and/or draining lymph nodes. The virus proliferates in CD150-expressing B and T lymphocytes, and viremia ensues. The adherens junction protein (PVRL4 or nectin 4) also serves as an MeV receptor but is found on the basolateral surface of respiratory epithelial cells; it is implicated in viral transmission at later stages of illness. Viremia and egress of MeV pathogenesis are shown in FIG. 2. Measles can also cause severe complications as shown in FIG. 3. Some of these complications affect the central nervous system (CNS). Measles inclusion body encephalitis (MIBE) and subacute sclerosing panencephalitis (SSPE) are both lethal complications of measles infection. MIBE may occur 1-9 months after viral infection. SSPE may appear several years post-infection. In one embodiment, the subject matter disclosed herein is focusing on clinical isolates from MIBE and SSPE central nervous system complications. In one embodiment, acute measles encephalitis (AME) is another complication of MeV. In the past, the rate of SSPE was thought to be 1:100000 cases, but recent data have shown that infection in children in their first year of life can lead to 1 case every 600 infections (Wendorf, K. A., et al. Clin. Infect. Dis. 2017, 65(2), 226-232). MIBE is a lethal CNS manifestation of measles in severely immune compromised patients. Data for HIV-infected patients with MeV encephalitis are shown in FIG. 4. Patient 8 was on HAART for 1 year prior. At the time of the report, patients 1 and 8 were still alive. All patients eventually died (unpublished data, personal comm., Diana Hardie).

### MV Pathology: Central Nervous System Complications

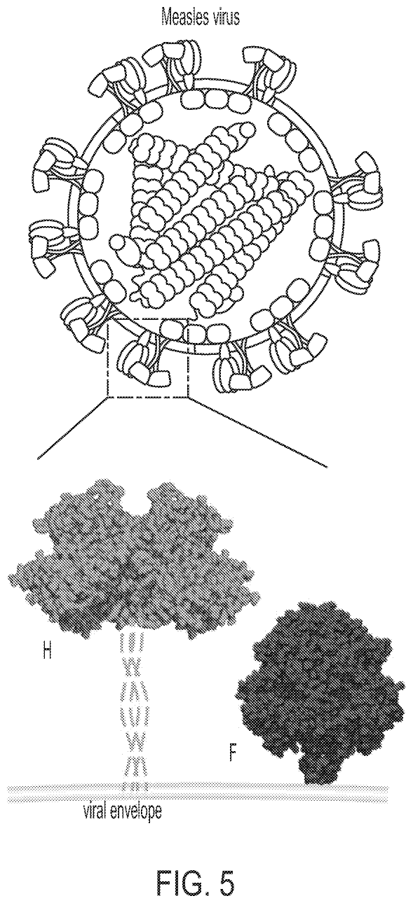

A long standing question is how measles infects and spreads in the CNS, because the CNS lacks known MV receptors. The fusion complex of paramyxoviruses has been described herein. Also described are CNS clinical isolates. Therefore, another question discussed herein is how the measles fusion complex adapts in the CNS. A schematic representation of the measles virus including the F and H proteins is shown in FIG. 5. The subject matter disclosed herein also relates to data from clinical isolate sequences from Patients 1 and 6 as shown in FIG. 4.

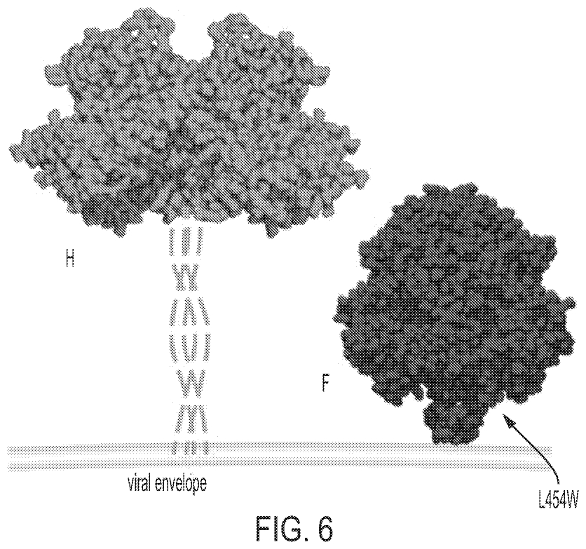

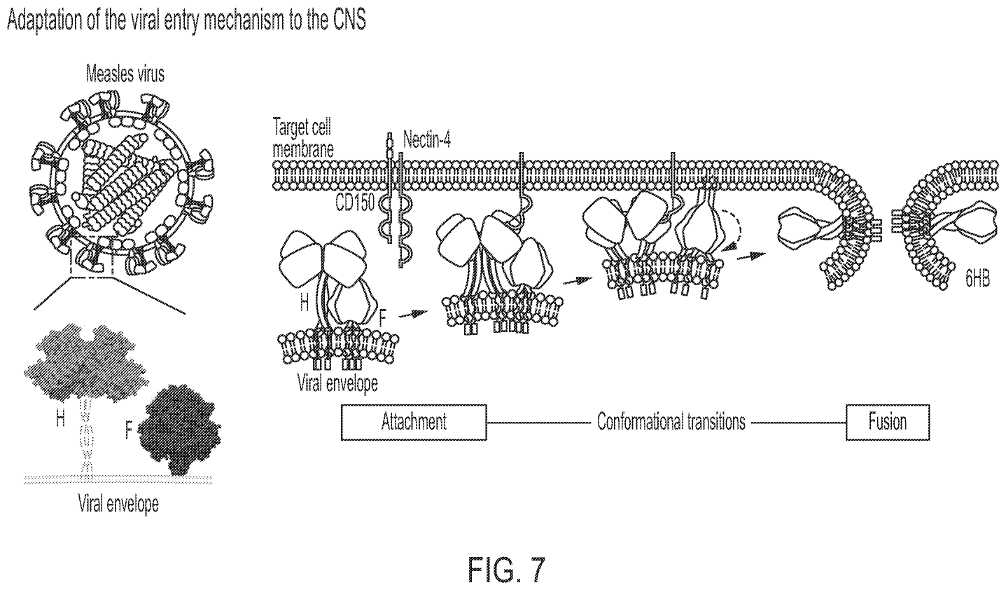

In one embodiment, the subject matter disclosed herein relates to characterization of the fusion complex of neuropathogenic MeV isolates recovered from the CNS of patients who suffered from MIBE during the SA outbreak in 2009/2010. MeV sequences were isolated from the postmortem brain tissue of two HIV-infected patients who were diagnosed with MIBE via positive MeV PCR during a measles epidemic in South Africa. Viral-genome sequencing revealed that in both cases, the MeV F gene contained the same nucleotide mutation as shown in FIG. 6, one that resulted in a leucine-to-tryptophan substitution at position 454 (L454W). The first patient was a 27-year-old woman who developed MIBE 3 months after her acute measles. Of note, the L454W mutation present in the virus isolated from her brain was not present in virus from the earlier blood samples obtained during her acute MV infection. The second patient was a 34-year-old woman who developed typical MIBE symptoms 3 weeks after acute MeV infection. Viral entry for wild-type measles into the CNS is tightly regulated as shown in FIG. 7. Wild-type MeV requires Nectin4 or CD150 receptors for the entry step. Therefore, one question that arises is how do SSPE and MIBE MeV strains enter and spread in the CNS?

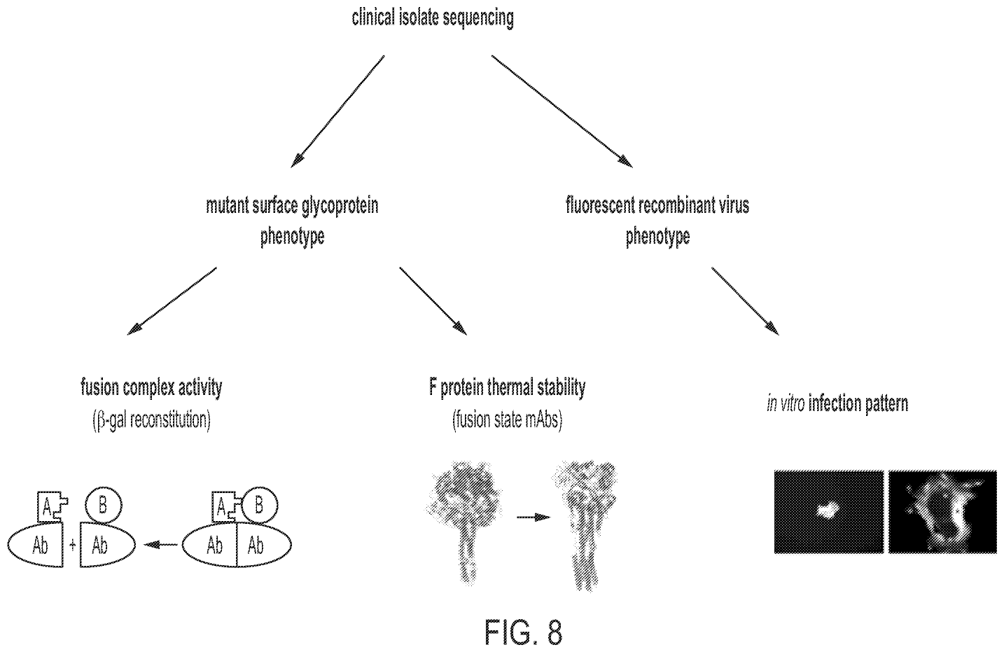

In one embodiment of the subject matter disclosed herein, the fusion machinery of neuropathogenic MeV isolates recovered from the CNS of patients who suffered from MIBE during the SA outbreak in 2009/2010 was characterized and one other question asked was how does this single amino acid mutation alter the measles fusion complex? Viral-genome sequencing revealed that in both cases, the MeV F gene contained the same nucleotide mutation, one that resulted in a leucine-to-tryptophan substitution at position 454 (L454W). Methodology of fusion complex analysis is shown in FIG. 8. We asked whether the L454W nucleotide mutation alters the activity of the MV fusion machinery.

### CNS Adapted Fusion Machinery: Altered Requirement for Receptor Engagement to Fuse

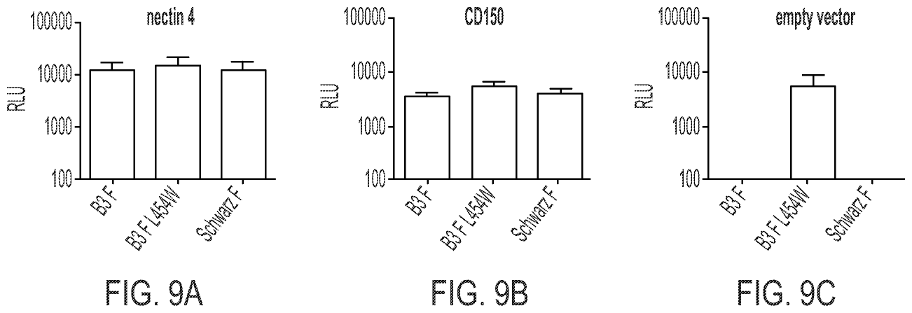

As shown in FIGS. 9A-B, in the presence of a known receptor, all F proteins demonstrate similar levels of fusion. As shown in FIG. 9C, F L454W induces fusion in the absence of a known receptor.

### Does the L454W Mutation Destabilize F?

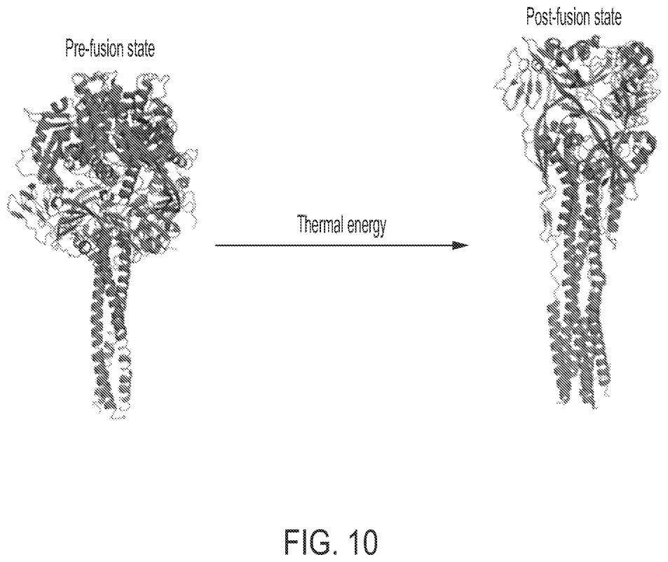

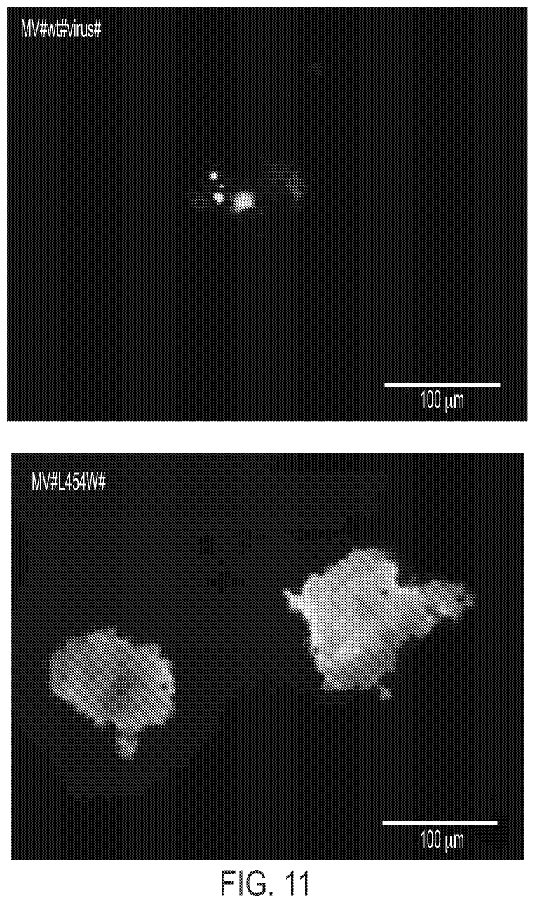

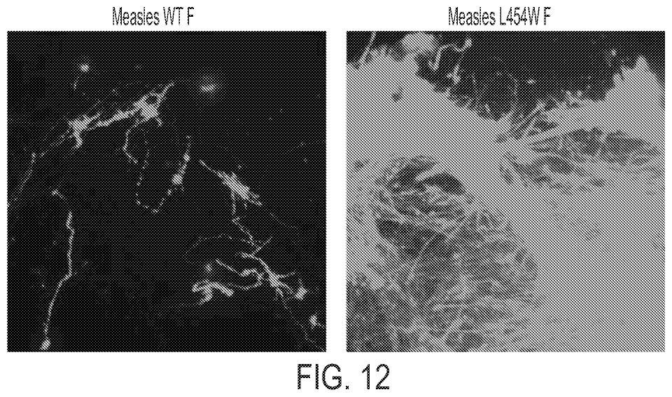

Thermal stability of the wild-type and CNS-adapted MeV Fs is disclosed herein. Cells expressing the indicated MV Fs (x-axis) were incubated overnight at 32° C. and then placed at indicated Ts. The cells were then incubated at 4° C. with mAbs vs. either the pre-fusion or the post-fusion state of the MeV F. Values on y-axis represent the relative luminescence units (RLU). F L454W is less thermally stable than wt F. Representations of the two states are shown in FIG. 10. The viruses bearing the CNS-derived F can spread in cells lacking MeV receptor. As shown in FIG. 11, cell-to-cell fusion induced by recombinant viruses. MIBE derived F L454W is capable of mediating fusion in the absence of known MeV receptor. Increase in fusion activity of MV F L454W over F Wild-Type is correlated to a decrease in stability. MV F L454W can promote fusion independent of H.

Is the L454W F Bearing Virus More Pathogenic than Wild-Type Virus?

Models used to answer this question include, but are not limited to human brain organoids, ex vivo tissue from mice and cotton rats. MeV infects the cotton rats and viral titer can be assessed. There is also a suckling mouse model transgenic for CD150 receptor which results in lethal infection with wild-type virus.

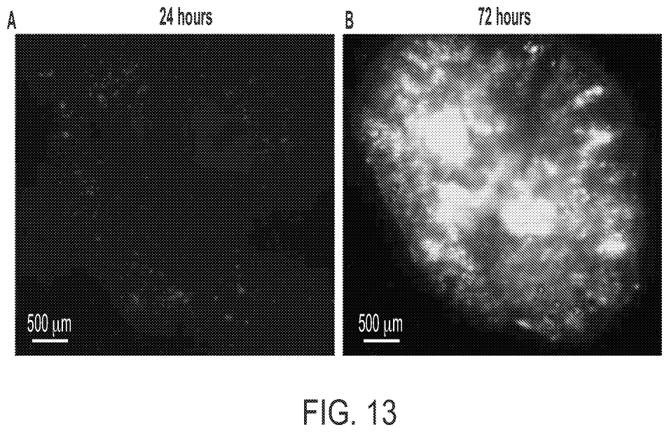

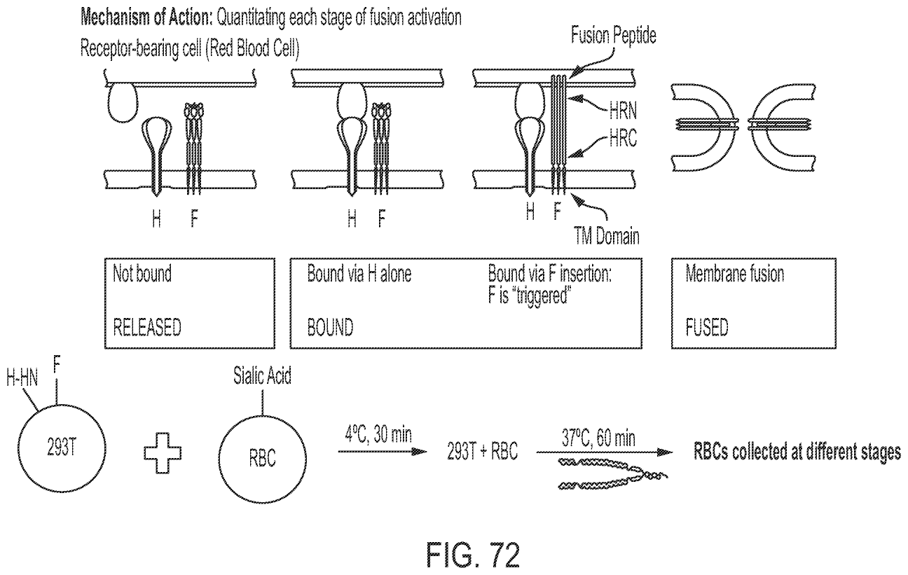

90-day old brain organoids were infected with measles wild-type F virus and L454W F bearing virus as shown in FIG. 12. FIG. 13 shows ex vivo tissue from mice (no receptor present). Ex vivo infection with wild-type versus virus bearing the L454W F: the CNS-adapted virus outcompetes the wild-type virus in organotypic brain cultures. Cerebellar slices from IFNAR KO murine brains were infected with wild-type virus bearing the EGFP at 5000 PFU/slice for 4 days. Red fluorescence related to the infection at either 24 or 96 hours observed by epifluorescence microscopy (scale bar=500 μm). MeV bearing L454W F dissemination in organotypic brain cultures (OBC). Cerebellar slices from IFNAR KO murine brains were infected with MeV-IC323-L454W F EGFP (green fluorescence), and with wild-type virus bearing the tdtomato (red fluorescence) at 5000 PFU/slice for 4 days. Green and red fluorescence related to the infection at either 24 (FIG. 13A) or 72 (FIG. 13B) hours observed by epifluorescence microscopy (scale bar=500 μm).

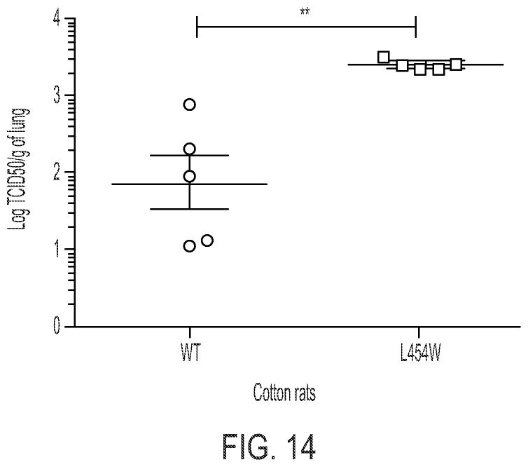

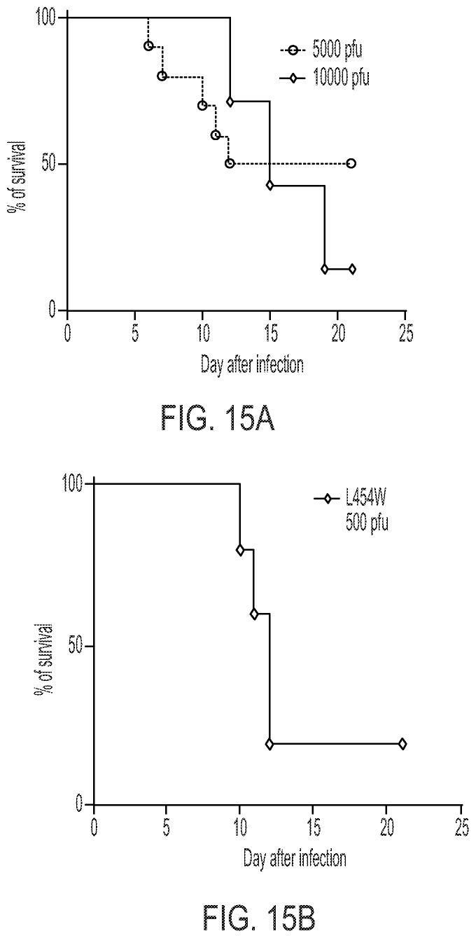

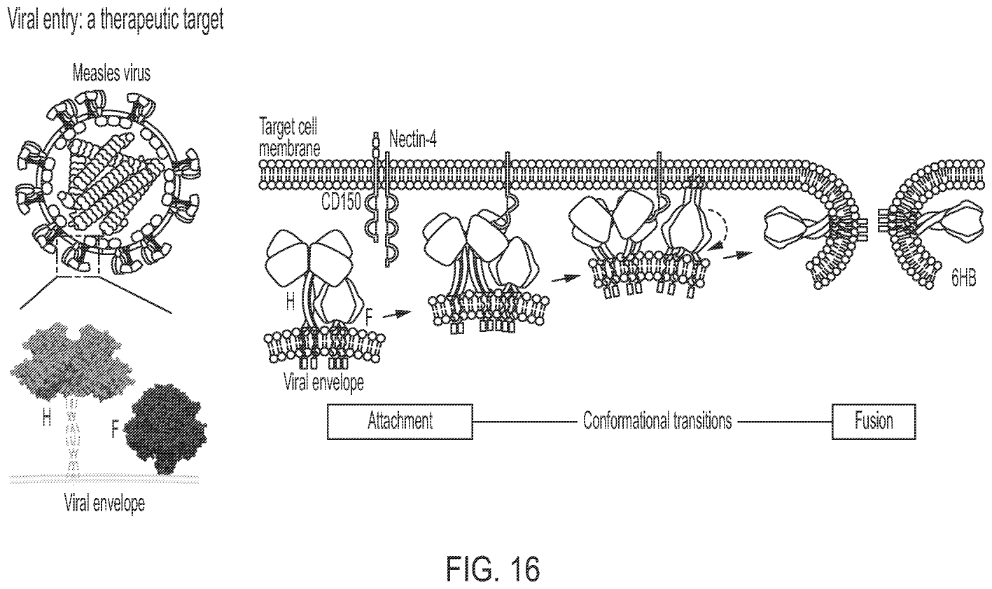

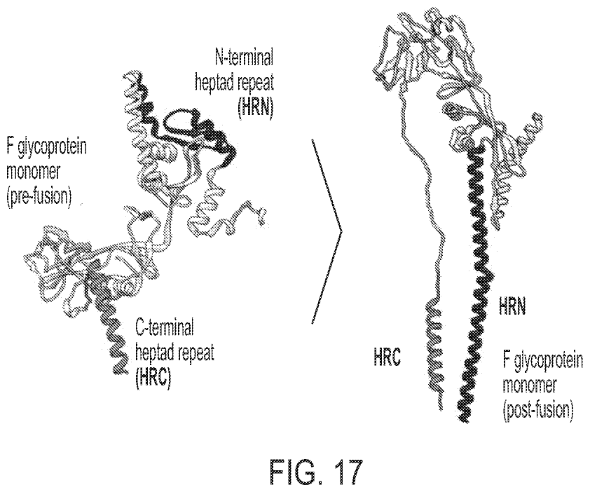

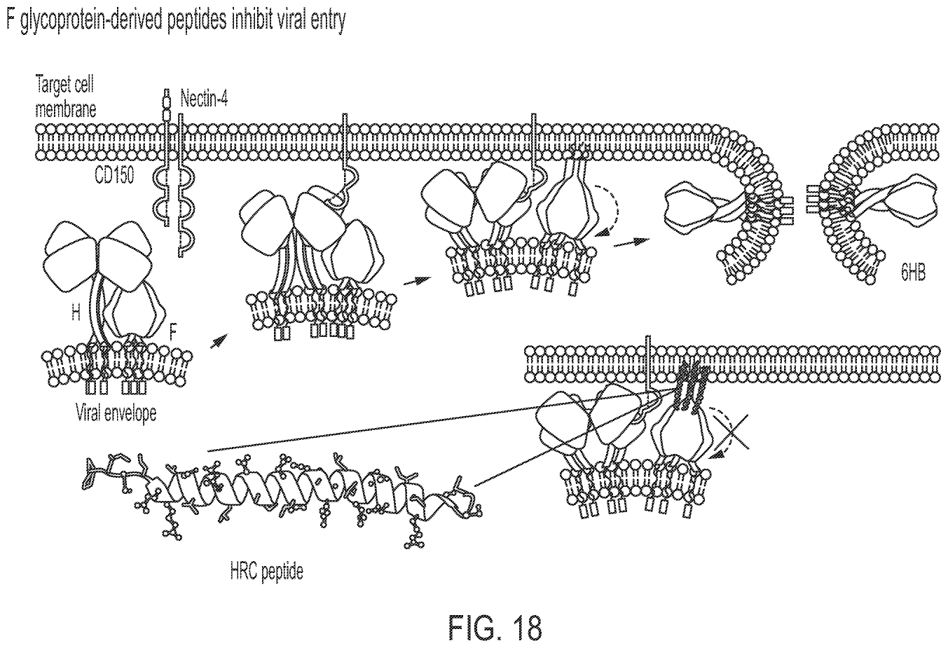

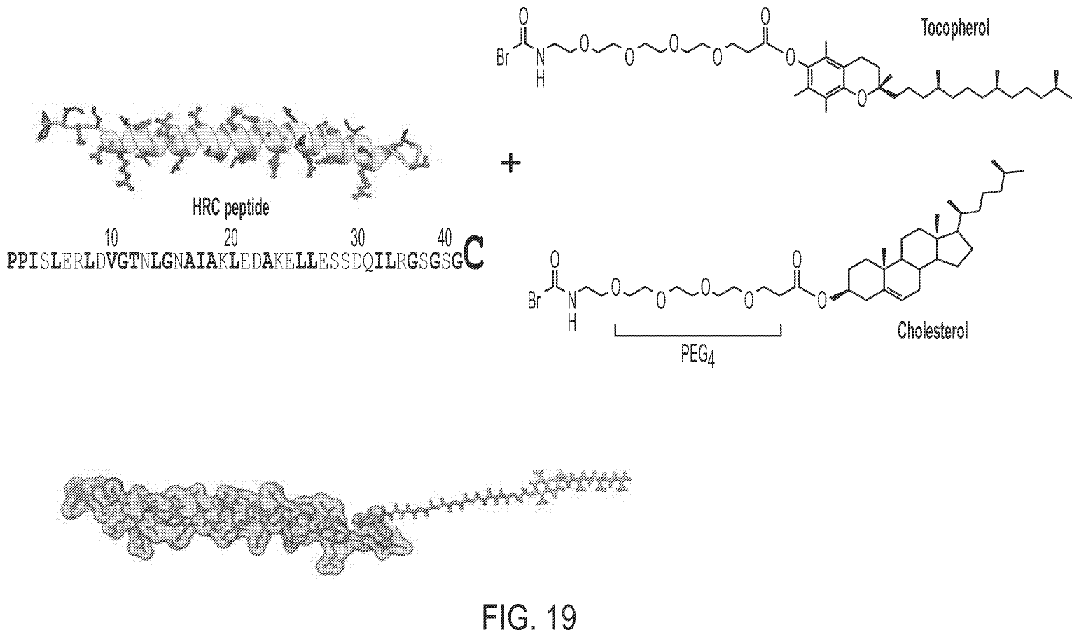

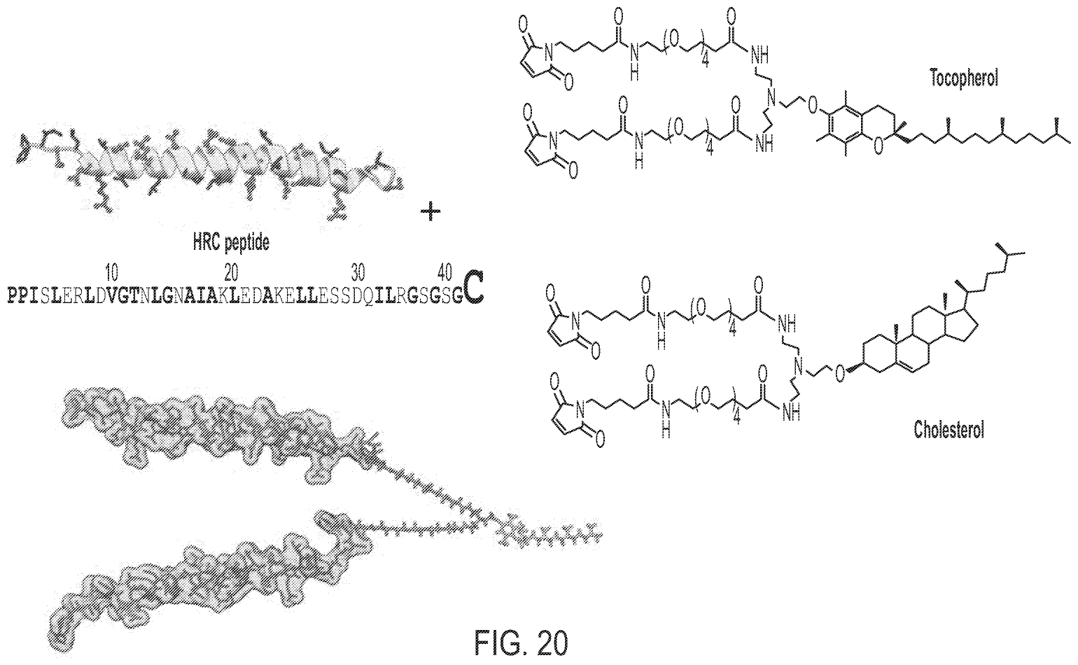

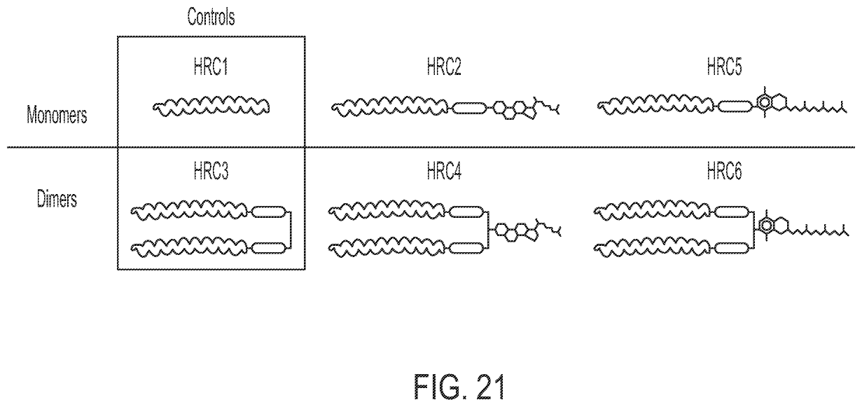

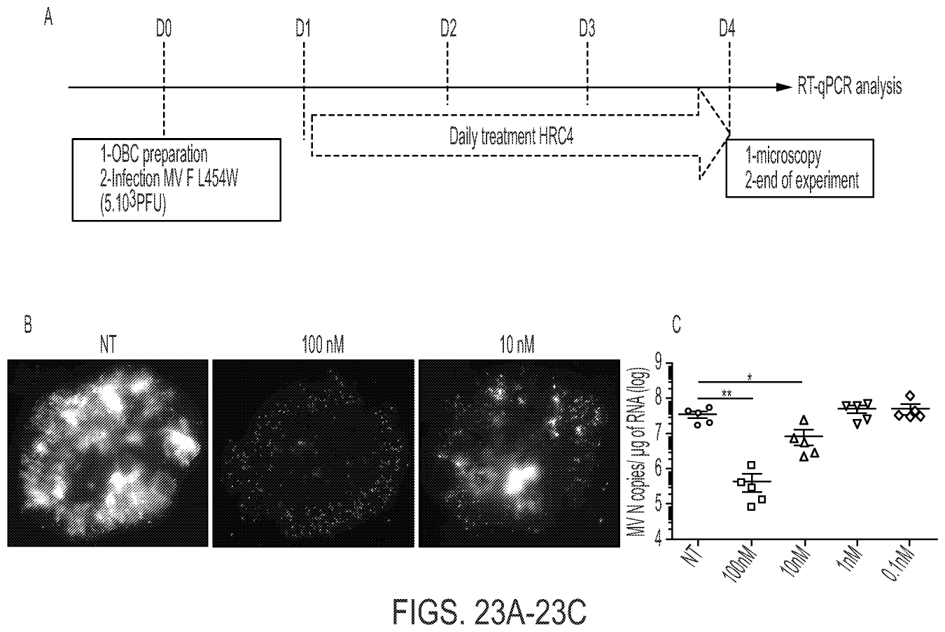

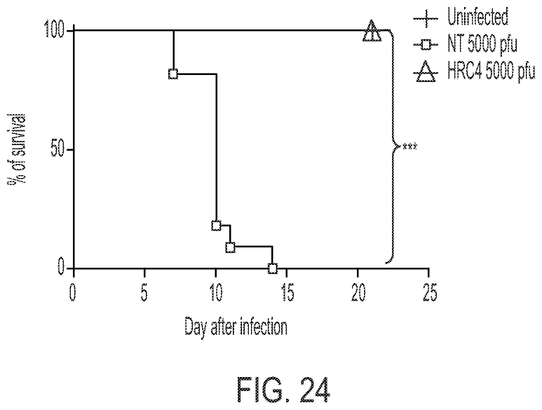

FIG. 14 shows MeV virus data in cotton rats. FIGS. 15A-B show survival rate data in mice. FIG. 16 shows viral entry as a therapeutic target. F glycoprotein derived peptides, as shown in FIG. 17, inhibit viral entry as shown in FIG. 18. FIG. 19 shows targeting HRC peptides toward lipid membranes. FIG. 20 shows improving HRC peptides' avidity towards F protein. FIG. 21 shows another embodiment of targeting HRC peptides towards lipid membranes. FIGS. 22A-B show that conjugated peptides are potent in vivo. The models used are cotton rats in FIG. 22A and SLAM:IFNARKO mice in FIG. 22B. The administration route is intranasal at a dosage of 6 mg/kg. FIGS. 23A-C show that MeV HRC4 peptide blocks viral spread ex vivo. The model used is ex vivo tissue from mice (without CD150) infected with L454W F bearing virus. FIG. 24 shows that intranasal administration of MeV HRC4 protects suckling mice from lethal infection with virus bearing L454W F.

Tables 1-3 provide non-limiting examples of peptides.

Table 1 provided a list of peptides and their modification. ^{a}(Amino acid residues are represented in single letter code; Ac—Acetylated N-terminus; GSGSG—linker containing five amino acids; D-FFG—D-amino acid has been used for the first phenyl alanine residue; Z-D-FFG-Z is carbobenzoxy attached to the N-terminus of D-FFG sequence; ^{b}(FIP—Fusion Inhibitory Peptide; HRC450-485—measles HRC derived peptide sequence starting from 450th amino acid and ending at 485th amino acid); ^{c}(Peg—polyethylene glycol; Chol—Cholesterol); ^{d}(Peptide—HRC450-485/FIP-HRC450-485/FIP).

TABLE 1

| | Peptide^{b} | Sequence of Peptide^{a} |
|---|---|---|
| Monomer | HRC₄₅₀₋₄₈₅ | Ac-PPISLERLDVGTNLGNAIAKLEDAKELLESSDQ |
| | | ILR-GSGSG-C (SEQ ID NO. 49) |
| | FIP-HRC₄₅₀₋₄₈₅ | Z-D-FFG-GSGSG-PPISLERLDVGTNLGNAIAKLEDAKELLESSDQ |
| | | ILR-GSGSG-C (SEQ ID NO. 50) |
| | FIP-HRC₄₅₀₋₄₈₅ | Z-D-FFG-GSGSG-PPISLERLDVGTNLGNAIAKLEDAKELLESSDQ |
| | _{(no GSGSG)} | ILR-C (SEQ ID NO. 51) |
| | FIP | Z-D-FFG-GSGSG-C (SEQ ID NO. 52) |
| | FIP_{(no GSGSG)} | Z-D-FFG-C (SEQ ID NO. 53) |
| | Lipid-Peptide | |
| | Conjugate | General Structure of Lipid-peptide Conjugate^{d} |
| Monomer Chol | HRC₄₅₀₋₄₈₅-chol FIP-HRC₄₅₀₋₄₈₅- chol FIP-chol | 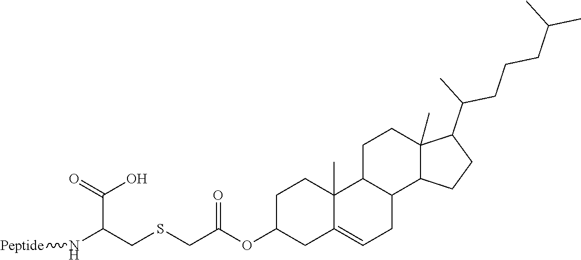 |
| ^{c}Monomer Peg Chol | HRC₄₅₀₋₄₈₅_peg₄- chol FIP-HRC₄₅₀₋₄₈₅- peg₄-chol FIP-peg₄-chol | 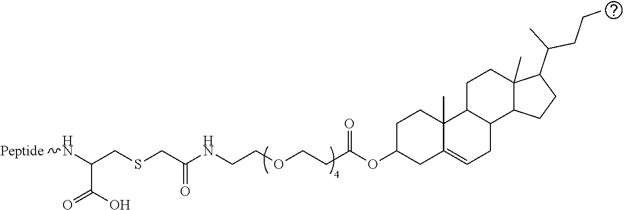 |
| Dimer Peg | [HRC₄₅₀₋₄₈₅]₂-peg₁₁ [FIP-HRC₄₅₀₋₄₈₅]₂- peg₁₁ [FIP]₂-peg₁₁ | 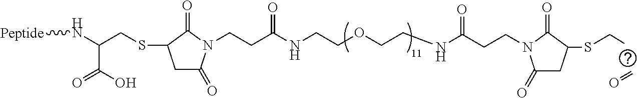 |
| Dimer Peg Chol | [HRC₄₅₀₋₄₈₅-peg₄]₂- chol [FIP-HRC₄₅₀₋₄₈₅- peg₄]₂-chol [FIP-peg₄]₂-chol [HRC₄₅₀₋₄₈₅-peg₄]₂- chol | 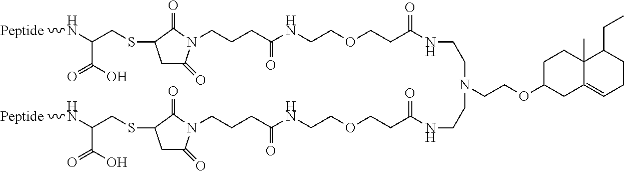 |

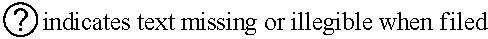

Table 2 shows inhibitory activity of FIP, HRC, and FIP-HRC peptides in fusion assays (salient peptides). The beta-galactosidase complementation-based fusion assay was performed. Briefly, 293T cells transiently transfected with Nectin-4 and the omega reporter subunit (target cells), were incubated for the indicated period with cells co-expressing viral glycoproteins (H and F) and the alpha reporter subunit (effector cells), in the presence or absence of fusion inhibitor peptides. In the absence of peptides, the fusion between the target and effector cells lead to the reconstitution of the beta-galactosidase activity that is quantified using the luminescence-based kit Galacto-Star™ β-Galactosidase Reporter Gene (ThermoFisher, US). In the presence of peptides, fusion is reduced and, as a result, the beta-galactosidase activity is reduced as well. Data are from at least three independent experiments.

TABLE 2

| | | Nectin-4 | |
|---|---|---|---|
| | Peptide | IC₅₀ nM | IC₉₀ nM |
| Monomer | HRC₄₅₀₋₄₈₅ | >10000 | >10000 |
| | FIP-HRC₄₅₀₋₄₈₅ | 4244.6 ± 811.5 | >10000 |
| | FIP | 9716.6 ± 2730 | >10000 |
| Monomer | HRC₄₅₀₋₄₈₅-Chol | 102.7 ± 10.2 | >7000 |
| Chol | FIP-HRC₄₅₀₋₄₈₅-chol | 213.8 ± 148 | 614.1 ± 244.7 |
| | FIP-chol | 4004.3 ± 1082.8 | 7615.1 ± 13 |
| Monomer | HRC₄₅₀₋₄₈₅-peg₄-Chol | 123 ± 10.5 | 855.7 ± 78.6 |
| Peg Chol | FIP-HRC₄₅₀₋₄₈₅-peg₄- | 213.8 ± 148 | 614.1 ± 244.7 |
| | chol | | |
| | FIP-peg₄-chol | 4004.3 ± 1082.8 | 7615.1 ± 13 |
| Dimer | [HRC₄₅₀₋₄₈₅]₂-peg₁₁ | >4000 | >10000 |
| Peg | [FIP-HRC₄₅₀₋₄₈₅]₂-peg₁₁ | 528.1 ± 635 | 1177 ± 979 |
| | [FIP]₂-peg_{11 (no GSGSG)} | >10000 | >10000 |
| Dimer | [HRC₄₅₀₋₄₈₅-peg₄]₂-chol | 125 ± 175.3 | 378.5 ± 299.8 |
| Peg Chol | [FIP-HRC₄₅₀₋₄₈₅-peg₄]₂- | 23 ± 13.18 | 105.1 ± 56.1 |
| | chol | | |
| | [FIP-peg₄]₂-chol | 452 ± 195.7 | 1844.8 ± 16.04 |

Table 3 shows inhibitory activity of FIP, HRC, and FIP-HRC peptides in fusion assays (extended version). The beta-galactosidase complementation-based fusion assay was performed as described previously. Briefly, 293T cells transiently transfected with either Nectin-4 or CD150 and the omega reporter subunit (target cells) were incubated for the indicated period with cells co-expressing viral glycoproteins (H and F) and the alpha reporter subunit (effector cells), in the presence or absence of fusion inhibitor peptides. In the absence of peptides, the fusion between the target and effector cells lead to the reconstitution of the beta-galactosidase activity that is quantified using the luminescence-based kit Galacto-Star™ 0-Galactosidase Reporter Gene (ThermoFisher, US). In the presence of peptides, fusion is reduced and, as a result, the beta-galactosidase activity is reduced as well. Data are from three independent experiments (except for *=9 replicates, § =6 replicates, and °=5 replicates).

TABLE 3

| | | Nectin4 | | CD150 | |
|---|---|---|---|---|---|
| | Peptide | IC₅₀ nM | IC₉₀ nM | IC₅₀ nM | IC₉₀ nM |
| Monomer | HRC₄₅₀₋₄₈₅ | >10000 | >10000 | ND | ND |
| | FIP-HRC₄₅₀₋₄₈₅ | 4244.6 ± 811.5 | >10000 | ND | ND |
| | FIP-HRC_{450-485 (no GSGSG)} | 2768 ± 1644.8 | 6370.6 ± 4184.1 | ND | ND |
| | FIP | 9716.6 ± 2730 | >10000 | ND | ND |
| Monomer | FIP-HRC₄₅₀₋₄₈₅ | >1000 | >1000 | >1000 | >1000 |
| N- | FIP-HRC_{450-485 (no GSGSG)} | >700 | >10000 | >1000 | >1000 |
| Maleimide | FIP (no GSGSG*) | >1000 | >1000 | >1000 | >1000 |
| Monomer | HRC₄₅₀₋₄₈₅-chol | 102.7 ± 10.2 | >1000 | ND | ND |
| Chol | FIP-HRC₄₅₀₋₄₈₅-chol § | 251.3 ± 191.5 | 445.6 ± 368.6 | 184.6 ± 46 | 845 ± 26.9 |
| | FIP-HRC₄₅₀₋₄₈₅-chol | 85.9 ± 56.26 | 539.7 ± 338.1 | 70.8 ± 65.32 | 388.6 ± 22.1 |
| | (no GSGSG*) | | | | |
| | FIP-chol | >10000 | >10000 | ND | ND |
| | FIP-chol (no GSGSG*) | >1000 | >1000 | >1000 | >1000 |
| Monomer | HRC₄₅₀₋₄₈₅-peg₄-chol | 123 ± 10.5 | 855.7 ± 78.6 | ND | ND |
| Peg Chol | FIP-HRC₄₅₀₋₄₈₅-peg₄- | 213.8 ± 148 | 614.1 ± 244.7 | 291.3 ± 293 | >700 |
| | chol | | | | |
| | FIP-HRC₄₅₀₋₄₈₅-peg₄- | 707 ± 507.4 | 824.7 ± 229.1 | 78.5 ± 33.2 | 278 ± 26.6 |
| | chol (no GSGSG*) | | | | |
| | FIP-peg₄-chol § | 4004.3 ± 1082.8 | 7615.1 ± 13 | ND | ND |
| | FIP-peg₄-chol (no | >700 | >1000 | >400 | >900 |
| | GSGSG*) | | | | |
| Dimer Peg | [HRC₄₅₀₋₄₈₅]₂-peg₁₁ | >4000 | >10000 | ND | ND |
| | [FIP-HRC₄₅₀₋₄₈₅]₂- | 528.1 ± 635 | 1177 ± 979 | 278 ± 229.1 | 916.5 ± 162 |
| | peg₁₁ | | | | |
| | [FIP-HRC₄₅₀₋₄₈₅]₂- | >700 | >1000 | 516.5 ± 105.3 | >1000 |
| | peg_{11 (no GSGSG)} | | | | |
| | [FIP]₂-peg_{11°} | >10000 | >10000 | >1000 | >1000 |
| | [FIP]₂-peg_{11 (no GSGSG)} | >700 | >1000 | >600 | >1000 |
| Dimer Peg | [HRC₄₅₀₋₄₈₅-peg₄]₂- | 125 ± 175.3 | 378.5 ± 299.8 | 100 ± 65.1 | 697.6 ± 22.27 |
| Chol | chol* | | | | |
| | [FIP-HRC₄₅₀₋₄₈₅-peg₄]₂- | 23 ± 13.18 | 105.1 ± 56.1 | 25.66 ± 11.7 | 124.5 ± 10.03 |
| | chol* | | | | |
| | [FIP-HRC₄₅₀₋₄₈₅-peg₄]₂- | 60.2 ± 65.45 | 219.56 ± 189 | 58.9 ± 69.85 | 286.6 ± 24.5 |
| | chol (no GSGSG*) | | | | |
| | [FIP-peg₄]₂-chol* | 452 ± 195.7 | 1844.8 ± 16.04 | 510 ± 360.2 | 927 ± 358.7 |
| | [FIP-peg₄]₂-chol (no | >1000 | >1000 | >700 | >1000 |
| | GSGSG*) | | | | |
| peg24 chol | [FIP-HRC450-485] | 720 ± 484.9 | 885.5 ± 102.7 | 725.3 ± 475.7 | >900 |
| | [FIP-HRC450-485] (no | 381.6 ± 271.1 | >700 | 480 ± 87.7 | >900 |
| | GSGSG*) | | | | |
| | [FIP] (no GSGSG*) | 113.6 ± 34.4 | 567.6 ± 186.2 | 89.5 ± 46.3 | 543.6 ± 34.8 |

*SEQ ID NO. 7

### Peptide Conjugation Induces Self-Association in Solution

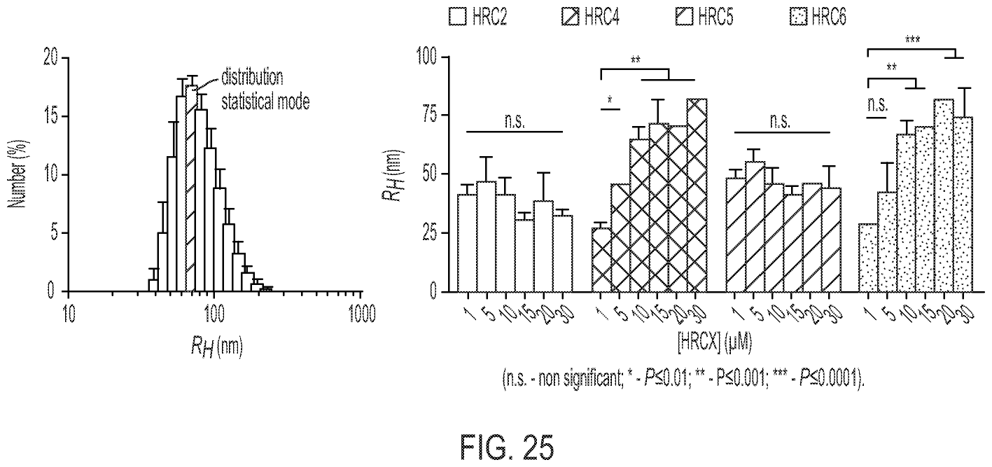

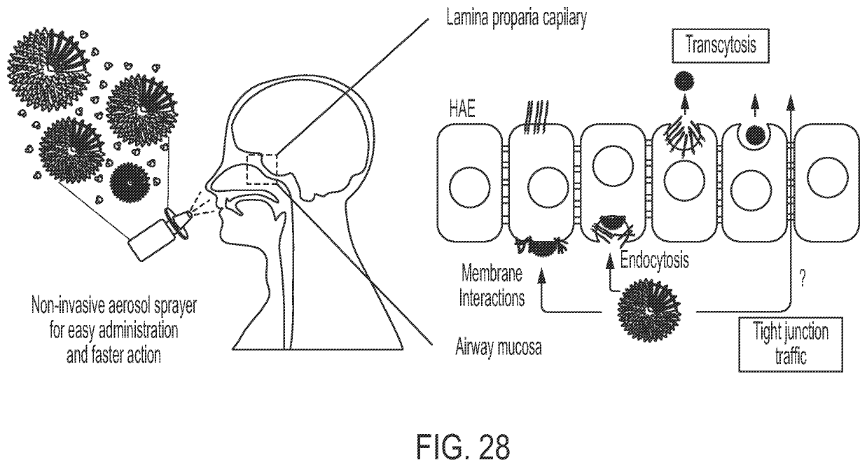

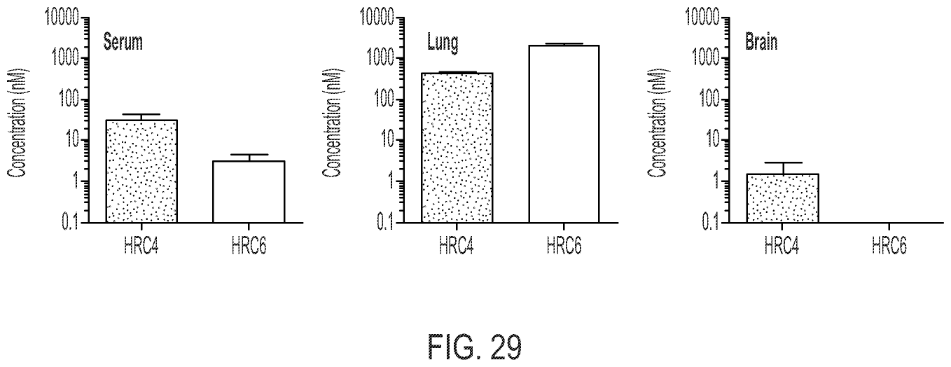

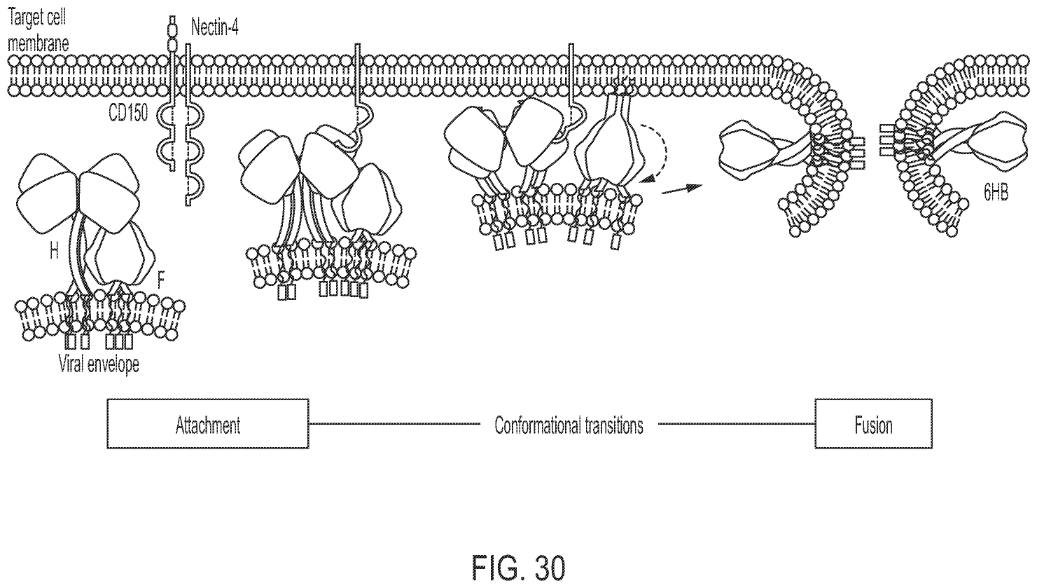

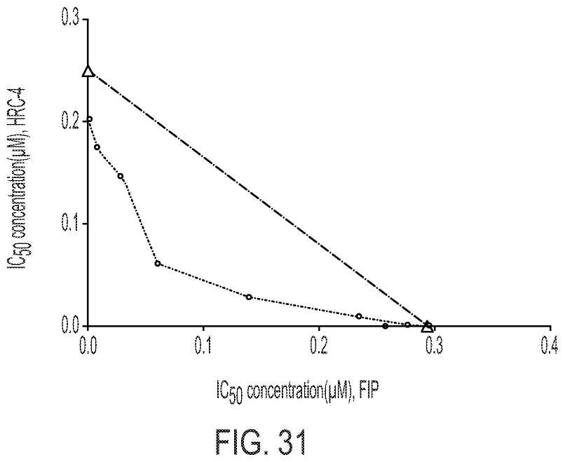

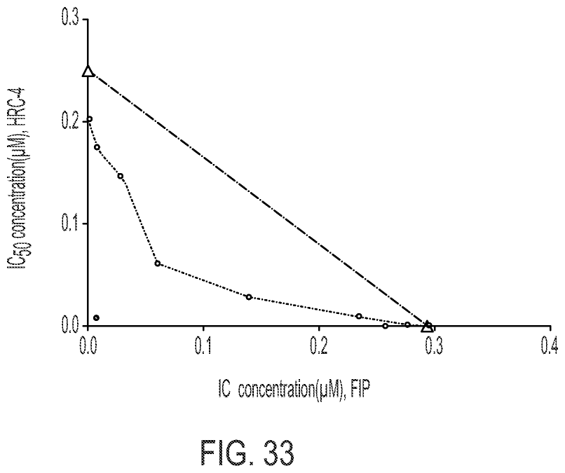

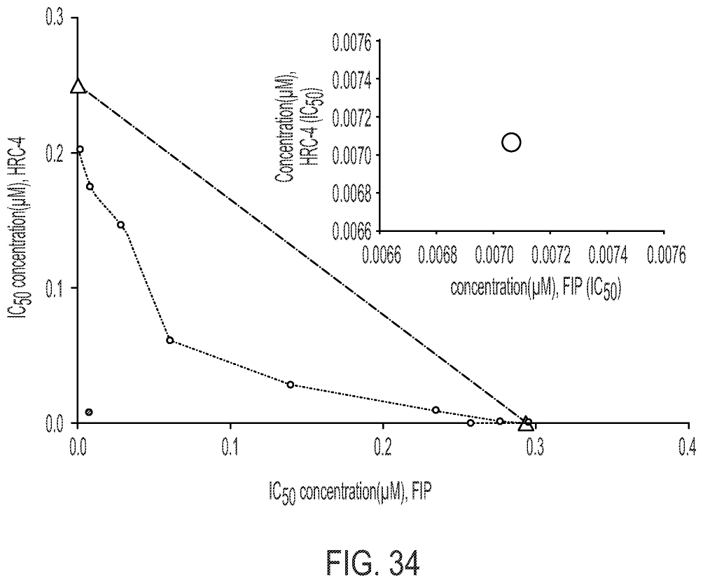

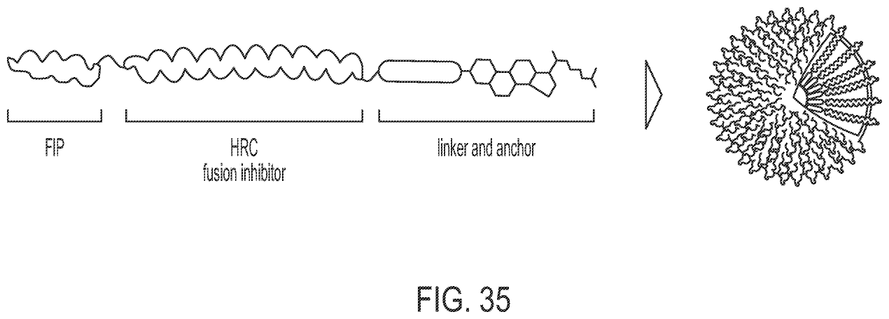

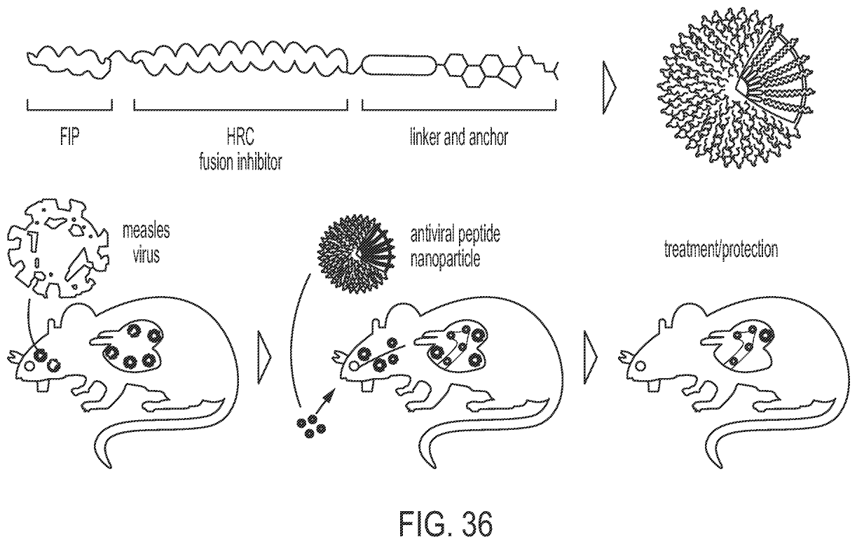

FIG. 25 shows that peptide particle size is in the nanomolar range. FIG. 26 shows that amphipathic structure drives self-assembly and nanoparticle formation. FIG. 27 shows that nanoparticles (dis)assemble at lipid membrane interfaces with peptide retention. FIG. 28 shows that nanoparticles are able to cross the HAE barrier and are bioavailable in vivo. By reaching relevant sites of infection, these prevent measles virus multiplication. FIG. 29 shows that conjugated peptides have improved biodistribution in the cotton rat model with intranasal administration route at 6 mg/kg. FIG. 30 shows combinatorial strategy where fusion inhibitory peptide (FIP) binds to the fusion protein and stabilizes the pre-fusion state of the measles F protein. FIG. 31 depicts an isobologram curve which shows synergism between FIP and HRC peptides (combinatorial drug testing: simultaneous treatment of 2 different compounds). FIG. 32 shows that FIP added to the HRC region enhances the antiviral activity. As shown in FIG. 33 and FIG. 34, when FIP and HRC are in the same structure, the potency transcends the synergism of the two inhibitors added together. FIGS. 35 and 36 show that FIP added to the HRC region enhances the antiviral activity.

Peptide antiviral approach against MV IC323eGFP summary:
Viral stock: IC323-EGFP, P3 (ALa) titre: 2×106 pfu/ml
Peptide stock: Peptide MeV HRC 4 concentration: 50 mg/ml
Treatment dose ˜1 mg/kg or ˜0.1 mg/kg, 24 h or 6 h pre-infection
Mice: SLAM IFNAR KO (3, 5 to 4 weeks old)
Intranasal infection (5 uL/nostril=>10 uL/mouse)
Infection dose=4×LD50
20.000 pfu/mouse

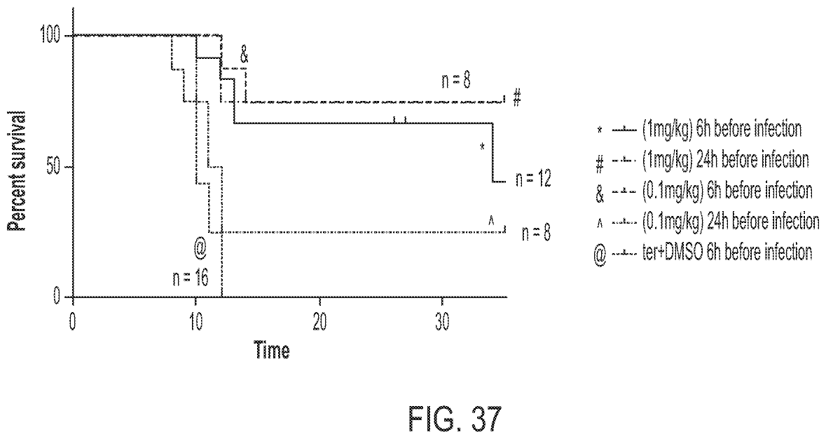

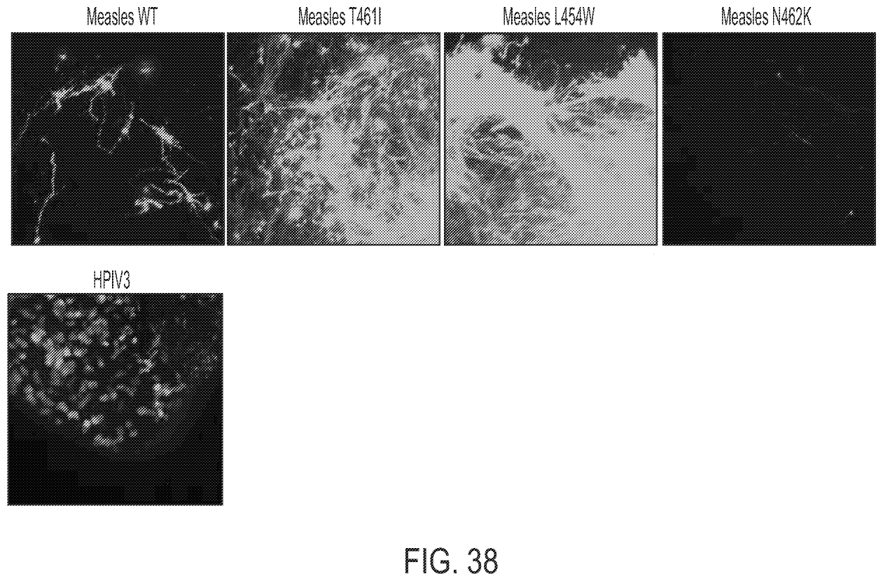

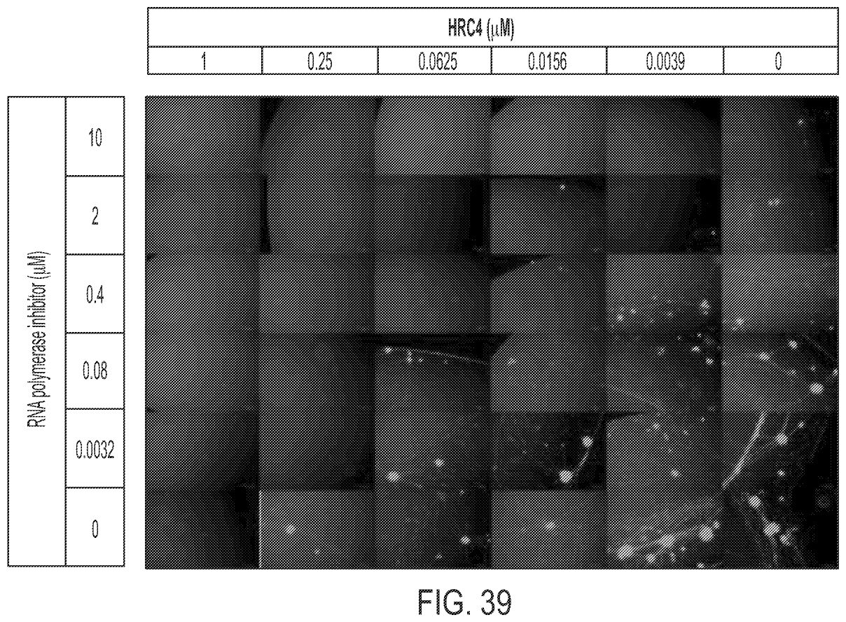

FIG. 37 shows survival proportions. Survival of SLAM IFNAR KO mice was 3.5 weeks. FIG. 38 shows various MeVs. FIG. 39 shows that MV HRC4 peptide and RNA polymerase inhibitor block MeV wild-type infection in human motor neurons. Days of infection: 8 days. Green fluorescent indicates MeV infection and it is directly proportional to viral infection.

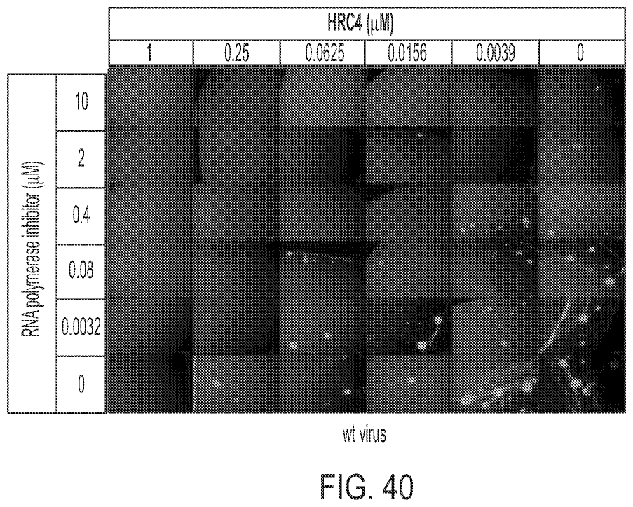

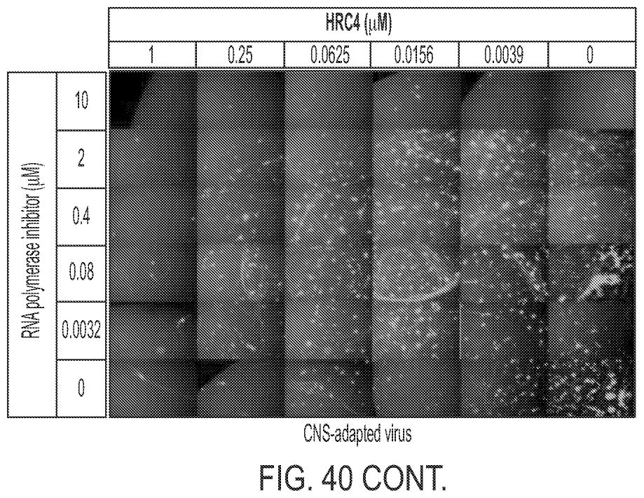

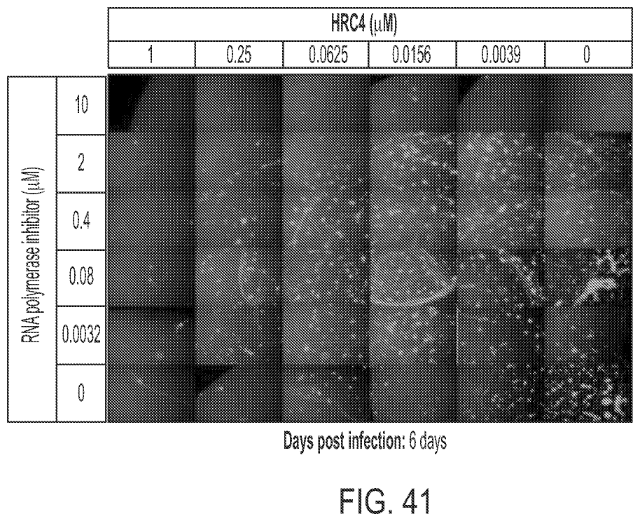

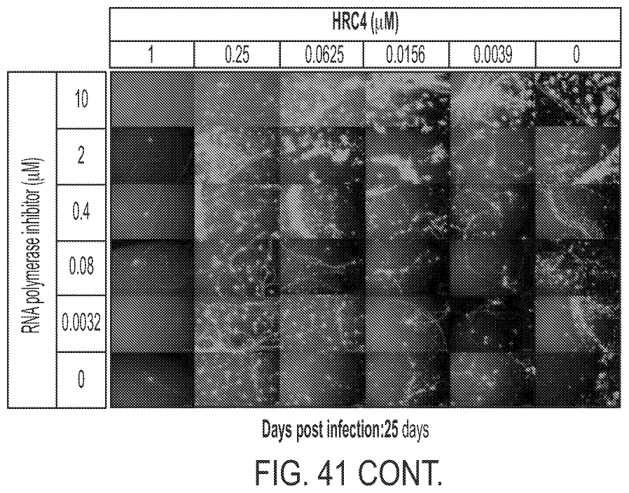

MeV HRC4 peptide and RNA polymerase inhibitor block the spread of virus in motor neurons. The information about the polymerase inhibitor has been described in Science Translational Medicine 16 Apr. 2014:Vol. 6, Issue 232, pp. 232. FIG. 40 shows HRC4 peptide and RNA polymerase inhibitor: wild-type and CNS-adapted viruses. FIG. 41 shows MeV HRC4 peptide and RNA polymerase inhibitor vs. CNS-adapted MV in human motor neurons. Over time, the polymerase inhibitor activity decreased.

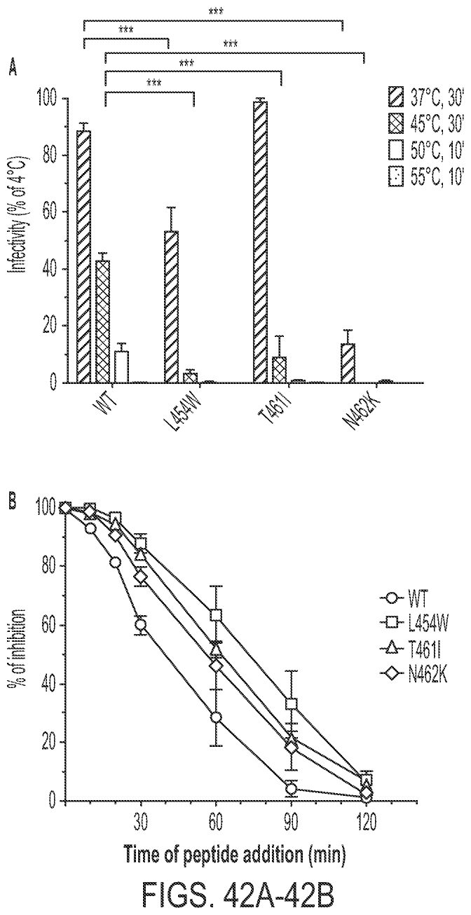

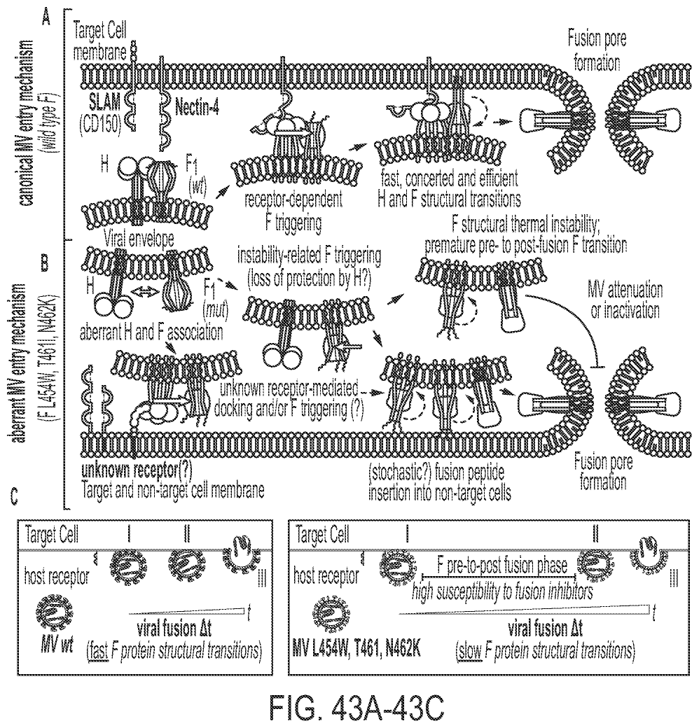

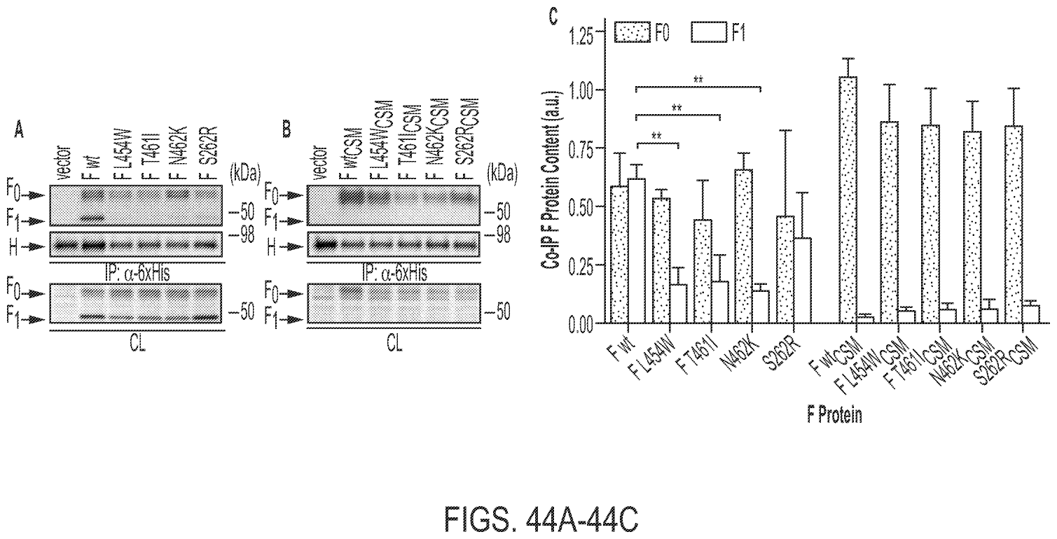

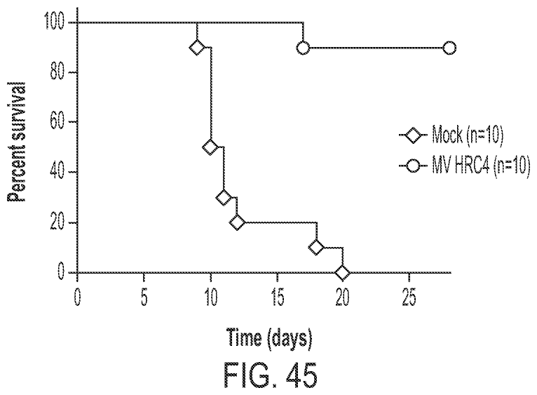

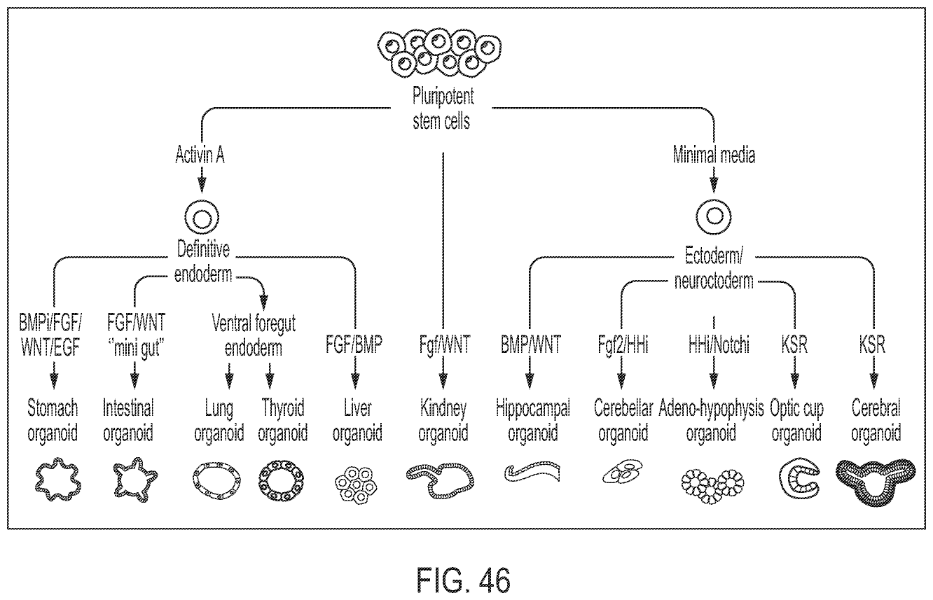

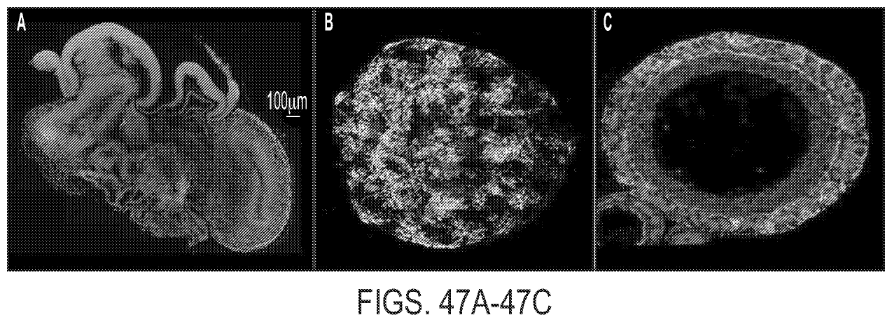

MeV HRC4 peptide blocks spread of virus only at the highest concentration. FIGS. 42A-B and FIG. 43A-C show steps in entry. FIGS. 44A-C show that H-F interaction is altered in L454W F. FIG. 45 shows that intranasal administration of MeV HRC4 protects IFNAR KO mice from lethal MeV encephalitis. FIG. 46 shows a schematic of the various organoids that can be grown from pluripotent stem cells and the developmental signals that are employed. FIGS. 47A-C shows a “mini-brain” generated from pluripotent stem cells. FIG. 47A shows a complex morphology with heterogeneous regions containing neural progenitors (SOX2, red) and neurons (TUJ1, green) is apparent (Lancaster et al., 2013). FIG. 47B shows an immunofluorescent image of an entire kidney organoid grown from pluripotent stem cells with patterned nephrons. Podocytes of the forming glomeruli (NPHS1, yellow), early proximal tubules (lotus tetragonolobus lectin, pink), and distal tubules/collecting ducts (E-Cadherin, green). FIG. 47C shows 3D reconstruction of the midsection of a human aSC-derived lung organoid stained for intermediate filaments of basal cells (green), the actin cytoskeleton (red), and nuclei (blue) and imaged by confocal microscopy.

### Post-Exposure Prophylaxis

In one embodiment, the subject matter disclosed herein relates to a preventive medical treatment started after exposure to the MeV in order to prevent the infection from occurring. In one embodiment, the subject matter disclosed herein relates to prophylaxis of subjects who have come into contact with MeV or are suspected to have come into contact with MeV. In one embodiment, said subjects can be administered post-exposure prophylaxis consisting of HRC peptides nanoparticles, such as HRC4 peptide nanoparticles. In one embodiment, subjects can be administered post-exposure prophylaxis consisting of FIP conjugated peptides, such as FIP conjugated HRC4 peptide nanoparticles. In one embodiment, the post-exposure prophylaxis can be administered intranasally via an internasal spray or any other suitable method know in the art. In one embodiment, the post-exposure prophylaxis can be administered subcutaneously via syringe or any other suitable method know in the art. In one embodiment, the subject matter disclosed herein can be adapted and applied to post-exposure prophylaxis for paramyxoviruses other than MeV, such as mumps. In one embodiment, the subject matter disclosed herein relates to a post-exposure prophylaxis approach of any virus by inhibiting viral fusion.

### Example 2

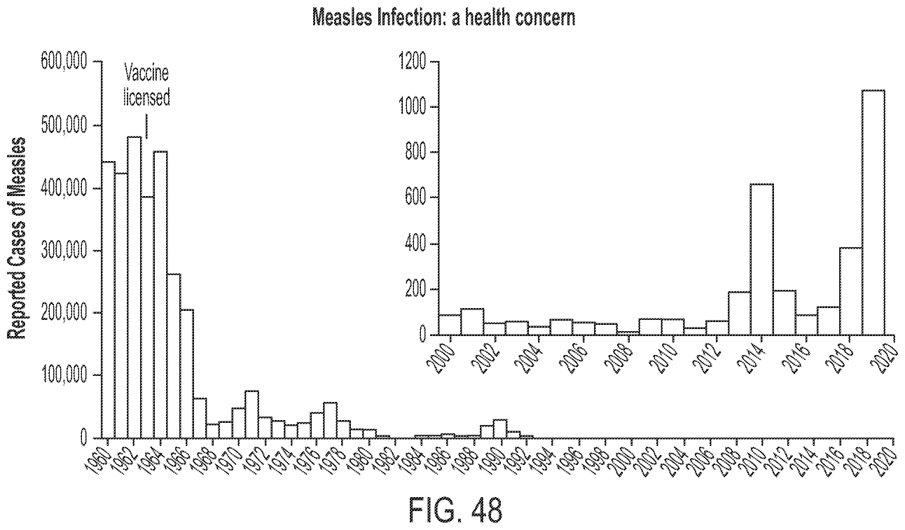

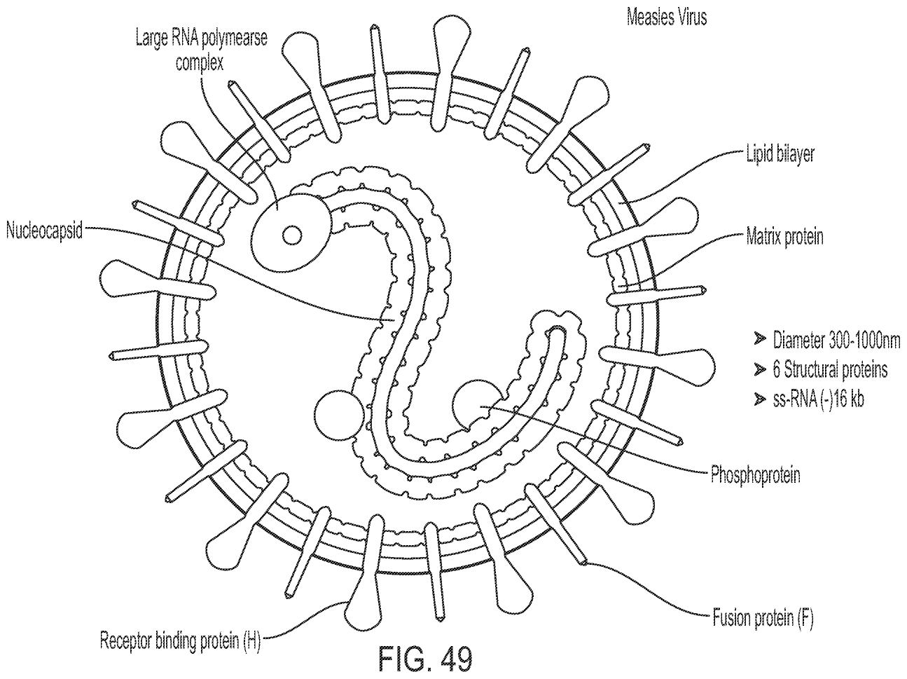

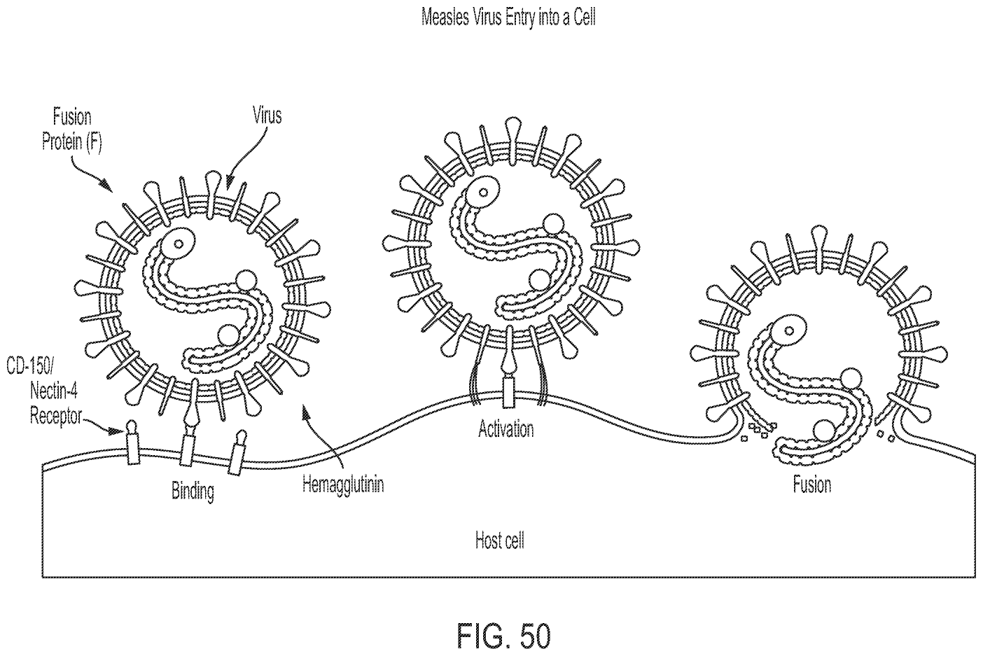

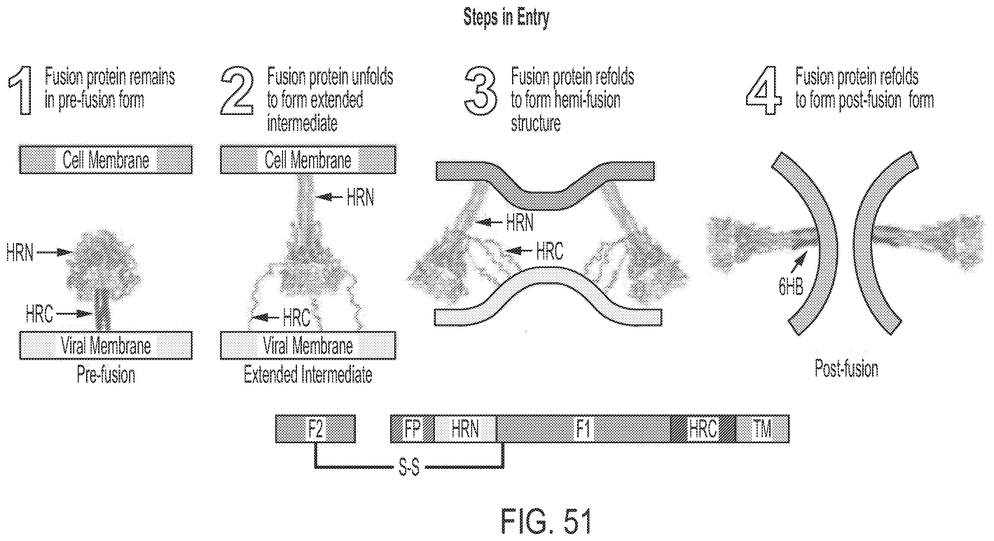

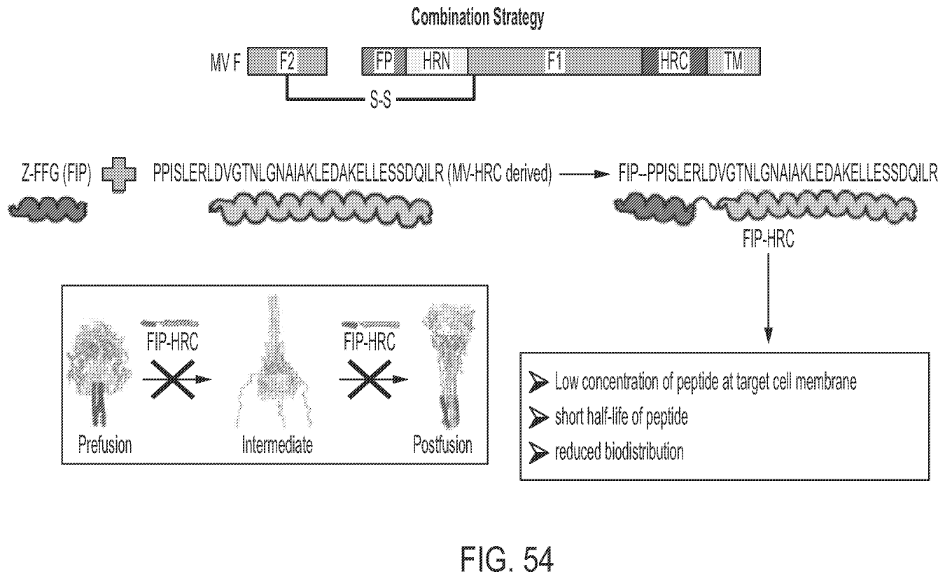

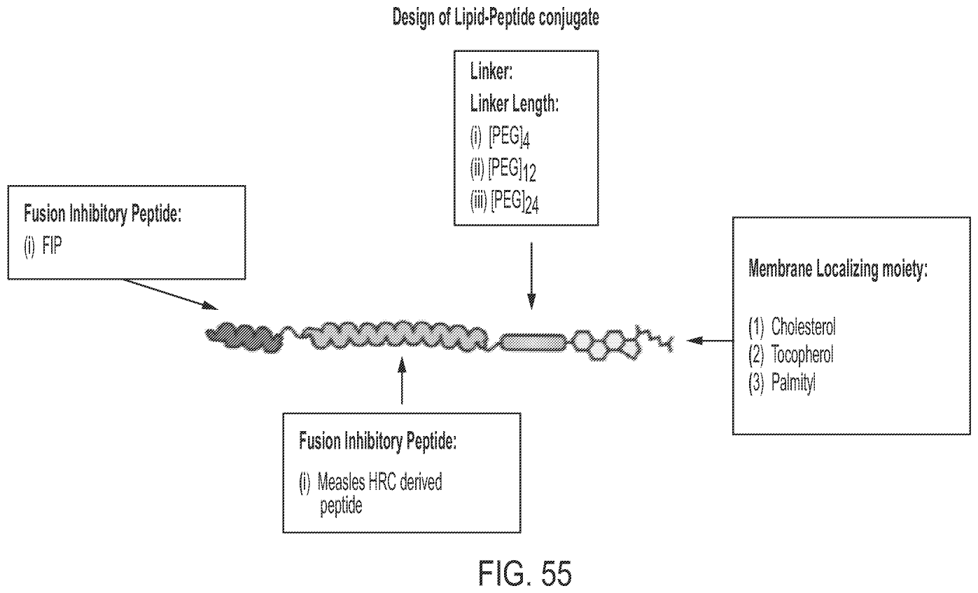

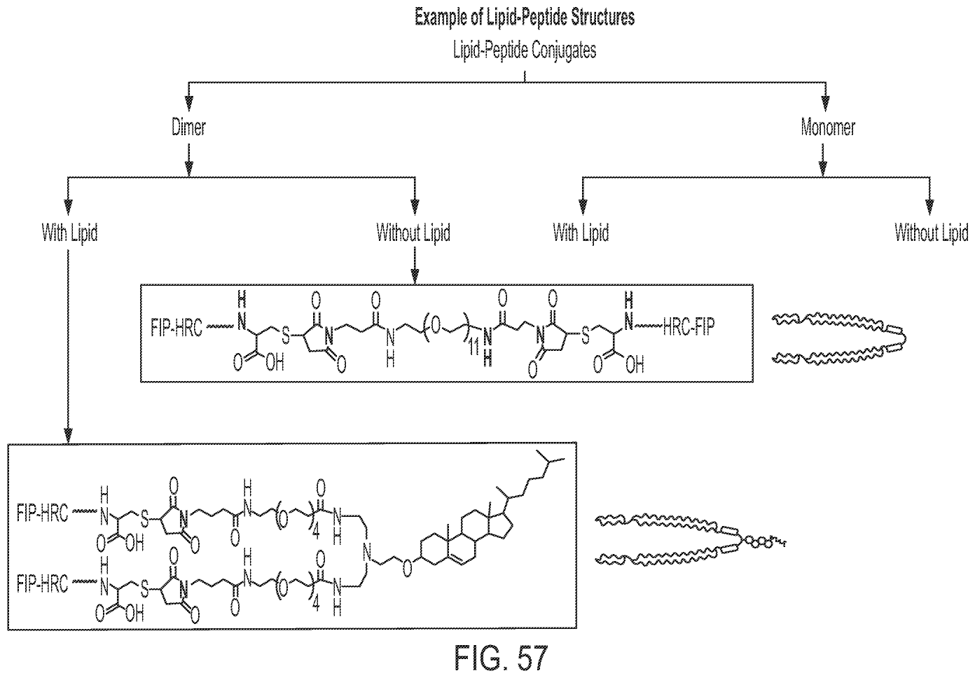

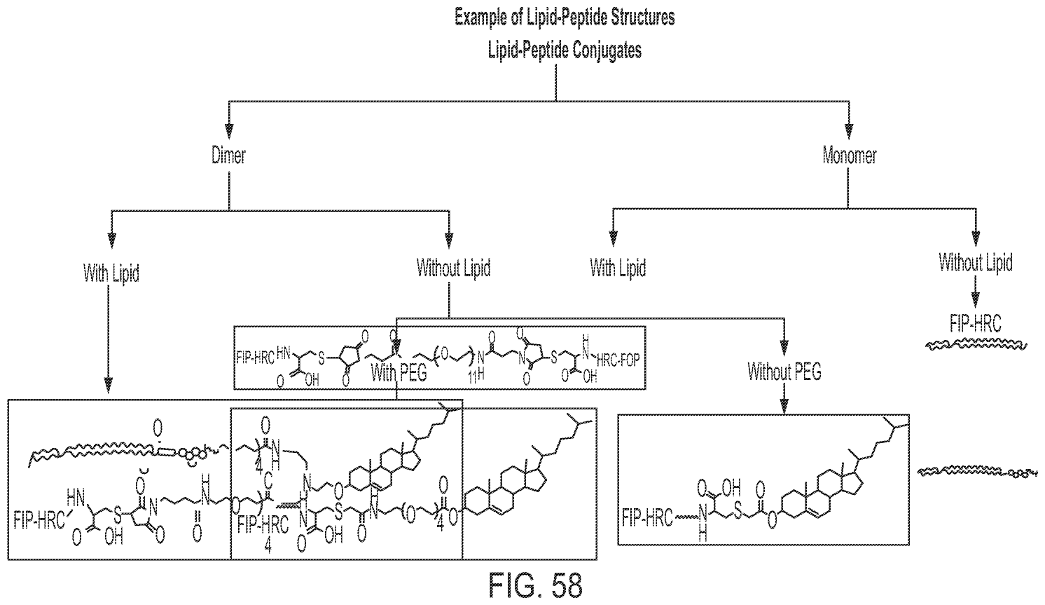

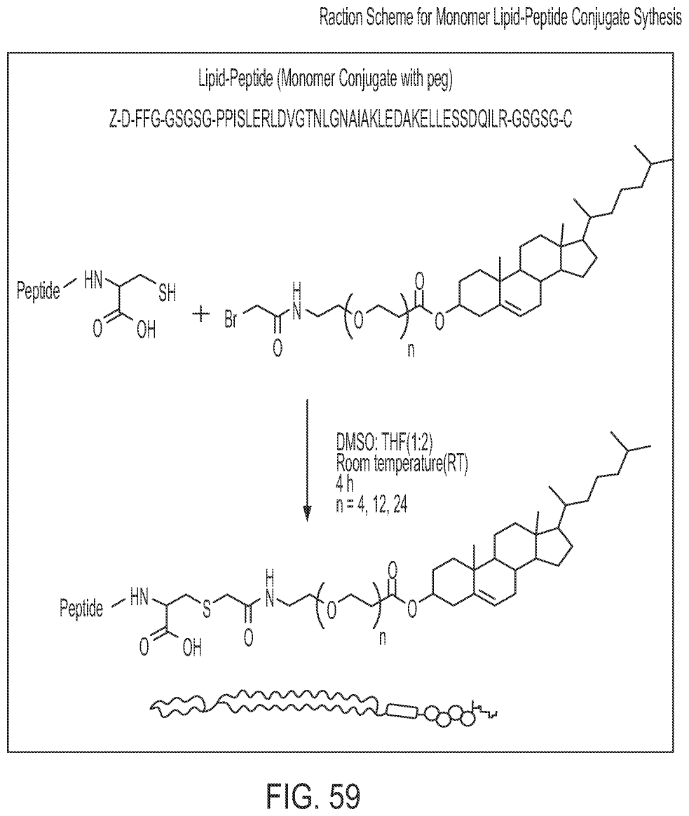

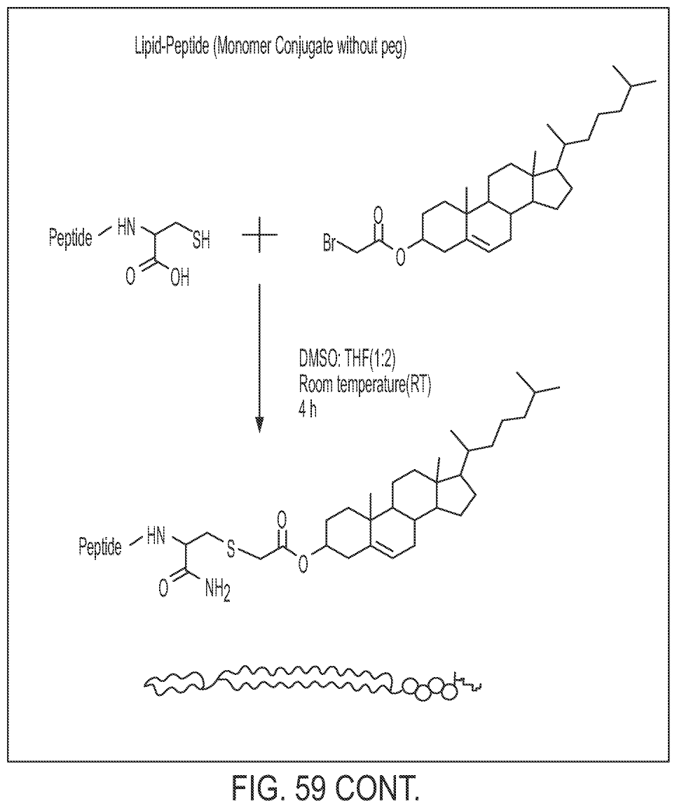

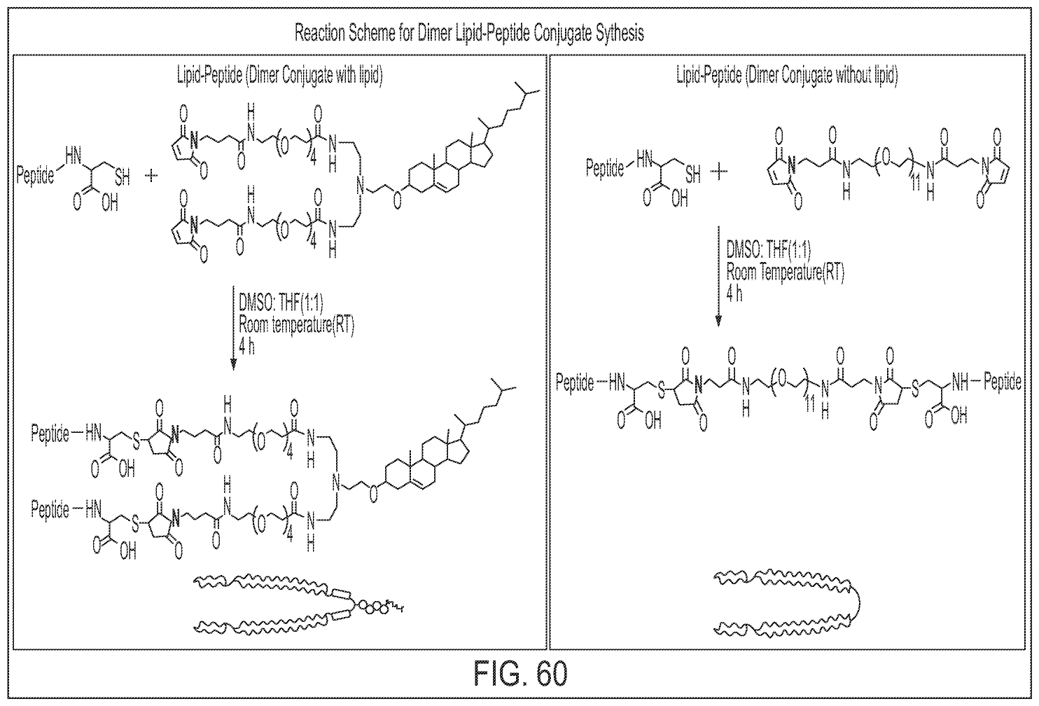

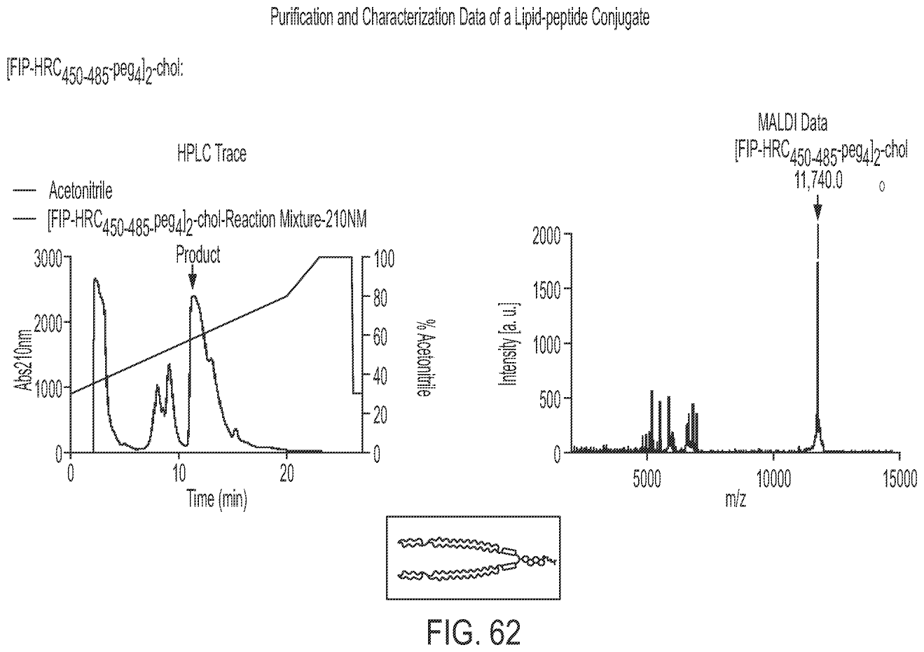

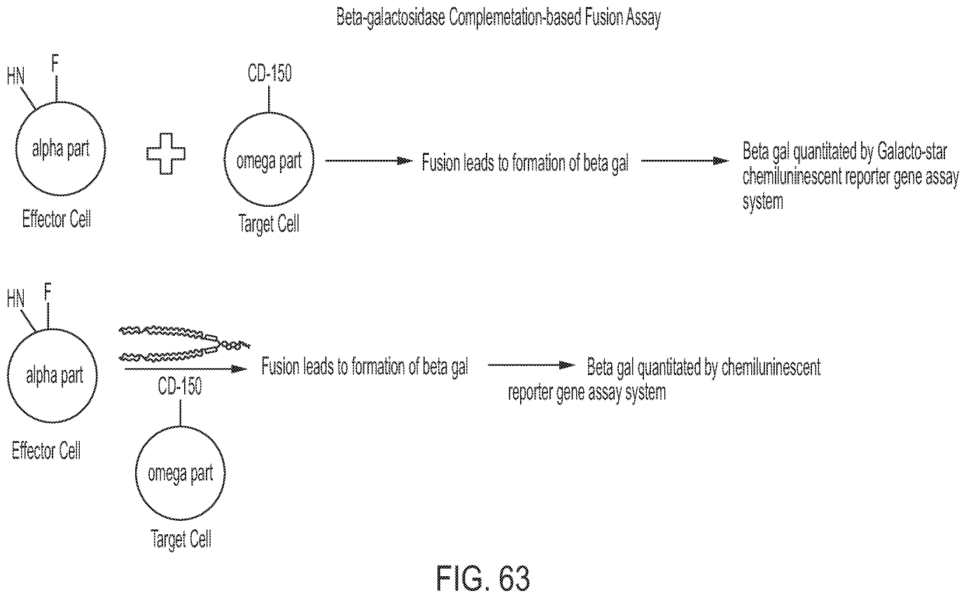

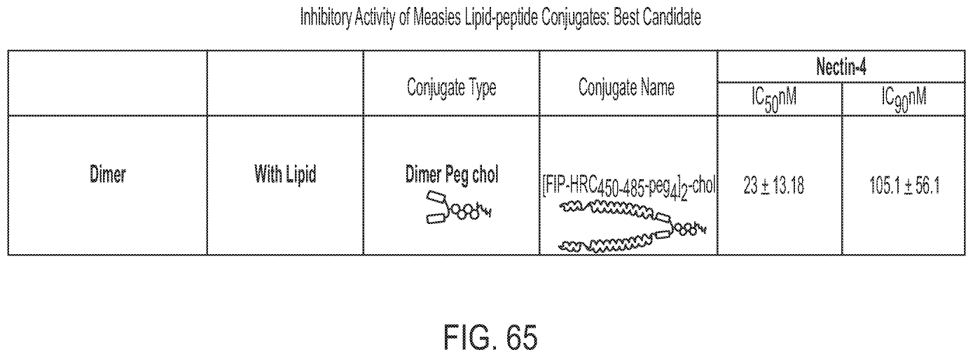

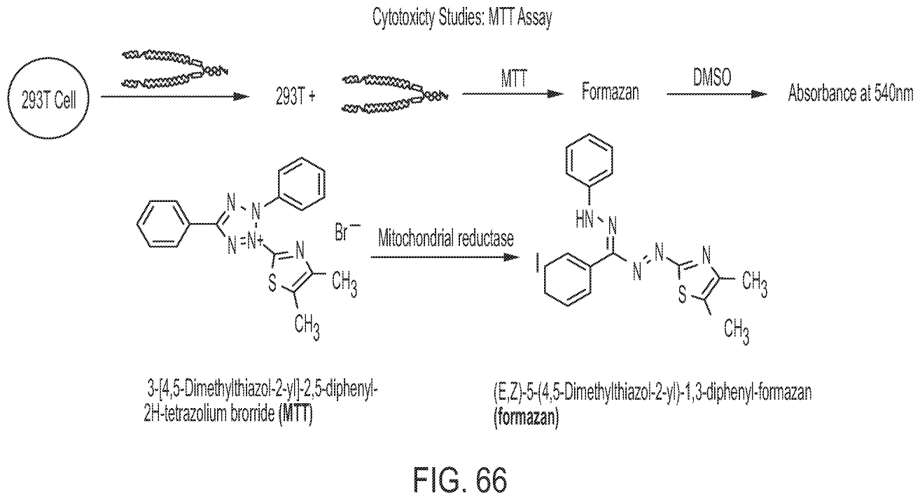

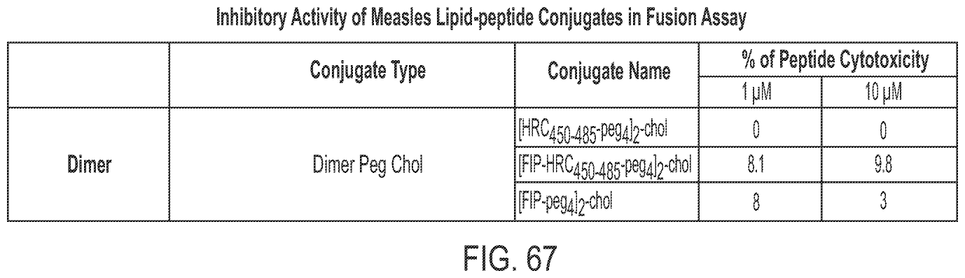

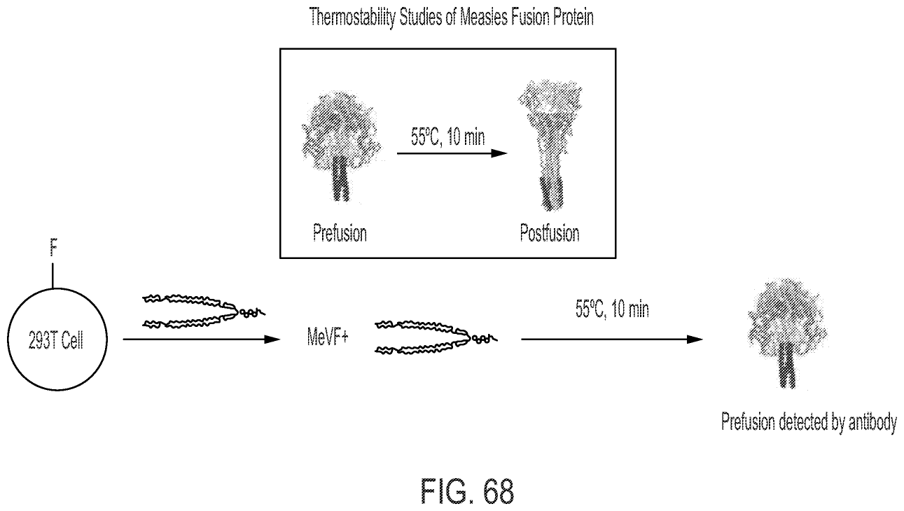

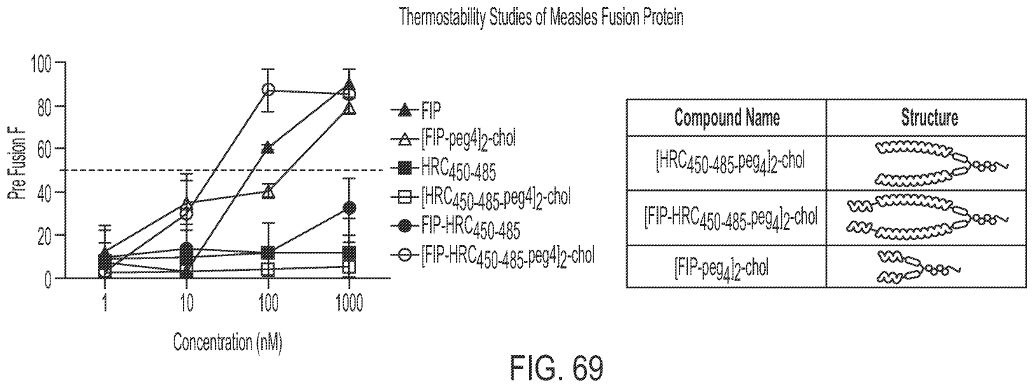

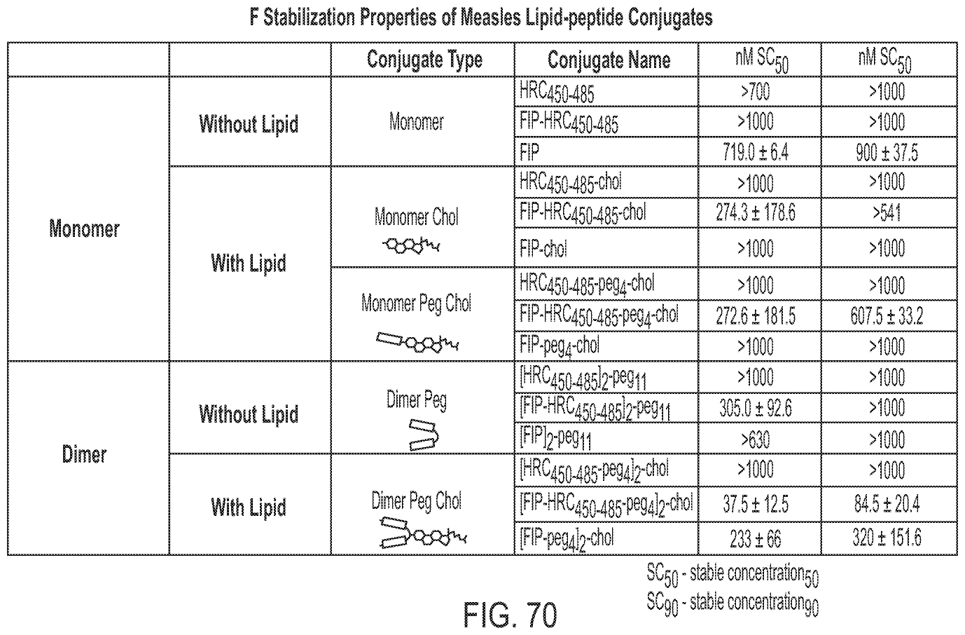

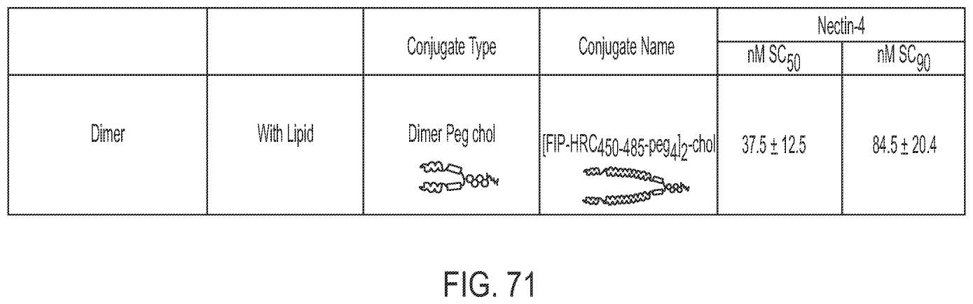

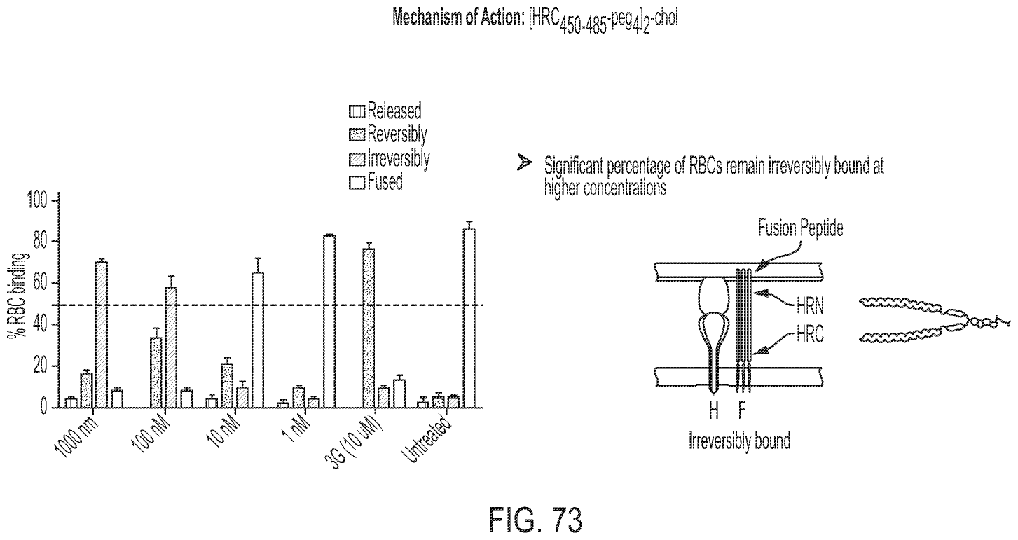

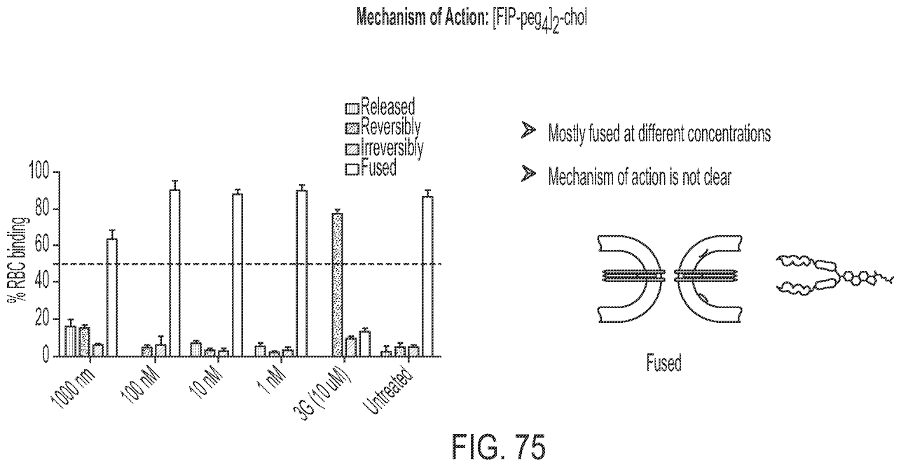

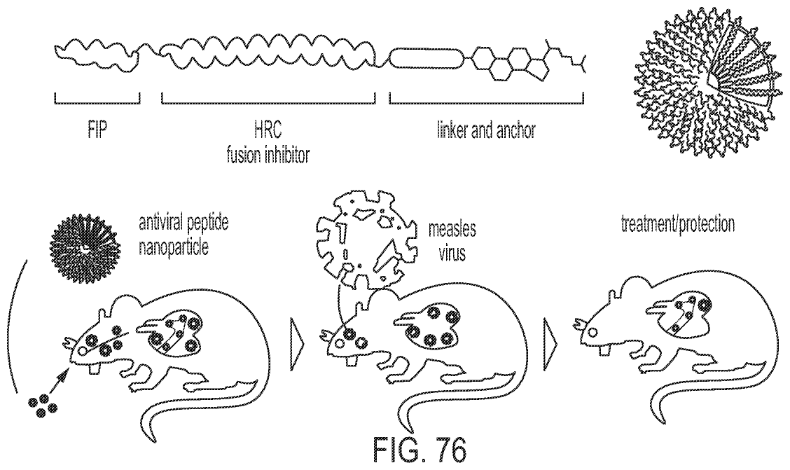

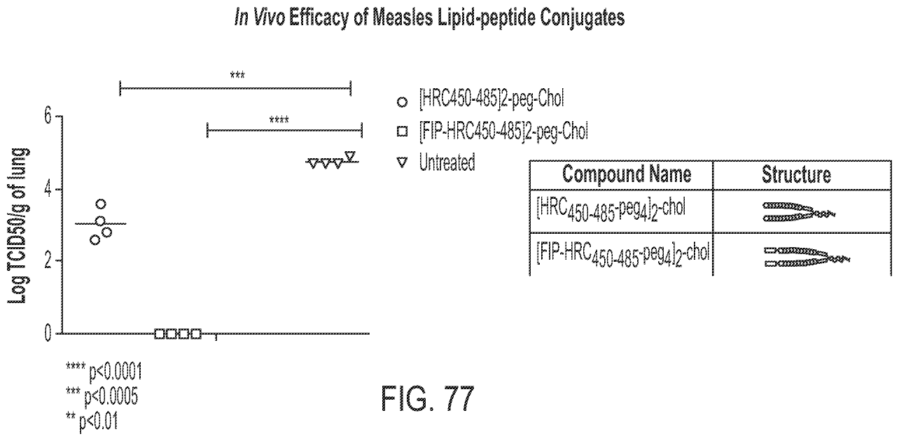

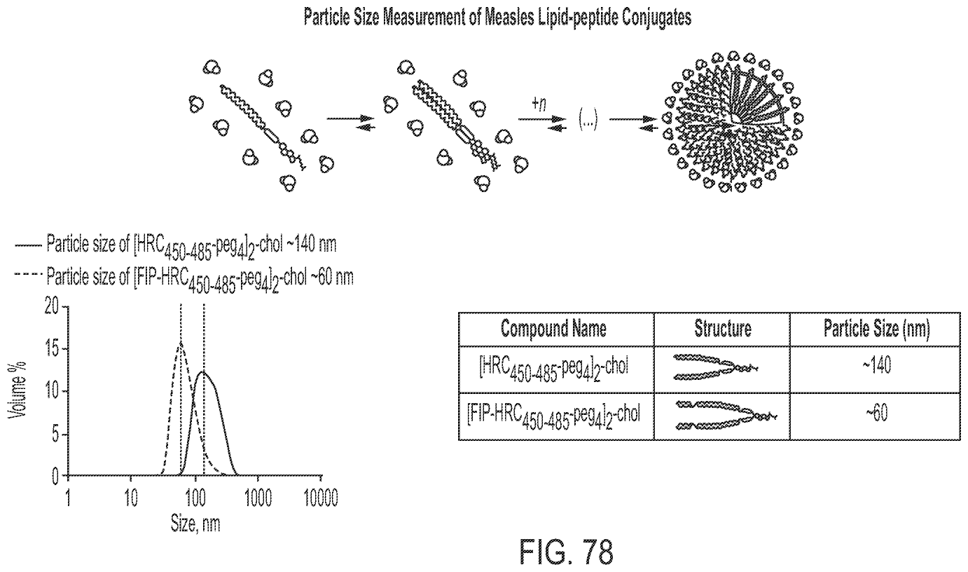

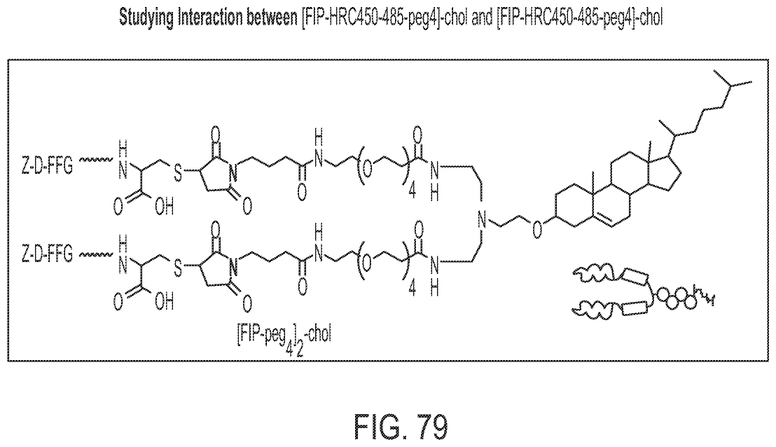

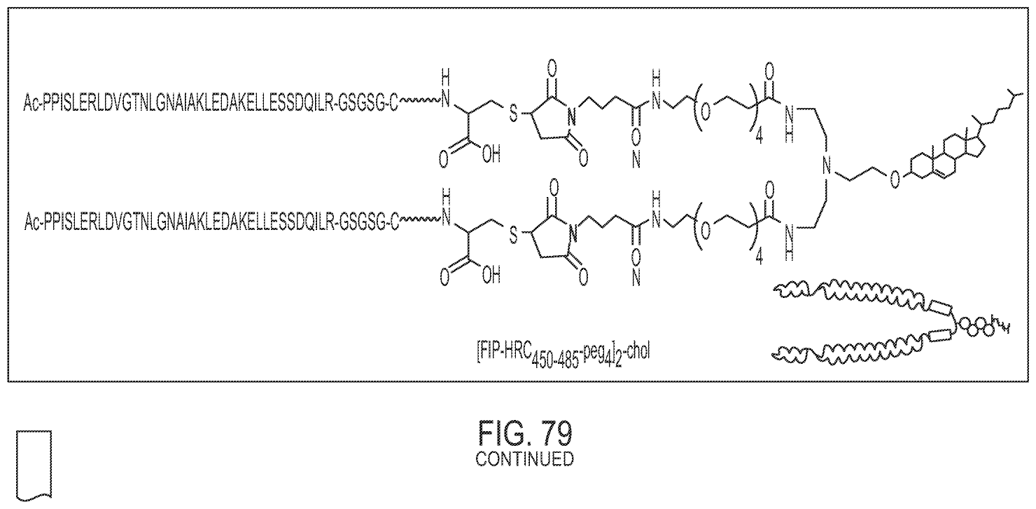

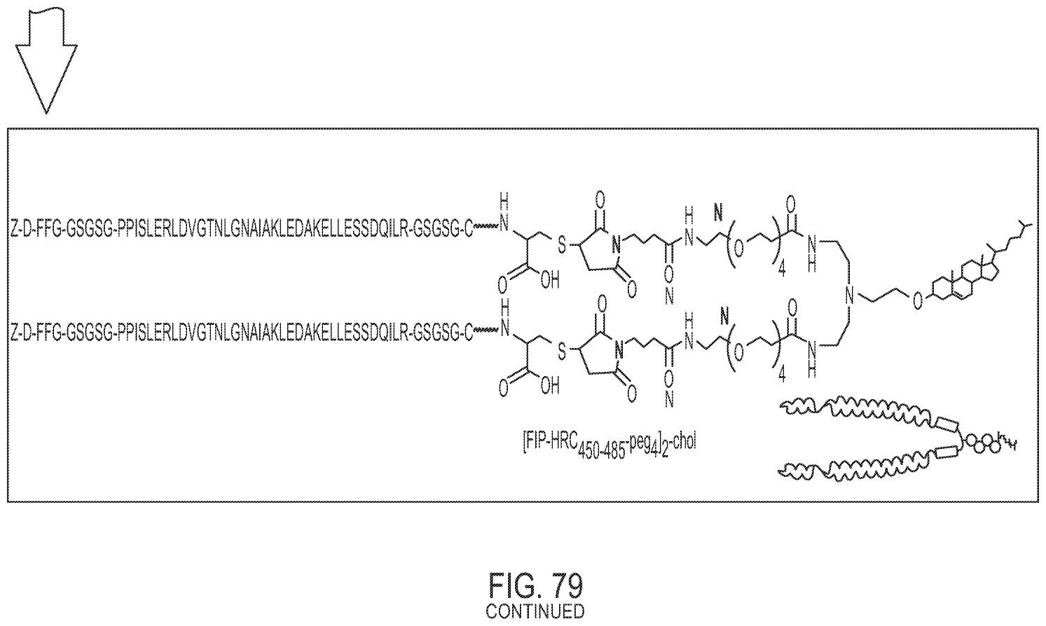

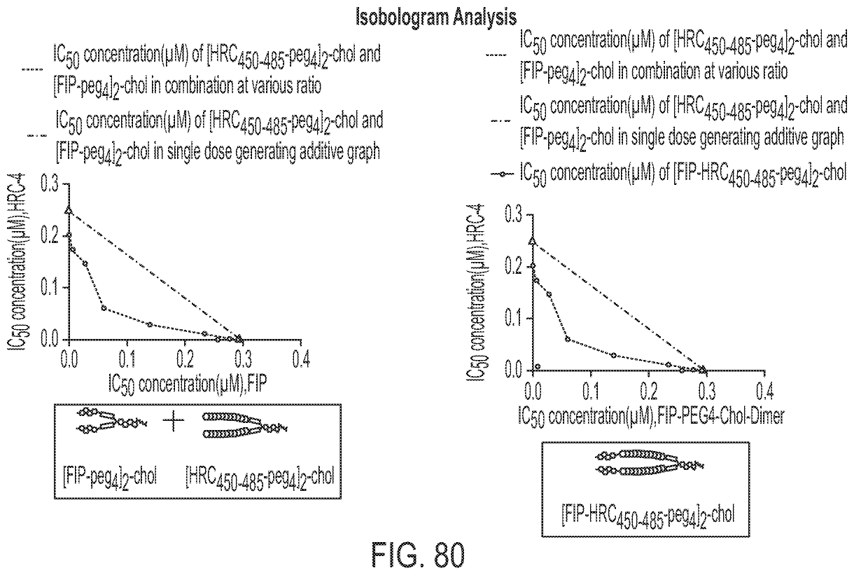

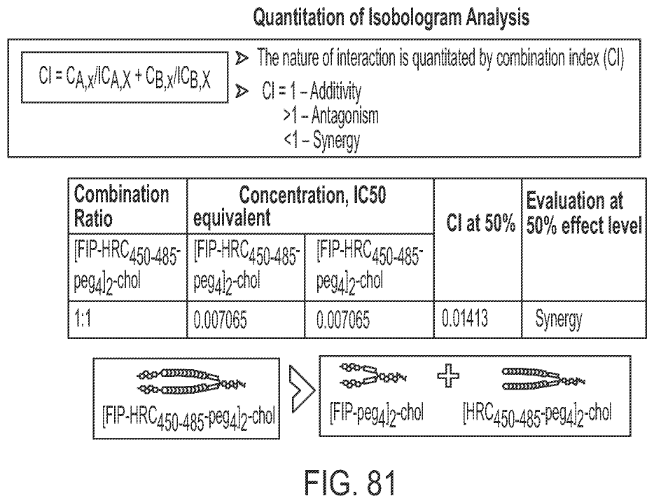

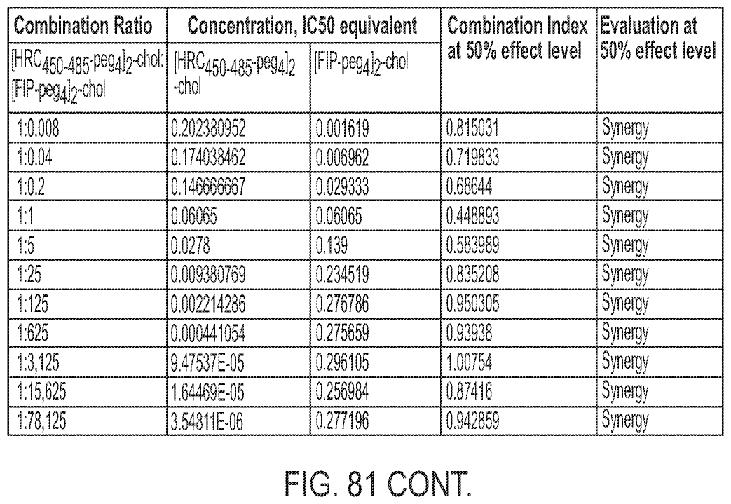

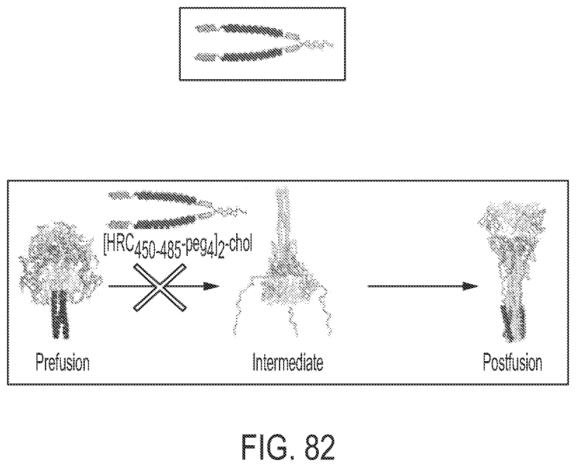

FIGS. 48-83 describe further embodiments of the invention, including certain lipid-peptide conjugates comprised of at least one fusion inhibitory peptide, a linker such as polyethylene glycol (PEG), and a membrane-localizing moiety. In some embodiments, the fusion inhibitory peptide is a measles fusion inhibitory peptide or a measles HRC-derived peptide. In some embodiments, the PEG linker is repeated or connected 4-24 times. In other embodiments, the membrane-localizing moiety is cholesterol, tocopherol, or palmityl. Further embodiments of these lipid-peptides conjugates may comprise, monomers or dimers, lipids or no lipids. In vitro and in vivo biological testing data from beta-galactosidase complementation-based fusion assays, MTT cytotoxicity assays, thermostability assays, and F stabilization assays show that a measles fusion inhibitory conjugate inhibits infection.

### Example 3

FIGS. 84-93 further describe the invention. FIG. 84 shows in vivo efficacy data. Intranasal administration of MeV-derived peptides protects cotton rats from MV infection. Cotton rats (n=4) were infected intranasally with MeV “WTFb” strain. Treatment was given intranasally 24 h and 12 h before infection in 100 ul (5 m/kg dose of peptides). The animals were euthanized 4 days post-infection. MeV titration of lung homogenates showed that the [FIP-HRC₄₅₀₋₄₈₅-peg₄]₂-cholesterol is the most potent inhibitor. [HRC₄₅₀₋₄₈₅-peg₄]₂-cholesterol reduces the viral titer compared to the untreated animals.

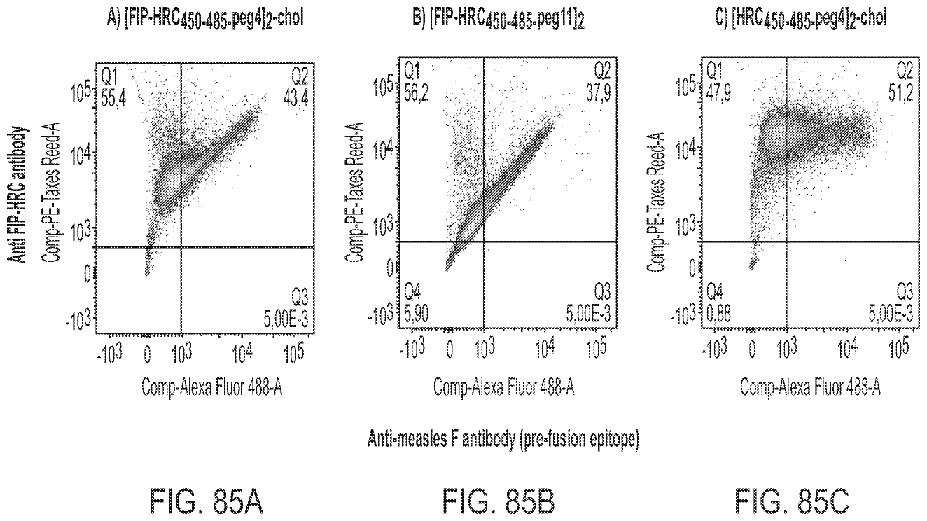

FIG. 85 shows FIP-HRC targets MeV F expressing cells. Flow cytometry—localization of [FIP-HRC450-485-peg4]2-chol peptide in HEK293T cells. HEK293T cells expressing MeV F were incubated with peptide (1 μM) for 60 min at 37° C. F protein and HRC-FIP peptide were stained with Alexa Fluor 488 (green, x-axis) and Alexa Fluor 594 (red, y-axis), respectively. Representative of three separate experiments. For the FIP-HRC with cholesterol, the HRC signal is directly proportional to the F signal (A) suggesting that the FIP-HEC is piloted to the F-expressing cells. FIP alone (without cholesterol) localizes the HRC to F expressing cells but with lower HRC signal (B). When lipid is present but FIP is missing (C) the HRC is not as specifically localized to the F-expressing cells.

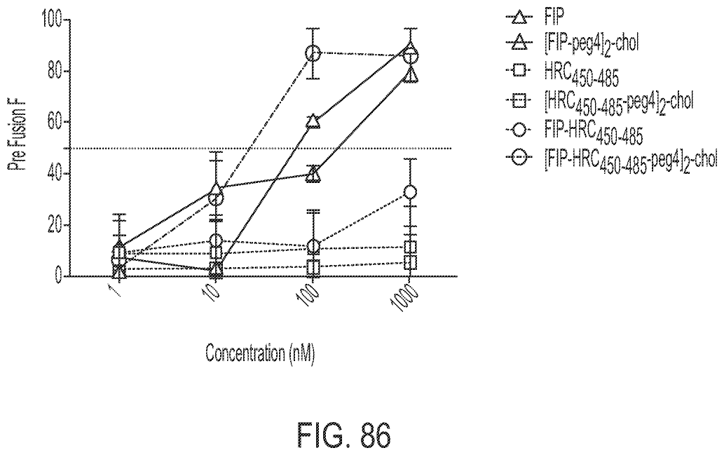

FIG. 86 shows that FIP-HRC stabilizes the measles F in its pre-fusion state. Thermal stability of the MeV F wild-type in presence of FIP, HRC, and FIP-HRC peptides. 293T cells expressing MeV F (“WT”) were incubated overnight at 37° C. The cells were then placed at 55° C. for 10 minutes in the presence of increasing concentrations of the indicated peptides. The cells were then incubated at 4° C. with pre-fusion conformation specific mouse mAb (77.4). Secondary antibody anti-mouse conjugated with Alexa 488 was used for detection. Stained cells were identified using a cell analyzer high content image system. The values on the y-axis indicate the % of positive cells compared to untreated cells, and represent the percentage of conformational antibody binding (reflecting the percentage of F in pre-fusion state.) The values are means (±SE) of results from three experiments. The [FIP-HRC450-485-peg4]2-cholesterol peptide is the most effective at stabilizing F in pre-fusion state.

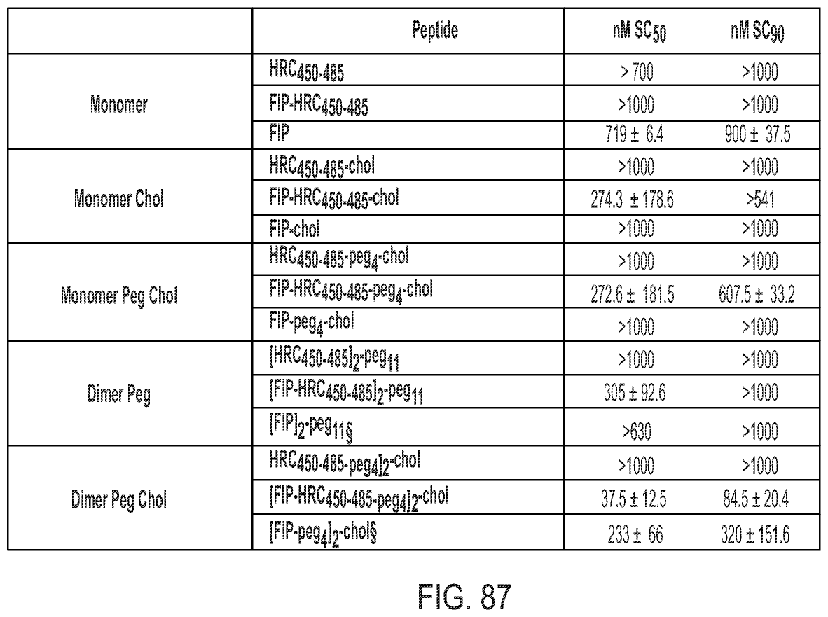

FIG. 87 shows thermal stability of the MeV F (wt) in presence of the indicated peptides. Concentration at which the pre-fusion epitope is 50% (“stable concentration50” or SC50) and 90% (“stable concentration90” or SC90) of the samples not incubated at 10 minutes at 55° C. The values are means (±SE) of results from at least three experiments. [FIP-HRC450-485-peg4]2-chol is the most effective F stabilizer.

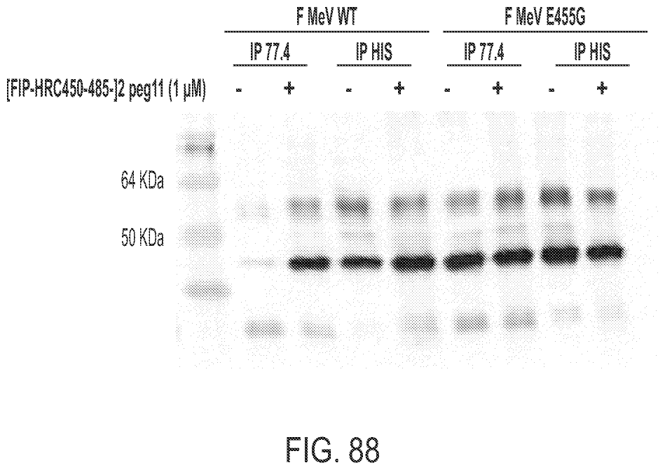

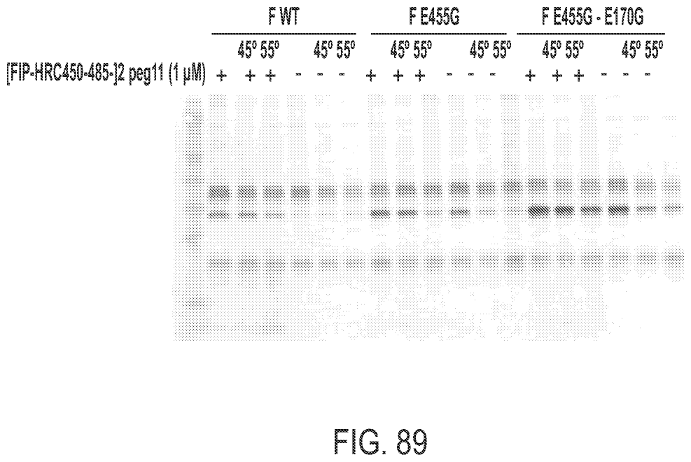

FIGS. 88-90 shows stabilization properties of the MeV peptides (on soluble F). Referring to FIG. 88, cells were transfected with two soluble forms of the MeV F. Wt F and the mutant E455G (inherently stabilized in pre-fusion state) were used. The cells were then incubated with or without the indicated peptides. 24 hours post transfection, aliquots of supernatant fluid were immune-precipitated using either the pre-fusion conformation specific mouse mAb (77.4) or anti-histidine (HIS) antibodies. Anti-HRC polyclonal antibodies were used for detection. The [FIP-HRC450-485-]2-peg11 peptides stabilize the wt soluble F (see lanes 1 vs. 2 and compare to stable E455G F, lane 5). Referring to FIG. 89, cells were transfected with three soluble forms of the MeV F. Wt F, the mutant E455G, the double mutant E170G E455G (both mutants are inherently stabilized in pre-fusion state when expressed as transmembrane protein on the cells) were used. The cells were then incubated with or without the indicated peptides at 37° C. 24 hours post transfection, 3 aliquots of supernatant fluid for each combination were transferred to either 4° C., 45° C., or 55° C. for 30′. The samples were then transferred to 4° C. and immune-precipitated using either the pre-fusion conformation specific mouse mAb (77.4). Anti-HRC polyclonal antibodies were used for detection. The [FIP-HRC450-485-]2-peg11 peptides stabilize all the soluble F protein at both 45° C. and 55° C. The mutant soluble F bearing the E455G and the E170G E455G mutations are both stable at 37° C. in pre-fusion state even in the absence of the [FIP-HRC450-485-]2-peg11.

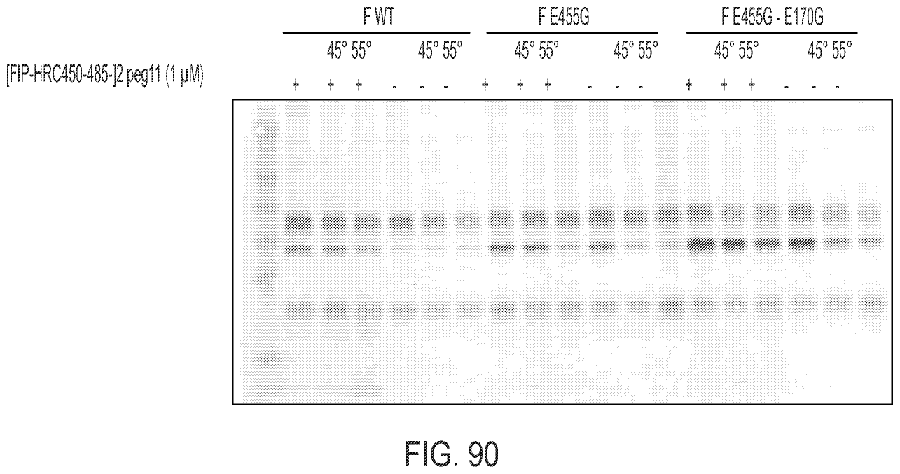

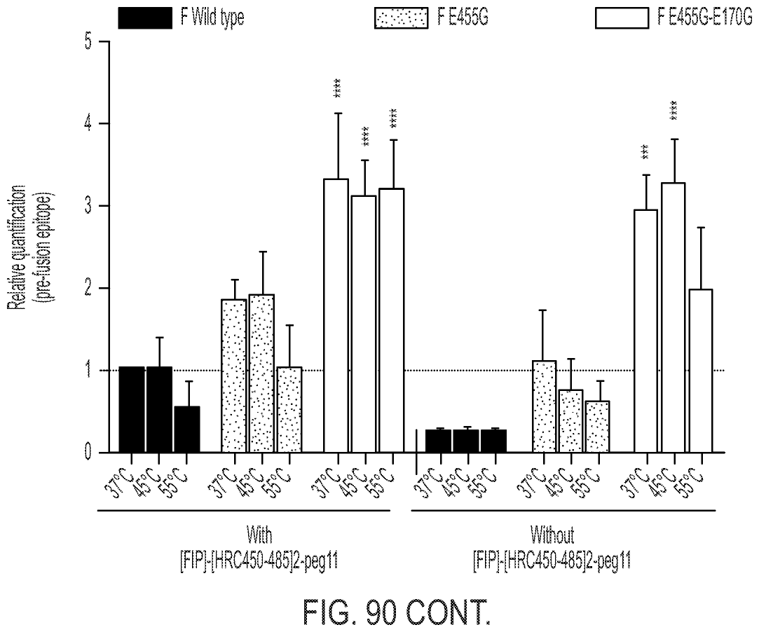

FIG. 90 shows a Western blot. 293T cells expressing soluble form to the measles virus (MeV) fusion protein (F) wt, E455G, or EG170-E455G with (+) or without (−) 1 uM of the indicated peptide were cultured for 24 hours. Supernatant fluids from the cultured cells were collected and were incubated 4° C., 45° C., or 55° C. for 30′. The samples were then transferred to 4° C. and immune-precipitated using either the pre-fusion conformation specific mouse mAb (77.4) that recognizes a pre-fusion epitope. (A) Precipitates were subjected to Western blot analysis using anti-MeV F HRC. (B) Densitometry measurements of immunoprecipitated MV F protein detected through Western blot analysis. Protein content was normalized to the MeV F wt in the presence of the [FIP-HRC450-485-]2-peg11. (n=3, mean±standard error). The [FIP-HRC450-485-]2-peg11 peptides stabilize all the soluble F protein at both 45° C. and 55° C. The mutant soluble F bearing the E455G and the E170G-E455G mutations are both stable at 37° C. in pre-fusion state even in the absence of the [FIP-HRC450-485-]2-peg11.

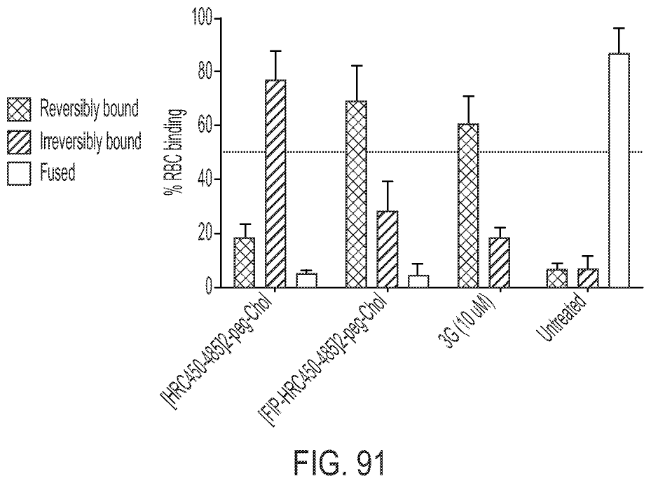

FIG. 91 shows FIP-HRC prevents F activation (a different mechanism from HRC that prevents F refolding). Monolayers of cells co-expressing H-HN T193A (a chimeric binding protein with the MeV stalk and the HPIV3 head, which binds sialic acid receptors but triggers MeV F) and MeV F (S262R, an easily activated F) were allowed to bind to sialic acid receptor-bearing red blood cells (RBCs) at 4° C. Upon transfer to 37° C., media containing the indicated compound or peptide (1 uM) were added for 60′. Then 10 mM zanamivir was added to release the RBCs that were reversibly bound (i.e., bound only by H-HN and not by F insertion). RBCs that are reversibly bound by HN-receptor interaction (orange), irreversibly bound by F insertion (blue), or fused (white) were quantified. The ordinate values are means (±SE) of results from triplicate experiments. [HRC450-485-peg4]2-chol blocks fusion after F insertion into the target cell (irreversibly bound, blue). [FIP-HRC450-485-peg4]2-chol blocks at the pre-fusion state (reversibly bound, orange). 3G is a small molecule that stabilizes the F in its pre-fusion state (reversibly bound RBC, orange). Zanamivir released all the RBC when added at the beginning of the 37° C. incubation.

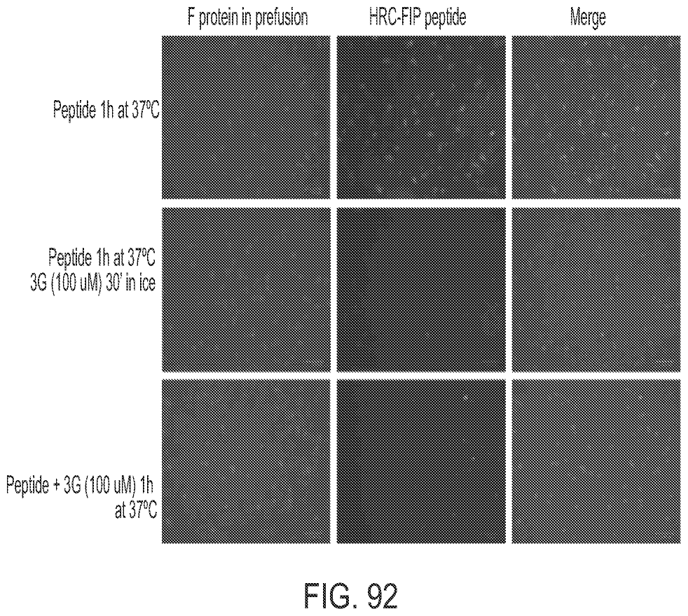

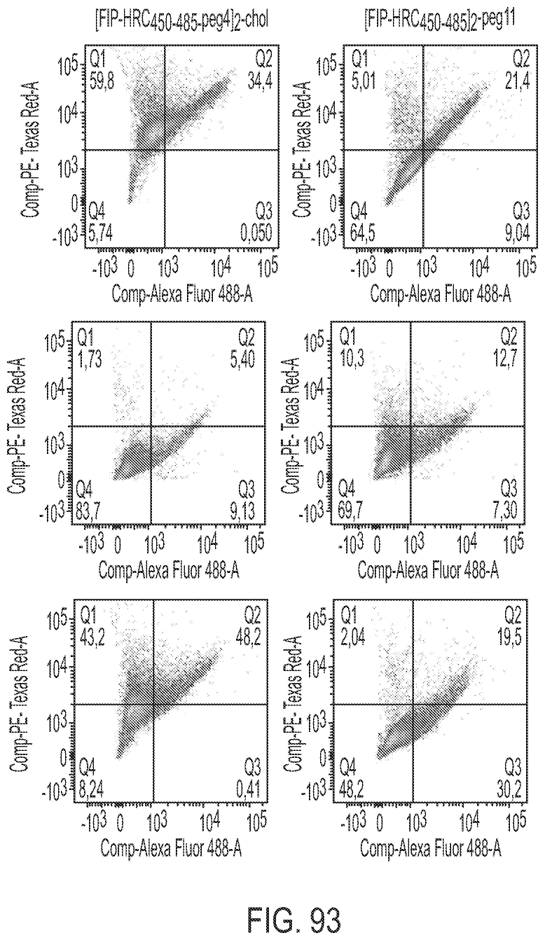

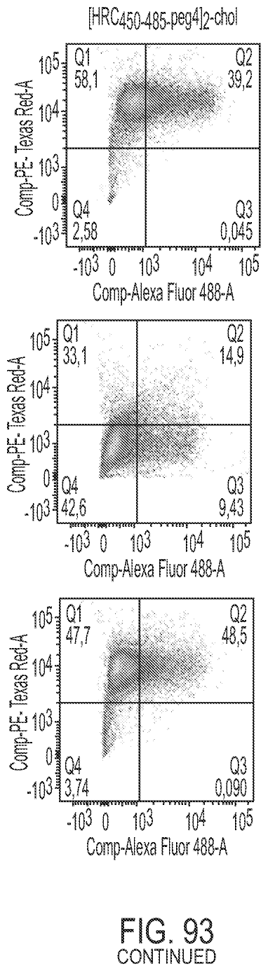

FIGS. 92-93 show FIP-HRC targets MeV F expressing cells. FIG. 92 shows localization of [FIP-HRC450-485-peg4]2-chol peptide in HEK293T cells. HEK293T cell cultures were incubated with peptide (1 μM) for 60 min, at 37° C. F protein and HRC-FIP peptide were stained with Alexa Fluor 488 (green) and Alexa Fluor 594 (red), respectively. The merged image shows colocalization. FIG. 93 shows FIP-HRC targets MeV F expressing cells, from three separate experiments.

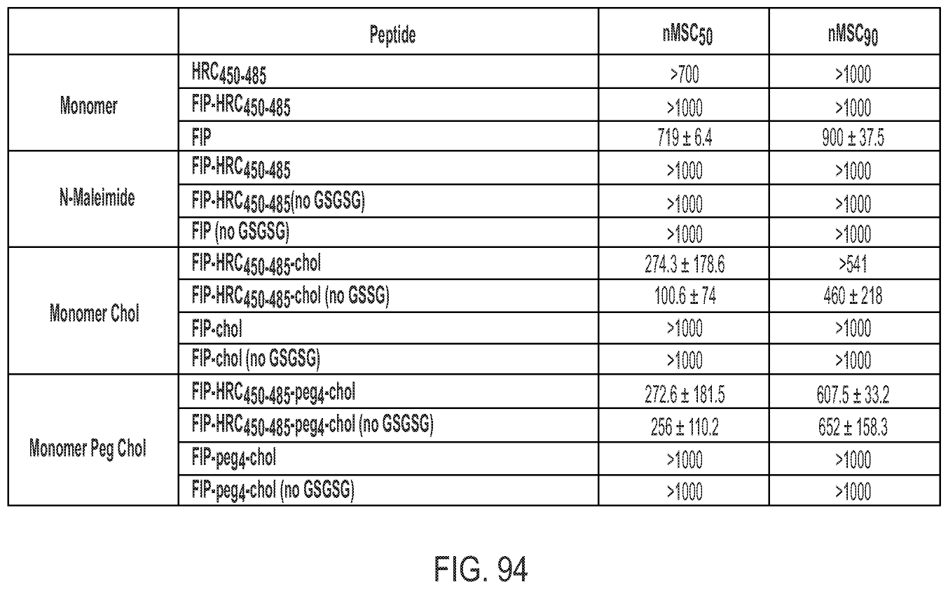

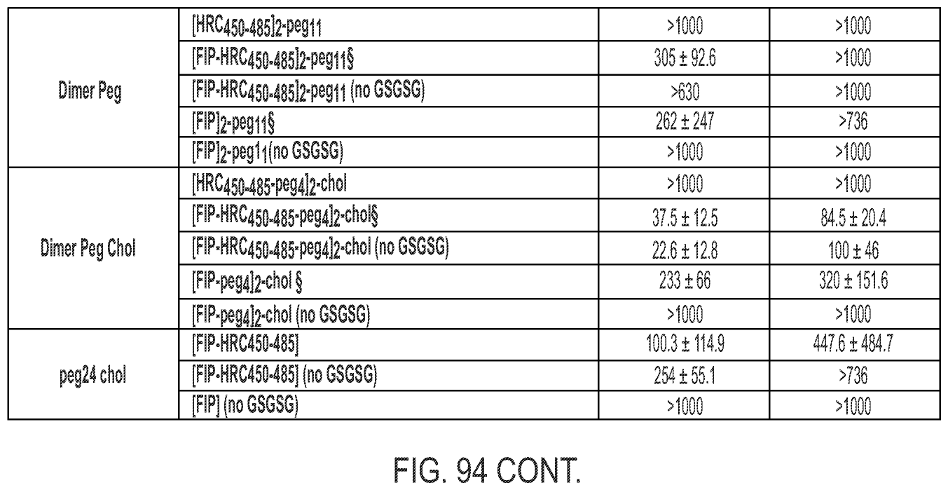

FIG. 94 shows thermal stability of the MeV F (WT) in presence of the indicated peptides. Concentration at which the pre-fusion epitope is 50% (“stable concentration50” or SC50) and 90% (“stable concentration900” or SC90) of the samples not incubated at 10 minutes at 55° C. The values are means (±SE) of results from at least three experiments. [FIP-HRC450-485-peg4]2-chol is the most effective F stabilizer.

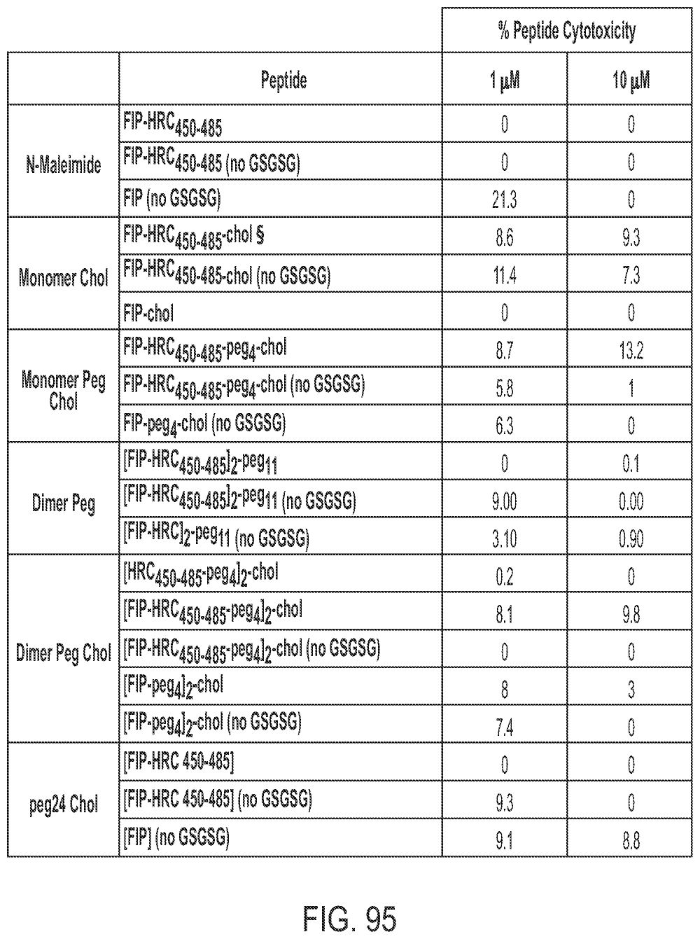

FIG. 95 shows cytotoxicity of the MeV peptides. MeV peptide cytotoxicity evaluated in 293T HEK cell cultures using a commercial MTT assay. The peptides are not toxic.

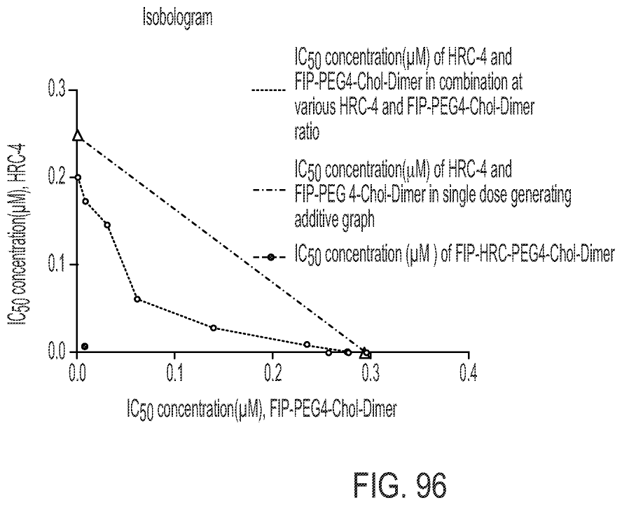

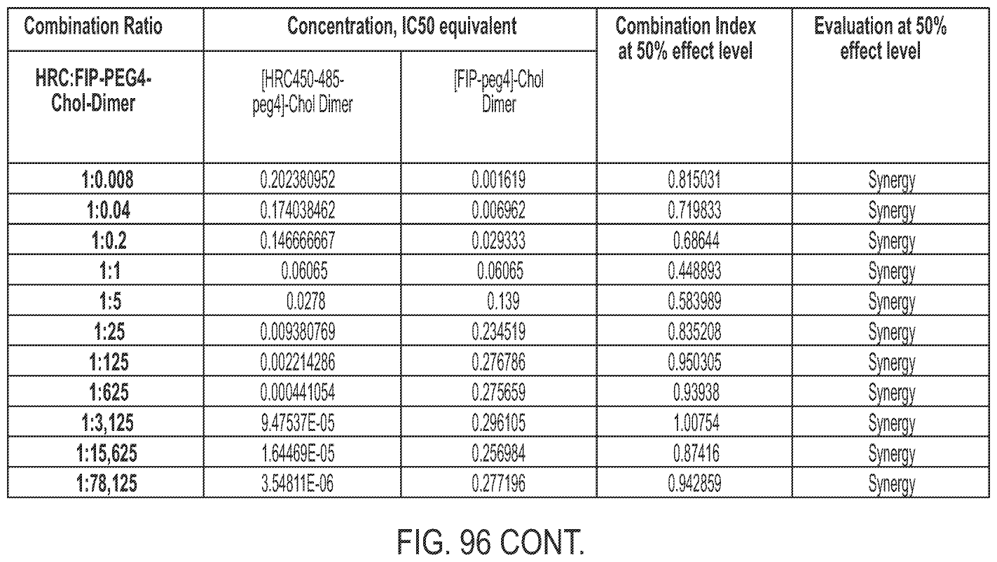

FIG. 96 shows synergism. Isobologram analysis of HRC+FIP. The diagonal line is the line of additivity. Experimental data points, represented by dots, located below, on, or above the line indicate synergy, additivity, or antagonism, respectively. The red dotted line is the curve generated from contributions of FIP and HRC at different ratios of the same two components. The blue dot represents the contribution of HRC-4 and FIP-PEG4-Chol-Dimer in FIP-HRC-PEG4-Chol-Dimer at IC50 concentration. The data are from three experiments. The table shows the results of isobologram analysis.

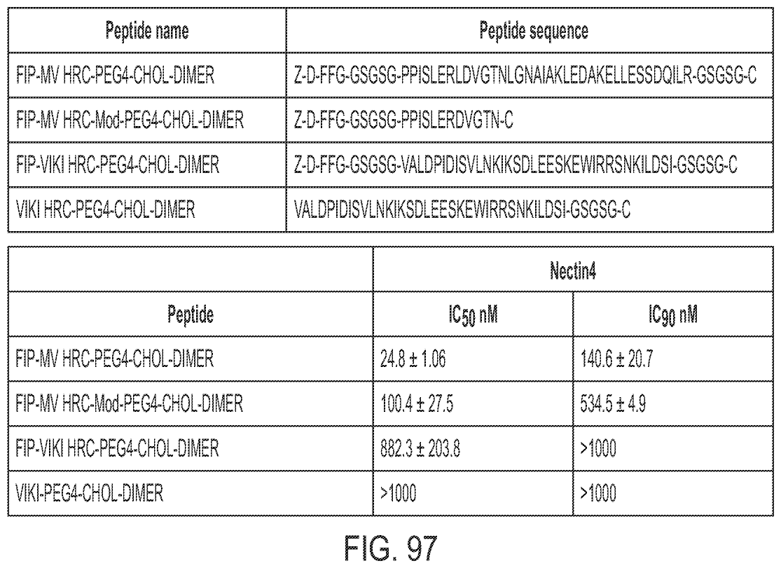

FIG. 97 shows potency of a FIP-HRC with 12 amino acids derived from the measles HRC. Instead of using the HRC peptide derived from the measles F, a HRC peptide derived from human parainfluenza 3 (HPIV3) F was used. This peptide was called VIKI. The VIKI peptide is very effective vs. HPIV3 and Nipah virus but is a weak inhibitor of measles. FIG. 97 shows the IC50 and IC90 of the peptides. The FIP-VIKI HRC-PEG4-CHOL-DIMER is significantly less potent that the FIP-MV HRC-PEG4-CHOL-DIMER. This indicates that the potency correlates with the amino acid sequence. A modified FIP-MeV HRC (FIP-MeV HRC-Mod-PEG4-CHOL-DIMER) is a FIP-HRC with 12 amino acids derived from the measles HRC. The FIP-MeV HRC with 12aa from the measles HRC region is more potent than the the FIP-VIKI.

### Example 4—FIP-HIV HRC

### Serum Neutralization Assay

Pseudovirus env and NL-Luc-AM vector are co-transfected in 293T cells using Effectine (Qiagen) reagent. Media is changed after 16 hours and supernatants are aliquoted and frozen down at −80° 32 hours later. Virus titrations are set up on TZM-BL cells to find the dilution that yields 100,000 counts per second of luciferase. Sera are thawed and heat inactivated at 56° for 1 hour. Samples not in use are stored at −20°. TZM-BL cells are seeded 16 hours prior to infection at a concentration of 1×104 cells/well, in opaque, white cell culture plates. Sera are thawed and spun down for 10 minutes at max speed. Sera are diluted 1:5 initially and spun 10 minutes at max speed through Spin-X (Costar) filter tubes. (This is not done for purified IgG).

Sera are serially diluted five more steps of 1:4 each. Control inhibitors are serially diluted 1:5 for 6 steps. Diluted sera and control inhibitors are transferred to preincubation plates. Sera from each animal is transferred to its own plate (110 ul per well). Each plate contains at least two virus control wells and one background well, which at this point are media only. Pseudoviruses are then thawed, diluted to a concentration previously determined to yield 2,000,000 CPS, and added (110 ul) to all wells except background wells, which receive media. Plates are incubated at 37° for 1 hour. The preincubation mixture is then combined with cells. Each well contains enough volume for two replicates (100 ul preincubation mixture to 100 ul cells). 16 hours post-infection, media can be aspirated from the cells and replaced if necessary. Day 3 post infection media is aspirated from cells and 50 ul Glo-Lysis Buffer (Promega) is added to each well. Assay plates are frozen for at least two hours at −80°. Assay plates are thawed and each well mixed with a multi-channel pipette. An equal amount (50 ul) of Bright-Glo substrate (Promega) is added to each well and luciferase counts are detected. Counts from background wells within each assay plate are subtracted from sample data and counts are plotted as percent inhibition, with virus control wells set at 100% growth.

### Pseudovirus Transfection Protocol

The protocol is for a 30 ml transfection in a T175 flask. For a T75 flask, cut all amounts in half. One Day Prior to Transfection: Seed 293T cells (ATCC, CRL-11268) to about 70% confluence in a T175 flask. Note: Splitting a fully confluent T175 flask 1:3 gives the desired cell concentration. Day of Transfection: Note: It's best to perform this transfection late in the day, to be closer to a 16 hour overnight incubation. Add 1.5 ml EC Buffer (Effectene kit—Qiagen, 301427) to a 15 ml tissue culture tube. Add 12 ug DNA to EC Buffer. For pseudovirus, add 12 ug env plasmid and 12 ug backbone vector (NL-Luc). Note: Most envs tested are most infectious as a 1:1 env: backbone transfection, however low titer envs could potentially be boosted by empirically determining their ideal ratio on a case by case basis (no more than 5× of either plasmid in the ratio). Note: NL-Luc-AM backbone has consistently proven to produce more infectious pseudovirus than the standard pNL4-3.Luc.R-E-(aidsreagent.org, #3418). Add 100 ul Enhancer (Effectene kit) and gently mix by swirling. Incubate 5 minutes at room temperature. Add 120 ul Effectene reagent (Effectene kit) and gently mix by swirling. Incubate 10 minutes at room temperature. Gently aspirate cell line media from 293T flask. Quickly add 10 ml Cell Line Media (DMEM, 10% FBS, Pen/Strep, Gln) to DNA complex in 15 ml tube. Note: It is reportedly not necessary to use Opti-Mem with Effectene. Quickly remove cell line media with DNA complex from tube and gently add to 293T flask. Note: 293T cells are not tightly attached to the flask—do not directly pipette media on top of cells. Incubate flask over night at 37 degrees. Day 1 Post-Transfection: Note: Perform washout first thing in the morning. Gently aspirate media from 293T cell flask. Gently add 30 ml new Cell Line Media to flask. Note: The Effectene transfection method is reportedly not toxic to cells—media is changed when transfecting pseudovirus as a precaution. Day 2 Post-Transfection: Note: Perform harvest late in the day for reportedly higher titers. Harvest can also be potentially pushed back to day 3. Remove viral supernatant from flasks to a 50 ml tube. Spin sample for 10 minutes at 1600 RPM. Note: Cryotubes (VWR, #66021-996) can be labeled at this time. Sterile filter viral supernatant through a Steriflip (Fisher, SCGP00525) or equivalent. Note: This step is performed to remove any remaining cell debris, but it can reportedly capture some virus on the filter. For low-titer virus, this step can be omitted. Quickly aliquot viral supernatant into cryotubes, 1 ml each. Immediately place samples into the −80° C. freezer. Note: The longer the virus remains at room temperature without cells, the lower the titer will be. It is not recommended to perform this harvest on more than one or two viruses at a time. Aliquoting may also be performed on ice.

TABLE 4
Summary of 072920EF

| | BG505 | B41 | 16055 | MN | VSV | MLV |
|---|---|---|---|---|---|---|
| IC₅₀ (nM): | | | | | | |
| FIP HIV | <4.9 | 13 | 13 | <4.9 | >5000 | >5000 |
| FIP HRC | 2541 | >5000 | >5000 | >5000 | >5000 | >5000 |
| FIP VI KI | >5000 | >5000 | >5000 | 1279 | >5000 | >5000 |
| FIP | >5000 | >5000 | >5000 | >5000 | >5000 | >5000 |
| HIV | <4.9 | 5.2 | <4.9 | <4.9 | >5000 | >5000 |
| [DL-36-Peg4]2-chol | 1236 | >5000 | 1413 | 954 | 1975 | >5000 |
| DMSO | >5000 | >5000 | >5000 | >5000 | >5000 | >5000 |
| IC₅₀ (ng/ml): | | | | | | |
| VRCO1 | 103 | 857 | 184 | 124 | >16000 | >16000 |

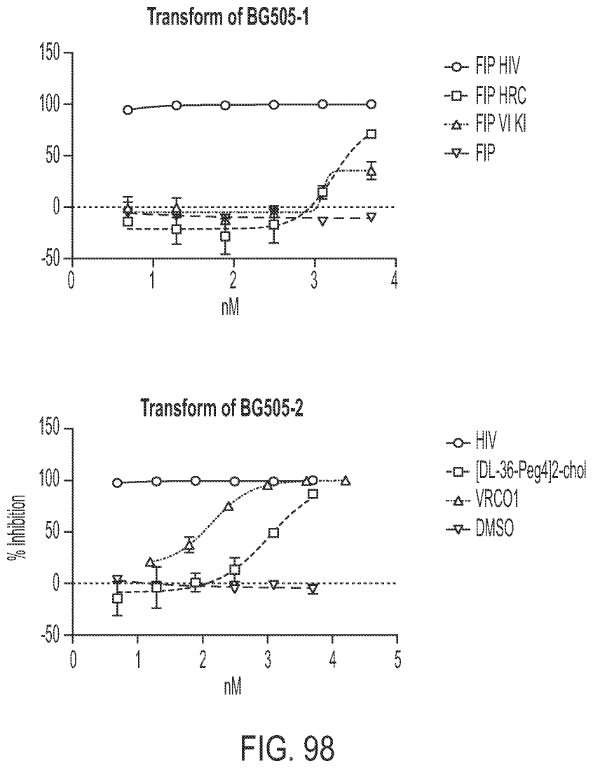

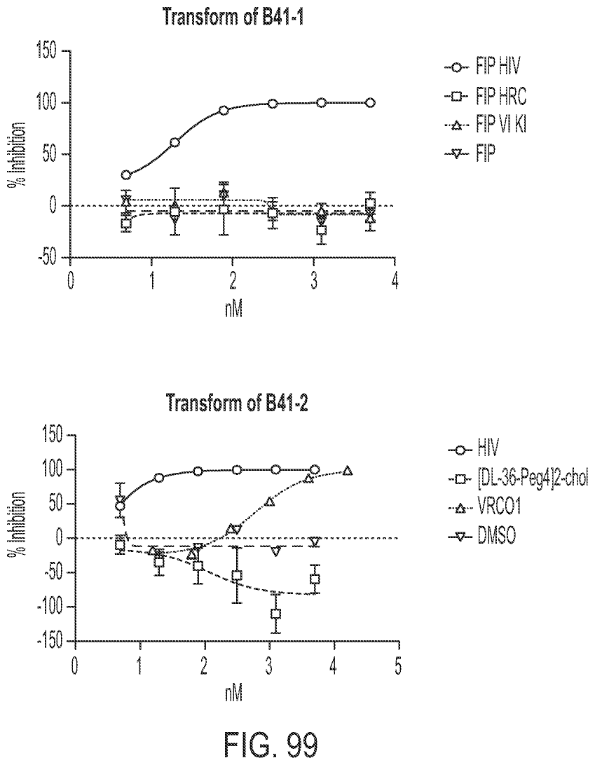

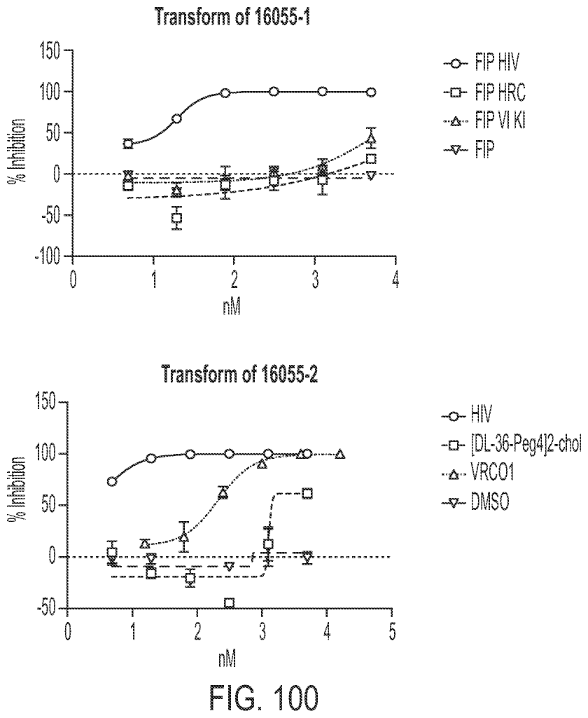

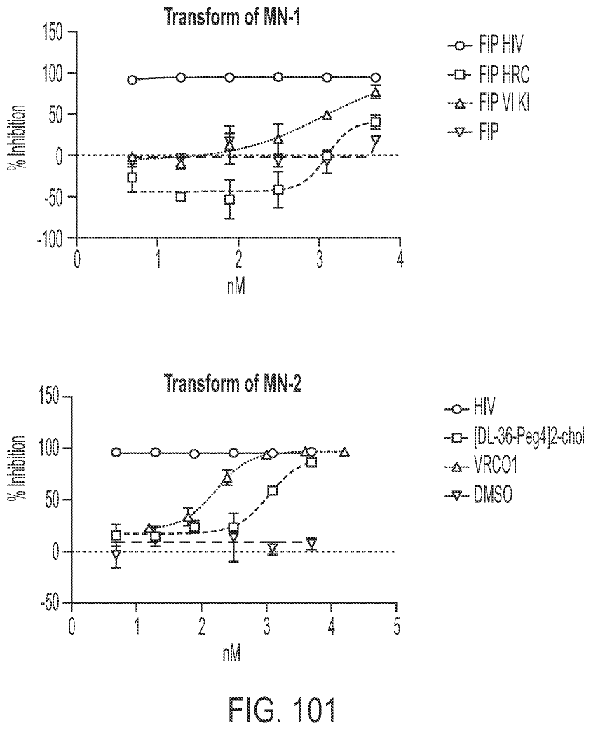

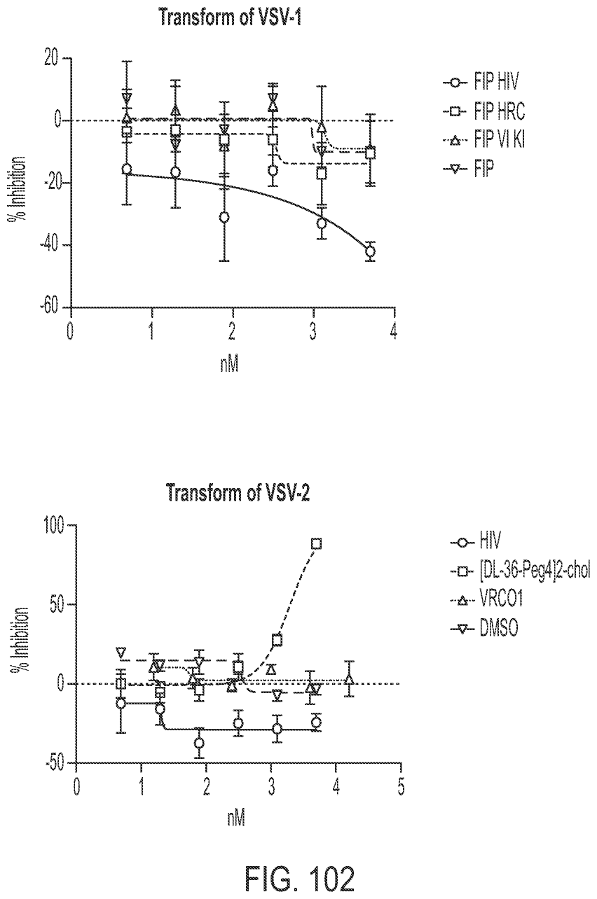

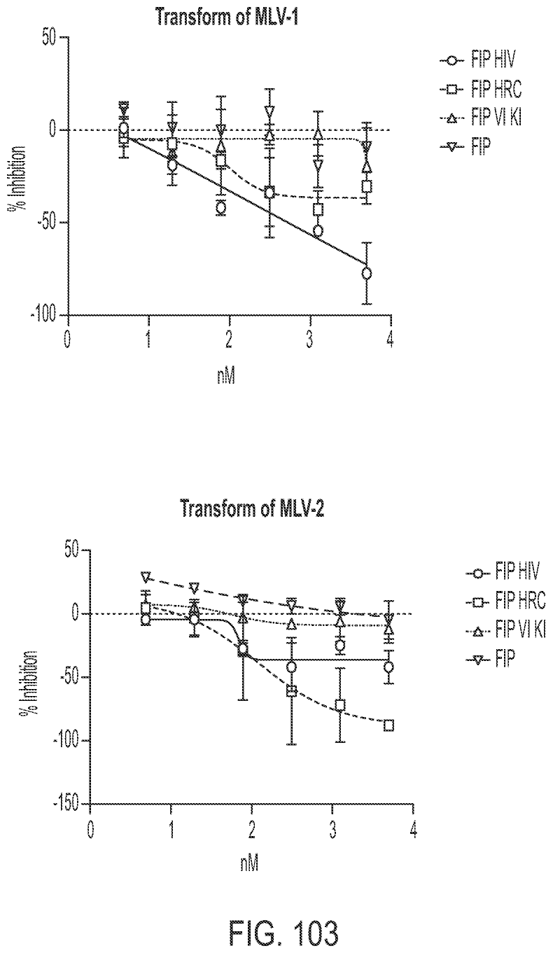

FIGS. 98-103 shows inhibition data for various viruses. FIG. 98 shows inhibition data for BG505 (HIV-1) strain using various lipid-peptide conjugates and positive and negative controls. FIG. 99 shows inhibition data for B41 (HIV-1 strain) using various lipid-peptide conjugates and positive and negative controls. FIG. 101 shows inhibition data for 16055 (HIV-1 strain) using various lipid-peptide conjugates and positive and negative controls. FIG. 101 shows inhibition data for MN (HIV-1 strain) using various lipid-peptide conjugates and positive and negative controls. FIG. 102 shows inhibition data for vesicular stomatitis virus (VSV) using various lipid-peptide conjugates and positive and negative controls. FIG. 103 shows inhibition data for murine leukemia viruses (MLV) using various lipid-peptide conjugates.

### Example 5—Molecular Features of the Measles Virus Viral Fusion Complex that Favor Infection and Spread in the Brain

### Abstract

A measles virus (MeV) isolate bearing a single amino acid change in the fusion protein (F)—L454W—was identified in patients who died of MeV central nervous system (CNS) infection. We analyzed whether this mutation confers a fitness advantage over wild type virus in the CNS, thereby contributing to disease in these patients. Using murine organotypic brain cultures (OBCs) and human brain organoids, we show that specific CNS adaptive mutations in F result in augmented spread of virus ex vivo, in association with an enhanced innate immune response. The spread of virus in brain tissue is blocked by an inhibitory peptide that targets F, supporting the notion that F is involved in dissemination within the CNS. A single mutation in MeV F alters the fusion complex to render the virus more neuropathogenic, allowing it to propagate in brain tissue even in the face of an innate immune response.

### Introduction

Despite the availability of a safe and effective measles virus (MeV) vaccine, MeV has not been eradicated and has caused 100,000-140,000 deaths globally every year since 2010 (Moss & Griffin, 2012; Perry et al., 2015; Simons et al., 2012). MeV eradication by vaccination is hindered in part by low vaccination coverage often related to unjustified parental concerns over vaccine safety (Jansen et al., 2003). Additionally, the vaccine is a live attenuated virus and cannot be used in severely immune-compromised people. Measles has been undergoing a global resurgence which may worsen as the SARS-CoV-2 pandemic reduces regular childhood vaccination coverage and may expose more vulnerable individuals to infection.

Upon initial infection, MeV infects activated CD150(SLAM)-expressing immune cells in the respiratory tract, thereby gaining access to the immune system (Tatsuo, Ono, Tanaka, & Yanagi, 2000). After reaching the draining lymph nodes, the virus proliferates in CD150-expressing lymphocytes and from there proceeds to cause viremia. Late in infection, MeV infects respiratory epithelial cells via nectin 4 expressed on the basolateral membranes of these cells; from this location MeV exits the host's respiratory tract and may be transmitted (Mühlebach et al., 2011; Noyce et al., 2011).

MeV can cause fatal complications days to years after the acute phase of the infection (Allen, McQuaid, McMahon, Kirk, & McConnell, 1996; Buchanan & Bonthius, 2012; Hosoya, 2006), when it infects the central nervous system (CNS). In a small percentage of cases, subacute sclerosing panencephalitis (SSPE) develops several years after the initial infection. SSPE is characterized by persistent infection of the brain associated with hypermutated MeV genomic RNA and viral transcripts and defective viral particle assembly (Cattaneo, Schmid, Billeter, Sheppard, & Udem, 1988; Rima & Duprex, 2005; Schmid et al., 1992). Measles inclusion body encephalitis (MIBE) occurs in immunocompromised patients weeks to months after infection with wild type (wt) viruses and, in rare cases, has occurred with previous versions of the attenuated MeV vaccine that are no longer used (Baldolli et al., 2016; Buchanan & Bonthius, 2012; Hughes, Jenney, Newton, Morris, & Klapper, 1993). MIBE may be associated with viral fusion complexes that mediate fusion without needing to engage with the receptor on the cell surface, referred to as hyperfusogenic MeV fusion complexes (Hardie, Albertyn, Heckmann, & Smuts, 2013; C. Mathieu et al., 2015). To date, the mechanisms governing MeV infection and spread in the CNS remain poorly understood, although CNS invasion seems to require the viral fusion (F) protein and thus may be targeted by fusion inhibitors (Hashiguchi et al., 2018; Makhortova et al., 2007; M. Watanabe et al., 2016; Young & Rall, 2009).

Infection of a cell by MeV starts with attachment to cell surface receptors, and entry is then mediated by the concerted actions of the MeV receptor binding (H) and F proteins on the surface of the virus. The H/F complex of MeV thus constitutes the viral fusion machinery that promotes entry into host cells (Chang & Dutch, 2012; Harrison, 2008). Infected cells synthesize F as a precursor (F0) that is cleaved within the cell to yield the pre-fusion F complex, comprised of three C-terminal F1 subunits that are associated via disulfide bonds with three N-terminal F2 subunits. New viral particles display this trimeric F structure kinetically trapped in a metastable conformation on the outer surface of the viral membrane (Hashiguchi et al., 2018). F is primed for fusion activation upon engagement of the H glycoprotein by a target cell surface entry receptor (i.e., CD150 or nectin 4 for wild-type strains) (Mühlebach et al., 2011; Noyce et al., 2011; Tatsuo et al., 2000). After receptor engagement, H triggers the pre-fusion F protein to undergo a structural transition, extending to insert its hydrophobic fusion peptide into the host cell membrane. F then refolds into a stable post-fusion 6-helix bundle structure, which brings the viral and target cell membranes together to initiate formation of the fusion pore. The ability of the F protein to refold and reach this post-fusion state relies on the interaction between two complementary heptad repeat (HR) regions localized at the N and C-termini of the protein (HRN and HRC, respectively). This step of fusion can be inhibited by peptides corresponding to these HR regions (Lambert et al., 1996).

CNS infection in several patients has been observed with isolates of MeV that bear F proteins with mutations in the HRC domain (Ayata et al., 2010; Hardie et al., 2013; Jurgens et al., 2015; Watanabe et al., 2013). The growth of some mutated viruses in other organs in vivo was impaired (S. Watanabe et al., 2015). A viral sequence recovered from two patients who died from MIBE contained F with an L454W mutation (Hardie et al., 2013) that conferred thermal lability to the metastable F. We have previously shown that this F mutation affects entry into target cells; recombinant MeV (IC323 strain) expressing green fluorescent protein and bearing the L454W F (MeV-IC323-EGFP-F L454W) spreads in cells that lack a known MeV receptor. In cell-to-cell fusion assays, F bearing the L454W mutation alone mediates fusion independently of the H protein (Jurgens et al., 2015). In contrast, other hyperfusogenic viruses are dependent on the H protein for membrane fusion (Sato et al., 2018).

The F L454W mutation that was identified in the MIBE patients could either have arisen de novo in the CNS (Hardie et al., 2013) or could have been present in the wt viral population and undergone positive selection in the CNS. The origin of this virus could not be determined, and it is unknown whether the CNS isolate with this F can infect other tissues. One report showed that a virus bearing L454W F can emerge under the selective pressure of certain fusion inhibitors (Ha et al., 2017), indicating that viruses bearing this neuropathogenic F protein can be found outside the CNS. In recent work, we showed that a virus bearing the L454W F grows better than wt in the lung of cotton rats, is tenfold more lethal in a suckling hCD150 tg mouse model of MeV CNS infection, and reaches the mouse CNS faster than wt MeV (Mathieu et al., 2019). However, the reason for the observed impact of this mutation and of the altered fusion complex in the brain has not been investigated.

In this series of experiments, we asked whether MeV bearing CNS-adapted fusion complexes are different from wt MeV in terms of growth and spread in two ex vivo models of CNS infection: murine organotypic brain cultures, and human brain organoids. The hyperfusogenic variants that are observed in cases of encephalitis spread more efficiently than wt in these models. The infection does not require any known measles receptor, and the extent of infection is inversely correlated with the stability of the MeV F (wt or mutant) in its pre-fusion state. Spread of virus is blocked by fusion inhibitors that inhibit re-folding of F. An innate immune response is induced, which, however, does not block viral spread. Viral evolution in the ex vivo models described above highlights the functional requirement of the MeV fusion complex for CNS adaptation.

### Results

### Measles Virus Bearing the F Glycoprotein L454W is not Stable in Cell Culture

We and others have previously described mutations in the MeV F glycoprotein (L454W, T461I, and N462K) associated with neuropathogenic measles strains that were either isolated from patients or generated in laboratory settings (Hardie et al., 2013; Jurgens et al., 2015; Mathieu et al., 2019; Watanabe et al., 2013). These mutations conferred decreased thermal stability to the prefusion metastable state of the F protein. To explore the molecular determinants for altered stability, these mutations were mapped onto x-ray structures of the pre-fusion (MeV F; PDB 5YXW; Hashiguchi et al., 2018) and post-fusion (HPIV3 F; PDB 1ZTM; Yin et al., 2005) conformations of F proteins (FIG. 104). The three mutations (L454W, T461I, and N462K) are all located within the HRC domain. Based on the pre-fusion structure, the L454W mutation would likely cause steric hindrance with T314 within the same protomer and/or L457 within an adjacent protomer. The T461I and N462K mutations occur in a well-ordered α-helical region of the HRC domain. In silico mutation of these residues would lead to steric clash with the adjacent protomer. Most notably, these three mutations occur in the portion of the HRC domain where the head and stalk regions of the pre-fusion conformation meet. Interactions at this junction are likely to be important for stabilizing the pre-fusion state, and consequently, mutations in this region could lead to decreased stability of the MeV-F prefusion structure, as our previous data suggest (Jurgens et al., 2015).

The MeV F L454W was found in two separate clinical cases. The mutation decreases the stability of the fusion protein, producing a hyperfusogenic phenotype that allows MeV spread in Vero cells even in the absence of known receptors. In order to assess the impact of such mutation on viral fitness, we generated a recombinant infectious clone of MeV IC323-EGFP bearing F L454W, and grew the recombinant virus in Vero-CD150 cells at either 37° C. or at 32° C. (the lower temperature stabilizes the F protein in its pre-fusion state (Jurgens et al., 2015)). In the course of this process of generating recombinant viruses bearing the L454W F by reverse genetics, a process expected to be routine, an unexpected set of mutations emerged. For both viral preparations titers remained low reaching only 2×10⁵ plaque forming units (pfu)/ml at 32° C. and ˜5×10⁵ pfu/ml at 37° C., compared to wt virus stocks which generally grow to over 5.10⁶ pfu/ml. We previously noted that the viral titer of the mutant is markedly reduced compared to wt virus at 37° C. (Mathieu et al., 2019), suggesting that the F protein harboring the L454W mutation is detrimental for viral growth in cell culture. After 3 passages, next generation sequencing of these two viral stocks rescued from the same initial plasmid but grown at different temperatures showed an additional mutation in the F protein in both: In addition to L454W present in 100% of the sequences, a G506E mutation emerged at 32° C. with an allele frequency of ˜36% and an E455G mutation emerged at 37° C. with an allele frequency of ˜22%. The E455G mutation can be observed in FIG. 104, but G506 is not resolved in the available crystal structures. We hypothesized that the instability of the L454W F variant favors the emergence of these two new mutations in cell culture. These unexpected mutations provided remarkable investigative tools as discussed below.

Growth of MeV-IC323-EGFP-F L454W Vs. Wt Virus in Brain Tissue in Absence of Known Receptor

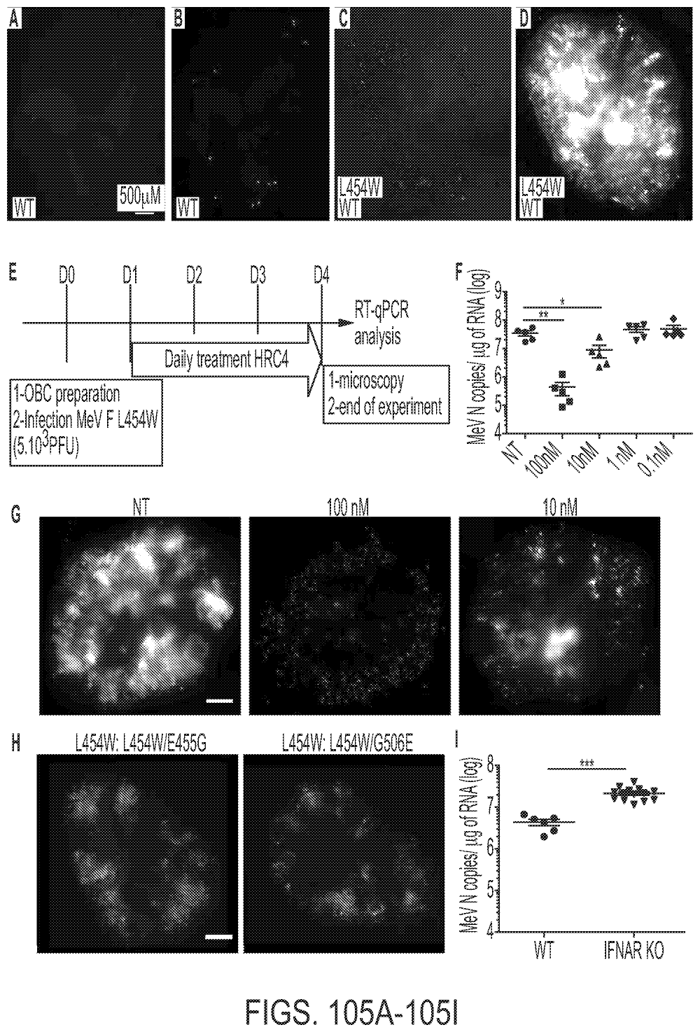

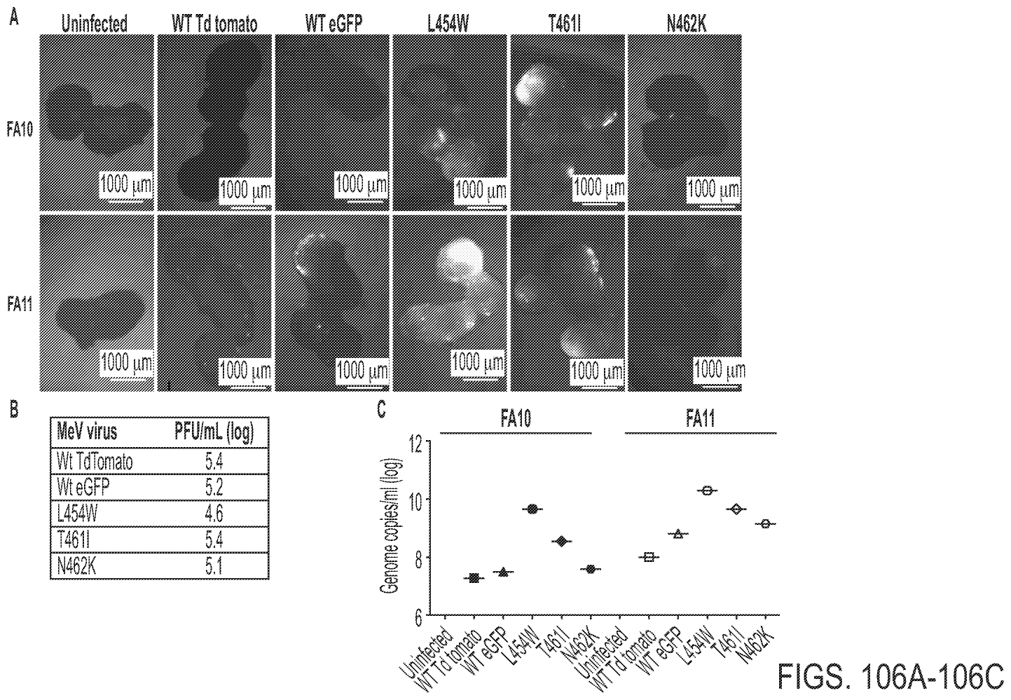

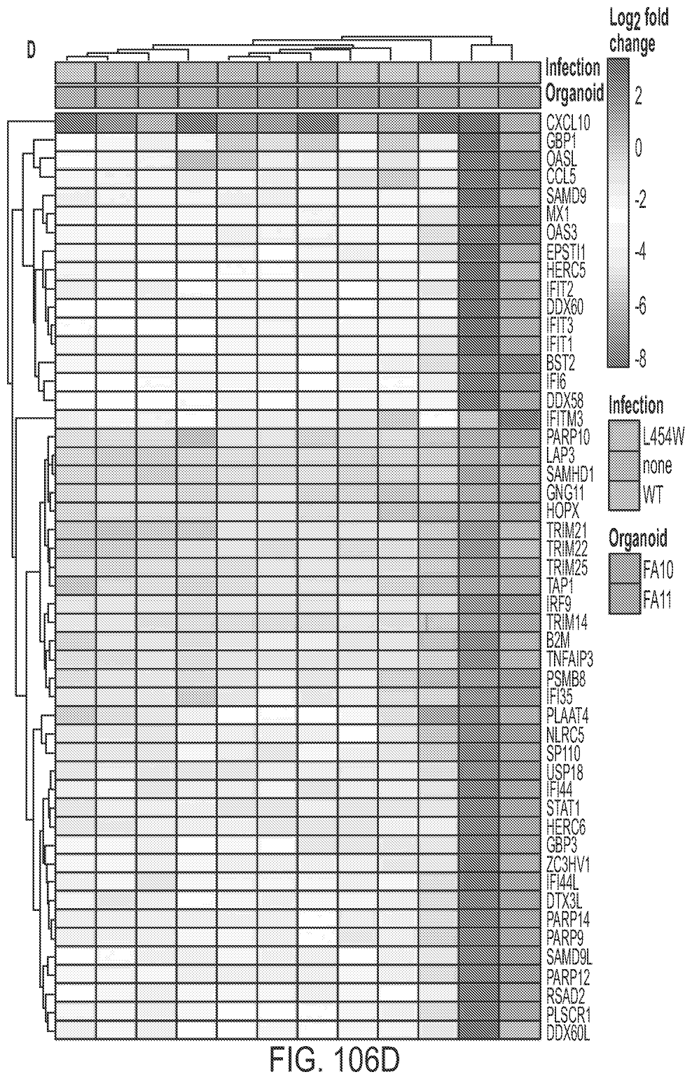

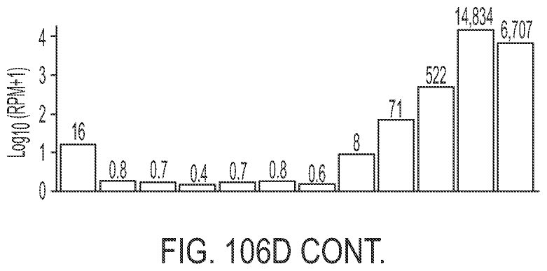

Since MeV virus with L454W F has been isolated from the CNS we postulated that it would be well adapted in two models of brain infection (murine and human). We hypothesized that this mutation would be under positive selective pressure in the brain. We previously had used mouse cerebellar organotypic brain cultures (OBC) from IFNAR1 knock-out (KO) mice to assess viral infection and spread in the four cell types present in the CNS (Welsch et al., 2017) but in the absence of antiviral effect related to type 1 IFN response. Hippocampal and cerebellum OBC from mice that express the human CD150 F1 transgene sustain wt virus infection and spread (J. C. Welsch et al., 2013), but when known MeV receptors are absent, the wt virus does not spread (Ferren et al., 2019; Welsch et al., 2017). In the experiment shown in FIGS. 105A-D, OBC were derived from IFNAR1KO mice that do not express any known measles receptor. The wt virus (expressing enhanced green fluorescent protein, EGFP) fails to spread over 96 h (FIGS. 106A and 106B). In FIG. 105C-D OBC from IFNAR1KO mice were co-infected with 5000 pfu of wt virus expressing a different fluorescent protein (the red fluorescent protein tdTomato) and MeV-IC323-EGFP-F L454W (bearing the additional G506E mutation that emerged in culture). Infection was monitored at 24 hours (FIG. 105C) and 96 hours (FIG. 105D). While the wt virus did not (as expected) efficiently spread in the tissues, the virus bearing L454W (EGFP) infected and spread, and the G506E mutation allele frequency increased from ˜36% to ˜70% showing the strong positive selection pressure for this mutation in F.

### Blocking Fusion Inhibits Spread of all Variants

MeV vaccine strain infection studies have suggested that interfering with F protein function can stop spread within the CNS (Makhortova et al., 2007). We previously showed that a MeV F derived dimeric cholesterol conjugated fusion inhibitory peptide (termed HRC4, that block F mediated fusion) blocks infection with wt MeV in vitro, ex vivo and in vivo in cotton rats and mice (Mathieu et al., 2015, 2019; Welsch et al., 2013). The HRC4 peptide's efficacy at blocking virus dissemination in the OBC tissue after exposure to virus was assessed in FIGS. 105E-G. The OBC used here were obtained from IFNAR1KO that did not express any known MeV receptor (Welsch et al., 2017).

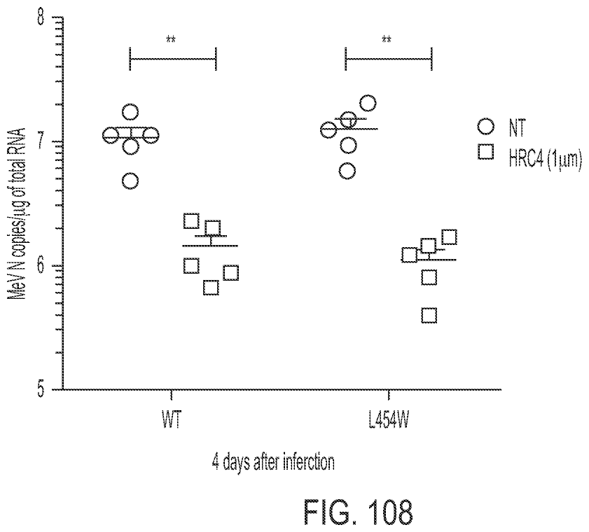

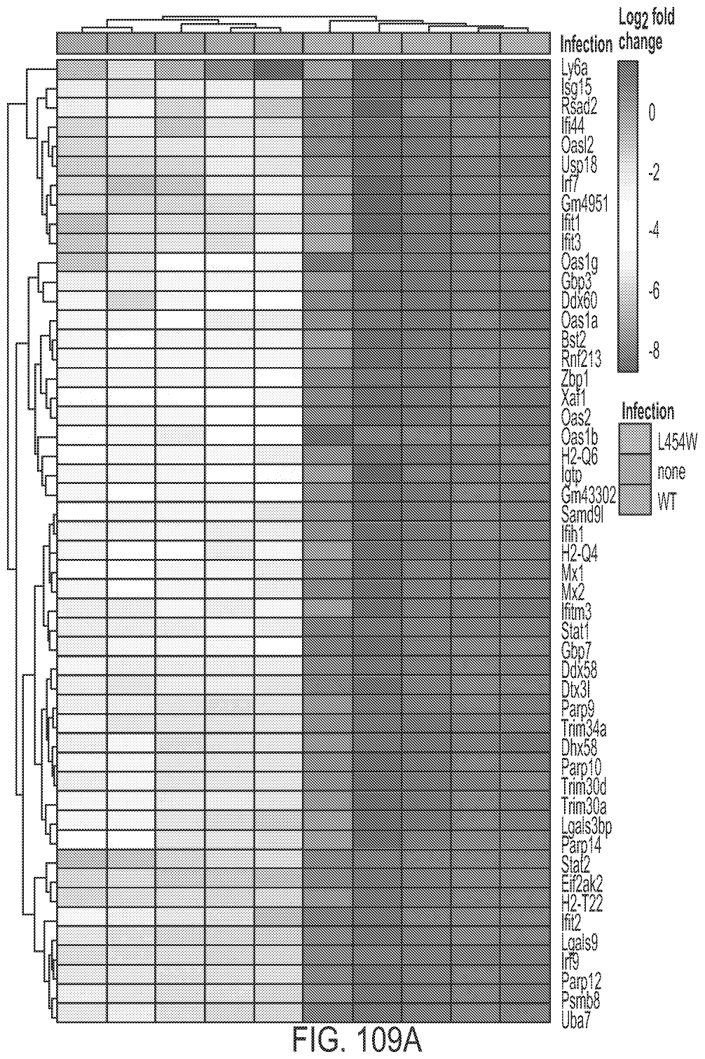

The amount of MeV N RNA copies—reflecting the viral load present in the OBC at the end of the experiment—was quantified using RT-qPCR. FIG. 105F shows the significant 2 log reduction of the viral load in the cultures treated post-infection with 100 nM of HRC4 peptide (**p=0.008, Mann-Whitney U-test) and a 4 fold significant reduction in the number of MeV N RNA copies in the group treated with 10 nM (*p=0.03, Mann-Whitney U-test), compared to the viral load in untreated samples. No significant variation of the viral load was observed in the groups treated with lower concentrations of fusion inhibitor. MeV IC323-EGFP-F L454W virus invaded the tissue, forming extensive areas of fusion throughout the culture four days after infection (FIG. 105G). The HRC4 peptide (either 100 nM or 10 nM) blocks the spread of MeV-IC323-EGFP-F L454W over the same time period (FIG. 105G). At the highest concentration used (100 nM) only isolated single infected cells were observed. The lower concentration (10 nM) was only partially inhibitory, with a few focal areas of dissemination were observed (FIG. 105G), and no significant differences between the lower concentrations (below 10 nM) and the untreated tissues were noted (FIG. 105F). Since the fusion inhibitor was added 24 hours after infection, these results indicate that the HRC4 peptides block cell-to-cell spread in three-dimensional CNS tissue, suggesting that MeV dissemination in brain tissue depends on the function of the fusion protein. Even when using OBC from IFNAR1KO mice that express the human CD150 (SLAM) receptor, enhancing viral spread in the CNS, the highest concentration of peptide blocked dissemination of MeV IC323-EGFP-F L454W at least as well as WT virus (FIG. 108).

### Evolution of the L454W Bearing Viruses in Immune Competent OBC

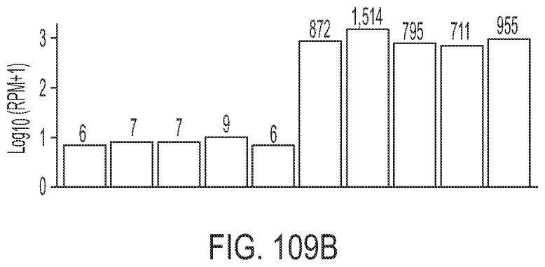

To evaluate viral evolution of the two viruses bearing the double population of L454W plus either L454W/E455G or L454W/G506E in the presence of fully competent mouse CNS we derived OBC from wt mice. OBC from C57BL/6 suckling mice were infected with MeV wt and the L454W F viruses (with either the additional L454W/E455G or L454W/G506E). We previously observed (Ferren et al., 2019) that wt virus did not spread in this model and we confirmed the finding here. In contrast both viruses bearing the L454W F spread efficiently in the C57BL/6 OBC and had similar levels of infection after 7 days (FIG. 105H). After 7 days, OBC were lysed for sequencing to assess transcriptome, viral evolution, and quantification of viral RNA. In C57BL/6 OBC, viruses bearing the L454W F induced a gene expression pattern associated with a strong interferon signaling compared to the OBC infected with wt virus (with minimal spread), where the gene pattern was indistinguishable from uninfected OBC (see FIG. 109 for differential gene expression analysis). Despite this strong innate immune response, the spread of L454W F bearing virus though reduced was not halted (see FIG. 105I). We then analyzed the viral sequence in these OBCs to determine whether the L454W/E455G and L454W/G506E mutations were conserved. Sequence analysis is presented in LAVA plots (Lin et al., 2019b) in the supplemental material; LAVA plot #1 shows the allele frequency for L454W/G506E in four samples (CM005-8) and for L454W/E455G in one sample (CM017). The L454W/G506E input virus had an allele frequency for the double mutant of ˜36% (observed in the viral preparation) that rose after 7 days in ex vivo tissues to 97%, 89%, 96%, and 78%. For the L454W/E455G, the input virus had an allele frequency for the double mutant of ˜22%, and in the sequence from the ex vivo the double mutant decreased to ˜4%.

Infection of Human Induced Pluripotent Stem Cell (hiPSC)-Derived Brain Organoids: CNS Adapted Variants Vs. Wt Virus

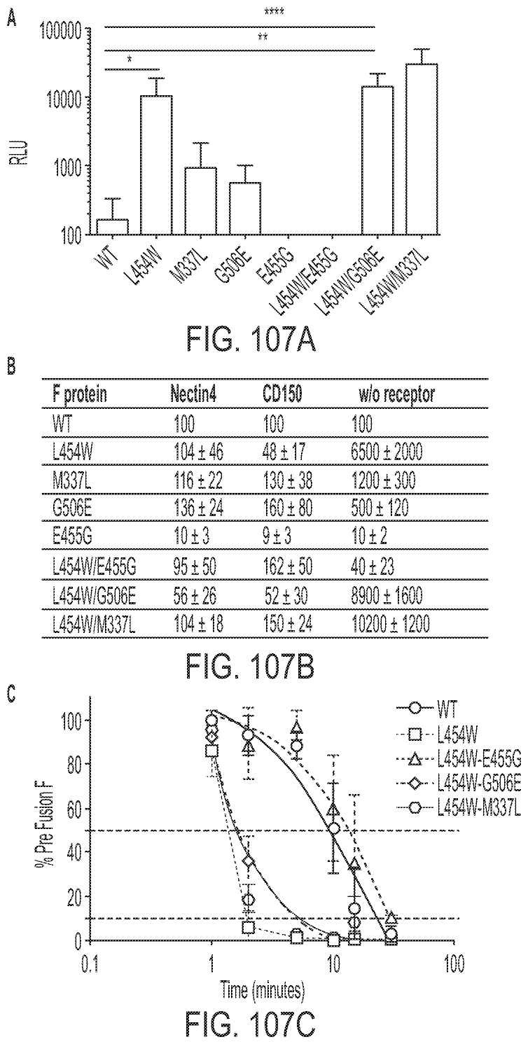

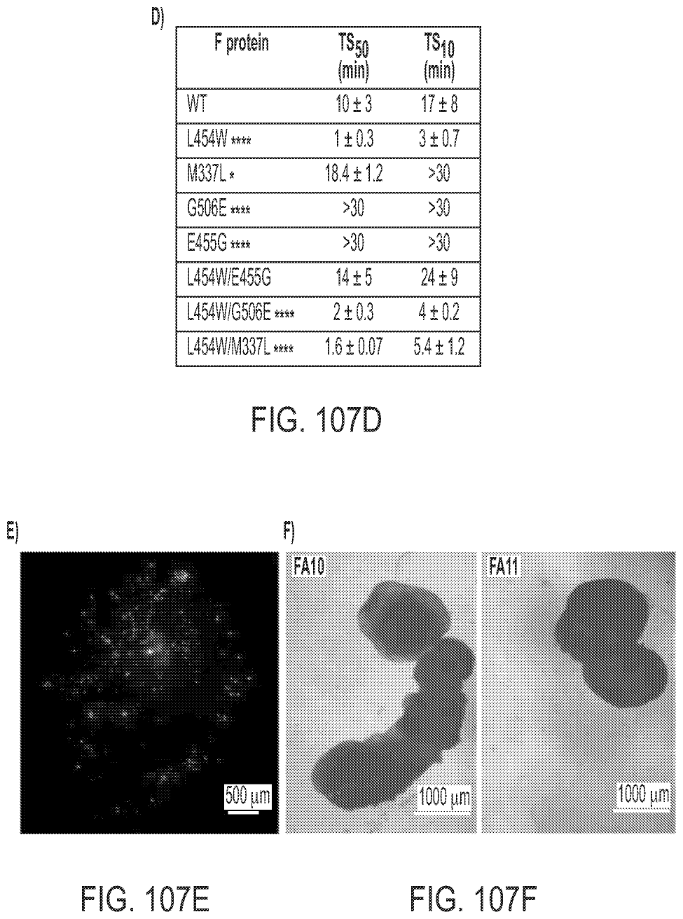

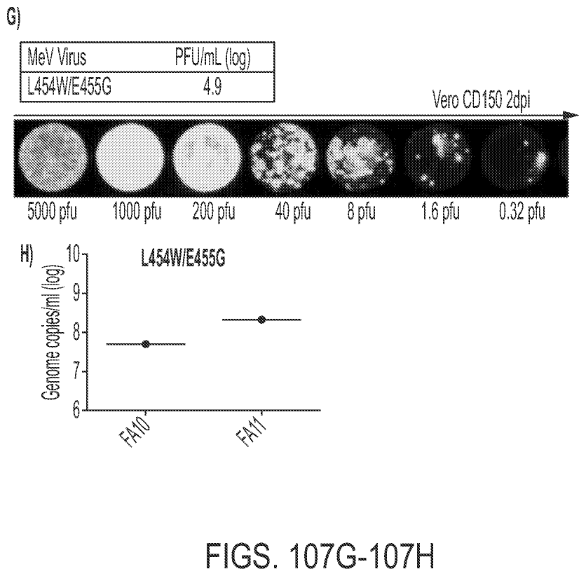

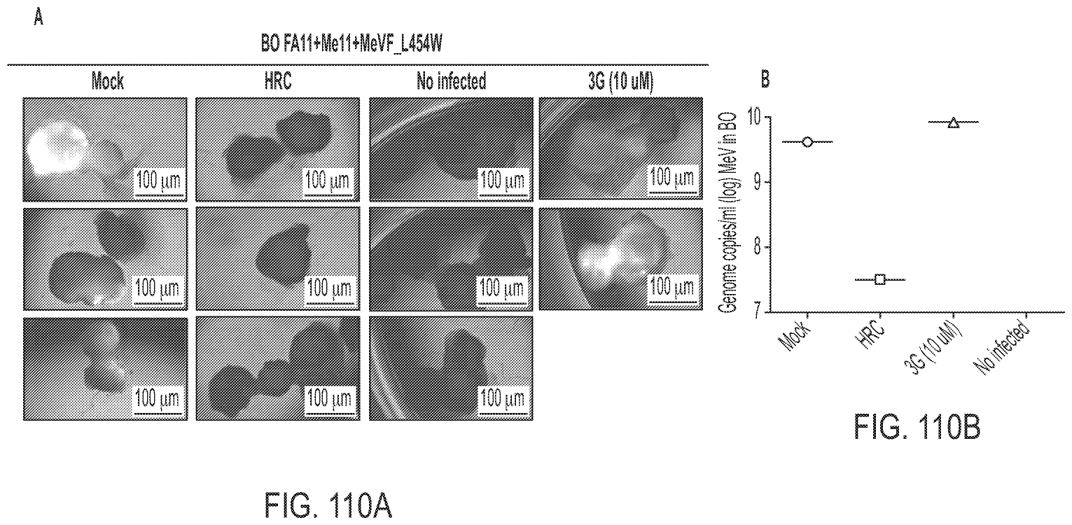

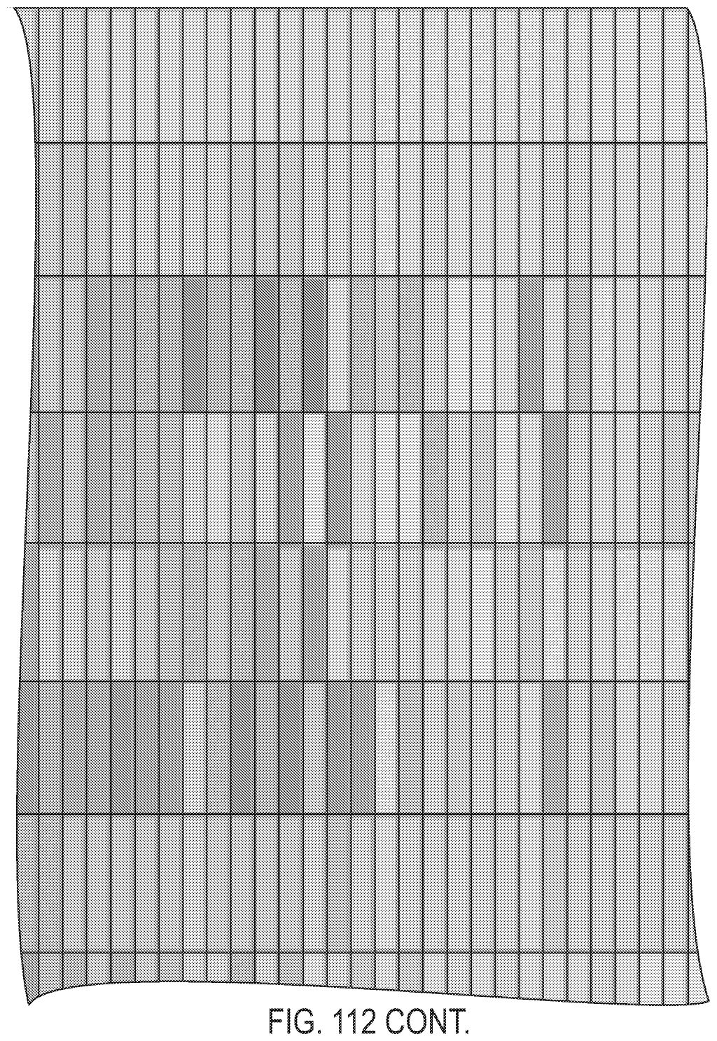

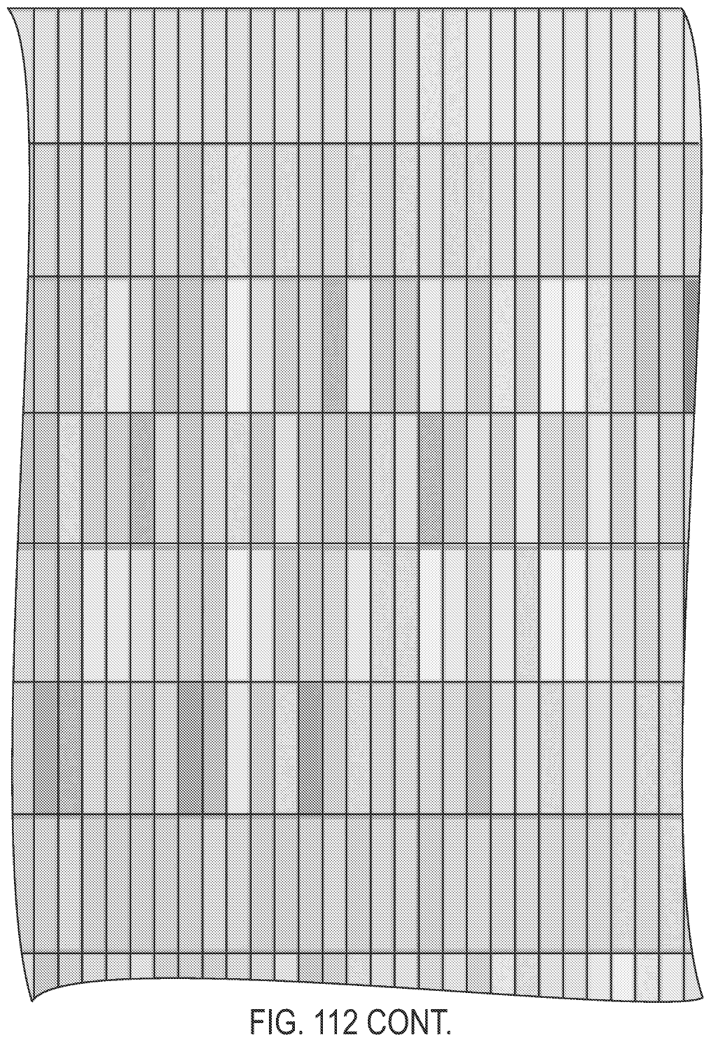

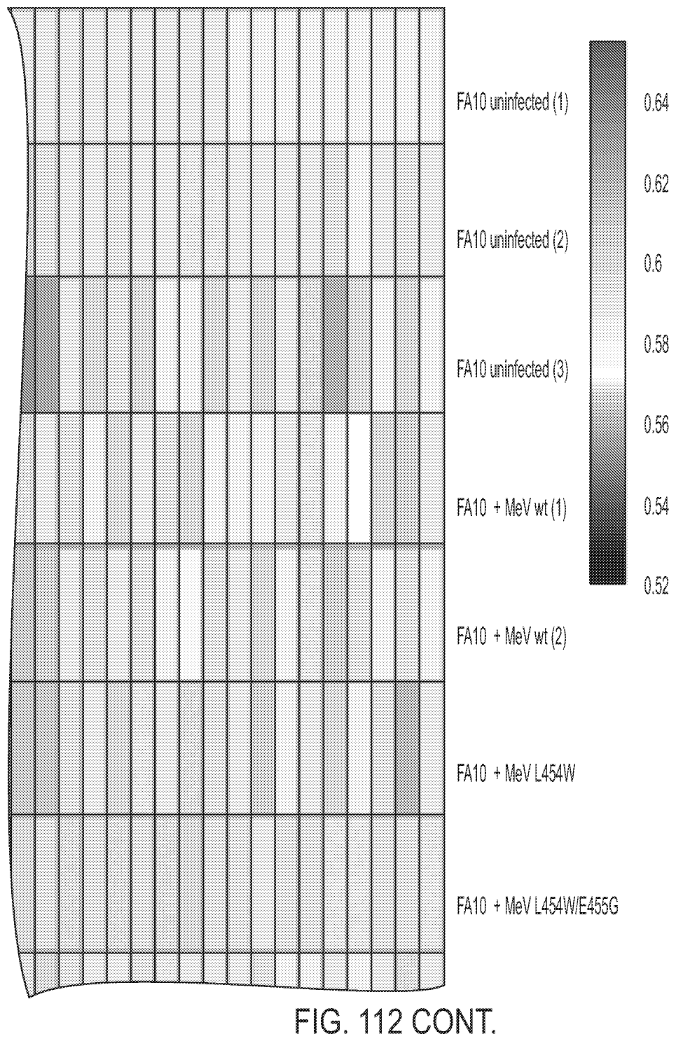

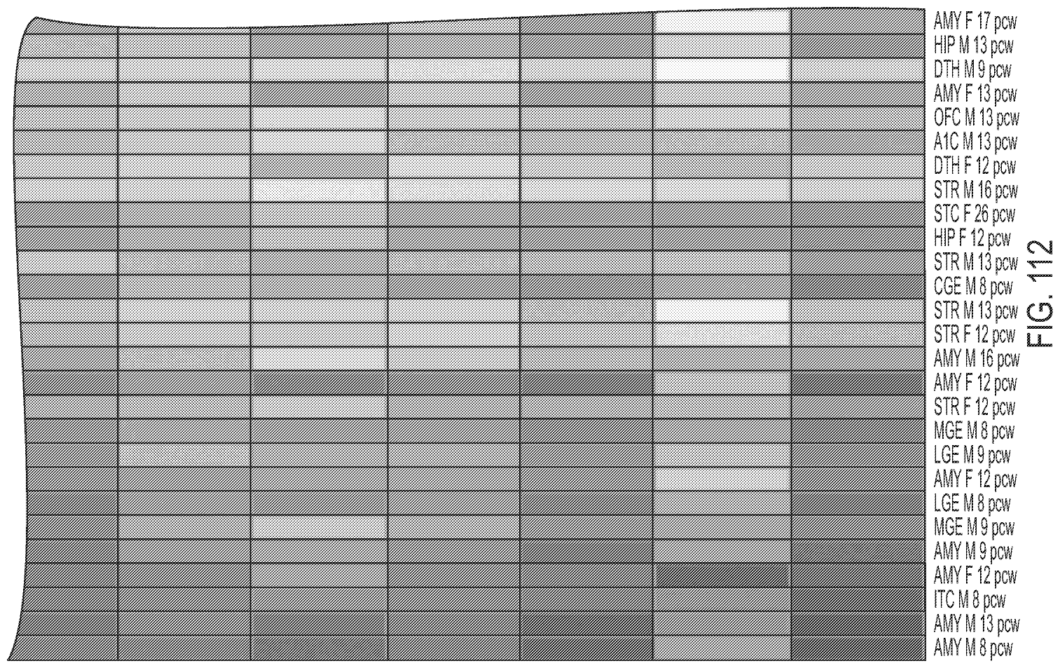

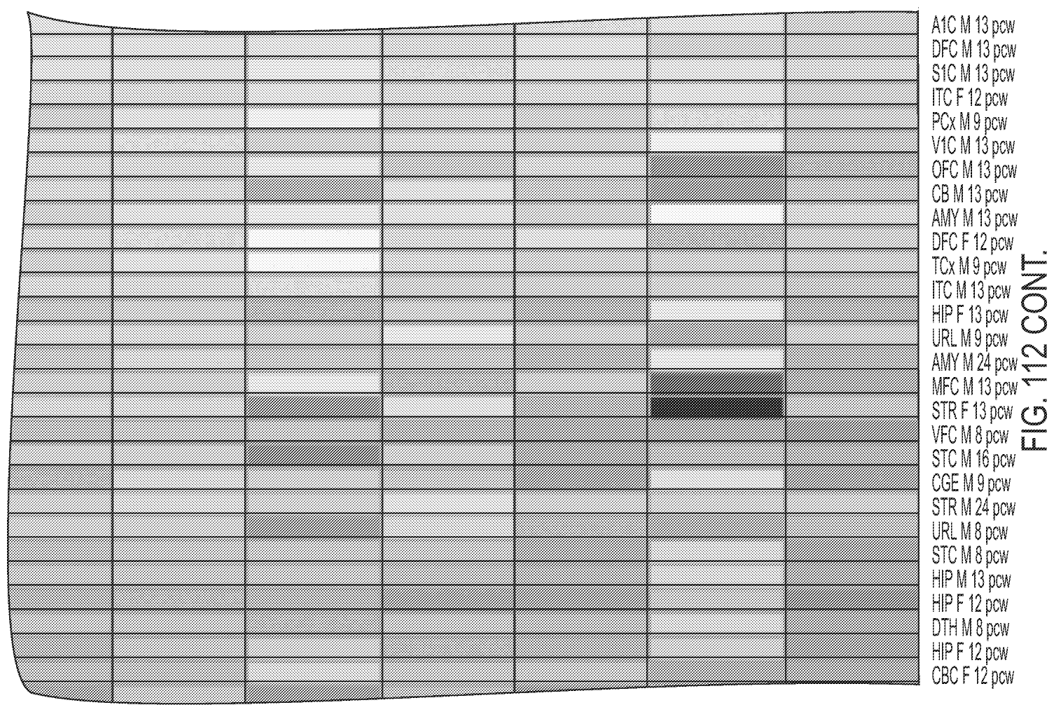

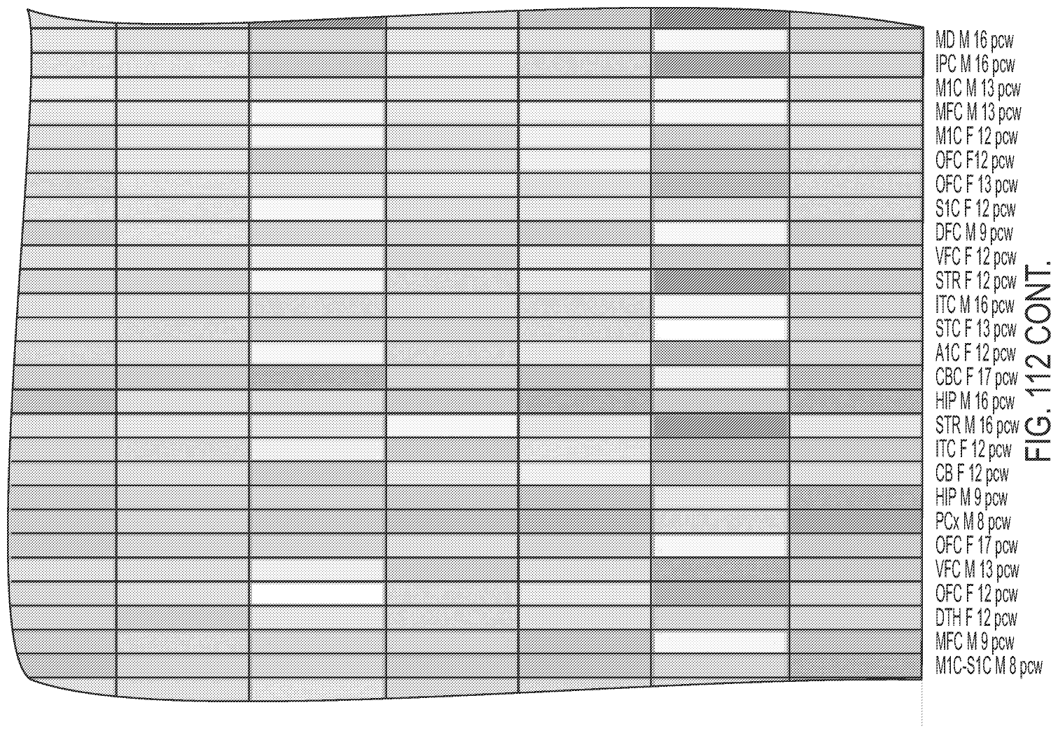

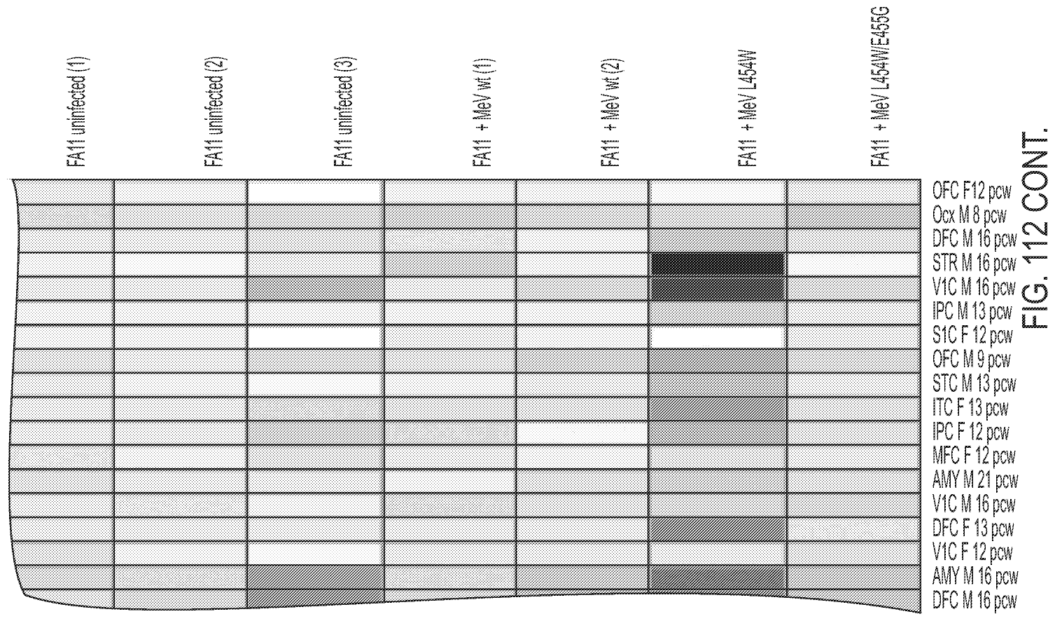

To extend the CNS modeling to human neural tissue, we differentiated hiPSC (from two separate donors, one male and one female, FA10 and FA11) into brain organoids (Lancaster and Knoblich, 2014). 90-day old organoids were infected with viruses bearing wt or L454W F. To analyze whether the alterations in viral spread in this model are related to the properties of F and not simply attributable to the individual mutations, organoids were also infected with MeV bearing F with mutations N462K (a lab adapted mutation previously found to grow efficiently in hamster brains) and T461I (previously found in a SSPE case) (Watanabe et al., 2013). These mutations are described in FIG. 104. Infection was monitored over a 10-day period and spread was assessed by monitoring fluorescence (FIG. 106A and FIG. 110 show pictures taken at day 10). To ensure that infection was performed with equal amounts of virus, the inoculum used for infection of human brain organoids was assessed in parallel in Vero cells expressing CD150 (Vero-CD150) as shown in FIG. 106B and FIG. 110. All the viral titers were similar (pfu/ml), with L454W mutant only slightly lower. In Vero-CD150 all the viruses efficiently spread and destroyed the cell monolayer within 3 days (data not shown). After 10 days, infected and uninfected human brain organoids were lysed for RNA sequencing to assess the transcriptome, to monitor viral evolution during organoid infection, and to quantify viral RNA (see FIGS. 107C-D, FIG. 112 and supplemental material).

The viruses bearing L454W F spread more efficiently in the human brain organoids than the virus bearing the wt F (FIGS. 106A and C) and were efficiently blocked by the HRC4 fusion inhibitor added 24 hours after infection (FIG. 110). A virus bearing an T461I mutated F (derived from an SSPE patient) also spread in the brain organoids. In contrast, the virus bearing the N462K F showed only a modest increase in spread compared to wt virus (FIGS. 106A and C).

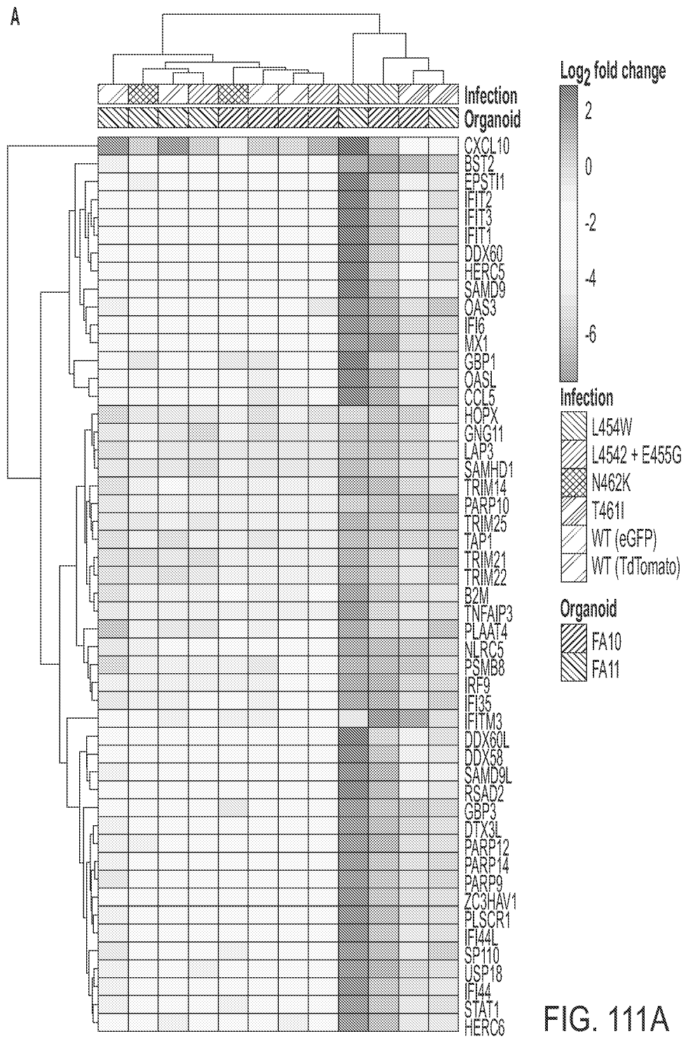

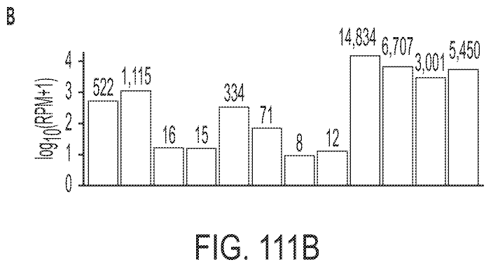

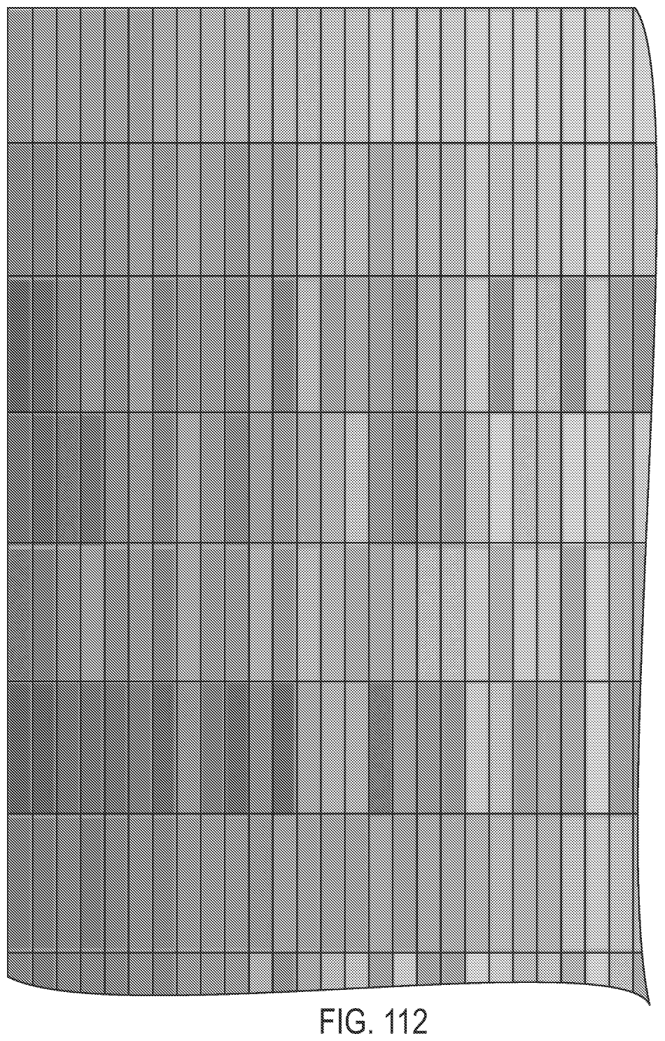

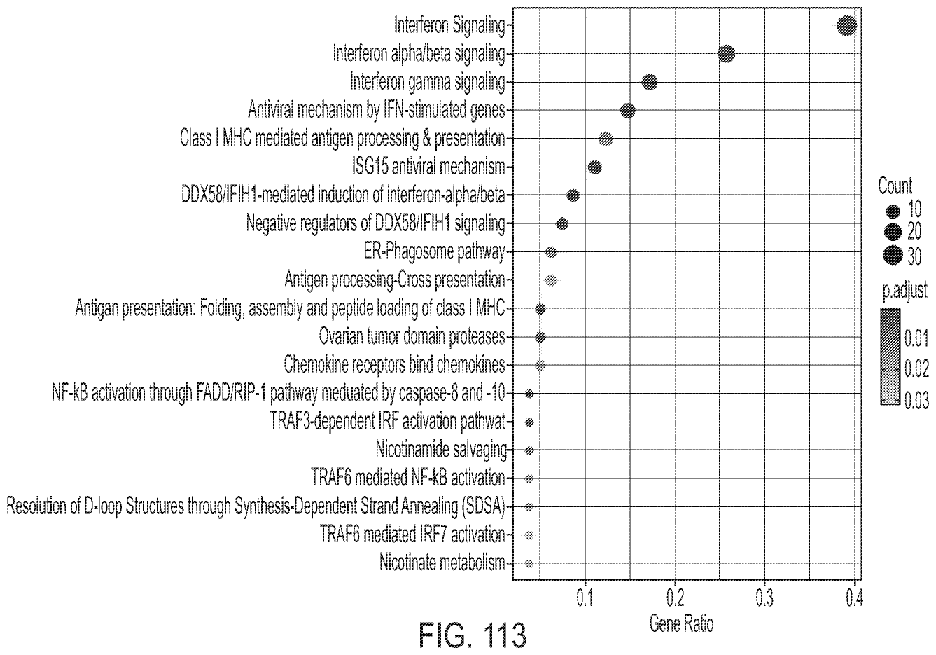

In FIG. 106D we compared the differential gene expression between cells that were uninfected or infected (with either wt virus or virus bearing L454W F proteins) in human brain organoids. MeV bearing L454W F was present at several-fold higher reads per million (RPM) values than MeV wt (FIG. 106D). The viruses bearing L454W F induced a gene expression pattern that was associated with interferon signaling, compared to organoids infected with wt virus and control samples (p=2.6×10-17, see FIG. 110 for the differential gene expression analysis). This response seems likely to be the consequence of the differences in viral growth, since the interferon response seems to correlate with viral count independently from the specific F mutation (FIG. 111 shows the differential expression from infection with all the MeV variants shown in FIG. 106A). For both sets of human brain organoids, the highest gene-level expression coefficient correlated with the youngest fetal development stages (8-13 post-conception weeks) and with brain tissues derived from the amygdala (FIG. 112), as noted previously (Luo et al., 2016; Qian et al., 2016). Pathway analysis of differentially expressed genes common to the two human brain organoid infection series revealed that a strong interferon response was evoked by measles infection in human brain organoids (FIG. 113).

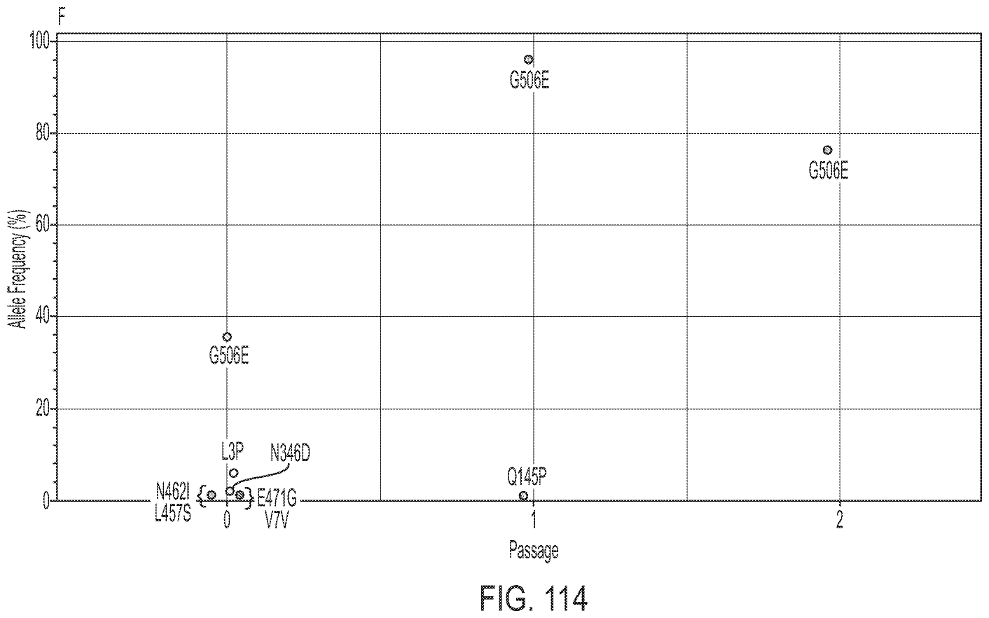

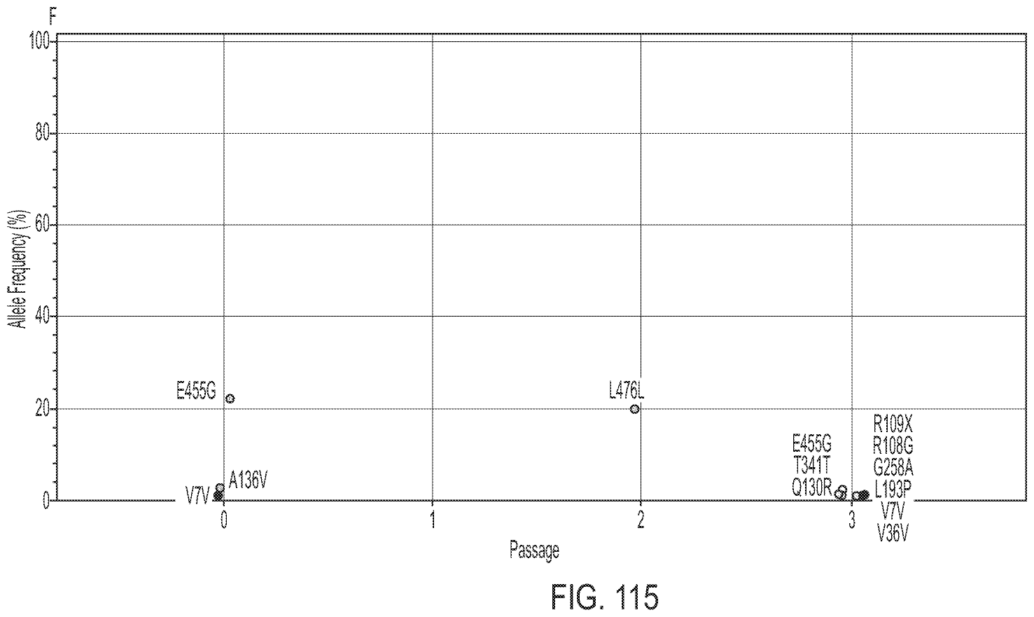

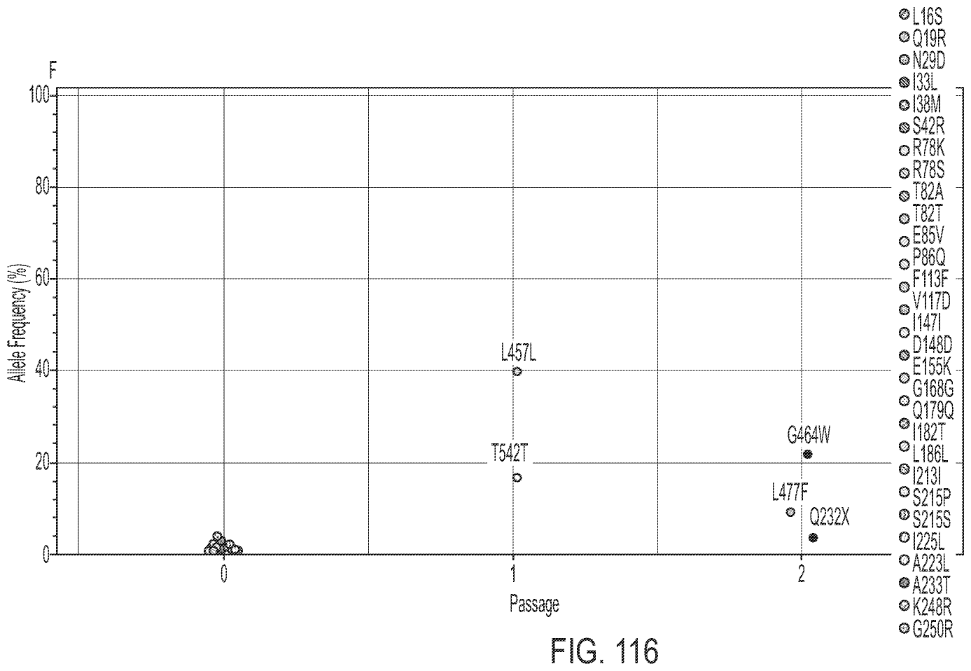

We then asked whether the two viral populations (L454W-L454W/E455G and L454W-L454W/G506E) evolved in this model as they had in the murine organotypic brain slices. The sequence analysis is presented in LAVA plots. In the case of the L454W-L454W/G506E population, the viral sequences emerging from the brain organoids were notable for the G506E variant in the F protein that increased from an allele frequency of ˜36% in the viral stock to ˜96% in FA10 and ˜78% in FA11 brain organoids (see data for FA10 in FIG. 114). The second viral stock (with L454W and L454W/E455G, where the double mutant was present at 22% allele frequency) was used to infect a second set of brain organoids (derived from FA11 iPSC). The L454W mutation was maintained and the allele frequency of the E455G F decreased to ˜2% in the organoids (see FIG. 115). Wild type virus did not show any remarkable alteration (see FIG. 116).

### The Additional Mutations in the L454W F Background Stabilize the Pre Fusion State of the F Protein

In both murine and human tissues we observed a positive selection of G506E in F and a negative selection of E455G. To understand the reason for this different selection of the two additional mutations, we assessed the functional properties of the F bearing the additional mutations, both alone and in combination with L454W using cells transfected with the cDNA of the indicated F proteins. The F bearing the L454W/G506E mutations was still significantly less stable than wt F in our functional assays, while the F bearing L454W/E455G was more stable than wt F (FIGS. 108A-B). The hallmark of the neuropathogenic variants is the ability to fuse without known viral receptor, and this phenotype was still present: the L454W/G506E F can mediate fusion without any of known receptors (CD150/SLAM or nectin 4). However, the L454W/E455G F, bearing the E455G mutation that dramatically decreased in frequency during organoid growth, does not mediate fusion without known receptor.

Based on the results from FIGS. 107A-B we then asked whether a homogenous viral population bearing the L454W/E455G F behaves like wt virus in the organotypic brain slices and in brain organoid models, and whether E455G would be subject to negative selection pressure in these neural tissues. We thus attempted to generate two new viruses bearing either the L454W/E455G F or the E455G F proteins and analyzed their growth and spread in brain tissue ex vivo. The recombinant virus bearing the double mutant L454W/E455G F grew similarly to wt virus in Vero-CD150. However, a recombinant virus bearing the singly mutated E455G F could not be recovered, suggesting it is detrimental in culture. To determine whether the double mutant has an advantage in brain, we coinfected with wt (red) and L454W/E455G (green) F-bearing viruses in OBC as in FIG. 105D. FIG. 107E shows the extent of viral spread 96 h post-infection. In clear contrast to the results shown FIG. 105D (where the L454W virus invaded the entire tissue, and wt infection was limited) the co-infection resulted in similar (limited) spread for both wt and L454W/E455G viruses.

To confirm the phenotype of the L454W/E455G virus in human brain organoids we infected 90-day old organoids (FIGS. 107F-H). Infection was monitored over a 10-day period and FIG. 107F shows fluorescent viral spread at day 10. To ensure that infection was performed with the intended amount of virus (5000 pfu/well), the inoculum used for infection of human brain organoids was assessed in parallel in Vero-CD150 as shown in FIG. 107G. The L454W/E455G F bearing virus had limited spread in both FA10 and FA11 hiPSC derived brain organoids after 10 days (FIG. 107F) but in Vero-CD150 spread and destroyed the cell monolayer within 2 days (FIG. 107G). After 10 days, infected human brain organoids were lysed for RNA sequencing to assess the transcriptome, to monitor viral evolution during organoid infection, and to quantify viral RNA and assess viral evolution (see FIG. 107H, FIG. 111, FIG. 113). The amount of viral genome of the infection from L454W/E455G was similar to what we observed for the wt virus (FIG. 106), and after 10 days, the double mutation remained stable (see LAVA plot #5). To confirm these findings, we differentiated another set of brain organoids from hiPSC FA11. A total of nine wells were infected with 1000 pfu/well of wt, L454W F (the mixed population L454W and L454W/E455G), and L4545/E455G F bearing viruses. Twenty days post-infection the brain organoids were lysed and RNA was extracted for viral sequencing. The data are presented in the LAVA plot #6. The wt virus had only a change in the P gene (R77C) with an allele frequency ˜30% in all the three samples. The L454W F bearing virus (with the mixed population L454W and L454W/E455G) in one sample totally eliminated the E455G mutation. In the second sample E455G remained at ˜24%, and the third one lost E455G and acquired an additional mutation (D538G) with a ˜23% frequency. The double mutant L454W/E455G remained stable without significant changes and cannot spread well in the brain organoids.

In light of the finding that an F-stabilizing mutation like G506E does not necessarily interfere with CNS adaptation, we searched for evidence of such a phenotype in vivo. A patient's virus isolated at the time of the original publication about the L454W mutation in F (Hardie et al., 2013) bore an additional mutation in the F protein (M337L). We assessed the effect of M337L on fusion and stability of F, both singly and in combination with L454W (FIGS. 107A and 107B). The expressed M337L F did not mediate fusion in the absence of receptor, and its pre-fusion state was more stable than wt F. The M337L/L454W F promoted fusion in the absence of receptor and was significantly less thermostable than wt F. Evolution in vivo led—through a different mutated residue—to a similar functional alteration as that caused by G506E. This pattern reflects a CNS specific pattern of adaptation.

### Discussion

In a South African MeV outbreak, 8 HIV-infected patients died of MeV CNS manifestations (Albertyn et al., 2011). We characterized the properties of one of the CNS adapted fusion complexes (composed of H and F) (Jurgens et al., 2015). The fusion complex sequences of MeV from the CNS of patients with MIBE are altered so that F is activated without a known entry receptor. The F proteins of the isolates from two separate patients contain one specific amino acid alteration at position 454 (L454W) that increases F's ability to mediate fusion with any heterotypic attachment protein, and markedly decreases F's thermal stability (Jurgens et al., 2015).

An unexpectedly informative set of mutations fortuitously emerged from the technical process of generating recombinant viruses bearing the L454W F by reverse genetics. The standard methods carried out at 37° C. resulted in a mixed population of viruses bearing either L454W F or L454W/E455G F. Lowering the temperature of viral production to 32° C. to stabilize the F resulted in a population containing both L454W and L454W/G506E. While E455 is located in the HRC domain (see FIG. 104), the residue G506 is in the transmembrane domain and is not resolved in the available crystal structures of MeV F. Placing a charged residue into the transmembrane domain would likely have a drastic impact on protein folding and activity and yet this mutation was positively selected in our ex vivo models. Our data show that the HRC mutation E455G results in an increased F stability and the L454W/E455G F (in distinction to L454W) requires engagement of H (MeV receptor binding protein) to receptor in order to mediate fusion. Thus, this second HRC domain mutation confers a requirement for H-receptor interaction. The G506E transmembrane domain mutation slightly restored F's stability, however the double mutant L454W/G506 F is still less stable than wt and mediates fusion in the absence of known receptor. Although arising as a result of a technical process, these compensatory mutations provided direct evidence that viruses that can fuse without known receptors are positively selected in the CNS.

The viruses bearing the T461I and N462K F proteins appear genetically stable in both cell culture and ex vivo (data not shown), however the virus bearing an N462K F, surprisingly did not grow as well as we would expect in human brain organoids. The double mutant L454W/G506E seems to have reached a “balance” that it is fit for both culture conditions. As we have observed for human parainfluenza virus type 3 (Iketani et al., 2018) isolation of viruses from clinical specimens in cell culture provides a selective pressure for viral evolution that may obscure authentic features of clinical strains. Direct sequence of clinical samples avoids incurring in these cell-line artifacts (Iketani et al., 2018). We previously described a virus from an SSPE case in which the consensus F (SSPE F) had 5 amino-acid changes (G168R, E170G, S262G, A440P, R520C, L550P) (Angius et al., 2019). The SSPE F was less thermally stable than the wt F and could mediate fusion in the absence of known receptor. When we assessed the effect of the mutations individually, we observed that no mutation alone could confer the fusion property. Some of the mutations increased the stability of the prefusion F (e.g., E170G) while others decreased it (e.g., S262G). We speculate that in vivo evolution leading to the fully CNS adapted SSPE F may have occurred in the clinical case.

Deep sequencing of one of the clinical samples where the L454W mutation in F was first identified revealed the presence of several other mutations in the F protein. One additional mutation in the F protein (M337L) had an allele frequency similar to that of L454W, and the F protein bearing the M337L/L454W matched the fusion phenotype of the F bearing the L454W/G506 mutations, demonstrating in vivo viral evolution towards similar F function to that observed in the brain organoids. These results suggest that different mutations that arrive at a similarly labile and receptor-independent F may be positively selected in the CNS. We mapped the M337L mutation onto the prefusion structure of MeV F. This mutation could potentially form hydrophobic interactions with L256 and L257 to stabilize the prefusion conformation of MeV F. A virus bearing the L454W/E455G F was successfully recovered in our experiments and behaved similarly to wt virus. Infection in brain organoids was limited (as for wt virus) and in the 10-20 days span of two separate experiments presented here did not result in negative selection of the mutation. It is possible than a longer infection could result in elimination of the E455G mutation or introduction of additional mutations. Despite several attempts, a virus bearing purely E455G F could not be recovered, and we gather that the increased stability of the E455G F may be detrimental to fitness.

### Figure Captions

FIG. 104 shows location of substitutions within the F protein from CNS-adapted virus. (A) Schematic of MeV F with fusion peptide (FP), N-terminal heptad repeat (HRN), C-terminal heptad repeat (HRC), transmembrane (TM), and cytoplasmic (CT) domains indicated. (B) Ribbon diagrams of the prefusion (left, MeV F; PDB 5YXW) and post-fusion (right, HPIV3 F; PDB 1ZTM) conformations. Five substitutions (M337L, L454W, E455G, T461I and N462K) in F protein structures are shown.

FIG. 105 shows ex vivo infection with wild type (wt) vs. virus bearing the L454W F: the CNS-adapted virus outcompetes the wt virus in organotypic brain cultures (OBC). (A-B) OBC from IFNARKO murine brains were infected with 5000 plaque forming unit (pfu)/slice wt virus bearing EGFP (green fl (C, D) OBC from IFNARKO murine brains were co-infected at 5000 plaque forming unit (pfu)/slice with wt virus bearing tdTomato (red fluorescence) and MeV-IC323-L454W F EGFP (green fluorescence) at 5000 pfu/slice and monitored over 96 hours. Photos were taken at 24 hours (C) and 96 hours (D). Scale bar=500 μm. (E-G) MeV F derived fusion inhibitor peptide (HRC4) inhibits the dissemination of MeV bearing L454W F in OBC. OBC from IFNARKO murine brains were infected with MeV-IC323-L454W F EGFP at 5000 pfu/slice for 4 days. OBC were treated at the indicated concentrations or left untreated (NT control) by adding HRC4 fusion inhibitory peptide 24, 48 and 72 h after initial infection. (E) Schematic of the procedure. (F) Total RNA was harvested from organotypic slices at 4 days post infection, and the level of MeV N gene expression was quantified by RT-qPCR. Results are expressed as means±standard deviations of cultures from 5 different mice (**, P<0.01; ***, P<0.001 [Mann-Whitney-U test]). (G) Green fluorescence related to infection was observed 4 dpi by epifluorescence microscopy in OBC treated at the indicated concentrations (scale bar=500 μm). (H) Ex vivo virus bearing the L454W F infection in fully immune competent OBC. OBC from C57/BL6 murine brains were infected with L454W F bearing virus (using both viral preparations, one with the additional E455G in F and the one with G506E in F) at 1000 pfu/slice for 7 days. Picture were taken at 4 days after infection as indicated. (I) L454W—bearing virus growth in wt and IFNARKO OBC. OBC from wt or IFNARKO murine brains were infected with 1000 PFU/slice MeV-IC323-L454W F EGFP for 7 days. Total RNA was harvested from OBC at 4 days post infection, and the level of MeV N gene expression was quantified by RT-qPCR. Results are expressed as means±standard deviations in cultures from at least 5 different mice (*, P<0.05; ***, P<0.001 [Mann-Whitney-U test]).

FIG. 106 shows CNS adapted MeV variants spread efficiently in human pluripotent stem cell (hiPSC) derived brain organoids. (A) Two separate sets of 90 day old human brain organoids (derived from two hiPSCs, FA10 and FA11) were infected with recombinant MeV viruses (with either EGFP or tdTomato fluorescent protein) bearing the indicated MeV fusion (F) proteins. For each virus, 3 separate wells each containing 2-4 organoids were infected (5,000 pfu/well). The brain organoids were monitored over time and the fluorescence shown here reflects the infection after 10 days. Bar=1000 μm. (B) Viral titer of the inoculum used for infection was assessed on Vero CD150 as pfu/ml (log). (C) Total RNA was harvested from the human brain organoids at 10 days post infection, and the level of MeV N gene expression was quantified by RT-qPCR. (D) RNA-Seq analysis of wt vs. L454W F bearing virus infection in brain organoids (data from three separate experiments). Seven replicates of uninfected and MeV infected brain organoids (n=6 uninfected, n=5 WT, n=2 L454W F-bearing virus) were transcriptionally profiled. RPM values for MeV for each sample are depicted below each heatmap. Raw counts were normalized across all samples and differential expression analysis performed. The 50 genes with the lowest adjusted p value between L454W and uninfected are depicted in the heatmap, colored by log 2 fold change of each sample relative to the mean normalized counts for each gene.

FIG. 107 shows fusion activity and thermal stability of MeV fusion (F) proteins bearing the indicated mutations. (A) Cell-to-cell fusion between HEK293T cells co-expressing the indicated MeV F proteins and MeV wt hemagglutinin (H) and HEK293T cells (without any known measles receptor) was assessed by a B-gal complementation assay. The values on the Y-axis are expressed as relative luminescence unit (RLU) averages (with standard error, SE) of results from three independent experiments.*p<0.05, **p<0.01,***p<0.001, ****, p<0.0001 (2way ANOVA). (B) Percent of fusion activity of MeV F protein (IC323) bearing the indicated mutations compared to wt F in the presence of nectin 4, CD150 or no receptor. (C) HEK293T cells were transfected with MeV F protein bearing the indicated mutations and incubated at 37° C. for 24 h, then raised to 55° C. for the indicated times (Xaxis). The values on the Y-axis represent the percentages of pre-fusion conformation specific antibody binding to the indicated F proteins (compared to the wt F protein at time zero). The values are the average of three independent experiments. ****, p<0.0001 (2way ANOVA results are summarized in FIG. 4D). (D) Time (minutes) at 55° C. that decreases the fraction of prefusion epitope to 50% (TS50) and to 10% (TS10) compared to wt F at time zero (100%). Data are averages from three experiments+/−SE. (E) OBC from IFNARKO murine brains were co-infected with 5000 pfu/slice of wt virus bearing tdTomato (red fluorescence) and MeV-IC323-L454W/E455G F EGFP (green fluorescence) and monitored over 96 hours. Photos were taken at 96 hours. Scale bar=500 μm. (F) Two separate sets of 90 day old human brain organoids (derived from two hiPSCs, FA10 and FA11) were infected with recombinant MeV viruses bearing the L4545W/E455G F protein. Three separate wells containing 2-4 organoids were infected (5000 pfu/well). The brain organoids were monitored over time and the fluorescence shown here reflects the infection after 10 days. Bar=1000 μm. (G) Viral titer of the viral inoculum used for infection was assessed on Vero CD150 (pfu/ml; log). Photos show the extent of infection after 2 days (pfu/well are indicated). (H) Total RNA was harvested from the human brain organoids at 10 days post infection, and the level of MeV N gene expression was quantified by RT-qPCR.

### REFERENCES

1. Ader, N., Brindley, M., Avila, M., Orvell, C., Horvat, B., Hiltensperger, G., Schneider-Schaulies, J., Vandevelde, M., Zurbriggen, A., Plemper, R. K., et al. (2013). Mechanism for active membrane fusion triggering by morbillivirus attachment protein. J. Virol. 87, 314-326.
2. Albertyn, C., van der Plas, H., Hardie, D., Candy, S., Tomoka, T., Leepan, E. B., and Heckmann, J. M. (2011). Silent casualties from the measles outbreak in South Africa. S. Afr. Med. J. 101, 313-314, 316-317.
3. Allen, I. V, McQuaid, S., McMahon, J., Kirk, J., and McConnell, R. (1996). The significance of measles virus antigen and genome distribution in the CNS in SSPE for mechanisms of viral spread and demyelination. Journal of Neuropathology and Experimental Neurology 55, 471-480.
4. Angius, F., Smuts, H., Rybkina, K., Stelitano, D., Eley, B., Wilmshurst, J., Ferren, M., Lalande, A., Mathieu, C., Moscona, A., et al. (2019). Analysis of a Subacute Sclerosing Panencephalitis Genotype B3 Virus from the 2009-2010 South African Measles Epidemic Shows That Hyperfusogenic F Proteins Contribute to Measles Virus Infection in the Brain. J. Virol. 93.
5. Avila, M., Alves, L., Khosravi, M., Ader-Ebert, N., Origgi, F., Schneider-Schaulies, J., Zurbriggen, A., Plemper, R. K., and Plattet, P. (2014). Molecular determinants defining the triggering range of prefusion F complexes of canine distemper virus. J. Virol. 88, 2951-2966.
6. Ayata, M., Takeuchi, K., Takeda, M., Ohgimoto, S., Kato, S., Sharma, L. B., Tanaka, M., Kuwamura, M., Ishida, H., and Ogura, H. (2010). The F gene of the Osaka-2 strain of measles virus derived from a case of subacute sclerosing panencephalitis is a major determinant of neurovirulence. J. Virol. 84, 11189-11199.
7. Baldolli, A., Dargère, S., Cardineau, E., Vabret, A., Dina, J., de La Blanchardiere, A., and Verdon, R. (2016). Measles inclusion-body encephalitis (MIBE) in a immunocompromised patient. Journal of Clinical Virology 81, 43-46.
8. Battles, M. B., Langedijk, J. P., Furmanova-Hollenstein, P., Chaiwatpongsakorn, S., Costello, H. M., Kwanten, L., Vranckx, L., Vink, P., Jaensch, S., Jonckers, T. H. M., et al. (2016). Molecular mechanism of respiratory syncytial virus fusion inhibitors. Nat. Chem. Biol. 12, 87-93.
9. Buchanan, R., and Bonthius, D. J. (2012). Measles Virus and Associated Central Nervous System Sequelae. Seminars in Pediatric Neurology 19, 107-114.
10. Cattaneo, R., Schmid, A., Billeter, M. A., Sheppard, R. D., and Udem, S. A. (1988). Multiple viral mutations rather than host factors cause defective measles virus gene expression in a subacute sclerosing panencephalitis cell line. J. Virol. 62, 1388-1397.
11. Chang, A., and Dutch, R. E. (2012). Paramyxovirus fusion and entry: multiple paths to a common end. Viruses 4, 613-636.
12. Cosby, S. L., and Brankin, B. (1995). Measles virus infection of cerebral endothelial cells and effect on their adhesive properties. Vet. Microbiol. 44, 135-139.
13. Delhaye, S., Paul, S., Blakqori, G., Minet, M., Weber, F., Staeheli, P., and Michiels, T. (2006). Neurons produce type I interferon during viral encephalitis. Proc. Natl. Acad. Sci. U.S.A. 103, 7835-7840.
14. Delpeut, S., Sawatsky, B., Wong, X.-X., Frenzke, M., Cattaneo, R., and von Messling, V. (2017). Nectin-4 Interactions Govern Measles Virus Virulence in a New Model of Pathogenesis, the Squirrel Monkey (Saimiri sciureus). Journal of Virology 91, e02490-16.
15. Devaux, P., Hodge, G., McChesney, M. B., and Cattaneo, R. (2008). Attenuation of V- or C-defective measles viruses: infection control by the inflammatory and interferon responses of rhesus monkeys. Journal of Virology 82, 5359-5367.
16. Devaux, P., Hudacek, A. W., Hodge, G., Reyes-del Valle, J., McChesney, M. B., and Cattaneo, R. (2011). A Recombinant Measles Virus Unable To Antagonize STAT1 Function Cannot Control Inflammation and Is Attenuated in Rhesus Monkeys. Journal of Virology 85, 348-356.
17. Esolen, L. M., Takahashi, K., Johnson, R. T., Vaisberg, A., Moench, T. R., Wesselingh, S. L., and Griffin, D. E. (1995). Brain endothelial cell infection in children with acute fatal measles. J. Clin. Invest. 96, 2478-2481.
18. Ferren, M., Horvat, B., and Mathieu, C. (2019). Measles Encephalitis: Towards New Therapeutics. Viruses 11.
19. Frenzke, M., Sawatsky, B., Wong, X. X., Delpeut, S., Mateo, M., Cattaneo, R., and von Messling, V. (2013). Nectin-4-dependent measles virus spread to the cynomolgus monkey tracheal epithelium: role of infected immune cells infiltrating the lamina propria. Journal of Virology 87, 2526-2534.
20. Generous, A. R., Harrison, O. J., Troyanovsky, R. B., Mateo, M., Navaratnarajah, C. K., Donohue, R. C., Pfaller, C. K., Alekhina, O., Sergeeva, A. P., Indra, I., et al. (2019). Trans-endocytosis elicited by nectins transfers cytoplasmic cargo, including infectious material, between cells. J. Cell. Sci. 132.
21. Griffin, D. E., Lin, W.-H., and Pan, C.-H. (2012). Measles virus, immune control, and persistence. FEMS Microbiol. Rev. 36, 649-662.
22. Ha, M. N., Delpeut, S., Noyce, R. S., Sisson, G., Black, K. M., Lin, L.-T., Bilimoria, D., Plemper, R. K., Prive, G. G., and Richardson, C. D. (2017). Mutations in the Fusion Protein of Measles Virus That Confer Resistance to the Membrane Fusion Inhibitors Carbobenzoxy-d-Phe-1-Phe-Gly and 4-Nitro-2-Phenylacetyl Amino-Benzamide. J. Virol. 91
23. Hardie, D. R., Albertyn, C., Heckmann, J. M., and Smuts, H. E. M. (2013). Molecular characterisation of virus in the brains of patients with measles inclusion body encephalitis (MIBE). Virol. J. 10, 283.
24. Harrison, S. C. (2008). Viral membrane fusion. Nat. Struct. Mol. Biol. 15, 690-698.
25. Hashiguchi, T., Fukuda, Y., Matsuoka, R., Kuroda, D., Kubota, M., Shirogane, Y., Watanabe, S.,
26. Tsumoto, K., Kohda, D., Plemper, R. K., et al. (2018). Structures of the prefusion form of measles virus fusion protein in complex with inhibitors. Proc. Natl. Acad. Sci. U.S.A. 115, 2496-2501.
27. Hosoya, M. (2006). Measles encephalitis: direct viral invasion or autoimmune-mediated inflammation? Intern. Med. 45, 841-842.
28. Hughes, I., Jenney, M. E., Newton, R. W., Morris, D. J., and Klapper, P. E. (1993). Measles encephalitis during immunosuppressive treatment for acute lymphoblastic leukaemia. Arch. Dis. Child. 68, 775-778.
29. Iketani, S., Shean, R. C., Ferren, M., Makhsous, N., Aquino, D. B., des Georges, A., Rima, B., Mathieu, C., Porotto, M., Moscona, A., et al. (2018). Viral Entry Properties Required for Fitness in Humans Are Lost through Rapid Genomic Change during Viral Isolation. MBio 9.
30. Jansen, V. a. A., Stollenwerk, N., Jensen, H. J., Ramsay, M. E., Edmunds, W. J., and Rhodes, C. J. (2003). Measles outbreaks in a population with declining vaccine uptake. Science 301, 804.
31. Jurgens, E. M., Mathieu, C., Palermo, L. M., Hardie, D., Horvat, B., Moscona, A., and Porotto, M. (2015). Measles fusion machinery is dysregulated in neuropathogenic variants. MBio 6.
32. Lambert, D. M., Barney, S., Lambert, A. L., Guthrie, K., Medinas, R., Davis, D. E., Bucy, T., Erickson, J., Merutka, G., and Petteway, S. R. (1996). Peptides from conserved regions of paramyxovirus fusion (F) proteins are potent inhibitors of viral fusion. Proc. Natl. Acad. Sci. U.S.A. 93, 2186-2191.
33. Lancaster, M. A., and Knoblich, J. A. (2014). Generation of cerebral organoids from human pluripotent stem cells. Nat Protoc 9, 2329-2340.
34. Lemon, K., de Vries, R. D., Mesman, A. W., McQuaid, S., van Amerongen, G., Yuksel, S., Ludlow, M., Rennick, L. J., Kuiken, T., Rima, B. K., et al. (2011). Early target cells of measles virus after aerosol infection of non-human primates. PLoS Pathogens 7, e1001263.
35. Leonard, V. H. J., Sinn, P. L., Hodge, G., Miest, T., Devaux, P., Oezguen, N., Braun, W., McCray, P. B., McChesney, M. B., Cattaneo, R., et al. (2008). Measles virus blind to its epithelial cell receptor remains virulent in rhesus monkeys but cannot cross the airway epithelium and is not shed. The Journal of Clinical Investigation 118, 2448-2458.
36. Leonard, V. H. J., Hodge, G., Reyes-Del Valle, J., McChesney, M. B., and Cattaneo, R. (2010). Measles virus selectively blind to signaling lymphocytic activation molecule (SLAM; CD150) is attenuated and induces strong adaptive immune responses in rhesus monkeys. Journal of Virology 84, 3413-3420.
37. Lin, J.-Y., Kuo, R.-L., and Huang, H.-I. (2019a). Activation of type I interferon antiviral response in human neural stem cells. Stem Cell Res Ther 10, 387.
38. Lin, M. J., Shean, R. C., Makhsous, N., and Greninger, A. L. (2019b). LAVA: a streamlined visualization tool for longitudinal analysis of viral alleles. BioRxiv 2019.12.17.879320.
39. Luo, C., Lancaster, M. A., Castanon, R., Nery, J. R., Knoblich, J. A., and Ecker, J. R. (2016). Cerebral Organoids Recapitulate Epigenomic Signatures of the Human Fetal Brain. Cell Rep 17, 3369-3384.
40. Makhortova, N. R., Askovich, P., Patterson, C. E., Gechman, L. A., Gerard, N. P., and Rall, G. F. (2007). Neurokinin-1 enables measles virus trans-synaptic spread in neurons. Virology 362, 235-244.
41. Mathieu, C., Huey, D., Jurgens, E., Welsch, J. C., DeVito, I., Talekar, A., Horvat, B., Niewiesk, S., Moscona, A., and Porotto, M. (2015). Prevention of measles virus infection by intranasal delivery of fusion inhibitor peptides. J. Virol. 89, 1143-1155.
42. Mathieu, C., Ferren, M., Jurgens, E., Dumont, C., Rybkina, K., Harder, O., Stelitano, D., Madeddu, S., Sanna, G., Schwartz, D., et al. (2019). Measles Virus Bearing Measles Inclusion Body Encephalitis—Derived Fusion Protein Is Pathogenic after Infection via the Respiratory Route. J. Virol. 93:e01862-18.
43. Moss, W. J., and Griffin, D. E. (2012). Measles. Lancet 379, 153-164.
44. Mühlebach, M. D., Mateo, M., Sinn, P. L., Prüfer, S., Uhlig, K. M., Leonard, V. H. J., Navaratnarajah, C. K., Frenzke, M., Wong, X. X., Sawatsky, B., et al. (2011). Adherens junction protein nectin-4 is the epithelial receptor for measles virus. Nature 480, 530-533.
45. Noyce, R. S., Bondre, D. G., Ha, M. N., Lin, L.-T., Sisson, G., Tsao, M.-S., and Richardson, C. D. (2011). Tumor cell marker PVRL4 (nectin 4) is an epithelial cell receptor for measles virus. PLoS Pathog. 7, e1002240.
46. Perry, R. T., Murray, J. S., Gacic-Dobo, M., Dabbagh, A., Mulders, M. N., Strebel, P. M., Okwo-Bele, J.-M., Rota, P. A., and Goodson, J. L. (2015). Progress toward regional measles elimination—worldwide, 2000-2014. MMWR Morb. Mortal. Wkly. Rep. 64, 1246-1251.
47. Plemper, R. K., Doyle, J., Sun, A., Prussia, A., Cheng, L.-T., Rota, P. A., Liotta, D. C., Snyder, J. P., and Compans, R. W. (2005). Design of a small-molecule entry inhibitor with activity against primary measles virus strains. Antimicrob. Agents Chemother. 49, 3755-3761.
48. Prussia, A. J., Plemper, R. K., and Snyder, J. P. (2008). Measles virus entry inhibitors: a structural proposal for mechanism of action and the development of resistance. Biochemistry 47, 13573—13583.
49. Qian, X., Nguyen, H. N., Song, M. M., Hadiono, C., Ogden, S. C., Hammack, C., Yao, B., Hamersky, G. R., Jacob, F., Zhong, C., et al. (2016). Brain-Region-Specific Organoids Using Mini-bioreactors for Modeling ZIKV Exposure. Cell 165, 1238-1254.
50. Rima, B. K., and Duprex, W. P. (2005). Molecular mechanisms of measles virus persistence. Virus Research 111, 132-147.
51. Sato, Y., Watanabe, S., Fukuda, Y., Hashiguchi, T., Yanagi, Y., and Ohno, S. (2018). Cell-to-Cell Measles Virus Spread between Human Neurons Is Dependent on Hemagglutinin and Hyperfusogenic Fusion Protein. J. Virol. 92:e02166-17.
52. Schmid, A., Spielhofer, P., Cattaneo, R., Baczko, K., ter Meulen, V., and Billeter, M. A. (1992). Subacute sclerosing panencephalitis is typically characterized by alterations in the fusion protein cytoplasmic domain of the persisting measles virus. Virology 188, 910-915.
53. Simons, E., Ferrari, M., Fricks, J., Wannemuehler, K., Anand, A., Burton, A., and Strebel, P. (2012). Assessment of the 2010 global measles mortality reduction goal: results from a model of surveillance data. Lancet 379, 2173-2178.
54. Sun, A., Prussia, A., Zhan, W., Murray, E. E., Doyle, J., Cheng, L.-T., Yoon, J.-J., Radchenko, E. V., Palyulin, V. A., Compans, R. W., et al. (2006). Nonpeptide inhibitors of measles virus entry. J. Med. Chem. 49, 5080-5092.
55. Tatsuo, H., Ono, N., Tanaka, K., and Yanagi, Y. (2000). SLAM (CDw150) is a cellular receptor for measles virus. Nature 406, 893-897.
56. Watanabe, M., Hashimoto, K., Abe, Y., Kodama, E. N., Nabika, R., Oishi, S., Ohara, S., Sato, M., Kawasaki, Y., Fujii, N., et al. (2016). A Novel Peptide Derived from the Fusion Protein Heptad Repeat Inhibits Replication of Subacute Sclerosing Panencephalitis Virus In Vitro and In Vivo. PLoS ONE 11, e0162823.
57. Watanabe, S., Shirogane, Y., Suzuki, S. O., Ikegame, S., Koga, R., and Yanagi, Y. (2013). Mutant fusion proteins with enhanced fusion activity promote measles virus spread in human neuronal cells and brains of suckling hamsters. J. Virol. 87, 2648-2659.
58. Watanabe, S., Ohno, S., Shirogane, Y., Suzuki, S. O., Koga, R., and Yanagi, Y. (2015). Measles virus mutants possessing the fusion protein with enhanced fusion activity spread effectively in neuronal cells, but not in other cells, without causing strong cytopathology. J. Virol. 89, 2710-2717.
59. Welsch, J. C., Talekar, A., Mathieu, C., Pessi, A., Moscona, A., Horvat, B., and Porotto, M. (2013). Fatal measles virus infection prevented by brain-penetrant fusion inhibitors. J. Virol. 87, 13785-13794.
60. Welsch, J. C., Lionnet, C., Terzian, C., Horvat, B., Gerlier, D., and Mathieu, C. (2017). Organotypic Brain Cultures: A Framework for Studying CNS Infection by Neurotropic Viruses and Screening Antiviral Drugs. Bio-Protocol 7, e2605.
61. Welsch, J. C., Charvet, B., Dussurgey, S., Allatif, O., Aurine, N., Horvat, B., Gerlier, D., and Mathieu, C. (2019). Type I Interferon Receptor Signaling Drives Selective Permissiveness of Astrocytes and Microglia to Measles Virus during Brain Infection. J. Virol. 93:e00618-19.
62. Young, V. A., and Rall, G. F. (2009). Making it to the synapse: measles virus spread in and among neurons. Curr. Top. Microbiol. Immunol. 330, 3-30.

### Methods

Peptides and chemicals. MV F derived fusion inhibitory peptides were previously described (Mathieu et al., 2015). Briefly 36-aa peptides derived from the heptad repeat region at the C-terminus of the MV F protein were synthesized. Dimeric cholesterol conjugated (HRC4) forms of the peptides were used in this study. N-(3-cyanophenyl)-2-phenylacetamide (also known as 3G) was commercially acquired from ZereneX Molecular Limited (UK). The purity of 3G was tested by high-pressure liquid chromatography (HPLC) and shown to be >95% pure.

Plasmids and reagents. The genes of MeV IC323 H and F proteins were codon optimized, synthesized, and sub cloned into the mammalian expression vector pCAGGS. Plasmids encoding nectin 4, CD150, were commercially acquired.

Cells. HEK293T (human kidney epithelial), 293-3-46 (Radecke and Billeter, 1995; Radecke et al., 1995), Vero and Vero-SLAM/CD150 (African green monkey kidney) cells were grown in Dulbecco's modified Eagle's medium (DMEM; Life Technologies; Thermo Fisher Scientific) supplemented with 10% fetal bovine serum (FBS, Life Technologies; Thermo Fisher Scientific) and antibiotics at 37° C. in 5% CO2. The 293-3-46 and Vero-SLAM/CD150 culture media were supplemented with 1 mg/ml Geneticin (Thermo Fisher Scientific).

Recombinant virus production and analysis. MeV IC323-EGFP (Hashimoto et al., 2002) is a recombinant virus expressing the gene encoding EGFP. All variants with the mutations T461I, N462K, and L454W were generated in the MeV IC323-EGFP background (using the plasmid encoding MeV IC323-EGFP kindly provided by Yanagi, Kyushu University, Fukyoka, Japan) using reverse genetics. MeV IC323-Td Tomato was generating by replacing the EGFP expression cassette by the sequence coding for tdTomato red fluorescent protein. MeV IC323 recombinant viruses were rescued in 293-3-46 cells as previously described (Radecke et al., 1995). The viral production of the virus bearing the L454W was performed at either 37° C. or 32° C. All viruses were propagated and titrated in Vero-SLAM/CD150 cells.

Structural modeling. Twenty models were produced for the wild-type (wt) measles virus fusion glycoprotein (MeV F) using the protein homology server Phyre2 (Kelley et al., 2015). Bad local geometries of each model for pre-fusion and post-fusion states of MeV F were manually fixed by the program XtalView (McRee, 1999). The resulting models were subsequently refined by CNS-1.3 (Brunger et al., 1998) against the electron density of SEVM and 1ZTM for pre-fusion and post-fusion states, respectively. The same methodology was used for production of models for all MV-F mutant proteins. All structural FIGS. were produced using PyMol (www.pymol.org/).

Beta-Galactosidase (Beta-Gal) complementation-based fusion assay. The Beta-Gal complementation-based fusion assay was performed as previously described (Jurgens et al., 2015). Briefly, 293T cells transiently transfected with the constructs indicated above and the omega reporter subunit were incubated for the indicated period with cells coexpressing viral glycoproteins and the alpha reporter subunit in the presence or not of MV F HRC derived fusion inhibitory peptide (Mathieu et al., 2015).

Cell surface staining with F-conformation-specific mAbs. 293T cells transiently transfected with viral glycoprotein constructs were incubated overnight at 37° C. in complete medium (DMEM, 10% FBS). 20 h post-transfection, cells were transferred to 55° C. and times indicated in the figures. Thereafter, cells were incubated with mouse monoclonal antibodies (mAbs) that specifically detect MeV F in its pre-fusion conformation (1:1,000) for 1 h on ice. Cells were washed with PBS and then incubated for 1 h on ice with Alexa-488 anti-mouse secondary antibody (1:500; Life Technologies). Cells were washed with PBS and then fixed for 10 min on ice with 4% paraformaldehyde (PFA) with 1:1000 dilution of DAPI (4′,6-diamidino-2-phenylindole; Thermo Fisher) for 60 min. Plates were washed, 0.01% sodium azide was added, and plates were imaged via the use of an IN Cell Analyzer. Percentages of positively recognized cells were determined using Cell Profiler.

Organotypic cerebellar culture preparation and treatment post infection. Cerebellar slices were prepared from IFNAR1KO (and SLAM/CD150 tg×IFNAR1KO) or C57/BL6 mice and maintained in culture as detailed elsewhere (Welsch et al., 2017). Briefly, cerebella were isolated from the brains of 7-day-old mice and cut with a McIlwain tissue chopper (WPI-Europe) to obtain 350-μm-thick progressive slices. The brain slices were then dissociated in cold Hibernate®-A/5 g/L D-Glucose/1× Kynurenic acid buffer and laid out on Millipore cell culture insert membranes (Millicell cell culture insert, 30 mm, hydrophilic polytetrafluoroethylene, Millipore). Slices were subsequently cultured in GlutaMAX minimal essential medium supplemented with 25% horse serum, 5 g/L glucose, 1% HEPES (all Thermo Fisher Scientific), and 0.1 mg/L human recombinant insulin (R&D Systems) at 37° C. in 5% CO2 in a humidified atmosphere. The medium was changed every day after the slicing procedure. Slices from 5 mice were infected on the day of slicing with MeV IC323-EGFP-F L454W virus (5.103 pfu/slice from IFNAR1KO and 100 pfu/slice from SLAM/CD150tg×IFNAR1KO mice). Cultures were then daily treated from day 1 to day 4 either with serial dilutions of HRC4 fusion inhibitor in Neurobasal medium or with vehicle (untreated condition; “NT”). 2 μl of 10000 nM, 1000 nM or 100 nM of HRC4 were added on top of each of the 5 slices in each well. After several minutes, the drops containing the peptides were completely absorbed and reached the lower compartment of the system that contains the feeding medium. The final concentration in the medium of culture (1 ml) was 100 nM, 10 nM or 1 nM. At each time point, slices were collected, RNA extracted and RT-qPCR performed as previously described (Welsch et al., 2013)

Human brain organoid differentiation. Cerebral organoids were generated as previously described (Lancaster and Knoblich, 2014) from two iPSC lines (FA0000010 and FA0000011, for brevity called FA10 and FA11). Briefly, cells were dissociated into single cell suspension with Accutase (Stem Cell Technologies, cat #7920) and seeded at 4,500 cells/well in 96-well low attachment U bottom plates, using EB media (DMEM/F12 (Thermo Fisher Scientific, cat. #11330-032) supplemented with 20% Knockout Serum Replacement (Thermo Fisher Scientific, cat. #10828-028), 3% ES quality batch-tested fetal bovine serum (Thermo Fisher Scientific, cat #10439024), 1% Glutamax (Thermo Fisher Scientific, cat. #35050-038), lx Non-Essential Amino Acids, 0.1 mM 2-mercaptoethanol, 4 ng/ml bFGF (R&D Systems, cat. #233FB01M), and 50 uM Y-27632. Fresh medium was replaced every other day till day 6. On day 6, EB media was replaced with Neural Induction (NI) media (DMEM/F12, 1× N2 Supplement, 1× Glutamax, 1× NEAA and 1 μg/ml Heparin (Sigma-Aldrich, cat #H3149)) and the organoids were transferred to 60 mm or 100 mm low-attachment plates. The organoids were allowed to form neuroepithelium tissue till day 11-14, with media change every other day. Between day 11-24, the organoids were coated with Matrigel droplets and allowed to gel by keeping at 37° C. for 30 min. Matrigel-coated organoids were transferred to differentiation media (1:1 DMEM/F12:Neurobasal, 0.5% N2 Supplement, 2% B27 Supplement without Vitamin A (Life Technologies, cat #12587010), 0.25% Insulin solution (Life Technologies, cat #12585014), 50 uM 2-mercaptoethanol, 1% Glutamax, 0.5% NEAA, 1% Penicillin-Streptomycin), for 4 days. After 4 days, organoids were transferred to differentiation media containing B27 Supplement with Vitamin A (Thermo Fisher Scientific, cat #17504-044). Brain organoids were cultured for additional 60 days with media change every 7 days, and then used for further experiments at 90 days or 270 days.

Brain organoid RNA-Seq and analysis. RNA from uninfected and infected brain organoids was extracted using Direct-zoltm RNA MicroPrep (Zymo) and submitted to the JP Sulzberger Columbia Genome Center for library preparation and sequencing. Strand-specific RNA-Seq libraries were prepared using a poly-A enrichment and were sequenced on an Illumina NovaSeq with paired end 2×100 reads. After quality and adapter trimming, transcript abundance quantification was performed using Kallisto version 0.44.0 (Bray et al., 2016) with GRCh38 as the reference genome.

To understand the brain organoid developmental stage, we used the BrainSpan dataset (22031440) Since BrainSpan offers normalized RPKM expression values based on Gencode v10 annotations, we mapped R1 sequencing reads to GRCh37 annotation of the human genome using bowtie2 (22388286) and quantified gene-level RPKM levels using featureCounts (Liao et al., 2014). Genes were filtered based on an arbitrary gene-level RPKM sum of greater than 1000 across all 539 RNA-Seq experiments (524 BrainSpan, 15 MeV infection). All-by-all correlation matrices of log 2-transformed RPKM values with a pseudocount of 1 were generated in R v3.6.2. The top 100 BrainSpan samples with the highest correlation coefficients with uninfected FA10 brain organoid replicate 1 were pulled and heatmap of correlation coefficients was generated using pheatmap (https://github.com/raivokolde/pheatmap).

Differential gene expression analysis was performed using the Kallisto transcript abundances and the R Bioconductor package DESeq2 (Love et al., 2014). Code for analysis is available at http://www.github.com/greninger-lab/MeV-brain-organoids. Low count filtering was performed on genes with an average of less than one count per sample. Batch effects and biological differences between organoids were incorporated into the design formula as confounders and normalization and differential expression analysis performed using DESeq2 default parameters. Expression of the 50 genes with the lowest Benjamini-Hochberg adjusted p value between F454W and uninfected organoids were plotted on a heatmap generated using the R package pheatmap, with bar graphs generated in R package ggplot. Enrichment of differentially expressed genes (padj<0.0001, absolute value log 2 fold change>1) between L454W and uninfected in KEGG pathways was evaluated using the R package ReactomePA (Yu and He, 2016).

To calculate reads per million (RPM) values for MeV reads each sample was aligned against the NC_001498 MeV reference sequence using Bowtie2 with default parameters (22388286). MeV RPM values were calculated using the number of mapped reads in the resulting BAM file

RNAseq of mouse brain slices. RNA extracted from brain slices was prepared and sequenced as for organoids, above. Reads were pseudoaligned by Kallisto v0.44 (ref) to mouse reference transcriptome mmGRCm38. Differential expression analysis was performed in DESeq2, incorporating batch effects into the design formula. Expression heatmap was generated as for brain organoids, above. MeV RPM were calculated as above.

Brain organoids genome specific RT-qPCR. Specific reverse transcription targeting the MeV Genomic strand were performed with SuperScript™ III First-Strand Synthesis System (Thermofisher), according to manufacturer instructions, on 500 ng of total RNA using reverse primer for human GAPDH and FW 5′ tagged-MeV primer 5′-gcagggcaatctcacaatcaggAAAACTGGTGTTCTACAACAA-3′ (SEQ ID NO. 54) containing MeV Sequence of the antigenomic strand with a TAG sequence from Nipah virus. Obtained cDNAs were then diluted 1:10. QPCR were then performed as formerly described (Iketani et al., 2018) using MeV Rev 5′-TGAAGGCCACTGCATT-3′ (SEQ ID NO. 55) and Tag FW 5′-gcagggcaatctcacaatcagg-3′ (SEQ ID NO. 56) primers. All results were normalized to human GAPDH deviation.

mNGS and variant calling. mNGS was performed as previously described (Iketani et al., 2018). Briefly, RNA was extracted from 50 μL of viral culture using the Quick-RNA Viral Kit (Zymo) and treated with TURBO DNase I (ThermoFisher). cDNA was generated from the DNase-treated RNA using Superscript IV Reverse Transcriptase (Thermo Fisher) and random hexamers (IDT), followed by second-strand synthesis via Sequenase Version 2.0 DNA Polymerase. The resulting double-stranded cDNA was then purified with the DNA Clean & Concentrator Kit (Zymo). Libraries were constructed from 2 μL of cDNA using the Nextera XT kit (Illumina) and sequenced on 1×192 bp Illumina MiSeq runs.

Sequencing reads were adapter and quality trimmed using Trimmomatic v0.38 (Bolger et al., 2014). Variants present at a frequency greater than 10% and coverage greater than 10× were identified with LAVA (github.com/greninger-lab/lava) using the MeV reference genome (NC_001498). All variants were manually confirmed by mapping sequencing reads to the same MeV reference strain in Geneious v11.1.4 (Kearse et al., 2012). Those variants present in intergenic region between the matrix and fusion proteins as well as in homopolymeric tracts were excluded from the analysis. Sequencing reads are available under NCBI BioProject number PRJNA594952.

Statistical analysis: The Mantel-Cox test was used for the survival comparison analysis. All other statistical comparisons were performed using the Mann-Whitney U-Test. All analyses were performed in GraphPad Prism5 software. Statistical analysis of RNAseq data was performed in R.

### REFERENCES FOR METHODS SECTION

1. Bolger, A. M., Lohse, M., and Usadel, B. (2014). Trimmomatic: a flexible trimmer for Illumina sequence data. Bioinforma. Oxf. Engl. 30, 2114-2120.
2. Bray, N. L., Pimentel, H., Melsted, P., and Pachter, L. (2016). Near-optimal probabilistic RNA-seq quantification. Nat. Biotechnol. 34, 525-527.
3. Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., et al. (1998). Crystallography & NMR system: A new software suite for macromolecular structure determination. Acta Crystallogr. D Biol. Crystallogr. 54, 905-921.
4. Hashimoto, K., Ono, N., Tatsuo, H., Minagawa, H., Takeda, M., Takeuchi, K., and Yanagi, Y. (2002). SLAM (CD150)-independent measles virus entry as revealed by recombinant virus expressing green fluorescent protein. J. Virol. 76, 6743-6749.
5. Iketani, S., Shean, R. C., Ferren, M., Makhsous, N., Aquino, D. B., des Georges, A., Rima, B., Mathieu, C., Porotto, M., Moscona, A., et al. (2018). Viral Entry Properties Required for Fitness in Humans Are Lost through Rapid Genomic Change during Viral Isolation. MBio 9.
6. Jurgens, E. M., Mathieu, C., Palermo, L. M., Hardie, D., Horvat, B., Moscona, A., and Porotto, M. (2015). Measles fusion machinery is dysregulated in neuropathogenic variants. MBio 6.
7. Kearse, M., Moir, R., Wilson, A., Stones-Havas, S., Cheung, M., Sturrock, S., Buxton, S., Cooper, A., Markowitz, S., Duran, C., et al. (2012). Geneious Basic: an integrated and extendable desktop software platform for the organization and analysis of sequence data. Bioinforma. Oxf. Engl. 28, 1647-1649.
8. Kelley, L. A., Mezulis, S., Yates, C. M., Wass, M. N., and Sternberg, M. J. E. (2015). The Phyre2 web portal for protein modeling, prediction and analysis. Nat. Protoc. 10, 845-858.
9. Lancaster, M. A., and Knoblich, J. A. (2014). Generation of cerebral organoids from human pluripotent stem cells. Nat. Protoc. 9, 2329-2340.
10. Langmead, B., and Salzberg, S. L. (2012). Fast gapped-read alignment with Bowtie 2. Nat. Methods 9, 357-359.
11. Liao, Y., Smyth, G. K., and Shi, W. (2014). featureCounts: an efficient general purpose program for assigning sequence reads to genomic features. Bioinforma. Oxf. Engl. 30, 923-930.
12. Love, M. I., Huber, W., and Anders, S. (2014). Moderated estimation of fold change and dispersion for RNA-seq data with DESeq2. Genome Biol. 15, 550.
13. Mathieu, C., Huey, D., Jurgens, E., Welsch, J. C., DeVito, I., Talekar, A., Horvat, B., Niewiesk, S., Moscona, A., and Porotto, M. (2015). Prevention of measles virus infection by intranasal delivery of fusion inhibitor peptides. J. Virol. 89, 1143-1155.
14. McRee, D. E. (1999). XtalView/Xfit—A versatile program for manipulating atomic coordinates and electron density. J. Struct. Biol. 125, 156-165.
15. Radecke, F., and Billeter, M. A. (1995). Appendix: measles virus antigenome and protein consensus sequences. Curr. Top. Microbiol. Immunol. 191, 181-192.
16. Radecke, F., Spielhofer, P., Schneider, H., Kaelin, K., Huber, M., Dotsch, C., Christiansen, G., and Billeter, M. A. (1995). Rescue of measles viruses from cloned DNA. EMBO J. 14, 5773-5784.
17. Welsch, J. C., Talekar, A., Mathieu, C., Pessi, A., Moscona, A., Horvat, B., and Porotto, M. (2013). Fatal measles virus infection prevented by brain-penetrant fusion inhibitors. J. Virol. 87, 13785-13794.
18. Welsch, J. C., Lionnet, C., Terzian, C., Horvat, B., Gerlier, D., and Mathieu, C. (2017). Organotypic Brain Cultures: A Framework for Studying CNS Infection by Neurotropic Viruses and Screening Antiviral Drugs. Bio-Protoc. 7, e2605.

### Example 6—Soluble F Protein Stabilized in Pre-Fusion State

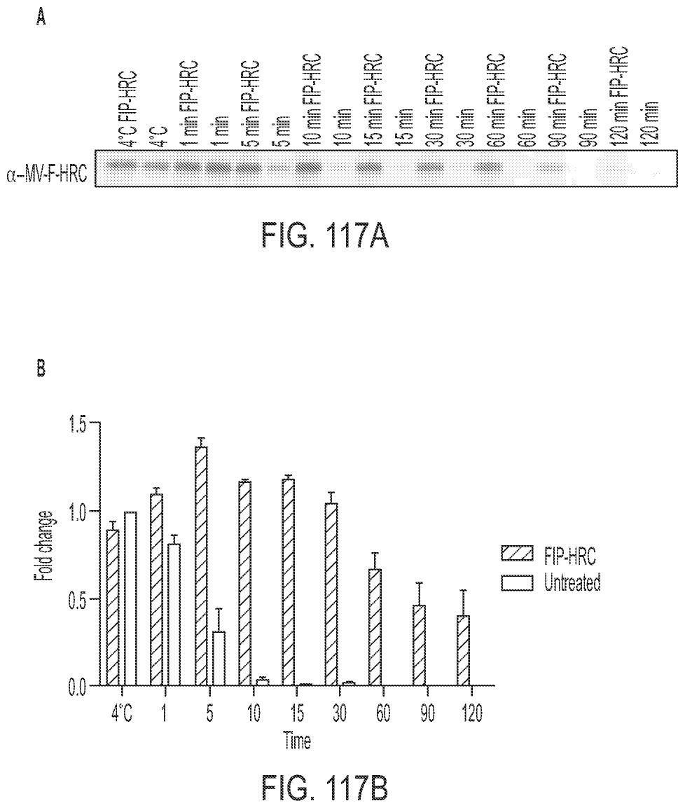

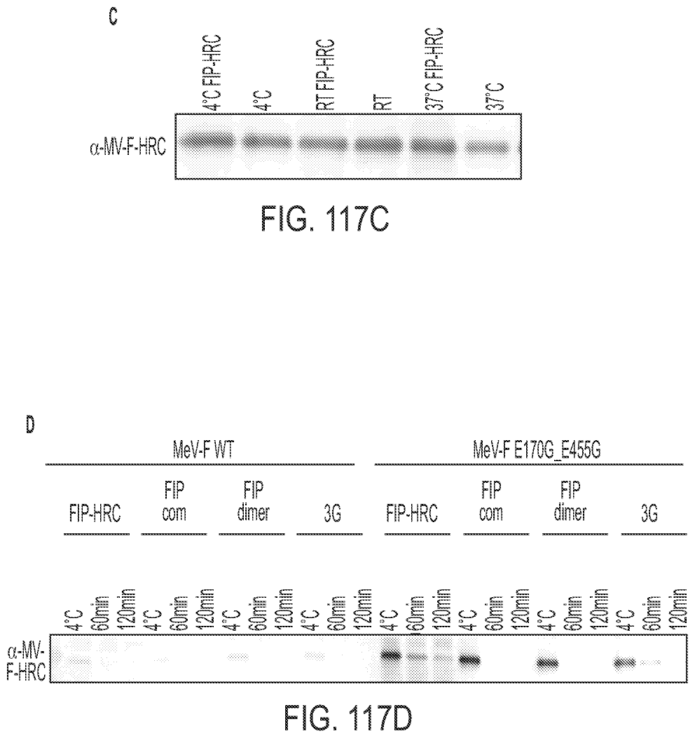

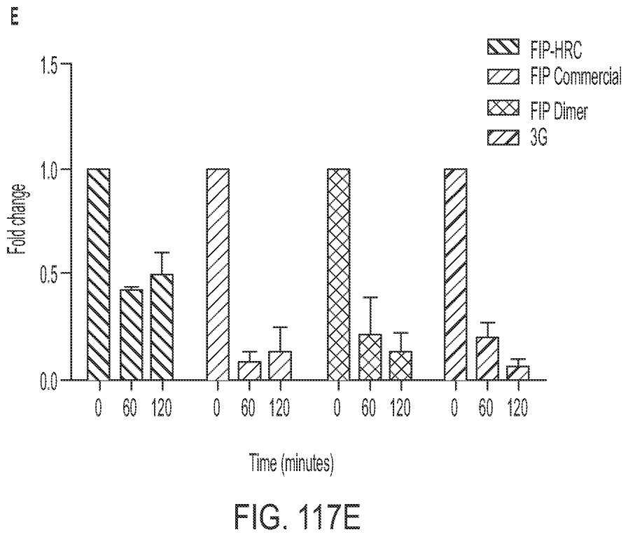

FIGS. 117A-E show data on the measles fusion protein stabilized by specific mutations E107G and E455G, and by the FIP-HRC (dimer without the cholesterol). A soluble F in its pre-fusion state can be used as a vaccine. FIGS. 117A-E show 1) that the F stabilized in the presence of the FIP-HRC remains in its pre-fusion state incubation at 55° C. for up to two hours; 2) that the F stabilized remains in its pre-fusion state for up to a week at 37° C.; 3) that wt F is partially stabilized by FIP-HRC peptides; and 4) that the F stabilization of the FIP-HRC is superior to commercially available stabilizer like the commercial FIP and 3 g.

1. An antiviral peptide conjugate comprising a fusion inhibitory peptide (FIP) and a C-terminal heptad repeat (HRC) peptide (FIP-HRC).

2. The antiviral peptide conjugate of claim 1, further comprising a membrane localizing moiety region.

3. The antiviral peptide conjugate of claim 2, wherein the membrane localizing moiety region comprises a membrane localizing moiety selected from the group consisting of cholesterol, tocopherol, and palmityl.

4. The antiviral peptide conjugate of claim 2, wherein the membrane localizing moiety region is conjugated to a C-terminus of the HRC peptide.

5. The antiviral peptide conjugate of claim 2, further comprising a linker region.

6. The antiviral peptide conjugate of claim 5, wherein the linker region comprises polyethylene glycol (PEG).

7. (canceled)

8. (canceled)

9. (canceled)

10. (canceled)

11. The antiviral peptide conjugate of claim 5, wherein the linker region is conjugated to the C-terminus of the HRC peptide and the membrane localizing moiety region is conjugated to the linker region.

12. The antiviral peptide conjugate of claim 11, wherein the linker region comprises polyethylene glycol (PEG).

13. (canceled)

14. (canceled)

15. The antiviral peptide conjugate according to claim 5, wherein the antiviral peptide comprises a dimer of the FIP region and the HRC peptide region.

16. The antiviral peptide conjugate according to claim 15, wherein the antiviral peptide comprises a first FIP-HRC peptide conjugated to the linker region and a second FIP-HRC peptide conjugated to the linker region.

17. The antiviral peptide conjugate according to claim 1, wherein the antiviral peptide further comprises a serine-glycine linker.

18. (canceled)

19. The antiviral peptide conjugate of claim 17, wherein the serine-glycine linker is located at the C-terminus of the HRC peptide.

20. (canceled)

21. (canceled)

22. (canceled)

23. (canceled)

24. The antiviral peptide conjugate according to claim 1, wherein the FIP peptide comprises the amino acid sequence FFG.

25. (canceled)

26. The antiviral peptide conjugate according to claim 1, wherein the HRC peptide conjugate comprises the amino acid sequence PPISLERLDVGTN or FFGPPISLERLDVGTN.

27. (canceled)

28. (canceled)

29. A nanoparticle comprising the antiviral peptide conjugate according to claim 1.

30. (canceled)

31. (canceled)

32. (canceled)

33. (canceled)

34. A method of treating measles, or post-infection measles prophylaxis, or treating HIV, or post-infection prophylaxis, comprising administering to a subject in need thereof an antiviral peptide conjugate of claim 1 comprising a fusion inhibitory peptide (FIP) and a C-terminal heptad (HRC) peptide (FIP-HRC).

35. (canceled)

36. (canceled)

37. (canceled)

38. (canceled)

39. (canceled)

40. (canceled)

41. (canceled)

42. (canceled)

43. A recombinant protein comprising a soluble stabilized measles F protein comprising an E445G mutation or comprising a E170G and a E455G double mutation.

44. (canceled)

45. (canceled)

46. (canceled)

47. (canceled)

48. (canceled)

49. A method of inducing an immune response to a measles virus by administering to a subject an immunogenic composition comprising the recombinant protein of claim 43.

50. (canceled)

51. (canceled)

52. (canceled)

53. (canceled)

54. (canceled)

55. (canceled)

56. (canceled)

57. (canceled)

58. (canceled)

59. The method of claim 34, wherein the HRC peptide is derived from HIV-gp41 (C34).

60. The method of claim 34, wherein the HRC peptide is derived from Measles virus.


SEQUENCE LISTING (58 sequences)

SEQ ID NO: 1 - protein, 494 aa, Measles morbillivirus
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTS

SEQ ID NO: 2 - protein, 546 aa, Artificial Sequence
  Description of Artificial Sequence Synthetic polypeptide
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSGRENLYFQGGGGGSGYIPEAPRDQAYVRKDGEWVLLSTFLGGTEGRHHHHHH

SEQ ID NO: 3 - protein, 494 aa, Artificial Sequence
  Description of Artificial Sequence Synthetic polypeptide
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLGRLDVGTNLGNAIAKLEDAKELLESSDQILRSMKGLSSTS

SEQ ID NO: 4 - protein, 546 aa, Artificial Sequence
  Description of Artificial Sequence Synthetic polypeptide
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLGRLDVGTNLGNAIAKLEDAKELLESSDQILRSMKGLSSTSGRENLYFQGGGGGSGYIPEAPRDQAYVRKDGEWVLLSTFLGGTEGRHHHHHH

SEQ ID NO: 5 - protein, 494 aa, Artificial Sequence
  Description of Artificial Sequence Synthetic polypeptide
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQGMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLGRLDVGTNLGNAIAKLEDAKELLESSDQILRSMKGLSSTS

SEQ ID NO: 6 - protein, 546 aa, Artificial Sequence
  Description of Artificial Sequence Synthetic polypeptide
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQGMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLGRLDVGTNLGNAIAKLEDAKELLESSDQILRSMKGLSSTSGRENLYFQGGGGGSGYIPEAPRDQAYVRKDGEWVLLSTFLGGTEGRHHHHHH

SEQ ID NO: 7 - protein, 5 aa, Artificial Sequence
  Description of Artificial Sequence Synthetic peptide
GSGSG

SEQ ID NO: 8 - protein, 36 aa, Measles morbillivirus
PPISLERLDVGTNLGNAIAKLEDAKELLESSDQILR

SEQ ID NO: 9 - protein, 13 aa, Measles morbillivirus
PPISLERLDVGTN

SEQ ID NO: 10 - protein, 39 aa, Artificial Sequence
  Description of Artificial Sequence Synthetic polypeptide
FFGPPISLERLDVGTNLGNAIAKLEDAKELLESSDQILR

SEQ ID NO: 11 - protein, 16 aa, Artificial Sequence
  Description of Artificial Sequence Synthetic peptide
FFGPPISLERLDVGTN

SEQ ID NO: 12 - protein, 34 aa, Human immunodeficiency virus
WMEWDREINNYTSLIHSLIEESQNQQEKNEQELL

SEQ ID NO: 13 - protein, 8 aa, Artificial Sequence
  Description of Artificial Sequence Synthetic peptide
FFGGSGSG

SEQ ID NO: 14 - protein, 8 aa, Artificial Sequence
  Description of Artificial Sequence Synthetic peptide
  MOD_RES (1)..(1): D-amino acid
FFGGSGSG

SEQ ID NO: 15 - protein, 5 aa, Artificial Sequence
  Description of Artificial Sequence Synthetic peptide
  MISC_FEATURE (1)..(5): One or more residues may be D-amino acids
GSGSG

SEQ ID NO: 16 - protein, 41 aa, Artificial Sequence
  Description of Artificial Sequence Synthetic polypeptide
PPISLERLDVGTNLGNAIAKLEDAKELLESSDQILRGSGSG

SEQ ID NO: 17 - protein, 18 aa, Artificial Sequence
  Description of Artificial Sequence Synthetic peptide
PPISLERLDVGTNGSGSG

SEQ ID NO: 18 - protein, 39 aa, Artificial Sequence
  Description of Artificial Sequence Synthetic polypeptide
WMEWDREINNYTSLIHSLIEESQNQQEKNEQELLGSGSG

SEQ ID NO: 19 - protein, 44 aa, Artificial Sequence
  Description of Artificial Sequence Synthetic polypeptide
FFGGSGSGPPISLERLDVGTNLGNAIAKLEDAKELLESSDQILR

SEQ ID NO: 20 - protein, 44 aa, Artificial Sequence
  Description of Artificial Sequence Synthetic polypeptide
FFGPPISLERLDVGTNLGNAIAKLEDAKELLESSDQILRGSGSG

SEQ ID NO: 21 - protein, 49 aa, Artificial Sequence
  Description of Artificial Sequence Synthetic polypeptide
FFGGSGSGPPISLERLDVGTNLGNAIAKLEDAKELLESSDQILRGSGSG

SEQ ID NO: 22 - protein, 43 aa, Artificial Sequence
  Description of Artificial Sequence Synthetic polypeptide
FFGGSGSGPPISLERLDVGTNLGNAIAKLEDAKELLESSDQIL

SEQ ID NO: 23 - protein, 40 aa, Artificial Sequence
  Description of Artificial Sequence Synthetic polypeptide
FFGPPISLERLDVGTNLGNAIAKLEDAKELLESSDQILRC

SEQ ID NO: 24 - protein, 44 aa, Artificial Sequence
  Description of Artificial Sequence Synthetic polypeptide
FFGGSGSGPPISLERLDVGTNLGNAIAKLEDAKELLESSDQILC

SEQ ID NO: 25 - protein, 45 aa, Artificial Sequence
  Description of Artificial Sequence Synthetic polypeptide
FFGPPISLERLDVGTNLGNAIAKLEDAKELLESSDQILRGSGSGC

SEQ ID NO: 26 - protein, 50 aa, Artificial Sequence
  Description of Artificial Sequence Synthetic polypeptide
FFGGSGSGPPISLERLDVGTNLGNAIAKLEDAKELLESSDQILRGSGSGC

SEQ ID NO: 27 - protein, 15 aa, Artificial Sequence
  Description of Artificial Sequence Synthetic peptide
FFGPPISLERDVGTN

SEQ ID NO: 28 - protein, 21 aa, Artificial Sequence
  Description of Artificial Sequence Synthetic peptide
FFGGSGSGPPISLERLDVGTN

SEQ ID NO: 29 - protein, 21 aa, Artificial Sequence
  Description of Artificial Sequence Synthetic peptide
FFGPPISLERLDVGTNGSGSG

SEQ ID NO: 30 - protein, 26 aa, Artificial Sequence
  Description of Artificial Sequence Synthetic peptide
FFGGSGSGPPISLERLDVGTNGSGSG

SEQ ID NO: 31 - protein, 49 aa, Artificial Sequence
  Description of Artificial Sequence Synthetic polypeptide
  MOD_RES (1)..(1): D-amino acid
FFGGSGSGVALDPIDISVLNKIKSDLEESKEWIRRSNKILDSIGSGSGC

SEQ ID NO: 32 - protein, 41 aa, Artificial Sequence
  Description of Artificial Sequence Synthetic polypeptide
VALDPIDISVLNKIKSDLEESKEWIRRSNKILDSIGSGSGC

SEQ ID NO: 33 - protein, 17 aa, Artificial Sequence
  Description of Artificial Sequence Synthetic peptide
FFGPPISLERLDVGTNC

SEQ ID NO: 34 - protein, 22 aa, Artificial Sequence
  Description of Artificial Sequence Synthetic peptide
FFGGSGSGPPISLERLDVGTNC

SEQ ID NO: 35 - protein, 22 aa, Artificial Sequence
  Description of Artificial Sequence Synthetic peptide
FFGPPISLERLDVGTNGSGSGC

SEQ ID NO: 36 - protein, 27 aa, Artificial Sequence
  Description of Artificial Sequence Synthetic peptide
FFGGSGSGPPISLERLDVGTNGSGSGC

SEQ ID NO: 37 - protein, 37 aa, Artificial Sequence
  Description of Artificial Sequence Synthetic polypeptide
FFGWMEWDREINNYTSLIHSLIEESQNQQEKNEQELL

SEQ ID NO: 38 - protein, 42 aa, Artificial Sequence
  Description of Artificial Sequence Synthetic polypeptide
FFGGSGSGWMEWDREINNYTSLIHSLIEESQNQQEKNEQELL

SEQ ID NO: 39 - protein, 42 aa, Artificial Sequence
  Description of Artificial Sequence Synthetic polypeptide
FFGWMEWDREINNYTSLIHSLIEESQNQQEKNEQELLGSGSG

SEQ ID NO: 40 - protein, 47 aa, Artificial Sequence
  Description of Artificial Sequence Synthetic polypeptide
FFGGSGSGWMEWDREINNYTSLIHSLIEESQNQQEKNEQELLGSGSG

SEQ ID NO: 41 - protein, 38 aa, Artificial Sequence
  Description of Artificial Sequence Synthetic polypeptide
FFGWMEWDREINNYTSLIHSLIEESQNQQEKNEQELLC

SEQ ID NO: 42 - protein, 43 aa, Artificial Sequence
  Description of Artificial Sequence Synthetic polypeptide
FFGGSGSGWMEWDREINNYTSLIHSLIEESQNQQEKNEQELLC

SEQ ID NO: 43 - protein, 43 aa, Artificial Sequence
  Description of Artificial Sequence Synthetic polypeptide
FFGWMEWDREINNYTSLIHSLIEESQNQQEKNEQELLGSGSGC

SEQ ID NO: 44 - protein, 48 aa, Artificial Sequence
  Description of Artificial Sequence Synthetic polypeptide
FFGGSGSGWMEWDREINNYTSLIHSLIEESQNQQEKNEQELLGSGSGC

SEQ ID NO: 45 - protein, 20 aa, Artificial Sequence
  Description of Artificial Sequence Synthetic peptide
  MISC_FEATURE (1)..(20): This sequence may encompass 1-10 “Gly Ser” repeating units
GSGSGSGSGSGSGSGSGSGS

SEQ ID NO: 46 - protein, 21 aa, Artificial Sequence
  Description of Artificial Sequence Synthetic peptide
  MISC_FEATURE (2)..(21): This region may encompass 1-10 “Ser Gly” repeating units
GSGSGSGSGSGSGSGSGSGSG

SEQ ID NO: 47 - protein, 21 aa, Artificial Sequence
  Description of Artificial Sequence Synthetic peptide
  MISC_FEATURE (2)..(21): This region may encompass 1-10 “Gly Ser” repeating units
SGSGSGSGSGSGSGSGSGSGS

SEQ ID NO: 48 - protein, 24 aa, Measles morbillivirus
MGLKVNVSAIFMAVLLTLQTPTGQ

SEQ ID NO: 49 - protein, 42 aa, Artificial Sequence
  Description of Artificial Sequence Synthetic polypeptide
PPISLERLDVGTNLGNAIAKLEDAKELLESSDQILRGSGSGC

SEQ ID NO: 50 - protein, 50 aa, Artificial Sequence; identical to SEQ ID NO: 26
  Description of Artificial Sequence Synthetic polypeptide
  MOD_RES (1)..(1): D-amino acid

SEQ ID NO: 51 - protein, 45 aa, Artificial Sequence
  Description of Artificial Sequence Synthetic polypeptide
  MOD_RES (1)..(1): D-amino acid
FFGGSGSGPPISLERLDVGTNLGNAIAKLEDAKELLESSDQILRC

SEQ ID NO: 52 - protein, 9 aa, Artificial Sequence
  Description of Artificial Sequence Synthetic peptide
  MOD_RES (1)..(1): D-amino acid
FFGGSGSGC

SEQ ID NO: 53 - protein, 4 aa, Artificial Sequence
  Description of Artificial Sequence Synthetic peptide
  MOD_RES (1)..(1): D-amino acid
FFGC

SEQ ID NO: 54 - DNA, 43 bp, Artificial Sequence
  Description of Artificial Sequence Synthetic primer
gcagggcaatctcacaatcaggaaaactggtgttctacaacaa

SEQ ID NO: 55 - DNA, 16 bp, Artificial Sequence
  Description of Artificial Sequence Synthetic primer
tgaaggccactgcatt

SEQ ID NO: 56 - DNA, 22 bp, Artificial Sequence
  Description of Artificial Sequence Synthetic primer
gcagggcaatctcacaatcagg

SEQ ID NO: 57 - protein, 6 aa, Artificial Sequence
  Description of Artificial Sequence Synthetic 6xHis tag
HHHHHH

SEQ ID NO: 58 - protein, 21 aa, Artificial Sequence
  Description of Artificial Sequence Synthetic peptide
  MOD_RES (1)..(1): D-amino acid
FFGGSGSGPPISLERDVGTNC